(19) United States
(12) Patent Application Publication
(10) Pub. No.: US 2026/0240927 A1
(43) Pub. Date: Aug. 20, 2026

(54) HEAT-TREATED EXTENDED-RELEASE FORMULATIONS

(71) Applicant: VitaKey Inc., Washington, DC (US)

(72) Inventors: Aaron C. Anselmo, Chapel Hill, NC (US); Anant Shankar Balijepalli, Durham, NC (US); Andrew D. Christenson, Raleigh, NC (US); Joshua K. DeGennaro, Cary, NC (US); Andrea Stamp, Brookline, MA (US); Ana Jaklenec, Lexington, MA (US); Catherine B. Reynolds, Mountain Brook, AL (US); Robert S. Langer, Newton, MA (US); Christina Lynn Makris, Durham, NC (US); Sarah Grace Adzema, Durham, NC (US); Thomas Pho, Durham, NC (US); Megan Elizabeth Reynolds, San Francisco, CA (US)

(21) Appl. No.: 19/187,926

(22) Filed: Apr. 23, 2025

Related U.S. Application Data
(63) Continuation of application No. PCT/US25/16653, filed on Feb. 20, 2025.

(51) Int. Cl. A61K 35/747; A23J 3/08; A61K 35/00; A61K 47/36; A61K 47/42

(52) U.S. Cl. CPC A61K 35/747; A23J 3/08; A61K 47/36; A61K 47/42; A61K 2035/11

(57) ABSTRACT

The present disclosure provides heat-treated extended-release formulations comprising one or more food components and one or more release modulators. The present example also provides methods of manufacturing heat-treated extended-release formulations.

### CROSS-REFERENCE TO RELATED APPLICATIONS

This application is a continuation of PCT International Application No. PCT/US25/16653, filed Feb. 20, 2025, which claims priority to U.S. Provisional Patent Application No. 63/572,899, filed Apr. 1, 2024, the content of each of which is incorporated herein by reference in its entirety.

### GOVERNMENT RIGHTS

This invention was made with government support under contract number HT942523C0037 awarded by the Department of Defense (DOD). The government therefore has certain rights in this invention.

### BACKGROUND

The health benefits of formulated foods, formulated beverages, formulated supplements, and their nutritional constituents to mammals such as humans have been studied for several decades. However, in many cases, the challenges associated with controlled delivery, absorption, nutrient absorption, and energy acquisition from formulated meals, foods, beverages, supplements, and/or their food components (e.g., nutritional constituents) remain unsolved. As such, formulated meals, foods, beverages, supplements, and/or their food components (e.g., nutritional constituents) have yet to realize their full potential.

### SUMMARY

The present disclosure relates to novel compositions, preparations, and methods for providing a controlled, extended, and/or delayed release of one or more payloads (e.g., one or more food components) to a subject (e.g., a human or animal) ingesting said compositions or preparations.

In some embodiments, the present disclosure provides a preparation comprising an extended-release composition comprising: a protein payload; and a release modulator. In some embodiments, a release modulator interacts with a protein payload to prevent access of water molecules and/or one or more enzymes to the protein payload in the preparation. In some embodiments, a protein payload is released in a subject over an extended period following ingestion of the preparation by a subject as compared to ingestion of the protein payload alone.

In some embodiments, a protein payload is heat-treated. In some embodiments, a protein payload is heat-treated at about 72° C. to about 112° C. for about 60 minutes to about 120 minutes; at about 77° C. to about 107° C. for about 75 minutes to about 105 minutes; or about 92° C. for about 90 minutes. In some embodiments, a protein payload is heat-treated at a pH of about 4 to about 8; about 4.5 to about 7.5; or about 5 to about 7.

In some embodiments, an extended-release composition of the present disclosure comprises 60% (w/w) to 85% (w/w) of a protein payload. In some embodiments, a protein payload comprises whey protein isolate, whey protein concentrate, pea protein isolate, pea protein concentrate, oat protein isolate, oat protein concentrate, soy protein isolate, soy protein concentrate, wheat protein isolate, wheat protein concentrate, egg protein isolate, egg protein concentrate, dairy protein, plant-based protein, casein, bovine serum albumin, ovalbumin, α-lactalbumin, β-lactoglobulin, collagen, glutanin, gliadin, kefirin, avenin, zein, silk, gelatin, hordein, sodium carboxymethylcellulose, or a combination thereof. In some embodiments, a protein payload comprises whey protein isolate, whey protein concentrate, casein, lactalbumin, β-lactoglobulin, or a combination thereof. In some embodiments, a protein payload comprises whey protein isolate, whey protein concentrate, pea protein isolate, oat protein isolate, soy protein isolate, wheat protein isolate, plant-based protein, or a combination thereof.

In some embodiments, an extended-release composition of the present disclosure comprises 15% (w/w) to 40% (w/w) of the release modulator. In some embodiments, a release modulator comprises a hydrocolloid. In some embodiments, a hydrocolloid comprises pectin, alginate, carrageenan, or a combination thereof. In some embodiments, an extended-release composition comprises 10% (w/w) to 40% (w/w) of pectin. In some embodiments, an extended-release composition comprises 0.75% (w/w) to 1.25% (w/w) alginate.

In some embodiments, a release modulator comprises a gelator. In some embodiments, a gelator comprises dicalcium phosphate, gluconolactone, calcium carbonate, or a combination thereof. In some embodiments, a release modulator comprises 7.5 parts or less of hydrocolloid by dry weight for every 1 part of gelator by dry weight.

In some embodiments, a release modulator comprises a carbohydrate. In some embodiments, a carbohydrate comprises hypromellose acetate succinate, rice starch, corn starch, inulin, oat fiber, glucose, tapioca polydextrose, maltodextrin, chitosan, sucrose palmitate, or a combination thereof.

In some embodiments, a release modulator comprises a surfactant. In some embodiments, a surfactant comprises Pluronic F68, Tween 85, triacetin, or Span 60.

In some embodiments, a release modulator comprises shellac. In some embodiments, a release modulator comprises gum arabic.

In some embodiments, an extended-release composition of the present disclosure comprises: 75% (w/w) to 85% (w/w) protein payload; 10% (w/w) to 20% (w/w) hydrocolloid; and 2% (w/w) to 10% (w/w) gelator.

In some embodiments, an extended-release composition of the present disclosure comprises: 75% (w/w) to 85% (w/w) heat-treated whey protein; 10% (w/w) to 20% (w/w) alginate; 0.5% (w/w) to 2% (w/w) gluconolactone; and 0.5% (w/w) to 2% (w/w) dicalcium phosphate.

In some embodiments, an extended-release composition of the present disclosure comprises: 75% (w/w) to 85% (w/w) heat-treated whey protein; 10% (w/w) to 20% (w/w) pectin or carrageenan; 0.5% (w/w) to 1.5% (w/w) alginate; 0.5% (w/w) to 2% (w/w) gluconolactone; 0.5% (w/w) to 2% (w/w) dicalcium phosphate; and 1.5% (w/w) to 6% (w/w) chitosan.

In some embodiments, a preparation of the present disclosure comprises one or more excipients. In some embodiments, a preparation comprises 0.01% (w/w) to 10% (w/w) of one or more excipients. In some embodiments, one or more excipients comprises an anti-caking aid. In some embodiments, an anti-caking aid comprises silica. In some embodiments, a preparation comprises 0.5% (w/w) to 5% (w/w) silica.

In some embodiments, one or more excipients comprises a sequestrant. In some embodiments, a sequestrant comprises sodium hexametaphosphate. In some embodiments, a preparation comprises 1% (w/w) to 4% (w/w) of sodium hexametaphosphate. In some embodiments, one or more excipients comprises chitosan. In some embodiments, a preparation comprises 2.5% (w/w) to 7.5% (w/w) of chitosan.

In some embodiments, one or more excipients comprises a stabilizer. In some embodiments, a stabilizer comprises xanthan gum. In some embodiments, a preparation comprises 0.01% (w/w) to 0.1% (w/w) of chitosan.

In some embodiments, a preparation of the present disclosure further comprises one or more coatings. In some embodiments, one or more coatings encapsulate the extended-release composition. In some embodiments, one or more coatings comprise a hydrophobic coat. In some embodiments, a hydrophobic coat comprises ethyl cellulose, a basic methacrylic copolymer, aqueous latex, or methylcellulose acetate succinate. In some embodiments, a hydrophilic coat encapsulates a hydrophobic coat. In some embodiments, a hydrophilic coat comprises hydroxypropyl methylcellulose or sodium alginate.

In some embodiments, a preparation of the present disclosure comprises one or more probiotics. In some embodiments, a preparation of the present disclosure comprises 1×10⁷ to 1×10¹⁰ CFUs of the one or more probiotics per gram of the preparation.

In some embodiments, a preparation comprises one or more additional components including a protein, a polypeptide, a peptide, an amino acid, a carbohydrate, a sugar, a monosaccharide, a polysaccharide, a dietary fiber, a fat, a fatty acid, a lipid, a nutrient, a nutraceutical, a macronutrient, a micronutrient, a vitamin, a mineral, a polypeptide, a carotenoid, an element, a ketone body, a prebiotic (e.g., a prebiotic fiber), a flavonoid, an antioxidant, an electrolyte, a salt, a circadian rhythm modulator, a supplement, a nootropic, an energy source, or a combination thereof. In some embodiments, a preparation comprises 0.01% (w/w) to 20% (w/w) of the one or more additional components. In some embodiments, one or more additional components comprise inulin, arabinoxylans, beta glucans, fructooligosaccharides, galactooligosaccharides, xyloglucans, psyllium, cellulose, guar gum, pectin, locust bean gum, hydroxypropylmethyl cellulose, alginate, resistant starch, polydextrose, resistant maltodextrin, mixed plant cell wall fibers, or a combination thereof.

In some embodiments, a preparation is characterized as having a Dv₅₀ less than 100 μm. In some embodiments, a preparation is characterized as having a water activity of less than 0.2. In some embodiments, a 100 mg/mL solution of a preparation in water at 20° C. is characterized as having a viscosity of 1-100 cP as measured using a 20 mm rotating disk. In some embodiments, a preparation is characterized as having a Hausner ratio of less than 1.5.

In some embodiments, an extended-release composition is characterized as having an extended duration of release of at least 2 hours, at least 4 hours, at least 6 hours, at least 12 hours, or at least 24 hours. In some embodiments, an extended-release composition does not release a maximum amount of releasable protein payload into a solution of simulated gastric fluid containing 60 units/ml of pepsin at 37° C. until after 2 hours of incubation in the solution, after 4 hours of incubation in the solution, or after 6 hours of incubation in the solution.

In some embodiments, the present disclosure additionally provides a food or beverage product comprising a preparation comprising an extended-release composition comprising: a protein payload; and a release modulator. In some embodiments, a food or beverage product is a protein powder, snack bar, carbonated beverage, salty snack, confectionary, sports drink, yogurt, or gummy.

In some embodiments, the present disclosure further provides a method of preparing a preparation comprising an extended-release composition comprising: a protein payload. In some embodiments, a method for preparing a preparation of the present disclosure comprises: (i) hydrating a protein payload to form a solubilized protein solution; (ii) hydrating a release modulator to form a solubilized release modulator solution; (iii) mixing the solubilized protein solution and the solubilized release modulator solution to form a mixed solution; (iv) incubating the mixed solution for 4 hours to 24 hours at 2° C. to 30° C.; (v) heat-treating the mixed solution to form a heat-treated solution; (vi) diluting the heat-treated solution with an acid to form a diluted heat-treated solution; (vii) homogenizing the diluted heat-treated solution to from a homogenate; (viii) filtering the homogenate form a filtered homogenate; (ix) spray drying the filtered homogenate to form a spray-dried preparation; lyophilizing the filtered homogenate to form a lyophilized preparation; or oven-drying the filtered homogenate to form a oven-dried preparation; and (x) milling the spray-dried preparation, lyophilized preparation, or oven-dried preparation to form an extended-release product.

In some embodiments, a solubilized protein solution in a method of the present disclosure comprises 2% (w/v) to 35% (w/v) of protein payload. In some embodiments, a solubilized protein solution in a method of the present disclosure is pH adjusted to pH 3 to pH 10. In some embodiments, a release modulator solution in a method of the present disclosure comprises 1% (w/v) to 10% (w/v) of release modulator. In some embodiments, 0.01% (w/v) to 5% (w/v) dicalcium phosphate is added to a mixed solution in a method of the present disclosure. In some embodiments, 0.01% (w/v) to 5% (w/v) gluconolactone is added to a mixed solution in a method of the present disclosure. In some embodiments, a mixed solution in a method of the present disclosure is heated for 0.5 hrs. to 5 hrs. at 85° C. to 100° C. In some embodiments, a heat-treated solution in a method of the present disclosure is diluted with 5% (v/v) to 200% (v/v) of 0.01% (w/v) to 2% (w/v) acetic acid. In some embodiments, 0.01% (w/v) to 3% (w/v) chitosan is added to a heat-treated solution in a method of the present disclosure. In some embodiments, a diluted heat-treated solution in a method of the present disclosure is homogenized at 3000 rpm to 20000 rpm for 1. min to 20 min. In some embodiments, a homogenate in a method of the present disclosure is passed through a 200 μm to 2000 μm screen filter. In some embodiments, 0.01% (w/v) to 0.5% (w/v) of one or more excipients is added to the filtered homogenate. In some embodiments, a method of the present disclosure comprises spray drying with an inlet temperature of 110° C. to 275° C., an outlet temperature of 60° C. to 95° C., an atomization pressure of 0.75 bar to 4 bar, or a combination thereof. In some embodiments, an extended-release product produced using a method of the present disclosure is characterized as having a moisture content under 6% (w/w). In some embodiments, a method of the present disclosure further comprises milling an extended-release product using a 100 μm to 1000 m screen filter to form a milled extended-release product.

### BRIEF DESCRIPTION OF THE DRAWING

Figures are presented herein for illustration purposes, not for limitation. The foregoing and other objects, aspects, features, and advantages of the disclosure will become more apparent and may be better understood by referring to the following description taken in conjunction with the accompanying drawings.

FIG. 1 is a flow diagram showing an exemplary production process for whey protein formulations characterized as having controlled, extended, and/or delayed release.

FIG. 2 is a line graph showing percent amino acid and/or peptide release as determined using a bicinchoninic acid (BCA) assay from a heat-treated (92° C. for 90 min.) whey protein isolate preparation placed in simulated gastric fluid with 60 U/ml of pepsin at 8%, 11%, or 15% (w/v).

FIG. 3 is a line graph showing percent amino acid and/or peptide release as determined using a BCA assay from a heat-treated (92° C. for 90 min. at pH 4.5. 6.3, or 7.5) whey protein isolate preparation at 11% (w/v) in simulated gastric fluid with 60 U/ml of pepsin.

FIG. 4 is a line graph showing percent amino acid and/or peptide release as determined using a BCA assay from a heat-treated (92° C. for 90 min. at pH 7.5) whey protein isolate preparation at 11% (w/v) heat-treated whey protein prepared at pH 7.5 in the presence of formulated with various release modulators (Tween 85, Span 60, Gum Arabic, and Triacetin) in simulated gastric fluid with 60 U/ml of pepsin.

FIG. 5A is a ribbon diagram showing whey protein isolate secondary structure.

FIG. 5B is an image of whey protein isolate in dry powder form.

FIG. 5C is a microscopy image of whey protein isolate in a dry powder form.

FIG. 5D is an image of whey protein isolate powder incorporated into a conventional milkshake (Muscle Milk® vanilla shake) at a ratio of 1:1 (w/v).

FIG. 5E is a line graph showing percent amino acid and/or peptide release as determined by a BCA assay from untreated whey protein isolate powder in 500 mL of simulated gastric fluid (SGF) with 60 U/mL pepsin, at a pH of 1.2 and at 37° C.

FIG. 5F is a line graph showing percent amino acid and/or peptide release as determined by a BCA assay from untreated whey protein isolate powder mixed with a conventional milkshake (Muscle Milk® vanilla shake) at a 1:1 ratio (w/v).

FIG. 6A is a diagram showing the chemical structure of Pluronic F68.

FIG. 6B is an image showing an 83% (w/w) heat-treated whey protein isolate preparation including 17% (w/w) Pluronic F68 in a dry powder form.

FIG. 6C is a microscopy image showing an 83% (w/w) heat-treated whey protein isolate preparation including 17% (w/w) Pluronic F68 in a dry powder form.

FIG. 6D is an image of an 83% (w/w) heat-treated whey protein isolate preparation including 17% (w/w) Pluronic F68 incorporated into a conventional milkshake (Muscle Milk® vanilla shake) at a ratio of 1:1 (w/v).

FIG. 6E is a line graph showing percent amino acid and/or peptide release as measured by a BCA assay from whey protein isolate powder and a 91% (w/w) heat-treated whey protein isolate preparation including 9% (w/w) Pluronic F68 in 500 mL of simulated gastric fluid with 60 U/ml pepsin, at pH 1.2, 37° C.

FIG. 6F is a line graph showing percent amino acid and/or peptide release as measured by a BCA assay from an 83% (w/w) heat-treated whey protein isolate preparation including 17% (w/w) Pluronic F68 incorporated into a conventional milkshake (Muscle Milk® vanilla shake) at a 1:1 (w/v) ratio (open circles) and from a conventional milkshake (Muscle Milk® vanilla shake) alone (closed circles).

FIG. 7A is a diagram showing the chemical structure of sucrose palmitate.

FIG. 7B is an image of a 91% (w/w) heat-treated whey protein isolate preparation including 9% w/w) sucrose palmitate in a dry powder form.

FIG. 7C is a microscopy image of a 91% (w/w) heat-treated whey protein isolate preparation including 9% (w/w) sucrose palmitate in a dry powder form.

FIG. 7D is an image of a 91% (w/w) heat-treated whey protein isolate preparation including sucrose palmitate incorporated into a conventional milkshake (Muscle Milk® vanilla shake) at a ratio of 1:1 (w/v).

FIG. 7E is a line graph showing percent amino acid and/or peptide release as measured by a BCA assay from whey protein isolate powder and a 91% (w/w) heat-treated whey protein isolate preparation including 9% (w/w) sucrose palmitate in 500 mL of simulated gastric fluid with 60 U/mL pepsin, at pH 1.2, 37° C.

FIG. 7F is a line graph showing percent amino acid and/or peptide release as measured by a BCA assay from a 91% (w/w) heat-treated whey protein isolate preparation including 9% (w/w) sucrose palmitate incorporated into a conventional milkshake (Muscle Milk® vanilla shake) at a 1:1 (w/v) ratio and from a conventional milkshake (Muscle Milk® vanilla shake) alone.

FIG. 8A is a diagram showing the chemical structure of glucose.

FIG. 8B is an image of a 91% (w/w) heat-treated whey protein isolate preparation including 9% (w/w) glucose in a dry powder form.

FIG. 8C is a microscopy image of a 91% (w/w) heat-treated whey protein isolate preparation including 9% (w/w) glucose in a dry powder form.

FIG. 8D is an image of a 91% (w/w) heat-treated whey protein isolate preparation including 9% (w/w) glucose incorporated into a conventional milkshake (Muscle Milk® vanilla shake) at a ratio of 1:1 (w/v).

FIG. 8E is a line graph showing percent amino acid and/or peptide release as measured by a BCA assay from a 91% (w/w) heat-treated whey protein isolate preparation including 9% (w/w) glucose in 500 mL of simulated gastric fluid with 60 U/mL pepsin, pH 1.2, 37° C.

FIG. 8F is a line graph showing percent amino acid and/or peptide release as measured by a BCA assay from a 91% (w/w) heat-treated whey protein isolate preparation including 9% (w/w) glucose incorporated into a conventional milkshake (Muscle Milk® vanilla shake) at a 1:1 (w/v) ratio and from conventional milkshake (Muscle Milk® vanilla shake) alone.

FIG. 9A is a diagram showing an exemplary coating process of heat-treated whey protein formulations to obtain a core-shell structure.

FIG. 9B is a graph showing particle size distribution for uncoated heat-treated whey-protein and an exemplary 85% (w/w) heat-treated whey protein isolate preparation coated with 15% (w/w) ETHOCEL Standard 300.

FIG. 9C is a microscopy image of unformulated whey protein isolate.

FIG. 9D is an image of heat-treated whey protein isolate incorporated into a conventional milkshake (Muscle Milk® chocolate shake) at a ratio of 1:1 (w/v).

FIG. 9E is a microscopy image of an uncoated heat-treated whey protein isolate preparation.

FIG. 9F is an image of an uncoated heat-treated whey protein isolate preparation incorporated into a conventional milkshake (Muscle Milk® chocolate shake) at a ratio of 1:1 (w/v).

FIG. 9G is a microscopy image of an 85% (w/w) heat treated whey protein isolate preparation coated with 15% (w/w) ETHOCEL Standard 300.

FIG. 9H is an image of 85% (w/w) heat treated whey protein isolate preparation coated with 15% (w/w) ETHOCEL standard 300 incorporated into a conventional milkshake (Muscle Milk® chocolate shake).

FIG. 9I is a line graph showing percent amino acid and/or peptide release as measured by a BCA assay from unformulated whey protein isolate, uncoated heat-treated whey protein isolate preparation, and an exemplary heat-treated whey protein isolate preparation coated with 15% (w/w) ETHOCEL standard 300, each mixed with a conventional milkshake (Muscle Milk® chocolate shake) at a 1:1 (w/v) ratio.

FIG. 10A is a flow diagram showing a simulated digestion of whey protein isolate in fasted state simulated gastric fluid (FaSSGF) with pepsin and subsequent analysis of resulting peptide digests via mass spectrometry.

FIG. 10B is an exemplary mass-spectrum of FaSSGF/pepsin digests of unformulated whey protein isolate and heat-treated whey protein isolate.

FIG. 10C is a diagram depicting characteristic peptide fragments of β-lactoglobulin “TKIPAVFK” from whey protein isolate.

FIG. 10D is an exemplary mass-spectrograph of whey protein isolate FaSSFG/pepsin digest or heat-treated whey protein isolate FaSSGF/pepsin digest with values of characteristic peaks for b₂ and y₂ fragments circled.

FIG. 11A is a line graph showing percent amino acid and/or peptide release as measured by a BCA assay from various heat-treated whey protein isolate preparations including water-soluble cellulose ether polymers (e.g., 91% (w/v) heat-treated whey protein isolate, 9% (w/v) Methocel™ 15LV (open circles); 91% (w/v) heat-treated whey protein isolate, 9% (w/v) Methocel™ 4C (open squares); 91% (w/v) heat-treated whey protein isolate, 9% (w/v) Methocel™ 15C (down-turned open triangles); 91% (w/v) heat-treated whey protein isolate, 9% (w/v) Methocel™ 4M (open diamonds)), amino acids (e.g., 91% (w/v) heat-treated whey protein isolate, 9% (w/v) glycine (up-turned open triangles); 91% (w/v) heat-treated whey protein isolate, 9% (w/v) lysine (open hexagons)), sodium alginate (e.g., 76% (w/w) heat-treated whey protein isolate, 6% (w/w) gluconolactone, 3% (w/w) calcium carbonate, 15% (w/w) Vivapharm™ PHR5 (closed circles); 76% (w/w) heat-treated whey protein isolate, 6% (w/w) gluconolactone, 3% (w/w) calcium carbonate, 15% (w/w) Vivapharm™ PH155 (closed squares); 76% (w/w) heat-treated whey protein isolate, 6% (w/w) gluconolactone, 3% (w/w) calcium carbonate, 15% (w/w) Vivapharm™ PH176 (down-turned closed triangles); 76% (w/w) heat-treated whey protein isolate, 6% (w/w) gluconolactone, 3% (w/w) dicalcium phosphate, 15% (w/w) Vivapharm™ PH155 (closed diamonds)), or low methoxyl amidated pectin (e.g., 76% (w/w) heat-treated whey protein isolate, 6% (w/w) gluconolactone, 3% (w/w) calcium carbonate, 15% (w/w) Pectner™ APC210 (up-turned closed triangles); 66% (w/w) heat-treated whey protein isolate, 5% (w/w) gluconolactone, 3% (w/w) calcium carbonate, 26% (w/w) Pectner™ APC220 (closed hexagons)) in 12 mL of simulated gastric fluid with 60 U/mL pepsin, at pH 1.2, 37° C.

FIG. 11B is a line graph showing percent amino acid and/or peptide release as measured by a BCA assay from a heat-treated whey protein isolate preparation including an amino acid (open circles; 91% (w/w) heat-treated whey protein isolate, 9% (w/w) glycine) and a heat-treated whey protein isolate powder preparation including a hydrocolloid (closed circles; 91% (w/w) heat-treated whey protein isolate, 9% (w/w) iota carrageenan) in 12 mL of simulated gastric fluid with 60 U/mL pepsin, at pH 1.2, 37° C.

FIG. 11C is a line graph showing percent amino acid and/or peptide release as measured by a BCA assay from a heat-treated whey protein isolate preparation including an amino acid (open circles; 91% (w/w) heat-treated whey protein isolate, 9% (w/w) glycine) and a heat-treated whey protein isolate powder preparation including a hydrocolloid (closed circles; 91% (w/w) heat-treated whey protein isolate, 9% (w/w) lambda carrageenan) in 12 mL of simulated gastric fluid with 60 U/mL pepsin, at pH 1.2, 37° C.

FIG. 11D is a line graph showing percent amino acid and/or peptide release as measured by a BCA assay from heat-treated whey protein isolate preparations including gluconolactone with a high methoxyl pectin (open circles; 76% (w/w) heat-treated whey protein isolate, 6% (w/w) gluconolactone, 3% (w/w) calcium carbonate, 15% (w/w) Pectner™ APC169B) or sodium alginate (closed circles; 76% (w/w) heat-treated whey protein isolate, 6% (w/w) gluconolactone, 3% (w/w) calcium carbonate, 15% (w/w) Vivapharm™ PHR5) in 12 mL of simulated gastric fluid with 60 U/mL pepsin, at pH 1.2, 37° C.

FIG. 11E is a line graph showing percent amino acid and/or peptide release as measured by a BCA assay from heat-treated whey protein isolate preparations including gluconolactone with a high methoxyl pectin (open circles; 76% (w/w) heat-treated whey protein isolate, 6% (w/w) gluconolactone, 3% (w/w) calcium carbonate, 15% (w/w) Pectner™ 169B) or sodium alginate (closed circles; 76% (w/w) heat-treated whey protein isolate, 6% (w/w) gluconolactone, 3% (w/w) calcium carbonate, 15% (w/w) Vivapharm™ PH155) in 12 mL of simulated gastric fluid with 60 U/mL pepsin, at pH 1.2, 37° C.

FIG. 11F is a line graph showing percent amino acid and/or peptide release as measured by a BCA assay from heat-treated whey protein isolate preparations including gluconolactone with a high methoxyl pectin (open circles; 76% (w/w) heat-treated whey protein isolate, 6% (w/w) gluconolactone, 3% (w/w) calcium carbonate, 15% (w/w) Pectner™ APC169B) or calcium-crosslinked low methoxyl amidated pectin (closed circles; 76% (w/w) heat-treated whey protein isolate, 6% (w/w) gluconolactone, 3% (w/w) calcium carbonate, 15% (w/w) Pectner™ APC210) in 12 mL of simulated gastric fluid with 60 U/mL pepsin, at pH 1.2, 37° C.

FIG. 11G is a line graph showing percent amino acid and/or peptide release as measured by a BCA assay from heat-treated whey protein isolate preparations including gluconolactone with a high methoxyl pectin (open circles; 76% (w/w) heat-treated whey protein isolate, 6% (w/w) gluconolactone, 3% (w/w) calcium carbonate, 15% (w/w) Pectner™ 169B) or sodium alginate (closed circles; 76% (w/w) heat-treated whey protein isolate, 6% (w/w) gluconolactone, 3% (w/w) calcium carbonate, 15% (w/w) Vivapharm™ PH176) in 12 mL of simulated gastric fluid with 60 U/mL pepsin, at pH 1.2, 37° C.

FIG. 11H is a line graph showing percent amino acid and/or peptide release as measured by a BCA assay from heat-treated whey protein isolate preparations including gluconolactone and calcium carbonate with a high methoxyl pectin (open circles; 76% (w/w) heat-treated whey protein isolate, 6% (w/w) gluconolactone, 3% (w/w) calcium carbonate, 15% (w/w) Pectner™ APC169B), gluconolactone and calcium carbonate with a low methoxyl amidated pectin (closed squares; 76% (w/w) heat-treated whey protein isolate, 6% (w/w) gluconolactone, 3% (w/w) calcium carbonate, 15% (w/w) Vivapharm™ PH176), or gluconolactone and dicalcium phosphate with a low methoxyl amidated pectin (closed circles; 76% (w/w) heat-treated whey protein isolate, 6% (w/w) gluconolactone, 3% (w/w) dicalcium phosphate, 15% (w/w) Vivapharm™ PH155) in 12 mL of simulated gastric fluid with 60 U/mL pepsin, at pH 1.2, 37° C.

FIG. 12A is a line graph showing percent amino acid and/or peptide release as measured by a BCA assay from a heat-treated whey protein isolate preparation including an amino acid (open circles; 91% (w/w) heat-treated whey protein isolate, 9% (w/w) glycine) and a heat-treated whey protein isolate preparation including a hypromellose (closed circles; 91% (w/w) heat-treated whey protein isolate, 9% (w/w) hypromellose acetate succinate) in 12 mL of simulated gastric fluid with 60 U/mL pepsin, at pH 1.2, 37° C.

FIG. 12B is a line graph showing percent amino acid and/or peptide release as measured by a BCA assay from a preparation including 71% (w/w) heat-treated whey protein isolate, 27% (w/w) nutritional yeast, 2% (w/w) transglutaminase (open circles), or a preparation including 71.4% (w/w) heat-treated whey protein isolate, 14.3% (w/w) maltodextrin DE16, 14.3% (w/w) functional rice starch (closed circles) in 12 mL of simulated gastric fluid with 60 U/mL pepsin, at pH 1.2, 37° C.

FIG. 12C is a line graph showing percent amino acid and/or peptide release as measured by a BCA assay from a preparation including 71% (w/w) heat-treated whey protein isolate, 27% (w/w) nutritional yeast, 2% (w/w) transglutaminase (open circles), or a preparation including 71.4% (w/w) heat-treated whey protein isolate, 14.3% (w/w) maltodextrin DE16, 14.3% (w/w) corn starch (closed circles) in 12 mL of simulated gastric fluid with 60 U/mL pepsin, at pH 1.2, 37° C.

FIG. 12D is a line graph showing percent amino acid and/or peptide release as measured by a BCA assay from a heat-treated whey protein isolate preparation including an amino acid (open circles; 91% (w/w) heat-treated whey protein isolate, 9% (w/w) glycine), or a preparation including 91% (w/w) heat-treated whey protein isolate, 9% (w/w) ORAFTI inulin GR (closed circles) in 12 mL of simulated gastric fluid with 60 U/mL pepsin, at pH 1.2, 37° C.

FIG. 12E is a line graph showing percent amino acid and/or peptide release as measured by a BCA assay from a heat-treated whey protein isolate preparation including an amino acid (open circles; 91% (w/w) heat-treated whey protein isolate, 9% (w/w) glycine), or a preparation including 91% (w/w) heat-treated whey protein isolate, 9% (w/w) PromOat Fiber (closed circles) in 12 mL of simulated gastric fluid with 60 U/mL pepsin, at pH 1.2, 37° C.

FIG. 12F is a line graph showing percent amino acid and/or peptide release as measured by a BCA assay from a heat-treated whey protein isolate preparation including an amino acid (open circles; 91% (w/w) heat-treated whey protein isolate, 9% (w/w) glycine), or a preparation including 91% (w/w) heat-treated whey protein isolate, 9% (w/w) glucose (closed circles) in 12 mL of simulated gastric fluid with 60 U/mL pepsin, at pH 1.2, 37° C.

FIG. 12G is a line graph showing percent amino acid and/or peptide release as measured by a BCA assay from a heat-treated whey protein isolate preparation including an amino acid (open circles; 91% (w/w) heat-treated whey protein isolate, 9% (w/w) glycine), or a preparation including 91% (w/w) heat-treated whey protein isolate, 9% (w/w) functional rice starch (closed circles) in 12 mL of simulated gastric fluid with 60 U/mL pepsin, at pH 1.2, 37° C.

FIG. 12H is a line graph showing percent amino acid and/or peptide release as measured by a BCA assay from a heat-treated whey protein isolate preparation including an amino acid (open circles; 91% (w/w) heat-treated whey protein isolate, 9% (w/w) glycine), or a preparation including 91% (w/w) heat-treated whey protein isolate, 9% (w/w) tapioca polydextrose (closed circles) in 12 mL of simulated gastric fluid with 60 U/mL pepsin, at pH 1.2, 37° C.

FIG. 13A is a line graph showing percent amino acid and/or peptide release as measured by a BCA assay from a preparation including 83% (w/w) heat-treated whey protein isolate, 8% (w/w) sodium silicate, 9% (w/w) calcium carbonate (open circles), or a preparation including 82.6% (w/w) heat-treated whey protein isolate, 15.1% (w/w) Vivapur™ alginate FD176, 1.5% (w/w) gluconolactone, 0.8% (w/w) dicalcium phosphate (closed circles) in 12 mL of simulated gastric fluid with 60 U/mL pepsin, at pH 1.2, 37° C.

FIG. 13B is a line graph showing percent amino acid and/or peptide release as measured by a BCA assay from a preparation including 83% (w/w) heat-treated whey protein isolate, 8% (w/w) sodium silicate, 9% (w/w) calcium carbonate (open circles), or a preparation including 80% (w/w) heat-treated whey protein isolate, 13.4% (w/w) Swanlac™ ASL, 0.9% (w/w) Vivapur™ alginate FD176, 2.9% (w/w) chitosan, 1.4% (w/w) gluconolactone, 1.4% (w/w) dicalcium phosphate (closed circles) in 12 mL of simulated gastric fluid with 60 U/mL pepsin, at pH 1.2, 37° C.

FIG. 14A is a graph showing particle diameter distributions (volume density %) of a preparation including 81.2% (w/w) heat-treated whey protein isolate, 16.2% (w/w) rice starch, 1% (w/w) chitosan, 1% (w/w) tannic acid, 0.6% w/w laccase.

FIG. 14B is a graph showing particle diameter distributions (volume density %) of a preparation including 78% (w/w) heat-treated whey protein isolate, 15% (w/w) Vivapharm™ PH176, 5% (w/w) dicalcium phosphate, 2% (w/w) gluconolactone.

FIG. 14C is a graph showing particle diameter distributions (volume density %) of a preparation including 71% (w/w) heat-treated whey protein isolate, 27% (w/w) nutritional yeast, 2% (w/w) transglutaminase.

FIG. 14D is a graph showing particle diameter distributions (volume density %) of a preparation including 82.4% (w/w) heat-treated whey protein isolate, 13.8% (w/w) Pectner™ APC169B, 0.9% (w/w) Vivapharm™ PH176, 1.45% (w/w) gluconolactone, 1.45% (w/w) calcium hydrogen phosphate anhydrous.

FIG. 15 is a flow diagram showing an exemplary production process for an exemplary heat-treated whey protein isolate formulation.

FIG. 16A is a photograph showing an exemplary preparation including 82.6% (w/w) heat-treated whey protein isolate, 15.1% (w/w) Vivapur™ alginate FD176, 1.5% (w/w) gluconolactone, 0.8% (w/w) dicalcium phosphate prepared according to an exemplary method as shown in the flow diagram of FIG. 15.

FIG. 16B is a photograph showing Crystal Lite™ powder.

FIG. 16C is a photograph showing a 2:1 mixture of Crystal Lite™ powder and exemplary preparation of FIG. 16A.

FIG. 16D is a photograph showing commercial protein powder.

FIG. 16E is a photograph showing a 1:2 mixture of commercial protein powder and exemplary preparation of FIG. 16A.

FIG. 17A is a line graph showing percent amino acid and/or peptide release as measured by a BCA assay from a preparation of FIG. 16A including 82.6% (w/w) heat-treated whey protein isolate, 15.1% (w/w) Vivapur™ alginate FD176, 1.5% (w/w) gluconolactone, 0.8% (w/w) dicalcium phosphate mixed with 2 parts of Cystal Lite™ as shown in FIG. 16C.

FIG. 17B is a line graph showing amino acid and/or peptide release as measured by a BCA assay from a preparation of FIG. 16A including 82.6% (w/w) heat-treated whey protein isolate, 15.1% (w/w) Vivapur™ alginate FD176, 1.5% (w/w) gluconolactone, 0.8% (w/w) dicalcium phosphate mixed with 1 part of commercial protein powder as shown in FIG. 16E.

FIG. 18A is a line graph showing amino acid and/or peptide release as measured by a BCA assay from a preparation of FIG. 16A including 82.6% (w/w) heat-treated whey protein isolate, 15.1% (w/w) Vivapur™ alginate FD176, 1.5% (w/w) gluconolactone, 0.8% (w/w) dicalcium phosphate stored for 14 days in various conditions (closed circle, −4° F., 40% relative humidity, vacuum, desiccant, mylar bag; closed square, 80° F., 40% relative humidity, vacuum, desiccant, mylar bag; open square, 100° F., 40% relative humidity, vacuum, desiccant, mylar bag; closed triangle, 80° F., 75% relative humidity, vacuum, desiccant, mylar bag; open triangle, 80° F., 40% relative humidity, nitrogen, desiccant, glass vial; open hexagon, 80° F., 40% relative humidity, nitrogen, desiccant, mylar bag; hatched hexagon, 80° F., 40% relative humidity, air, desiccant, mylar bag; closed star, 80° F., 40% relative humidity, vacuum, no desiccant, mylar bag) or without storage (open circle) in 500 mL of simulated gastric fluid with 60 U/mL pepsin, at pH 1.2, 37° C.

FIG. 18B is a line graph showing amino acid and/or peptide release as measured by a BCA assay from a preparation of FIG. 16A including 82.6% (w/w) heat-treated whey protein isolate, 15.1% (w/w) Vivapur™ alginate FD176, 1.5% (w/w) gluconolactone, 0.8% (w/w) dicalcium phosphate stored for 28 days in various conditions (closed circle, −4° F., 40% relative humidity, vacuum, desiccant, mylar bag; closed square, 80° F., 40% relative humidity, vacuum, desiccant, mylar bag; open square, 100° F., 40% relative humidity, vacuum, desiccant, mylar bag; closed triangle, 80° F., 75% relative humidity, vacuum, desiccant, mylar bag; open triangle, 80° F., 40% relative humidity, nitrogen, desiccant, glass vial; open hexagon, 80° F., 40% relative humidity, nitrogen, desiccant, mylar bag; hatched hexagon, 80° F., 40% relative humidity, air, desiccant, mylar bag; closed star, 80° F., 40% relative humidity, vacuum, no desiccant, mylar bag) or without storage (open circle) in 500 mL of simulated gastric fluid with 60 U/mL pepsin, at pH 1.2, 37° C.

FIG. 19 is a line graph showing percent amino acid and/or peptide release as measured by a BCA assay from various exemplary preparations (i) closed circles, 70% (w/w) heat-treated whey protein isolate, 23.5% (w/w) Pectner™ APC210, 1.6% (w/w) Vivapur™ alginate FD176, 2.4% (w/w) gluconolactone, 2.4% (w/w) dicalcium phosphate; (ii) open circles, 75% (w/w) heat-treated whey protein isolate, 19.6% (w/w) Pectner™ APC210, 1.3% (w/w) Vivapur™ alginate FD176, 2% (w/w) gluconolactone, 2% (w/w) dicalcium phosphate; (iii) closed squares, 80% (w/w) heat-treated whey protein isolate, 15.7% (w/w) Pectner™ APC210, 1% (w/w) Vivapur™ alginate FD176, 1.6% (w/w) gluconolactone, 1.6% (w/w) dicalcium phosphate; (iv) open squares, 85% (w/w) heat-treated whey protein isolate, 11.7% (w/w) Pectner™ APC210, 0.8% (w/w) Vivapur™ alginate FD176, 1.2% (w/w) gluconolactone, 1.2% (w/w) dicalcium phosphate; (v) closed diamond, 90% (w/w) heat-treated whey protein isolate, 7.8% (w/w) Pectner™ APC210, 0.5% (w/w) Vivapur™ alginate FD176, 0.8% (w/w) gluconolactone, 0.8% (w/w) dicalcium phosphate) in 500 mL of simulated gastric fluid with 60 U/mL pepsin, at pH 1.2, 37° C.

FIG. 20A is a line graph showing percent amino acid and/or peptide release as measured by a BCA assay from various exemplary preparations (closed circle, 90.8 (w/w) heat-treated whey protein isolate, 5% (w/w) Pectner™ APC210, 1% (w/w) Vivapur™ alginate FD176, 1.6% (w/w) gluconolactone, 1.6% (w/w) dicalcium phosphate; closed square, 86% (w/w) heat-treated whey protein isolate, 10% (w/w) Pectner™ APC210, 1% (w/w) Vivapur™ alginate FD176, 1.5% (w/w) gluconolactone, 1.5% (w/w) dicalcium phosphate; closed triangle, 81.2% (w/w) heat-treated whey protein isolate, 15% (w/w) Pectner™ APC210, 0.9% (w/w) Vivapur™ alginate FD176, 1.4% (w/w) gluconolactone, 1.4% (w/w) dicalcium phosphate) in 12 mL of simulated gastric fluid with 60 U/mL pepsin, at pH 1.2, 37° C.

FIG. 20B is a line graph showing percent amino acid and/or peptide release as measured by a BCA assay from various exemplary preparations (closed circle, 83 (w/w) heat-treated whey protein isolate, 13.9% (w/w) Pectner™ APC210, 0.2% (w/w) Vivapur™ alginate FD176, 1.4% (w/w) gluconolactone, 1.5% (w/w) dicalcium phosphate; open circle, 81.9% (w/w) heat-treated whey protein isolate, 13.7% (w/w) Pectner™ APC210, 1.5% (w/w) Vivapur™ alginate FD176, 1.4% (w/w) gluconolactone, 1.4% (w/w) dicalcium phosphate; closed square, 80.7% (w/w) heat-treated whey protein isolate, 13.5% (w/w) Pectner™ APC210, 3% (w/w) Vivapur™ alginate FD176, 1.4% (w/w) gluconolactone, 1.4% (w/w) dicalcium phosphate) in 12 mL of simulated gastric fluid with 60 U/mL pepsin, at pH 1.2, 37° C.

FIG. 20C is a line graph showing percent amino acid and/or peptide release as measured by a BCA assay from various exemplary preparations (closed circle, 82.4% (w/w) heat-treated whey protein isolate, 9.9% (w/w) Pectner™ APC210, 0.7% (w/w) Vivapur™ alginate FD176, 3.5% (w/w) gluconolactone, 3.5% (w/w) dicalcium phosphate; open circle, 82.4% (w/w) heat-treated whey protein isolate, 11.4% (w/w) Pectner™ APC210, 0.8% (w/w) Vivapur™ alginate FD176, 2.7% (w/w) gluconolactone, 2.7% (w/w) dicalcium phosphate; closed square, 82.4% (w/w) heat-treated whey protein isolate, 12.8% (w/w) Pectner™ APC210, 0.9% (w/w) Vivapur™ alginate FD176, 2% (w/w) gluconolactone, 1.9% (w/w) dicalcium phosphate); open square, 82.4% (w/w) heat-treated whey protein isolate, 13.5% (w/w) Pectner™ APC210, 0.9% (w/w) Vivapur™ alginate FD176, 1.6% (w/w) gluconolactone, 1.6% (w/w) dicalcium phosphate); closed diamond, 82.4% (w/w) heat-treated whey protein isolate, 14.3% (w/w) Pectner™ APC210, 1% (w/w) Vivapur™ alginate FD176, 1.1% (w/w) gluconolactone, 1.2% (w/w) dicalcium phosphate); open diamond, 82.4% (w/w) heat-treated whey protein isolate, 14.5% (w/w) Pectner™ APC210, 1% (w/w) Vivapur™ alginate FD176, 1% (w/w) gluconolactone, 1% (w/w) dicalcium phosphate) in 12 mL of simulated gastric fluid with 60 U/mL pepsin, at pH 1.2, 37° C.

FIG. 21A is a graph showing particle diameter distributions (volume density %) of untreated whey protein isolate (open circle); heat-treated whey protein isolate (Dv₅₀>100 μm; open square); heat-treated whey protein isolate (Dv₅₀<100 μm; closed circle); and an exemplary preparation including 80% (w/w) heat-treated whey protein isolate (Glanbia Provon 190), 13.4% (w/w) kappa carrageenan, 0.86% (w/w) Vivapur™ alginate FD176, 2.9% (w/w) chitolytic, 1.4% (w/w) gluconolactone, 1.5% (w/w) dicalcium phosphate (Dv₅₀<100 μm; closed square).

FIG. 21B is a line graph showing percent amino acid and/or peptide release as measured by a BCA assay from untreated whey protein isolate (open circle); heat-treated whey protein isolate (Dv₅₀>100 μm; open square); heat-treated whey protein isolate (Dv₅₀<100 μm; closed circle); and a formulation including 80% (w/w) heat-treated whey protein isolate (Glanbia Provon 190), 13.4% (w/w) kappa carrageenan, 0.86% (w/w) Vivapur™ alginate FD176, 2.9% (w/w) chitolytic, 1.4% (w/w) gluconolactone, 1.5% (w/w) dicalcium phosphate (Dv₅₀<100 μm; closed square).

FIG. 22 is a bar graph showing the viscosity (cP) of exemplary preparations including: 82.4% (w/w) heat-treated whey protein isolate, 13.8% (w/w) Pectner™ APC169B, 0.9% (w/w) Vivapharm™ alginate PH176, 1.45% (w/w) gluconolactone, 1.45% (w/w) calcium hydrogen phosphate anhydrous (Formulation A); 80% (w/w) heat-treated whey protein isolate (Glanbia Provon 190), 13.4% (w/w) Pectner™ APC210, 0.86% (w/w) Vivapur™ alginate FD176, 2.9% (w/w) chitolytic, 1.4% (w/w) gluconolactone, 1.4% (w/w) dicalcium phosphate (Formulation B), or untreated whey protein (Premier whey protein (vanilla) (Formulation C)).

FIG. 23A is a graph showing particle diameter distributions (volume density %) of an exemplary preparation including 80.2% (w/w) heat-treated whey protein isolate, 13.3% (w/w) Pectner™ APC210, 1% (w/w) Vivapur™ alginate FD176, 3.2% (w/w) gluconolactone, 2.1% (w/w) dicalcium phosphate.

FIG. 23B is a line graph showing percent amino acid and/or peptide release as measured by a BCA assay from an exemplary preparation including 78.3% (w/w) heat-treated whey protein isolate, 13.1% (w/w) Pectner™ APC210, 1% (w/w) Vivapur™ alginate FD176, 2.4% (w/w) Sipernat 22s, 3.1% (w/w) gluconolactone, 2.1% (w/w) dicalcium phosphate in 500 mL of simulated gastric fluid with 60 U/mL pepsin, at pH 1.2, 37° C.

FIG. 23C is a graph showing particle diameter distributions (volume density %) of an exemplary preparation including 78.3% (w/w) heat-treated whey protein isolate, 13.1% (w/w) Pectner™ APC210, 1% (w/w) Vivapur™ alginate FD176, 2.4% (w/w) Sipernat 22s, 3.1% (w/w) gluconolactone, 2.1% (w/w) dicalcium phosphate.

FIG. 23D is a line graph showing percent amino acid and/or peptide release as measured by a BCA assay from an exemplary preparation including 78.3% (w/w) heat-treated whey protein isolate, 13.1% (w/w) Pectner™ APC210, 1% (w/w) Vivapur™ alginate FD176, 1.2% (w/w) Sipernat 22s, 1.2% (w/w) aquafaba, 3.1% (w/w) gluconolactone, 2.1% (w/w) dicalcium phosphate in 500 mL of simulated gastric fluid with 60 U/mL pepsin, at pH 1.2, 37° C.

FIG. 23E is a graph showing particle diameter distributions (volume density %) of an exemplary preparation including 78.3% (w/w) heat-treated whey protein isolate, 13.1% (w/w) Pectner™ APC210, 1% (w/w) Vivapur™ alginate FD176, 1.2% (w/w) Sipernat 22s, 1.2% (w/w) aquafaba, 3.1% (w/w) gluconolactone, 2.1% (w/w) dicalcium phosphate.

FIG. 23F is a line graph showing percent amino acid and/or peptide release as measured by a BCA assay from an exemplary preparation including 77% (w/w) heat-treated whey protein isolate, 12.8% (w/w) Pectner™ APC210, 1% (w/w) Vivapur™ alginate FD176, 1.2% (w/w) chitosan, 1.2% (w/w) Sipernat 22s, 1.2% (w/w) aquafaba, 3.1% (w/w) gluconolactone, 2% (w/w) dicalcium phosphate in 500 mL of simulated gastric fluid with 60 U/mL pepsin, at pH 1.2, 37° C.

FIG. 23G is a graph showing particle diameter distributions (volume density %) of an exemplary preparation including 77% (w/w) heat-treated whey protein isolate, 12.8% (w/w) Pectner™ APC210, 1% (w/w) Vivapur™ alginate FD176, 1.2% (w/w) chitosan, 1.2% (w/w) Sipernat 22s, 1.2% (w/w) aquafaba, 3.1% (w/w) gluconolactone, 2% (w/w) dicalcium phosphate.

FIG. 23H is a line graph showing percent amino acid and/or peptide release as measured by a BCA assay from an exemplary preparation including 77% (w/w) heat-treated whey protein isolate, 12.8% (w/w) Pectner™ APC210, 1% (w/w) Vivapur™ alginate FD176, 1.2% (w/w) chitosan, 1.2% (w/w) HiCap100, 1.2% (w/w) aquafaba, 3.1% (w/w) gluconolactone, 2% (w/w) dicalcium phosphate in 500 mL of simulated gastric fluid with 60 U/mL pepsin, at pH 1.2, 37° C.

FIG. 23I is a graph showing particle diameter distributions (volume density %) of an exemplary preparation including 77% (w/w) heat-treated whey protein isolate, 12.8% (w/w) Pectner™ APC210, 1% (w/w) Vivapur™ alginate FD176, 1.2% (w/w) chitosan, 1.2% (w/w) HiCap100, 1.2% (w/w) aquafaba, 3.1% (w/w) gluconolactone, 2% (w/w) dicalcium phosphate.

FIG. 23J is a line graph showing percent amino acid and/or peptide release as measured by a BCA assay from an exemplary preparation including 83.8% (w/w) heat-treated whey protein isolate, 4% (w/w) Ticaloid 710H, 1% (w/w) Vivapur™ alginate FD176, 5.5% (w/w) chitosan, 3.4% (w/w) gluconolactone, 2.2% (w/w) dicalcium phosphate in 500 mL of simulated gastric fluid with 60 U/mL pepsin, at pH 1.2, 37° C.

FIG. 23K is a graph showing particle diameter distributions (volume density %) of an exemplary preparation including 83.8% (w/w) heat-treated whey protein isolate, 4% (w/w) Ticaloid 710H, 1% (w/w) Vivapur™ alginate FD176, 5.5% (w/w) chitosan, 3.4% (w/w) gluconolactone, 2.2% (w/w) dicalcium phosphate.

FIG. 23L is a line graph showing percent amino acid and/or peptide release as measured by a BCA assay from an exemplary preparation including 81.1% (w/w) heat-treated whey protein isolate, 3.9% (w/w) Ticaloid 710H, 1% (w/w) Vivapur™ alginate FD176, 5.4% (w/w) chitosan, 2.6% (w/w) Sipernat 50s, 3.3% (w/w) gluconolactone, 2.2% (w/w) dicalcium phosphate in 500 mL of simulated gastric fluid with 60 U/mL pepsin, at pH 1.2, 37° C.

FIG. 23M is a graph showing particle diameter distributions (volume density %) of an exemplary preparation including 81.1% (w/w) heat-treated whey protein isolate, 3.9% (w/w) Ticaloid 710H, 1% (w/w) Vivapur™ alginate FD176, 5.4% (w/w) chitosan, 2.6% (w/w) Sipernat 50s, 3.3% (w/w) gluconolactone, 2.2% (w/w) dicalcium phosphate.

FIG. 23N is a graph showing particle diameter distributions (volume density %) of an exemplary preparation including 81.1% (w/w) heat-treated whey protein isolate, 3.9% (w/w) Ticaloid 710H, 1% (w/w) Vivapur™ alginate FD176, 5.4% (w/w) chitosan, 1.3% (w/w) Sipernat 50s, 1.3% (w/w) aquafaba, 3.3% (w/w) gluconolactone, 2.2% (w/w) dicalcium phosphate.

FIG. 24 is a bar graph showing the permeation capacity (%) of amino acids across a human intestinal epithelial cell layer in a Transwell® cell culture insert after 30 min, 120 min, and 240 min incubation following application of various samples to the apical side of the cell layer. Samples applied were pH-neutralized gastric fluid digests (500 mL gastric fluid with 60 U/mL pepsin, at pH 1.2, 37° C.) of untreated whey protein isolate (after 1080 min. incubation), heat-treated whey protein isolate (after 1080 min. incubation), or an exemplary preparation including 80% (w/w) heat-treated whey protein isolate (Glanbia™ Provon 190), 13.4% (w/w) Pectner™ APC210, 0.86% (w/w) Vivapur™ alginate FD176, 2.9% (w/w) chitosan, 1.4% (w/w) gluconolactone, 1.4% (w/w) dicalcium phosphate (after incubation at various time points (e.g., between 5 min, and 1080 min.)).

FIG. 25 is a bar graph showing the viscosity of exemplary preparations after mixture into water at a concentration of 100 mg/mL as compared to the viscosity of unformulated whey protein isolate and commercial whey protein powder. The white bar (Glanbia) is unformulated whey protein isolate; the grey bar (VK2) is an exemplary preparation including 78.3% (w/w) heat-treated whey protein isolate, 13.1% (w/w) Pectner™ APC210, 1% (w/w) Vivapur™ Alginate FD176, 3.1% (w/w) gluconolactone, 2.1% (w/w) Dicalcium phosphate, 2.4% (w/w) Sipernat™ 22S; the checkered bar (VK8) is an exemplary preparation including 76.4% (w/w) heat-treated Glanbia Provon™ 190 whey protein isolate, 12.8% (w/w) Pectner™ APC 210, 0.9% (w/w) Vivapur™ FD 176, 2.0% (w/w) gluconolactone, 2.0% (w/w) dicalcium phosphate, and 5.9% (w/w) Chitolytic chitosan; the vertical line bar (VK8+3% SHMP) is an exemplary preparation including 74.1% (w/w) heat-treated Glanbia Provon™ 190 whey protein isolate, 12.4% (w/w) Pectner™ APC 210, 0.9% (w/w) Vivapur™ FD 176, 1.9% (w/w) gluconolactone, 2.0% (w/w) dicalcium phosphate, 5.7% (w/w) Chitolytic chitosan, and 3% (w/w) sodium hexametaphosphate; and the black bar is commercial Muscle Milk vanilla protein powder.

FIG. 26 is a line graph showing the percent amino acid and/or peptide release as measured by a BCA assay from exemplary preparations including 74.1% (w/w) heat-treated Glanbia Provon™ 190 whey protein isolate, 12.4% (w/w) Pectner™ APC 210, 0.9% (w/w) Vivapur™ FD 176, 1.9% (w/w) gluconolactone, 2.0% (w/w) dicalcium phosphate, 5.7% (w/w) Chitolytic chitosan, and 3% (w/w) sodium hexametaphosphate (closed squares); 76.4% (w/w) heat-treated Glanbia Provon™ 190 whey protein isolate, 12.8% (w/w) Pectner™ APC 210, 0.9% (w/w) Vivapur™ FD 176, 2.0% (w/w) gluconolactone, 2.0% (w/w) dicalcium phosphate, and 5.9% (w/w) Chitolytic chitosan (closed circles); and 97% (w/w) unformulated whey protein isolate with 3% (w/w) sodium hexametaphosphate (open squares).

### DEFINITIONS

Unless defined otherwise, technical and scientific terms used herein have the same meaning as is commonly understood to which the claimed subject matter belongs. In the event that there are a plurality of definitions for terms herein, those in this section prevail.

It is to be understood that the general description and the detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Definition of standard chemistry terms may be found in reference works, including but not limited to, Carey and Sundberg “Advanced Organic Chemistry 4th Ed.” Vols. A (2000) and B (2001), Plenum Press, New York.

As used herein, the symbol “<” means less than or fewer than. As used herein, the symbol “>” means more than.

As used herein, the term “about” or “approximately” means within 10%, preferably within 10%, and more preferably within 5% of a given value or range.

Ambient: The term “ambient”, as used herein, refers to a typical indoor (e.g., climate-controlled) temperature, usually within a range of about 18° C. to about 32° C., and/or typical indoor (e.g., climate-controlled) humidity, usually within a range of about 30% to 50%. In some embodiments, ambient temperature is within a range of about 20° C. to about 30° C. In some embodiments, ambient temperature is 25±5° C. In some embodiments, ambient temperature is approximately 21° C. In some embodiments, ambient temperature is 18° C. In some embodiments, ambient temperature is 19° C. In some embodiments, ambient temperature is 20° C. In some embodiments, ambient temperature is 21° C. In some embodiments, ambient temperature is 22° C. In some embodiments, ambient temperature is 23° C. In some embodiments, ambient temperature is 24° C. In some embodiments, ambient temperature is 25° C. In some embodiments, ambient temperature is 26° C. In some embodiments, ambient temperature is 27° C. In some embodiments, ambient temperature is 28° C. In some embodiments, ambient temperature is 29° C. In some embodiments, ambient temperature is 30° C. In some embodiments, ambient may be used to describe outdoor conditions, and may include temperatures ranging from about 15° C. to about 40° C., or from about 25° C. to about 40° C. In some embodiments, ambient humidity is within a range of about 35% to about 45%. In some embodiments, ambient humidity is 35%. In some embodiments, ambient humidity is 36%. In some embodiments, ambient humidity is 37%. In some embodiments, ambient humidity is 38%. In some embodiments, ambient humidity is 39%. In some embodiments, ambient humidity is 40%. In some embodiments, ambient humidity is 41%. In some embodiments, ambient humidity is 42%. In some embodiments, ambient humidity is 43%. In some embodiments, ambient humidity is 44%. In some embodiments, ambient humidity is 45%.

Beverage: As used herein, the term “beverage” is used to refer to a potable liquid (e.g., that can be ingested, swallowed, drunk, or consumed by a person or animal without material risk to the person or animal). For example, a beverage can be or comprise beer, juice, milk, sports drink, tea, water, soda, yogurt, etc. In some embodiments, a “beverage” may be or comprise a pharmaceutical formulation in liquid form.

Biocompatible: As used herein, the term “biocompatible” is used to describe a characteristic of not causing significant detectable harm to living tissue when placed in contact therewith e.g., in vivo. In certain embodiments, materials are “biocompatible” if they are not significantly toxic to cells, e.g., when contacted therewith in a relevant amount and/or under relevant conditions such as over a relevant period of time. In certain embodiments, materials are “biocompatible” if their addition to cells in vitro results in less than or equal to 20% cell death, and/or their administration in vivo does not induce significant inflammation or other adverse effects.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Controlled release: As used herein, the term “controlled release” refers to release of one or more components (e.g., one or more food components) where the release is temporally regulated, locationally regulated, environmentally regulated or a combination thereof. In some embodiments, “controlled release” may additionally refer to regulation of the magnitude to which one or more components (e.g., one or more food components) is released.

Degradation: As used herein, the term “degradation” refers to a change in chemical structure and often involves breakage of at least one chemical bond. To say that a chemical compound is degraded typically means that the chemical structure of the chemical compound has changed (e.g., a chemical bond is broken). Common mechanisms of degradation include, for example, oxidation, hydrolysis, isomerization, fragmentation, or a combination thereof.

Delayed release: As used herein, the term “delayed release” is used to refer to a postponed release of one or more component (e.g., one or more food components) by a formulation, composition, and/or preparation beyond: a pre-determined time, a time in which the one or more components would naturally be released, a time in which the one or more components would be released in the absence of a manipulation, or a combination thereof.

Delivery: As used herein, the term “delivery” is used to refer to the carrying and/or deposition and/or moving of nutrients (e.g., macronutrients, micronutrients, ketones, flavanols, prebiotics, etc.) and/or encapsulants to particular location (e.g., into and/or throughout the body). In some embodiments, for example, delivery may refer to payload delivery to the epithelial cells in the gastrointestinal tract. In some embodiments, for example, delivery may refer to payload delivery to into the blood stream (e.g., systemic absorption). In some embodiments, for example, delivery may refer to ingestion at the point of consumption for a shelf-stable food composition containing a nutrient payload.

Diameter: As used herein, the term “diameter” is used to refer to the longest distance from one end of a particle to another end of the particle. Those skilled in the art will appreciate that a variety of techniques are available for use in characterizing particle diameters (i.e., particle sizes). In some embodiments, for example, size of particles (e.g., diameter of particles) can be measured by a Coulter Counter. In some embodiments, for example, size of particles (e.g., diameter of particles) can be measured by a Malvern Mastersizer. In some embodiments, a population of particles is characterized by an average size (e.g., D[3,2], D[4,3], etc.) and/or by particular characteristics of size distribution (e.g., absence of particles above or below particular sizes [e.g., Dv10, Dv20, Dv30, Dv40, Dv50, Dv60, Dv70, Dv80, Dv90, Dv99, etc.], a unimodal, bimodal, or multimodal distribution, etc.).

Dissolution Solvent: As used herein, the term “dissolution solvent” refers to a fluid in which a solute dissolves or into which a component (e.g., a food component) is released. In some embodiments, dissolution solvents include fluids naturally found within one or more gastrointestinal compartments, simulated gastric fluids, simulated intestinal fluids, water or phosphate buffered saline.

Dispersity: As used herein, the term “dispersity” is used to refer to the breadth of particle size distribution relative to the average particle size. In some embodiments, for example, size of particles (e.g., diameter of particles) can be measured by a Coulter Counter. In some embodiments, for example, size of particles (e.g., diameter of particles) can be measured by a Malvern Mastersizer. In some embodiments, the population of particles is characterized by, for example, an average size (e.g., Dv50) and, for example, a corresponding standard deviation. In some embodiments, the dispersity of a population of particles refers to double (e.g., 2-fold) the ratio of standard deviation (e.g., σ) to average particle diameter (e.g., Dv50).

Elastic modulus: As used herein, the term “elastic modulus” is used to refer to a measure of how much stress is needed to deform a gel, hydrocolloid, matrix, interpenetrating polymer network, or release modulator, without permanently changing its shape.

Encapsulant: As used herein, the term “encapsulant” is used to refer to anything that is used to encapsulate a payload. For example, in some embodiments of the present disclosure, a payload component (e.g., a nutrient component) is described as being encapsulated by an encapsulant (e.g., polymer component, food component, material component, etc.).

Encapsulated: As used herein, the term “encapsulated” is used to refer to a characteristic of being physically associated with, and in some embodiments partly or wholly covered or coated. For example, in some embodiments of the present disclosure, a payload component (e.g., a microbe component and/or a nutrient component) is described as being encapsulated by a polymer component.

Excipient: As used herein, the term “excipient” is used to refer to any substance in a composition and/or preparation other than one or more food components that is included in the manufacturing process of a composition and/or preparation or is contained in a final product. In some embodiment, an excipient includes a substance that stabilizes a composition and/or preparation (e.g., a stabilizer). In some embodiments, an excipient includes agents that prevent clumping (e.g., anti-caking agents). In some embodiments, an excipient includes a substance that binds or removes another substance (e.g., a sequestrant).

Extended-release: As used herein, the term “extended-release” is used to refer to a prolonged (i.e., lengthened) period of time during which a composition and/or formulation continues to release one or more components (e.g., one or more food components) beyond: a pre-determined time, a time in which the one or more components would naturally be released, a time in which the one or more components would be released in the absence of a manipulation, or a combination thereof. In some embodiments, “extended-release” refers to release of a food component (e.g., payload) over a period of at least 2 hours, at least 4 hours, at least 6 hours, at least 12 hours, at least 18 hours, or at least 24 hours,

Extended-release Product: As used herein, the term “extended-release product” is used to refer to a ingestible product (e.g., formulated food or formulated beverage) incorporating one or more formulated composition and/or formulated preparation that provides health benefits (e.g., gastrointestinal health, cardiovascular health, neurological health, endocrine health, musculoskeletal health, dermatological health, immune health; energy; nutrition; performance; general well-being) resulting from controlled, delayed, or extended release, absorption, spatial access, concentration, and/or residence time of nutrients.

Flowability: As used herein, the term “flowability” is used to refer to the ability a material, substance, composition, or preparation to move or flow.

Food: As used herein, the term “food” is used to refer to an edible solid (e.g., that can be ingested, swallowed, chewed, or consumed by a person or animal without material risk to the person or animal). For example, food include, but is not limited to, protein powder, whey powder, baby formula, dry food powder, protein bar, snack bar, yogurt, supplements, bread, candy, cake, cereal, chip, chocolate, confectionary, cookie, food additive, gummy, ice cream, kefir, rice, pasta, dry food, nutrition supplement, packaged food, pet feed, pet food, protein powder, sachet, salad dressing, salty snack, seed, smoothie, spice, Meal Ready-to-Eat (MRE), frozen food, medical food, fermented food, protein shakes, drink mix powders, dairy milk, oat milk, almond milk, soy milk, coffee grinds, Meal Ready-to-Drink (RTD), ready-to-drink low phenylalanine medical food, electrolyte beverages, sports beverages, water, hard seltzers, alcoholic seltzers, beer, wine, soda, coffee, tea, fermented beverages, carbonated beverage, Gatorade™, Truly™, Ensure™, PKU Sphere™ Liquid. In some embodiments, a “food” may be or comprise a pharmaceutical formulation in solid form. In some embodiments, a “food” may generally refer to a food and/or beverage product. In some embodiments, a “food” may generally refer to an edible object that is intended to confer a benefit to a subject (e.g., gastrointestinal health, cardiovascular health, neurological health, endocrine health, musculoskeletal health, dermatological health, immune health; energy; nutrition; performance; general well-being).

Food Component: As used herein, the term “food component” is used to refer to a component of a food and/or beverage that may or may not provide a nutritional benefit to a subject when consumed. In some embodiments, a food component may include a protein, a polypeptide, a peptide, an amino acid, a carbohydrate, a sugar, a monosaccharide, a polysaccharide, a dietary fiber, a fat, a fatty acid, a lipid, a nutrient, a nutraceutical, a macronutrient, a micronutrient, a vitamin, a mineral, a polypeptide, a carotenoid, an element, a ketone body, a prebiotic (e.g., a prebiotic fiber), a flavonoid, an antioxidant, an electrolyte, a salt, a circadian rhythm modulator, a supplement, a nootropic, an energy source, or a combination thereof

Food and/or Beverage Product: As used herein, the term “food and/or beverage product,” refers to an ingestible food, and/or combination of one or more ingestible foods, that is commercially available to a subject. Example food and/or beverage products include, but are not limited to, protein powder, whey powder, baby formula, dry food powder, protein bar, snack bar, yogurt, supplements, bread, candy, cake, cereal, chip, chocolate, confectionary, cookie, food additive, gummy, ice cream, kefir, rice, pasta, dry food, nutrition supplement, packaged food, pet feed, pet food, protein powder, sachet, salad dressing, salty snack, seed, smoothie, spice, Meal Ready-to-Eat (MRE), frozen food, medical food, fermented food, protein shakes, drink mix powders, dairy milk, oat milk, almond milk, soy milk, coffee grinds, Meal Ready-to-Drink (RTD), ready-to-drink low phenylalanine medical food, electrolyte beverages, sports beverages, water, hard seltzers, alcoholic seltzers, beer, wine, soda, coffee, tea, fermented beverages, carbonated beverage, Gatorade™, Truly™, Ensure™, PKU Sphere™ Liquid.

Formulated Beverages: As used herein, the term “formulated beverages” is used to refer to an ingestible liquid (e.g., that can be ingested, swallowed, drank, or consumed by a person or animal without material risk to the person or animal) that provides health benefits (e.g., gastrointestinal health, cardiovascular health, neurological health, endocrine health, musculoskeletal health, dermatological health, immune health; energy; nutrition; performance; general well-being) resulting from controlled, delayed, or extended release, absorption, spatial access, concentration, and/or residence time of nutrients. Examples of formulated beverages include, but are not limited to, protein shakes, drink mix powders, dairy milk, oat milk, almond milk, soy milk, coffee grinds, Meal Ready-to-Drink (RTD), ready-to-drink low phenylalanine medical food, electrolyte beverages, sports beverages, water, hard seltzers, alcoholic seltzers, beer, wine, soda, coffee, tea, fermented beverages, carbonated beverage, Gatorade™, Truly™, Ensure™, PKU Sphere™ Liquid. In some embodiments, a formulated beverage may incorporate a formulated composition and/or formulated preparation.

Formulated Foods: As used herein, the term “formulated foods” is used to refer to an edible solid (e.g., that can be ingested, swallowed, chewed, or consumed by a person or animal without material risk to the person or animal) that provides health benefits (e.g., gastrointestinal health, cardiovascular health, neurological health, endocrine health, musculoskeletal health, dermatological health, immune health; energy; nutrition; performance; general well-being) resulting from controlled, delayed, or extended release, absorption, spatial access, concentration, and/or residence time of nutrients. Examples of formulated foods include, but are not limited to, protein powder, whey powder, baby formula, dry food powder, protein bar, snack bar, yogurt, supplements, bread, candy, cake, cereal, chip, chocolate, confectionary, cookie, food additive, gummy, ice cream, kefir, rice, pasta, dry food, nutrition supplement, packaged food, pet feed, pet food, protein powder, sachet, salad dressing, salty snack, seed, smoothie, spice, Meal Ready-to-Eat (MRE), frozen food, medical food, fermented food, protein shakes, drink mix powders, dairy milk, oat milk, almond milk, soy milk, coffee grinds, Meal Ready-to-Drink (RTD), ready-to-drink low phenylalanine medical food, electrolyte beverages, sports beverages, water, hard seltzers, alcoholic seltzers, beer, wine, soda, coffee, tea, fermented beverages, carbonated beverage, Gatorade™, Truly™, Ensure™, PKU Sphere™ Liquid.

Formulated Composition: As used herein, the term “formulated composition” (also referred to as a “food and/or beverage composition,” “formulated ingestible,” or “extended-release composition”) is used to refer to an edible mixture of two or more components (e.g., one or more food components and one or more release modulators) that can be ingested, drank, swallowed, chewed, or consumed by a subject (e.g., person or animal) without material risk to the subject and that provides health benefits (e.g., gastrointestinal health, cardiovascular health, neurological health, endocrine health, musculoskeletal health, dermatological health, immune health; energy; nutrition; performance; general well-being) resulting from controlled, delayed, or extended release, absorption, spatial access, concentration, and/or residence time of nutrients. In some embodiments, a formulated composition may be in the form of a particle, microparticle, coated particle, coated, microparticle, core-shell, or a matrix.

Formulated Meals: As used herein, the term “formulated meals” is used to refer to a solid meal (e.g., that can be ingested, swallowed, chewed, or consumed by a person or animal without material risk to the person or animal) such as microwavable meals, freshly prepared meals, frozen meals, MREs, etc., that provides health benefits (e.g., gastrointestinal health, cardiovascular health, neurological health, endocrine health, musculoskeletal health, dermatological health, immune health; energy; nutrition; performance; general well-being) resulting from controlled, delayed, or extended release, absorption, spatial access, concentration, and/or residence time of nutrients. In some embodiments, a formulated meal may incorporate a formulated composition and/or formulated preparation.

Formulated Preparation: As used herein, the term “formulated preparation” is used to refer to an edible mixture of two or more formulated compositions (e.g., two or more particles, microparticles, coated particles, coated microparticles, core-shells, or matrices) that can be ingested, drank, swallowed, chewed, or consumed by a subject (e.g., person or animal) without material risk to the subject and that provides health benefits (e.g., gastrointestinal health, cardiovascular health, neurological health, endocrine health, musculoskeletal health, dermatological health, immune health; energy; nutrition; performance; general well-being) resulting from controlled, delayed, or extended release, absorption, spatial access, concentration, and/or residence time of nutrients. In some embodiments, a formulated composition may be in the form of a particle, microparticle, coated particle, coated, microparticle, core-shell, or a matrix. In some embodiments, a formulated preparation may include one or more excipients, probiotics, prebiotics, postbiotics, or additional components.

Formulated Supplements: As used herein, the term “formulated supplements” is used to refer to an edible dosage form (e.g., that can be ingested, swallowed, chewed, or consumed by a subject (e.g., a person or animal) without material risk to the subject) such as a powder (e.g., protein powder, whey protein powder, etc.), pill, capsule, tablet, etc. that provides health benefits (e.g., gastrointestinal health, cardiovascular health, neurological health, endocrine health, musculoskeletal health, dermatological health, immune health; energy; nutrition; performance; general well-being) resulting from controlled, delayed, or extended release, absorption, spatial access, concentration, and/or residence time of nutrients.

Formulation: As used herein, the term “formulation” is used to refer to a combination of two or more components, where the amounts and/or relative proportions of the two or more components is known and/or determined.

Gelator: As used herein, the term “gelator” refers to a substance that forms a three-dimensional network (i.e., a gel) that traps a large volume of solvent. As used herein, the term “gelator” further refers to a mixture of dicalcium phosphate and gluconolactone that forms a gel.

Hard Seltzers: As used herein, the term “hard seltzer” is used to refer to an ingestible liquid that contains alcohol and carbonated water.

HLB: As used herein, the term “HLB” is used to refer to the hydrophilic lipophilic balance that is an inherent property of, for example, a nonionic surfactant. In some embodiments, the HLB value of a given non-ionic surfactant is obtained from a commonly accessible tabular source. In some embodiments, non-ionic surfactants characterized as having a low HLB value (e.g., <8) are compatible emulsifiers for lipid systems. In some embodiments, nonionic surfactants characterized as having a high HLB value (e.g., >15) are compatible emulsifiers for aqueous systems. In some embodiments, non-ionic surfactants characterized as having an intermediate HLB value (e.g., >8 and <15) are compatible emulsifiers with both lipid and aqueous systems.

Homogeneous: As used herein, the term “homogeneous” means of substantially uniform structure and/or composition throughout.

Hydrocolloid: As used herein, the term “hydrocolloid” refers to a group of polysaccharides and/or proteins that form gels or viscous solutions in water. As used herein, the term “hydrocolloid” further refers to a mixture of pectins, alginates, and carrageenans.

Hydrophobic: As used herein, the term “hydrophobic” is used to refer to the propensity of a material to reject association, chemically and/or physically, with water. In some embodiments, a material characterized as being hydrophobic is biologically derived and/or synthetically derived. In some embodiments, a material characterized as being hydrophobic is a lipid, protein, and/or carbohydrate. In some embodiments, a material characterized as being hydrophobic is a polymer and/or small molecule. Alternatively, or additionally, in some embodiments, composites, mixtures, blends, or super-structures of several materials are collectively referred to as hydrophobic based on their observed propensity to reject association, chemically and/or physically, with water.

Incorporation: As used herein, the term “incorporation” is used to refer to a characteristic of being physically associated with, and in some embodiments, dispersed within, embedded within, or mixed in a bulk material (e.g., a lipid matrix component).

Layer: As used herein, the term “layer” typically refers to a material disposed above or below a distinguishable material. In some embodiments, a particular entity or preparation (e.g., particle preparation) is described as “layered” if it is prepared via a process in which a first material is laid down and then a second material is applied atop or underneath the first material (e.g., as by dipping or spraying, etc.); in some such embodiments, physical or chemical distinctness of layers may be maintained over time, whereas in some such embodiments, physical or chemical distinctness of layers may decay over time, at least at layer interface(s). Alternatively or additionally, in some embodiments, a particular sample or preparation may be described as layered, independent of its mode of preparation, so long as at a particular point in time and/or using a particular mode of assessment, distinct materials can be identified in a layered structure. In some embodiments, a “layered” particle may include one or more layers that wholly encapsulate a material below. In some embodiments, a “layered” particle may include one or more layers that does not wholly encapsulate a material below. In some embodiments, at least one layer of a layered preparation is or comprises a polymer, e.g., a hydrophobic polymer or hydrophilic polymer. In some embodiments, each layer of a layered preparation is or comprises a polymer, e.g., a pH responsive polymer or a temperature-responsive polymer.

Lipid: As used herein, the term “lipid” is used to refer to a class of chemical structures characterized as hydrophobic materials. In some embodiments, a lipid material is derived from a biological source. In other embodiments, a lipid material is derived from a synthetic source. In some embodiments, a lipid is comprised of one or more aliphatic alcohols and/or acids linked by glycerol and/or glycol moieties. In other embodiments, a lipid is comprised of aliphatic chains, linear conjugated, aromatic, and/or cyclic aliphatic moieties. In some embodiments, a lipid refers to a pure chemical entity. In other embodiments, a lipid refers to a mixture of several pure chemical entities. For example, lipids include, but are not limited to: paraffin wax, montan wax, microcrystalline wax, polyethylene wax, petrolatum wax, ozokerite wax, ceresin wax, beeswax, lanolin wax, spermaceti wax, tallow wax, lac wax, Chinese insect wax, ambergris wax, soy wax, carnauba wax, candelilla wax, coconut wax, palm kernel wax, rice bran wax, butyric acid, n-butanol, pentanoic acid, n-pentanol, hexanoic acid, n-hexanol, heptanoic acid, n-heptanol, caprylic acid, n-octanol, nonanoic acid, n-nonanol, capric acid, n-decanol, lauric acid, n-dodecanol, myristic acid, n-tetradecanol, palmitic acid, n-hexadecanol, stearic acid, n-octadecanol, arachidonic acid, n-icosanol, fatty alcohol monoglyceride ethers, fatty acid monoglyceride esters, fatty alcohol diglyceride ethers, fatty acid diglyceride esters, fatty alcohol triglyceride ethers, fatty acid triglyceride esters, fatty alcohol glycol monoether, fatty acid glycol monoesters, fatty alcohol glycol diethers, fatty acid glycol diesters, fatty alcohol poly(glycerol) ethers, fatty acid poly(glycerol) esters, fatty alcohol poly(glycol) ethers, fatty acid poly(glycol) esters, coconut oil, corn oil, cottonseed oil, olive oil, palm oil, peanut oil, rapeseed oil, safflower oil, sesame oil, soybean oil, sunflower oil, almond oil, pine nut oil, cashew oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, cholesterol, cholenic acid, ursolic acid, or betulinic acid.

Lyophilized: As used herein, the term “lyophilized” is used to refer to the end-product of a process by which water is removed from a material via sublimation. In some embodiments, prior to sublimation of water, the material is cooled to <−10° C., <−20° C., <−30° C., <−40° C., <−50° C., <−60° C., and/or <−70° C. In some embodiments, prior to the sublimation of water, the pressure is lowered to <200 torr, <150 torr, <100 torr, <50 torr, <10 torr, <5 torr, and/or <1 torr. Those skilled in the art recognize that the cooling temperature and pressure influence the physicochemical properties of the end product; it is understood that “lyophilized” encompasses all suitable manners of cooling and vacuum protocol.

Medical Foods: As used herein, the term “medical foods” is used to refer to an edible dosage form (e.g., that can be ingested, swallowed, chewed, or consumed by a person or animal without material risk to the person or animal) such as a pill, capsule, tablet, etc. that provides health benefits resulting from controlled release, absorption, spatial access, concentration, and/or residence time of nutrients.

Nutraceutical: As used herein, the terms “nutraceutical” or “nutraceutical composition” refer to a substance or material that is or comprises a nutraceutical agent (e.g., a nutraceutical). Those skilled in the art will be aware of a variety of agents understood in the art to be nutraceutical agents such as, for example, agents that are or comprise one or more antioxidants, macronutrients, micronutrients, minerals, prebiotics, probiotics, probiotic powders, probiotic ingredients, probiotic food ingredients, probiotic supplement ingredients, prebiotics, vitamins, or combinations thereof. In some embodiments, a nutraceutical is or comprises a carotenoid compound such as alpha-lipoic acid, astaxanthin, adonixanthin, adonirubin, beta-carotene, coenzyme Q10, lutein, lycopene, or zeaxanthin. In some embodiments, a nutraceutical is or comprises a vitamin such as vitamin D. In some embodiments, a nutraceutical is a natural product, and in certain such embodiments it is a product produced by plants. In some embodiments, nutraceutical agents are compounds that have been reported or demonstrated to confer a benefit or provide protection against a disease in an animal or a plant. In some cases, nutraceuticals may be used to improve health, delay the aging process, protect against chronic diseases, increase life expectancy, or support the structure or function of the body of an animal, such as a human, a pet animal, an agricultural animal, or another domesticated animal. As such, as used in the present disclosure, the terms “nutraceutical composition,” “food preparation,” “food composition,” “particle preparation,” etc. may all be generally understood to describe compositions, preparations, and/or particles that include one or more food components (for example, encapsulated food component(s)).

Nutrient: As used herein, the term “nutrient” is used to refer to a nutraceutical, a macronutrient, a carbohydrate, a sugar, a polysaccharide, a dietary fiber, a fat, a fatty acid, a lipid, a short-chain fatty acid, a protein, an amino acid, a peptide, a micronutrient, a vitamin, a mineral, a carotenoid, an element, a ketone body, a prebiotic, a probiotic, a postbiotic, a bacteria, a yeast, a polyphenol, a flavonoid, an antioxidant, an electrolyte, a salt, a circadian rhythm modulator, a supplement, a nootropic, and/or a source of energy.

Overfortification: As used herein, the term “overfortification” is used to refer to the addition of a nutrient in excess of a label claim, due to expected nutrient degradation or loss of stability during manufacturing, processing, and/or shelf-life. This additional amount of nutrient is used to account for losses during manufacturing, processing, and/or shelf-life to still meet the nutrient label claim.

Particle: As used herein, the term “particle” is used to refer to a discrete physical entity, typically having a size (e.g., a longest cross-section, such as a diameter) within a range. For example, a particle can have a size of about 5-3000 μm, about 5-2000 μm, about 5-1000 μm, about 5-500 μm, about 5-50 μm, about 5-300 μm, about 5-200 μm, about 5-100 μm, about 5-50 μm, about 5-25 μm, or about 5-10 μm. In some embodiments, a particle may describe or include animal pellets ranging in size up to 1 mm, 5 mm, 10 mm, 25 mm, and even about 50 mm (about 2 inches) in diameter. A “particle” is not limited to a particular shape or form, for example, having a cross-section shape of a sphere, an oval, a triangle, a square, a hexagon, or an irregular shape. In some cases, particles can be solid particles. In some cases, particles can be liquid particles. In some cases, particles can be gel or gel-like particles. In some cases, particles may have a particle-in-particle structure wherein a layer of one material (e.g., one type of polymer component) encapsulates another material (e.g., another type of polymer component, which may itself encapsulate yet another, or rather may be or comprise a “core”—e.g., a polymer matrix core—of the particle).

Parts per million (ppm): As used herein, 1 ppm (“parts per million”) is equivalent to 1 milligram per liter (mg/L) or 1 milligram per kilogram (mg/kg).

Payload: In general, the term “payload”, as used herein, refers to an agent that may be delivered or transported by association with another entity. In some embodiments, such association may be or include a covalent linkage; in some embodiments such association may be or include non-covalent interactions. In some embodiments, association may be direct; in some embodiments, association may be indirect. In some embodiments, a payload comprises one or more food components. The term “payload” is not limited to a particular chemical identity or type; for example, in some embodiments, a payload may be or comprise, for example, an entity of any chemical class including, for example, a nutrient, a lipid, a metal, a nucleic acid, a polypeptide, a saccharide (e.g., a polysaccharide), small molecule, or a combination or complex thereof. In some embodiments, a nutrient may include a lipid, a saccharide, a protein, etc. In some embodiments, a payload may be or comprise a biological modifier, a detectable agent (e.g., a dye, a fluorophore, a radiolabel, etc.), a detecting agent, a nutrient, a therapeutic agent, etc., or a combination thereof. In some embodiments, a payload may be or comprise a cell or organism, or a fraction, extract, or component thereof. In some embodiments, a payload may be or comprise a natural product in that it is found in and/or is obtained from nature; alternatively or additionally, in some embodiments, the term may be used to refer to one or more entities that is man-made in that it is designed, engineered, and/or produced through action of the hand of man and/or is not found in nature. In some embodiments, a payload may be or comprise an agent in isolated or pure form; in some embodiments, such agent may be in crude form.

pH Responsive: The term “pH-responsive” is used to refer to certain polymer components as described herein, and in particular means that the relevant polymer component is characterized in that one or more aspects of its structure or arrangement is altered when exposed to a change in pH condition (e.g., to a particular pH and/or to a pH change of particular magnitude). In some embodiments, a polymer component is considered to be “pH-responsive” if, when the relevant polymer component is associated with a payload component in a particle preparation as described herein, the particle preparation releases the payload component under specific pH conditions. In some embodiments, >90% of payload component is released from a particle preparation that includes a pH-responsive polymer component within 15 minutes when the particle preparation is exposed to a particular defined pH condition (e.g., within a range of defined pH values and/or at a specific pH value); in some embodiments, such release results when such contacting occurs at temperatures between 33-40° C., and in aqueous-based buffers of ionic strength ranging from 0.001-0.151 M (e.g., water, simulated gastric fluid, gastric fluid, simulated intestinal fluid, intestinal fluid) with osmolality between 1-615 mOsm/kg. In some embodiments, a pH-responsive polymer component is one that degrades when exposed to a particular pH or pH change. Alternatively or additionally, in some embodiments, a pH-responsive polymer component is one that becomes soluble, or significantly (e.g., (e.g., by at least about 5%) increases its solubility when exposed to a particular pH level, or pH change. In some embodiments, a pH-responsive polymer component includes one or more moieties whose protonation state changes at the relevant pH or in response to the relevant pH change. For example, in some embodiments, a pH responsive polymer component includes one or more amine moieties that become protonated upon exposure to a relevant pH or pH chance.

Polyphenols: As used herein, the term “polyphenol” is used to refer to naturally occurring organic compounds, comprising one or multiple aromatic groups with one or more hydroxyl groups or hydroxyl derivatives (e.g., methoxyl, ethoxyl, acetyl, etc.) and/or deriving from the shikimate, phenylpropanoid, and/or polyketide pathways. For example, a polyphenol may be phenolic acids, flavonoids, stilbenes, and lignans, antioxidants, tannins, and/or combinations thereof.

Prebiotic: As used herein, the term “prebiotic” is used to refer to a nondigestible food ingredient that promotes the growth of beneficial microorganisms in the intestines.

Reference: As used herein describes a standard or control relative to which a comparison is made. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Release: As used herein, the term “release” refers to the separation and solubilization of one or more food components into a dissolution solvent.

Release profile: As used herein, the term “release profile” refers to a characteristic pattern (e.g., a curve) by which one or more components (e.g., one or more food components) is released by a composition or formulation.

Residual solvent: As used herein, the term “residual solvent” refers to a solvent that remains in a material after manufacture or processing of the material. In some embodiments, level of residual solvent is assessed by HPLC, mass spec, NMR, FTIR, and/or gas chromatography.

Satiety: As used herein, the term “satiety” refers to being full and/or sated; for example, feeling satisfied due to ingestion of a food and/or beverage composition or having a desire removed following ingestion of a food and/or beverage composition.

Satisfaction: As used herein, the term “satisfaction,” refers to a sensation of contentment experienced by a subject.

Stable: The terms “stable,” and “stability,” when applied to compositions herein, means that the compositions maintain (e.g., as determined by one or more analytical assessments) one or more aspects of their physical structure and/or performance characteristic(s) (e.g., activity) over a period of time and/or under a designated set of conditions. When an assessed composition is a particle composition, in some embodiments, as will be clear from context to those skilled in the art, the term “stable” refers to maintenance of a characteristic such as average particle size, maximum and/or minimum particle size, range of particle sizes, and/or distribution of particle sizes (i.e., the percentage of particles above a designated size and/or outside a designated range of sizes) over a period of time and/or under a designated set of conditions. For food and/or beverage compositions, stable often refers to maintenance or preservation of delivery functions (e.g., controlled release, extended-release, controlled residence time, extended residence time, etc.).

Sustained release: As used herein, the term “sustained release” refers a period of time in which the release of one or more components (e.g., one or more food components) is continuous and at a relatively constant rate.

Temperature-responsive: As used herein, the term “temperature-responsive” is used to refer to certain polymer component(s) as described herein, and in particular means that the relevant polymer component is characterized in that one or more aspects of its structure or arrangement is altered when exposed to a change in temperature condition (e.g., to a particular temperature and/or to a temperature change of particular magnitude). In some embodiments, a polymer component is considered to be “temperature-responsive” if, when the relevant polymer component is associated with a payload component in a particle preparation as described herein, amorphous regions of the polymer component experience a transition from a rigid state (e.g., glassy state) to a more fluid-like flexible state (e.g., more conducive to flow), at a temperature close to the point of transition from the solid state to rubbery state (e.g., glass transition).

Water activity: As used herein, “water activity” of a material is an indication (e.g., a measurement) of how much free (i.e., available to bind or react) water is present in the material and is typically determined as the ratio of the vapor pressure of water in a material (p) to the vapor pressure of pure water (p0) at the same temperature. For example, a water activity of 0.80 means the vapor pressure is 80 percent of that of pure water. Water activity typically increases with temperature. Those skilled in the art will be familiar with three basic water activity measurement systems: Preventive Electrolytic Hygrometers (REH), Capacitance Hygrometers, and Dew Point Hygrometers (sometimes called chilled mirror).

Unformulated: As used herein, the term “unformulated,” is used to refer to a composition or component that has not been modified and/or combined with one or more other compositions or components.

Untreated: As used herein, the term “untreated,” refers to a composition or component (e.g., a food component) that has not been subject to a particular modification and/or manipulation (i.e., treatment).

### DETAILED DESCRIPTION OF CERTAIN EMBODIMENTS

The present disclosure provides formulated compositions for providing one of more food components to a subject and related technologies (e.g., methods).

### I. Formulated Compositions

The present disclosure, among other things, provides formulated compositions comprising one or more food components and one or more release modulators. In some embodiments, a formulated composition of the present disclosure is characterized as having controlled release of one or more food components from the formulated composition. In some embodiments, a formulated composition of the present disclosure is characterized as having extended-release of one or more food components from the formulated composition.

### A. Food Components

In some embodiments, a formulated composition of the present disclosure comprises one or more food components.

In some embodiments, a formulated composition of the present disclosure comprises 1 or more food components. In some embodiments, a formulated composition of the present disclosure comprises 1 to 25, 5 to 25, 10 to 25, 15 to 25, 20 to 25, 1 to 20, 5 to 20, 10 to 20, 15 to 20, 1 to 15, 5 to 15, 10 to 15, 1 to 10, 5 to 10, or 1 to 5 food components. For example, in some embodiments, a formulated composition comprises 1, 2, 3, 4, 5, 6, 7, 8, 9, 10, 11, 12, 13, 14, 15, 16, 17, 18, 19, 20, 21, 22, 23, 24, or 25 food components.

In some embodiments, a food component comprises a protein, a polypeptide, a peptide, an amino acid, a carbohydrate, a sugar, a monosaccharide, a polysaccharide, a dietary fiber, a fat, a fatty acid, a lipid, a nutrient, a nutraceutical, a macronutrient, a micronutrient, a vitamin, a mineral, a polypeptide, a carotenoid, an element, a ketone body, a prebiotic (e.g., a prebiotic fiber), a flavonoid, an antioxidant, an electrolyte, a salt, a circadian rhythm modulator, a supplement, a nootropic, an energy source, or a combination thereof.

In some embodiments, one or more food components comprise a protein (e.g., a protein payload). In some embodiments, one or more food components comprise whey protein isolate, whey protein concentrate, pea protein isolate, pea protein concentrate, oat protein isolate, oat protein concentrate, soy protein isolate, soy protein concentrate, wheat protein isolate, wheat protein concentrate, egg protein isolate, egg protein concentrate, dairy protein, plant-based protein, casein, bovine serum albumin, ovalbumin, α-lactalbumin, β-lactoglobulin, collagen, glutanin, gliadin, kefirin, avenin, zein, silk, gelatin, hordein, legumin, one or more digest fragments thereof, or a combination thereof.

In some embodiments, one or more food components comprise a peptide. In some embodiments, one or more food components comprise aspartame, GLP-1, GLP-2, collagen, sermorelin, tesamorelin, lenomorelin, anamorelin, ipamorelin, macimorelin, ghrelin, tabimorelin, alexamorelin, GHRP-1, GHRP-2, GHRP-3, GHRP-4, GHRP-5, GHRP-6, hexarelin, one or more digest fragments thereof, or a combination thereof.

In some embodiments, one or more food components comprise an amino acid. In some embodiments, one or more food components comprise alanine, arginine, asparagine, aspartic acid, cysteine, selenocysteine, glutamine, glutamic acid, glycine, histidine, isoleucine, leucine, lysine, methionine, phenylalanine, proline, serine, threonine, tryptophan, tyrosine, valine, norvaline, norleucine, pipecolic acid, ornithine, homocysteine, homoserine, isovaline, sarcosine, or a combination thereof.

In some embodiments, one or more food components comprise a carbohydrate. In some embodiments, one or more food components comprise glucose, fructose, mannitol, allulose, sorbitol, xylitol, erythritol, lactitol, galactose, sucrose, maltodextrin, isomaltulose, glycogen, chitosan, guar gum, pullulan, cellulose, dextrins, amylose, amylopectin, pectin, inulin, lignin, chitin, xanthan gum, sodium alginate, potassium alginate, calcium alginate, ammonium alginate, propylene glycol alginate, sodium hyaluronate, potassium hyaluronate, calcium hyaluronate, agar, agarose, carrageenan, raffinose, cellulose acetate, methyl cellulose, ethyl cellulose, cellulose acetate propionate, cellulose acetate butyrate, cellulose acetate succinate, hydroxypropyl methylcellulose, hydroxypropyl cellulose, sodium carboxymethylcellulose, or a combination thereof.

In some embodiments, one or more food components comprise a dietary fiber. In some embodiments, one or more food components comprise cellulose, dextrin, amylose, amylopectin, pectin, inulin, lignin, chitin, xanthan gum, sodium alginate, potassium alginate, calcium alginate, ammonium alginate, propylene glycol alginate, sodium hyaluronate, potassium hyaluronate, calcium hyaluronate, agar, agarose, carrageenan, raffinose, cellulose acetate, methyl cellulose, ethyl cellulose, cellulose acetate propionate, cellulose acetate butyrate, cellulose acetate succinate, hydroxypropyl methylcellulose, hydroxypropyl cellulose, sodium carboxymethylcellulose, naturally occurring fiber (e.g., vegetable fiber, whole grain fiber, fruit fiber, cereal bran fiber, flaked cereal fiber, flour fiber), β-glucan soluble fiber, psyllium husk, guar gum, locust bean gum, mixed plant cell wall fibers (e.g., sugar cane fiber, apple fiber), arabinoxylan, alginate, inulin-type fructans, high amylose starch, galactooligosaccharide, polydextrose, maltodextrin, cross-linked phosphorylated RS4, glucomannan, acacia (gum Arabic), or a combination thereof.

In some embodiments, one or more food components comprise a fat. In some embodiments, one or more food components comprise a paraffin wax, montan wax, microcrystalline wax, polyethylene wax, petrolatum wax, ozokerite wax, ceresin wax, beeswax, lanolin wax, spermaceti wax, tallow wax, lac wax, Chinese insect wax, ambergris wax, soy wax, carnauba wax, candelilla wax, coconut wax, palm kernel wax, rice bran wax, butyric acid, n-butanol, pentanoic acid, n-pentanol, hexanoic acid, n-hexanol, heptanoic acid, n-heptanol, caprylic acid, n-octanol, nonanoic acid, n-nonanol, capric acid, n-decanol, lauric acid, n-dodecanol, myristic acid, n-tetradecanol, palmitic acid, n-hexadecanol, stearic acid, n-octadecanol, arachidonic acid, n-icosanol, fatty alcohol monoglyceride ethers, fatty acid monoglyceride esters, fatty alcohol diglyceride ethers, fatty acid diglyceride esters, fatty alcohol triglyceride ethers, fatty acid triglyceride esters, fatty alcohol glycol monoether, fatty acid glycol monoesters, fatty alcohol glycol diethers, fatty acid glycol diesters, fatty alcohol poly(glycerol) ethers, fatty acid poly(glycerol) esters, fatty alcohol poly(glycol) ethers, fatty acid poly(glycol) esters, coconut oil, corn oil, cottonseed oil, olive oil, palm oil, peanut oil, rapeseed oil, safflower oil, sesame oil, soybean oil, sunflower oil, almond oil, pine nut oil, cashew oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, cholesterol, cholenic acid, ursolic acid, and/or betulinic acid.

In some embodiments, one or more food components comprise a fatty acid. In some embodiments, one or more food components comprise a short-chain fatty acid, medium-chain fatty acid, long-chain fatty acid, polyunsaturated fatty acid, docosahexaenoic acid, arachidonic acid, linoleic acid, linolenic acid, oleic acid, parinaric acid, rumenic acid, eicosapentaenoic acid, acetate, propionate, butyrate, or a combination thereof.

In some embodiments, one or more food components comprise a carotenoid. In some embodiments, one or more food components comprise alpha-lipoic acid, lycopene, β-carotene, lutein, zeaxanthin, adonixxanthin, adonirubin, meso-zeaxanthin, astaxanthin, capsanthin, citroxanthin, echinenone, astacein, bixin, crocetin, peridin, or a combination thereof.

In some embodiments, one or more food components comprise a vitamin. In some embodiments, one or more food components comprise trans-retinol, trans-β-carotene, thiamine, riboflavin, niacin, niacinamide, nicotinamide riboside, pantothenic acid, pyridoxine, pyridoxamine, pyridoxal, biotin, folic acid, cyanocobalamin, hydroxocobalamin, methylcobalamin, adenosylcobalamin, ascorbic acid, cholecalciferol, ergocalciferol, tocopherol, tocotrienol, phylloquinone, menaquinone, or a combination thereof.

In some embodiments, one or more food components comprise an antioxidant or polyphenol. In some embodiments, one or more food components comprise tannic acid, ellagitannin, apigenin, luteolin, tangeritin, isorhamnetin, kaempferol, myricetin, quercetin, rutin, eriodictyol, genipin, hesperetin, naringenin, catechin, gallocatechin, epicatechin, epigallocatechin, theaflavin, daidzein, genistein, glycitein, resveratrol, pterostilbene, hydroxytyrosol, cyanidin, delphinidin, malvidin, pelargonidin, peonidin, petunidin, chicoric acid, chlorogenic acid, cinnamic acid, ellagic acid, gallic acid, sinapic acid, rosmarinic acid, salicylic acid, curcumin, piperine, silymarin, silybin, eugenol, betanin, or a combination thereof.

In some embodiments, one or more food components comprise an element. In some embodiments, one or more food components comprise calcium, chromium, cobalt, copper, iodine, iron, magnesium, manganese, molybdenum, potassium, selenium, sodium, zinc, or a combination thereof.

In some embodiments, one or more food components comprise a salt and/or mineral. In some embodiments, one or more food components comprise sodium chloride, potassium chloride, iron oxide, calcium chloride, calcium carbonate, calcium hydroxyapatite, potassium iodide, sodium iodide, potassium fluoride, sodium fluoride, or a combination thereof.

In some embodiments, one or more food components comprise a cofactor. In some embodiments, one or more food components comprise nicotinamide adenine dinucleotide, flavin adenine dinucleotide, adenosine triphosphate, S-adenosylmethionine, Coenzyme Q, glutathione, heme, lipoamide, molybdopterin, tetrahydrobiopterin, or a combination thereof.

In some embodiments, one or more food components comprise a metabolic intermediate. In some embodiments, a metabolic intermediate is a ketone body. Those skilled in the art will appreciate that metabolic intermediates (e.g., ketone bodies) offer an alternative energy source to digestible carbohydrates, fats, and proteins. Without wishing to be bound by any particular theory, ingestion of a formulation of the present disclosure comprising one or more metabolic intermediates may provide energy (e.g., calories) to said subject while mitigating one or more metabolic responses (e.g., insulin release, drowsiness, fat storage). In some embodiments, one or more food components comprise acetoacetate, R-β-hydroxybutyl R-β-hydroxybutyrate, β-hydroxybutyrate, R-3-hydroxybutyl R-3-hydroxybutyrate monoester, 1,3-butanediol, or a combination thereof.

In some embodiments, one or more food components comprise a circadian rhythm modulator. In some embodiments, one or more food components comprise melatonin, methylcobalamin, adrafinil, cathine, cathinone, dextroamphetamine, ephedrine, epinephrine, armodafinil, modafinil, phenylethylamine, synephrine, theanine, 5-hydroxytryptophan, caffeine, theobromine, taurine, or a combination thereof.

In some embodiments, a formulated composition of the present disclosure comprises a complex mixture of one or more food components that recapitulates the food components, or a portion of food components, found in a prepared meal, cooked meat, cooked vegetables, cooed dairy products, raw meat, raw vegetables, raw dairy products, microbial products, or botanical extracts.

In some embodiments, one or more food components is purified using techniques including, but not limited to, liquid-liquid extraction, solid phase extraction, steam distillation, liquid chromatography, vacuum distillation, filtration, or a combination thereof.

In some embodiments, one or more food components is characterized as maintaining and/or supporting the health of a subject that has ingested said formulation. In some embodiments, maintaining and/or supporting the health of a subject includes, but is not limited to, supporting an endogenous enzymatic or metabolic process (e.g., metals, cofactors, vitamins), resisting oxidation (e.g., antioxidants), supporting bone and/or tooth health (e.g., minerals), supporting growth (e.g., amino acids, peptides, proteins), supporting digestive function (e.g., dietary fiber), supporting sensory tissue (e.g., carotenoids), supporting microbiome health (e.g., polyphenols, prebiotics), supporting circadian rhythm, supporting psychological well-being (e.g., lipids), supporting energy requirements (e.g., caloric intake), or a combination thereof.

In some embodiments, a formulated composition of the present disclosure comprises, on a dry weight basis, at least 50% of one or more food components. In some embodiments, a formulated composition comprises, on a dry weight basis, about 50% to 99%, about 55% to 99%, about 60% to 99%, about 65% to 99%, about 70% to 99%, about 75% to 99%, about 80% to 99%, about 85% to 99%, about 90% to 99%, about 95% to 99%, about 50% to 98%, about 55% to 98%, about 60% to 98%, about 65% to 98%, about 70% to 98%, about 75% to 98%, about 80% to 98%, about 85% to 98%, about 90% to 98%, about 95% to 98%, about 50% to 97%, about 55% to 97%, about 60% to 97%, about 65% to 97%, about 70% to 97%, about 75% to 97%, about 80% to 97%, about 85% to 97%, about 90% to 97%, about 95% to 97%, about 50% to 96%, about 55% to 96%, about 60% to 96%, about 65% to 96%, about 70% to 96%, about 75% to 96%, about 80% to 96%, about 85% to 96%, about 90% to 96%, about 95% to 96%, about 50% to 95%, about 55% to 95%, about 60% to 95%, about 65% to 95%, about 70% to 95%, about 75% to 95%, about 80% to 95%, about 85% to 95%, about 90% to 95%, 50% to 90%, about 55% to 90%, about 60% to 90%, about 65% to 90%, about 70% to 90%, about 75% to 90%, about 80% to 90%, about 85% to 90%, about 50% to 85%, about 55% to 85%, about 60% to 85%, about 65% to 85%, about 70% to 85%, about 75% to 85%, about 80% to 85%, about 50% to 80%, about 55% to 80%, about 60% to 80%, about 65% to 80%, about 70% to 80%, about 75% to 80%, about 50% to 75%, about 55% to 75%, about 60% to 75%, about 65% to 75%, about 70% to 75%, about 50% to 70%, about 55% to 70%, about 60% to 70%, about 65% to 70%, about 50% to 65%, about 55% to 65%, about 60% to 65%, about 50% to 60%, about 55% to 60%, or about 50% to 55% of one or more food components. For example, in some embodiments, a formulated composition comprises about 60% to 98% (w/w) of one or more food components.

For example, in some embodiments, a formulated composition of the present disclosure comprises about 50% (w/w), about 50.5% (w/w), about 51% (w/w), about 51.5% (w/w), about 52% (w/w), about 52.5% (w/w), about 53% (w/w), about 53.5% (w/w), about 54% (w/w), about 54.5% (w/w), about 55% (w/w), about 55.5% (w/w), about 56% (w/w), about 56.5% (w/w), about 57% (w/w), about 57.5% (w/w), about 58% (w/w), about 58.5% (w/w), about 59% (w/w), about 59.5% (w/w), about 60% (w/w), about 60.5% (w/w), about 61% (w/w), about 61.5% (w/w), about 62% (w/w), about 62.5% (w/w), about 63% (w/w), about 63.5% (w/w), about 64% (w/w), about 64.5% (w/w), about 65% (w/w), about 65.5% (w/w), about 66% (w/w), about 66.5% (w/w), about 67% (w/w), about 67.5% (w/w), about 68% (w/w), about 68.5% (w/w), about 69% (w/w), about 69.5% (w/w), about 70% (w/w), about 70.5% (w/w), about 71% (w/w), about 71.5% (w/w), about 72% (w/w), about 72.5% (w/w), about 73% (w/w), about 73.5% (w/w), about 74% (w/w), about 74.5% (w/w), about 75% (w/w), about 75.5% (w/w), about 76% (w/w), about 76.5% (w/w), about 77% (w/w), about 77.5% (w/w), about 78% (w/w), about 78.5% (w/w), about 79% (w/w), about 79.5% (w/w), about 80% (w/w), about 80.5% (w/w), about 81% (w/w), about 81.5% (w/w), about 82% (w/w), about 82.5% (w/w), about 83% (w/w), about 83.5% (w/w), about 84% (w/w), about 84.5% (w/w), about 85% (w/w), about 85.5% (w/w), about 86% (w/w), about 86.5% (w/w), about 87% (w/w), about 87.5% (w/w), about 88% (w/w), about 88.5% (w/w), about 89% (w/w), about 89.5% (w/w), about 90% (w/w), about 90.5% (w/w), about 91% (w/w), about 91.5% (w/w), about 92% (w/w), about 92.5% (w/w), about 93% (w/w), about 93.5% (w/w), about 94% (w/w), about 94.5% (w/w), about 95% (w/w), about 95.5% (w/w), about 96% (w/w), about 96.5% (w/w), about 97% (w/w), about 97.5% (w/w), about 98% (w/w), about 98.5% (w/w), about 99% (w/w), or about 99.5% (w/w) of one or more food components.

In some embodiments, one or more food components is physically and/or chemically arranged in a formulated composition in a manner that controls the release of one or more food components from a formulation. In some embodiments, one or more food components is physically and/or chemically arranged in a formulated composition in a manner that extends the duration of release of one or more food components from the formulation.

In some embodiments, the physical and/or chemical properties of one or more food component controls the release of one or more food components from a formulation. In some embodiments, the physical and/or chemical properties of one or more food component extends the duration of release of one or more food components from a formulation. In some embodiments, the mucoadhesion of one or more food component controls the release of one or more food components from a formulation. In some embodiments, the mucoadhesion of one or more food component extends the duration of release of one or more food components from a formulation. In some embodiments, the bioadhesion of one or more food component controls the release of one or more food components from a formulation. In some embodiments, the bioadhesion of one or more food component extends the duration of release of one or more food components from a formulation. In some embodiments, the buoyancy of one or more food component controls the release of one or more food components from a formulation. In some embodiments, the buoyancy of one or more food component extends the duration of release of one or more food components from a formulation. In some embodiments, the physical size of one or more food component controls the release of one or more food components from a formulation. In some embodiments, the physical size of one or more food component extends the duration of release of one or more food components from a formulation. In some embodiments, the physical shape of one or more food component controls the release of one or more food components from a formulation. In some embodiments, the physical shape of one or more food component extends the duration of release of one or more food components from a formulation.

In some embodiments, the physical and/or chemical properties of one or more food component prevents the access of water to one or more food components in a formulated composition. In some embodiments, the physical and/or chemical properties of one or more food component prevents the access of an enzyme (e.g., digestive enzyme) to one or more food components in a formulated composition.

In some embodiments, the physical and/or chemical interaction of one food component with one or more release modulators controls the release of one or more food components from a formulation. In some embodiments, the physical and/or chemical properties of one food component with one or more release modulators extends the duration of release of one or more food components from a formulation.

In some embodiments, the physical and/or chemical interaction of one food component with one or more other food components controls the release of one or more food components from a formulation. In some embodiments, the physical and/or chemical properties of one food component with one or more other food components extends the duration of release of one or more food components from a formulation.

In some embodiments, one or more food components are responsive to one or more environmental factors, wherein the response controls the release of one or more food components from a formulation. In some embodiments, one or more food components are responsive to one or more environmental factors, wherein the response extends the duration of release of one or more food components from a formulation. In some embodiments, one or more environmental factors include, but is not limited to, pH, temperature, water, mechanical forces, chemicals (e.g., chemicals exogenous or endogenous to a subject that ingests a formulation of the present disclosure), enzymes (e.g., enzymes exogenous or endogenous to a subject that ingests a formulation of the present disclosure), and osmotic pressure.

In some embodiments, a formulated composition of the present disclosure comprises one or more food components in an amount sufficient to satisfy an hourly, daily, weekly, and/or monthly, nutritional requirement of a subject that has ingested the formulation. In some embodiments, a formulated composition of the present disclosure comprises one or more food components in an amount sufficient to satisfy an hourly, daily, weekly, and/or monthly caloric requirement of a subject that has ingested the formulation.

In some embodiments, a formulated composition of the present disclosure comprises one or more food components in an amount having a caloric value of about 250 kcal to 4500 kcal, about 500 kcal to 4500 kcal, about 750 kcal to 4500 kcal, about 1000 kcal to 4500 kcal, about 1250 kcal to 4500 kcal, about 1500 kcal to 4500 kcal, about 1750 kcal to 4500 kcal, about 2000 kcal to 4500 kcal, about 2250 kcal to 4500 kcal, about 2500 kcal to 4500 kcal, about 2750 kcal to 4500 kcal, about 3000 kcal to 4500 kcal, about 3250 kcal to 4500 kcal, about 3500 kcal to 4500 kcal, about 3750 kcal to 4500 kcal, about 4000 kcal to 4500 kcal, about 4250 kcal to 4500 kcal, about 250 kcal to 4000 kcal, about 500 kcal to 4000 kcal, about 750 kcal to 4000 kcal, about 1000 kcal to 4000 kcal, about 1250 kcal to 4000 kcal, about 1500 kcal to 4000 kcal, about 1750 kcal to 4000 kcal, about 2000 kcal to 4000 kcal, about 2250 kcal to 4000 kcal, about 2500 kcal to 4000 kcal, about 2750 kcal to 4000 kcal, about 3000 kcal to 4000 kcal, about 3250 kcal to 4000 kcal, about 3500 kcal to 4000 kcal, about 3750 kcal to 4000 kcal, about 250 kcal to 3500 kcal, about 500 kcal to 3500 kcal, about 750 kcal to 3500 kcal, about 1000 kcal to 3500 kcal, about 1250 kcal to 3500 kcal, about 1500 kcal to 3500 kcal, about 1750 kcal to 3500 kcal, about 2000 kcal to 3500 kcal, about 2250 kcal to 3500 kcal, about 2500 kcal to 3500 kcal, about 2750 kcal to 3500 kcal, about 3000 kcal to 3500 kcal, about 3250 kcal to 3500 kcal, about 250 kcal to 3000 kcal, about 500 kcal to 3000 kcal, about 750 kcal to 3000 kcal, about 1000 kcal to 3000 kcal, about 1250 kcal to 3000 kcal, about 1500 kcal to 3000 kcal, about 1750 kcal to 3000 kcal, about 2000 kcal to 3000 kcal, about 2250 kcal to 3000 kcal, about 2500 kcal to 3000 kcal, about 2750 kcal to 3000 kcal, about 250 kcal to 2500 kcal, about 500 kcal to 2500 kcal, about 750 kcal to 2500 kcal, about 1000 kcal to 2500 kcal, about 1250 kcal to 2500 kcal, about 1500 kcal to 2500 kcal, about 1750 kcal to 2500 kcal, about 2000 kcal to 2500 kcal, about 2250 kcal to 2500 kcal, about 250 kcal to 2000 kcal, about 500 kcal to 2000 kcal, about 750 kcal to 2000 kcal, about 1000 kcal to 2000 kcal, about 1250 kcal to 2000 kcal, about 1500 kcal to 2000 kcal, about 1750 kcal to 2000 kcal, about 250 kcal to 1500 kcal, about 500 kcal to 1500 kcal, about 750 kcal to 1500 kcal, about 1000 kcal to 1500 kcal, about 1250 kcal to 1500 kcal, about 250 kcal to 1000 kcal, about 500 kcal to 1000 kcal, about 750 kcal to 1000 kcal, or about 250 kcal to 500 kcal.

Those skilled in the art will recognize that caloric content can be a surrogate measure for the amount of energy provided by one or more food components. In some embodiments, caloric content can be quantified using calorimetry. In some embodiments, the caloric content of one or more food components is a measure of the theoretical maximum carbon content available for oxidation (e.g., to metabolically harness via aerobic respiration). Additionally, without wishing to be bound by any particular theory, it is contemplated that, while correlated, the caloric value of one or more food components is not necessarily indicative of a nutritional benefit.

In some embodiments, a formulated composition of the present disclosure comprises one or more food components in an amount sufficient to support microbiome health in a subject ingesting said formulation. Without wishing to be bound by any particular theory, promoting microbiome health includes supporting the growth, proliferation, and/or diversity of commensal microbiota of a subject ingesting a formulation of the present disclosure.

In some embodiments, a formulated composition of the present disclosure comprises one or more food components in an amount sufficient to promote osmotic balance when released into an anatomical compartment of a subject ingesting said formulation. Without wishing to be bound by any particular theory, promoting osmotic balance includes maintaining the content of electrolytes in bodily fluids (e.g., interstitial fluid, luminal fluid, blood, urine, tears, and/or sweat) of a subject ingesting a formulation of the present disclosure.

### 1. Heat-Treatment

In some embodiments, a formulated composition of the present disclosure comprises one or more food components that has been heat-treated.

In some embodiments, one or more food components is heated to about 60° C. to about 140° C. In some embodiments, one or more food components is heated to about 60° C. to about 130° C. In some embodiments, one or more food components is heated to about 60° C. to about 120° C. In some embodiments, one or more food components is heated to about 60° C. to about 110° C. In some embodiments, one or more food components is heated to about 60° C. to about 100° C. In some embodiments, one or more food components is heated to about 60° C. to about 95° C. In some embodiments, one or more food components is heated to about 60° C. to about 90° C. In some embodiments, one or more food components is heated to about 60° C. to about 80° C. In some embodiments, one or more food components is heated to about 60° C. to about 70° C. In some embodiments, one or more food components is heated to about 70° C. to about 140° C. In some embodiments, one or more food components is heated to about 70° C. to about 130° C. In some embodiments, one or more food components is heated to about 70° C. to about 120° C. In some embodiments, one or more food components is heated to about 70° C. to about 110° C. In some embodiments, one or more food components is heated to about 70° C. to about 100° C. In some embodiments, one or more food components is heated to about 70° C. to about 95° C. In some embodiments, one or more food components is heated to about 70° C. to about 90° C. In some embodiments, one or more food components is heated to about 70° C. to about 80° C. In some embodiments, one or more food components is heated to about 80° C. to about 140° C. In some embodiments, one or more food components is heated to about 80° C. to about 130° C. In some embodiments, one or more food components is heated to about 80° C. to about 120° C. In some embodiments, one or more food components is heated to about 80° C. to about 110° C. In some embodiments, one or more food components is heated to about 80° C. to about 100° C. In some embodiments, one or more food components is heated to about 80° C. to about 95° C. In some embodiments, one or more food components is heated to about 80° C. to about 90° C. In some embodiments, one or more food components is heated to about 90° C. to about 140° C. In some embodiments, one or more food components is heated to about 90° C. to about 130° C. In some embodiments, one or more food components is heated to about 90° C. to about 120° C. In some embodiments, one or more food components is heated to about 90° C. to about 110° C. In some embodiments, one or more food components is heated to about 90° C. to about 100° C. In some embodiments, one or more food components is heated to about 90° C. to about 95° C. In some embodiments, one or more food components is heated to about 95° C. to about 140° C. In some embodiments, one or more food components is heated to about 95° C. to about 130° C. In some embodiments, one or more food components is heated to about 95° C. to about 120° C. In some embodiments, one or more food components is heated to about 95° C. to about 110° C. In some embodiments, one or more food components is heated to about 95° C. to about 100° C. In some embodiments, one or more food components is heated to about 100° C. to about 140° C. In some embodiments, one or more food components is heated to about 100° C. to about 130° C. In some embodiments, one or more food components is heated to about 100° C. to about 120° C. In some embodiments, one or more food components is heated to about 100° C. to about 110° C. In some embodiments, one or more food components is heated to about 110° C. to about 140° C. In some embodiments, one or more food components is heated to about 110° C. to about 130° C. In some embodiments, one or more food components is heated to about 110° C. to about 120° C. In some embodiments, one or more food components is heated to about 120° C. to about 140° C. In some embodiments, one or more food components is heated to about 120° C. to about 130° C. In some embodiments, one or more food components is heated to about 130° C. to about 140° C.

In some embodiments, one or more food components is heated to about 85° C. In some embodiments, one or more food components is heated to about 90° C. In some embodiments, one or more food components is heated to about 92° C. In some embodiments, one or more food components is heated to about 94° C. In some embodiments, one or more food components is heated to about 95° C. In some embodiments, one or more food components is heated to about 96° C. In some embodiments, one or more food components is heated to about 85° C. In some embodiments, one or more food components is heated to about 100° C. In some embodiments, one or more food components is heated to about 105° C.

In some embodiments, one or more food components is heated for about 60 min to about 120 min. In some embodiments, one or more food components is heated for about 60 min to about 110 min. In some embodiments, one or more food components is heated for about 60 min to about 100 min. In some embodiments, one or more food components is heated for about 60 min to about 90 min. In some embodiments, one or more food components is heated for about 60 min to about 80 min. In some embodiments, one or more food components is heated for about 60 min to about 70 min. In some embodiments, one or more food components is heated for about 70 min to about 120 min. In some embodiments, one or more food components is heated for about 70 min to about 110 min. In some embodiments, one or more food components is heated for about 70 min to about 100 min. In some embodiments, one or more food components is heated for about 70 min to about 90 min. In some embodiments, one or more food components is heated for about 70 min to about 80 min. In some embodiments, one or more food components is heated for about 80 min to about 120 min. In some embodiments, one or more food components is heated for about 80 min to about 110 min. In some embodiments, one or more food components is heated for about 80 min to about 100 min. In some embodiments, one or more food components is heated for about 80 min to about 90 min. In some embodiments, one or more food components is heated for about 90 min to about 120 min. In some embodiments, one or more food components is heated for about 90 min to about 110 min. In some embodiments, one or more food components is heated for about 90 min to about 100 min. In some embodiments, one or more food components is heated for about 100 min to about 120 min. In some embodiments, one or more food components is heated for about 100 min to about 110 min. In some embodiments, one or more food components is heated for about 110 min to about 120 min.

In some embodiments, one or more food components is heated for about 60 min. In some embodiments, one or more food components is heated for about 70 min. In some embodiments, one or more food components is heated for about 80 min. In some embodiments, one or more food components is heated for about 90 min. In some embodiments, one or more food components is heated for about 100 min. In some embodiments, one or more food components is heated for about 110 min. In some embodiments, one or more food components is heated for about 120 min.

In some embodiments, one or more food components is heat-treated at a pH of about 5 to about 8. In some embodiments, one or more food components is heat-treated at a pH of about 5.5 to about 8. In some embodiments, one or more food components is heat-treated at a pH of about 6 to about 8. In some embodiments, one or more food components is heat-treated at a pH of about 6.5 to about 8. In some embodiments, one or more food components is heat-treated at a pH of about 7 to about 8. In some embodiments, one or more food components is heat-treated at a pH of about 7.5 to about 8. In some embodiments, one or more food components is heat-treated at a pH of about 5 to about 7.5. In some embodiments, one or more food components is heat-treated at a pH of about 5.5 to about 7.5. In some embodiments, one or more food components is heat-treated at a pH of about 6 to about 7.5. In some embodiments, one or more food components is heat-treated at a pH of about 6.5 to about 7.5. In some embodiments, one or more food components is heat-treated at a pH of about 7 to about 7.5. In some embodiments, one or more food components is heat-treated at a pH of about 5 to about 7. In some embodiments, one or more food components is heat-treated at a pH of about 5.5 to about 7. In some embodiments, one or more food components is heat-treated at a pH of about 6 to about 7. In some embodiments, one or more food components is heat-treated at a pH of about 6.5 to about 7. In some embodiments, one or more food components is heat-treated at a pH of about 5 to about 6.5. In some embodiments, one or more food components is heat-treated at a pH of about 5.5 to about 6.5. In some embodiments, one or more food components is heat-treated at a pH of about 6 to about 6.5. In some embodiments, one or more food components is heat-treated at a pH of about 5 to about 6. In some embodiments, one or more food components is heat-treated at a pH of about 5.5 to about 6. In some embodiments, one or more food components is heat-treated at a pH of about 5 to about 5.5.

In some embodiments, one or more food components is heat-treated at a pH of about 5. In some embodiments, one or more food components is heat-treated at a pH of about 5.5. In some embodiments, one or more food components is heat-treated at a pH of about 6. In some embodiments, one or more food components is heat-treated at a pH of about 6.3. In some embodiments, one or more food components is heat-treated at a pH of about 6.5. In some embodiments, one or more food components is heat-treated at a pH of about 6.8. In some embodiments, one or more food components is heat-treated at a pH of about 7. In some embodiments, one or more food components is heat-treated at a pH of about 7.5. In some embodiments, one or more food components is heat-treated at a pH of about 8.

In some embodiments, one or more food components comprises a heat-treated protein, polypeptide, peptide, amino acid, carbohydrate, sugar, monosaccharide, polysaccharide, dietary fiber, fat, fatty acid, lipid, nutrient, nutraceutical, macronutrient, micronutrient, vitamin, mineral, polypeptide, carotenoid, element, ketone body, prebiotic (e.g., prebiotic fiber), flavonoid, antioxidant, electrolyte, salt, circadian rhythm modulator, supplement, nootropic, or energy source.

In some embodiments, a formulated composition of the present disclosure comprises about 50% to 99% (w/w), about 55% to 99% (w/w), about 60% to 99% (w/w), about 65% to 99% (w/w), about 70% to 99% (w/w), about 75% to 99% (w/w), about 80% to 99% (w/w), about 85% to 99% (w/w), about 90% to 99% (w/w), about 95% to 99% (w/w), about 50% to 98% (w/w), about 55% to 98% (w/w), about 60% to 98% (w/w), about 65% to 98% (w/w), about 70% to 98% (w/w), about 75% to 98% (w/w), about 80% to 98% (w/w), about 85% to 98% (w/w), about 90% to 98% (w/w), about 95% to 98% (w/w), about 50% to 97% (w/w), about 55% to 97% (w/w), about 60% to 97% (w/w), about 65% to 97% (w/w), about 70% to 97% (w/w), about 75% to 97% (w/w), about 80% to 97% (w/w), about 85% to 97% (w/w), about 90% to 97% (w/w), about 95% to 97% (w/w), about 50% to 96% (w/w), about 55% to 96% (w/w), about 60% to 96% (w/w), about 65% to 96% (w/w), about 70% to 96% (w/w), about 75% to 96% (w/w), about 80% to 96% (w/w), about 85% to 96% (w/w), about 90% to 96% (w/w), about 95% to 96% (w/w), about 50% to 95% (w/w), about 55% to 95% (w/w), about 60% to 95% (w/w), about 65% to 95% (w/w), about 70% to 95% (w/w), about 75% to 95% (w/w), about 80% to 95% (w/w), about 85% to 95% (w/w), about 90% to 95% (w/w), 50% to 90% (w/w), about 55% to 90% (w/w), about 60% to 90% (w/w), about 65% to 90% (w/w), about 70% to 90% (w/w), about 75% to 90% (w/w), about 80% to 90% (w/w), about 85% to 90% (w/w), about 50% to 85% (w/w), about 55% to 85% (w/w), about 60% to 85% (w/w), about 65% to 85% (w/w), about 70% to 85% (w/w), about 75% to 85% (w/w), about 80% to 85% (w/w), about 50% to 80% (w/w), about 55% to 80% (w/w), about 60% to 80% (w/w), about 65% to 80% (w/w), about 70% to 80% (w/w), about 75% to 80% (w/w), about 50% to 75% (w/w), about 55% to 75% (w/w), about 60% to 75% (w/w), about 65% to 75% (w/w), about 70% to 75% (w/w), about 50% to 70% (w/w), about 55% to 70% (w/w), about 60% to 70% (w/w), about 65% to 70% (w/w), about 50% to 65% (w/w), about 55% to 65% (w/w), about 60% to 65% (w/w), about 50% to 60% (w/w), about 55% to 60% (w/w), or about 50% to 55% of one or more heat-treated food components. For example, in some embodiments, a formulated composition comprises about 60% to 98% (w/w) of one or more heat-treated food components.

In some embodiments, one or more food components comprises a heat-treated protein. In some embodiments, a heat treatment comprises heating an about 1% to 30% (w/v), 5% to 30% (w/v), 10% to 30% (w/v), 15% to 30% (w/v), 20% to 30% (w/v), 25% to 30% (w/v), 1% to 25% (w/v), 5% to 25% (w/v), 10% to 25% (w/v), 15% to 25% (w/v), 20% to 25% (w/v), 1% to 20% (w/v), 5% to 20% (w/v), 10% to 20% (w/v), 15% to 20% (w/v), 1% to 15% (w/v), 5% to 15% (w/v), 10% to 15% (w/v), 1% to 10% (w/v), 5% to 10% (w/v), or 1% to 5% (w/v) solution of protein. For example, in some embodiments, a heat treatment comprises heating an about 8% to 15% (w/v) solution of protein.

In some embodiments, a heat treatment comprises heating a protein solution to a temperature of about 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., 140° C. to 150° C., 50° C. to 100° C., 60° C. to 100° C., 70° C. to 100° C., 80° C. to 100° C., or 90° C. to 100° C. For example, in some embodiments, a heat treatment comprises heating a protein solution to a temperature of about 60° C. to 140° C.

In some embodiments, a heat treatment comprises heating a protein solution for about 20 min. to 160 min., 40 min. to 160 min., 60 min. to 160 min., 80 min. to 160 min., 100 min. to 160 min., 120 min. to 160 min., 140 min. to 160 min., 20 min. to 140 min., 40 min. to 140 min., 60 min. to 140 min., 80 min. to 140 min., 100 min. to 140 min., 120 min. to 140 min., 20 min. to 120 min., 40 min. to 120 min., 60 min. to 120 min., 80 min. to 120 min., 100 min. to 120 min., 20 min. to 100 min., 40 min. to 100 min., 60 min. to 100 min., 80 min. to 100 min., 20 min. to 80 min., 40 min. to 80 min., 60 min. to 80 min., 20 min. to 60 min., 40 min. to 60 min., or 20 min. to 40 min. For example, in some embodiments, a heat treatment comprises heating a protein solution for about 60 min. to 120 min.

In some embodiments, a heat treatment comprises heating a protein solution at a pH of about 4 to 10, 5 to 10, 6 to 10, 7 to 10, 8 to 10, 9 to 10, 4 to 9, 5 to 9, 6 to 9, 7 to 9, 8 to 9, 4 to 8, 5 to 8, 6 to 8, 7 to 8, 4 to 7, 5 to 7, 6 to 7, 4 to 6, 5 to 6, or 4 to 5. For example, in some embodiments, a heat treatment comprises heating a protein solution at a pH of about 5 to 8.

In some embodiments, a heat treatment comprises heating an about 8% to 15% (w/v) solution of protein at pH 5 to 8, to a temperature of about 60° C. to 140° C. for about 60 min. to 120 min.

In some embodiments, a heat treatment comprises heating a protein solution that is left standing (e.g., no agitation). In some embodiments, a heat treatment comprises heating a protein solution that is agitated by techniques including, but not limited to, overhead stirring, high shear homogenization, high pressure homogenization, ultrasonic homogenization, magnetic stir bar, or a combination thereof.

Without wishing to be bound by any particular theory, in some embodiments, heat-treating a protein solution at a concentration of about 8% to 15% (w/v) in the presence of one or more volatile acids achieves formulated compositions characterized as having an extended duration of release of the heat-treated protein.

In some embodiments, a formulated composition of the present disclosure comprises about 50% to 99% (w/w), about 55% to 99% (w/w), about 60% to 99% (w/w), about 65% to 99% (w/w), about 70% to 99% (w/w), about 75% to 99% (w/w), about 80% to 99% (w/w), about 85% to 99% (w/w), about 90% to 99% (w/w), about 95% to 99% (w/w), about 50% to 98% (w/w), about 55% to 98% (w/w), about 60% to 98% (w/w), about 65% to 98% (w/w), about 70% to 98% (w/w), about 75% to 98% (w/w), about 80% to 98% (w/w), about 85% to 98% (w/w), about 90% to 98% (w/w), about 95% to 98% (w/w), about 50% to 97% (w/w), about 55% to 97% (w/w), about 60% to 97% (w/w), about 65% to 97% (w/w), about 70% to 97% (w/w), about 75% to 97% (w/w), about 80% to 97% (w/w), about 85% to 97% (w/w), about 90% to 97% (w/w), about 95% to 97% (w/w), about 50% to 96% (w/w), about 55% to 96% (w/w), about 60% to 96% (w/w), about 65% to 96% (w/w), about 70% to 96% (w/w), about 75% to 96% (w/w), about 80% to 96% (w/w), about 85% to 96% (w/w), about 90% to 96% (w/w), about 95% to 96% (w/w), about 50% to 95% (w/w), about 55% to 95% (w/w), about 60% to 95% (w/w), about 65% to 95% (w/w), about 70% to 95% (w/w), about 75% to 95% (w/w), about 80% to 95% (w/w), about 85% to 95% (w/w), about 90% to 95% (w/w), 50% to 90% (w/w), about 55% to 90% (w/w), about 60% to 90% (w/w), about 65% to 90% (w/w), about 70% to 90% (w/w), about 75% to 90% (w/w), about 80% to 90% (w/w), about 85% to 90% (w/w), about 50% to 85% (w/w), about 55% to 85% (w/w), about 60% to 85% (w/w), about 65% to 85% (w/w), about 70% to 85% (w/w), about 75% to 85% (w/w), about 80% to 85% (w/w), about 50% to 80% (w/w), about 55% to 80% (w/w), about 60% to 80% (w/w), about 65% to 80% (w/w), about 70% to 80% (w/w), about 75% to 80% (w/w), about 50% to 75% (w/w), about 55% to 75% (w/w), about 60% to 75% (w/w), about 65% to 75% (w/w), about 70% to 75% (w/w), about 50% to 70% (w/w), about 55% to 70% (w/w), about 60% to 70% (w/w), about 65% to 70% (w/w), about 50% to 65% (w/w), about 55% to 65% (w/w), about 60% to 65% (w/w), about 50% to 60% (w/w), about 55% to 60% (w/w), or about 50% to 55% of a heat-treated protein. For example, in some embodiments, a formulated composition comprises about 60% to 98% (w/w) of a heat-treated protein.

In some embodiments, a formulated composition of the present disclosure is heat-treated. In some embodiments, a formulated composition is heat-treated at about 70° C. to about 120° C., about 80° C. to about 120° C., about 90° C. to about 120° C., about 100° C. to about 120° C., about 110° C. to about 120° C., about 70° C. to about 110° C., about 80° C. to about 110° C., about 90° C. to about 110° C., about 100° C. to about 110° C., about 70° C. to about 100° C., about 80° C. to about 100° C., about 90° C. to about 100° C., about 70° C. to about 90° C., about 80° C. to about 90° C., or about 70° C. to about 80° C. For example, in some embodiments, a formulated composition is heat-treated at about 70° C., about 72° C., about 74° C., about 76° C., about 78° C., about 80° C., about 82° C., about 84° C., about 86° C., about 88° C., about 90° C., about 92° C., about 94° C., about 96° C., about 98° C., about 100° C., about 102° C., about 104° C., about 106° C., about 108° C., about 110° C., about 112° C., about 114° C., about 116° C., about 118° C., or about 120° C.

In some embodiments, a formulated composition is heat-treated for about 60 minutes to about 120 minutes, about 70 minutes to about 120 minutes, about 80 minutes to about 120 minutes, about 90 minutes to about 120 minutes, about 100 minutes to about 120 minutes, about 110 minutes to about 120 minutes, about 60 minutes to about 110 minutes, about 70 minutes to about 110 minutes, about 80 minutes to about 110 minutes, about 90 minutes to about 110 minutes, about 100 minutes to about 110 minutes, about 60 minutes to about 100 minutes, about 70 minutes to about 100 minutes, about 80 minutes to about 100 minutes, about 90 minutes to about 100 minutes, about 60 minutes to about 90 minutes, about 70 minutes to about 90 minutes, about 80 minutes to about 90 minutes, about 60 minutes to about 80 minutes, about 70 minutes to about 80 minutes, or about 60 minutes to about 70 minutes. For example, in some embodiments, a formulated composition is heat-treated for about 60 minutes, about 65 minutes, about 70 minutes, about 75 minutes, about 80 minutes, about 85 minutes, about 90 minutes, about 100 minutes, about 105 minutes, about 110 minutes, about 115 minutes, or about 120 minutes.

In some embodiments, a formulated composition is heat-treated at about pH 4 to about pH 8, about pH 4.5 to pH 8, about pH 5 to pH 8, about pH 5.5 to pH 8, about pH 6 to pH 8, about pH 6.5 to pH 8, about pH 7 to pH 8, about pH 7.5 to pH 8, about pH 4 to about pH 7.5, about pH 4.5 to pH 7.5, about pH 5 to pH 7.5, about pH 5.5 to pH 7.5, about pH 6 to pH 7.5, about pH 6.5 to pH 7.5, about pH 7 to pH 7.5, about pH 4 to about pH 7, about pH 4.5 to pH 7, about pH 5 to pH 7, about pH 5.5 to pH 7, about pH 6 to pH 7, about pH 6.5 to pH 7, about pH 4 to about pH 6.5, about pH 4.5 to pH 6.5, about pH 5 to pH 6.5, about pH 5.5 to pH 6.5, about pH 6 to pH 6.5, about pH 4 to about pH 6.5, about pH 4.5 to pH 6.5, about pH 5 to pH 6.5, about pH 5.5 to pH 6.5, about pH 6 to pH 6.5, about pH 4 to about pH 6, about pH 4.5 to pH 6, about pH 5 to pH 6, about pH 5.5 to pH 6, about pH 4 to about pH 5.5, about pH 4.5 to pH 5.5, about pH 5 to pH 5.5, about pH 4 to about pH 5, about pH 4.5 to pH 5, or about pH 4 to about pH 4.5. In some embodiments, a formulated composition is heat-treated at about pH 4, about pH 4.5, about pH 5, about pH 5.5, about pH 6, about pH 6.5, about pH 7, about pH 7.5, or about pH 8.

### B. Release Modulators

In some embodiments, a formulated composition of the present disclosure comprises one or more release modulators. In some embodiments, a release modulator can control the release of one or more food components from a formulated composition of the present disclosure. In some embodiments, a release modulator can extend the duration of release of one or more food components from a formulated composition of the present disclosure.

In some embodiments, a release modulator can interact with one or more food components to prevent water from accessing or interacting with the one or more food components. For example, in some embodiments, a release modulator can interact with a protein (e.g., a heat-treated protein) in a formulated composition to prevent water from accessing or interacting with one or more food components in said formulation.

In some embodiments, a release modulator can interact with one or more food components to prevent an enzyme (e.g., a digestive enzyme) from accessing or interacting with the one or more food components. For example, in some embodiments, a release modulator can interact with a protein (e.g., a heat-treated protein) in a formulated composition to prevent an enzyme (e.g., a digestive enzyme) from accessing or interacting with one or more food components in said formulation.

In some embodiments, a release modulator may comprise one or more release modulator components. In some embodiments, one or more release modulators and/or release modulator components comprise Pluronic™ F68, sucrose palmitate, triacetin, gum arabic, Tween™ 85, Span™ 60, glucose, dicalcium phosphate, calcium carbonate, gluconolactone, sodium alginate, low methoxyl pectins, shellac, hypromellose acetate succinate, rice starch, maltodextrins, oat fiber, inulin, carrageenans, or a combination thereof. In some embodiments, a release modulator is Pluronic™ F68. In some embodiments, a release modulator is sucrose palmitate. In some embodiments, a release modulator is glucose. In some embodiments, a release modulator is triacetin. In some embodiments, a release modulator is gum arabic. In some embodiments, a release modulator is Tween™ 85. In some embodiments, a release modulator is Span™ 60.

In some embodiments, a formulated composition of the present disclosure comprises one or more release modulators that chemically interact with one or more food components. For example, in some embodiments, one or more release modulators chemically interact with a heat-treated protein. In some embodiments, one or release modulators comprising hypromellose acetate succinate, rice starch, glucose, maltodextrins, oat fiber, inulin, carrageenans, or a combination thereof, chemically interact with a heat-treated protein (e.g., heat-treated whey protein isolate).

In some embodiments, a formulated composition of the present disclosure comprises 1 or more release modulators. In some embodiments, a formulated composition of the present disclosure comprises 1 to 25, 5 to 25, 10 to 25, 15 to 25, 20 to 25, 1 to 20, 5 to 20, 10 to 20, 15 to 20, 1 to 15, 5 to 15, 10 to 15, 1 to 10, 5 to 10, or 1 to 5 release modulators or release modulator components. For example, in some embodiments, a formulated composition of the present disclosure comprises 1, 2, 3, 4, 5, 6, 7, 8, 9, 10, 11, 12, 13, 14, 15, 16, 17, 18, 19, 20, 21, 22, 23, 24, or 25 release modulators or release modulator components.

In some embodiments, a formulated composition of the present disclosure comprises about 1% to 50% (w/w), 2% to 50%, 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 1% to 45% (w/w), 2% to 45%, 5% to 45% (w/w), 10% to 45% (w/w), 15% to 45% (w/w), 20% to 45% (w/w), 25% to 45% (w/w), 30% to 45% (w/w), 35% to 45% (w/w), 40% to 45% (w/w), 1% to 40% (w/w), 2% to 40%, 5% to 40% (w/w), 10% to 40% (w/w), 15% to 40% (w/w), 20% to 40% (w/w), 25% to 40% (w/w), 30% to 40% (w/w), 35% to 40% (w/w), 1% to 35% (w/w), 2% to 35%, 5% to 35% (w/w), 10% to 35% (w/w), 15% to 35% (w/w), 20% to 35% (w/w), 25% to 35% (w/w), 30% to 35% (w/w), 1% to 30% (w/w), 2% to 30%, 5% to 30% (w/w), 10% to 30% (w/w), 15% to 30% (w/w), 20% to 30% (w/w), 25% to 30% (w/w), 1% to 25% (w/w), 2% to 25%, 5% to 25% (w/w), 10% to 25% (w/w), 15% to 25% (w/w), 20% to 25% (w/w), 1% to 20% (w/w), 2% to 20%, 5% to 20% (w/w), 10% to 20% (w/w), 15% to 20% (w/w), 1% to 15% (w/w), 2% to 150%, 5% to 15% (w/w), 10% to 15% (w/w), 1% to 10% (w/w), 5% to 10% (w/w), 1% to 5% (w/w) or 2% to 5% (w/w) of one or more release modulators. For example, in some embodiments, a formulated composition of the present disclosure comprises about 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), or 50% (w/w) of one or more release modulators.

In some embodiments, one or more release modulators may be characterized as having one or more physicochemical properties (e.g., pH, viscosity, osmolarity, etc.) that are conducive to mixture with one or more food components (e.g., more homogeneous incorporation), long-term storage, incorporation into one or more food and/or beverage products (e.g., protein powder), or a combination thereof.

### 1. Interpenetrating Polymer Networks

In some embodiments, one or more release modulator comprises an interpenetrating polymer network. In some embodiments, two or more release modulator components form an interpenetrating polymer network. In some embodiments, one or more release modulator components capable of forming interpenetrating polymer networks comprise dicalcium phosphate, calcium carbonate, gluconolactone, sodium alginate, low methoxyl pectins, shellac, carrageenans, or a combination thereof. In some embodiments, an interpenetrating network comprises a hydrogel. In some embodiments, an interpenetrating polymer network comprises an alginate hydrogel. In some embodiments, an interpenetrating polymer network comprises a hydrogel and an additional release modular component. In some embodiments, an interpenetrating polymer network comprises a hydrogel and 2 additional release modular components. In some embodiments, an interpenetrating polymer network comprises a hydrogel and 3 additional release modular components. In some embodiments, an interpenetrating polymer network comprises a hydrogel and 4 additional release modular components. In some embodiments an interpenetrating polymer network may physically and/or chemically interact with one or more food components in a formulated composition. In some embodiments an interpenetrating polymer network does not physically and/or chemically interact with one or more food components in a formulated composition. In some embodiments, an interpenetrating network controls the release of one or more food components from a formulated composition of the present disclosure. In some embodiments, an interpenetrating network extends the duration of release of one or more food components from a formulated composition of the present disclosure.

In some embodiments, mixture of one or more food components with one or more release modulators forms a gel. In some embodiments, the elastic modulus of an aqueous mixture of one or more food components and one or more release modulators increases by less than 2-fold over a period of at least about 30 minutes to 12 hours, at least about 1 hour to 12 hours, at least about 2 hours to 12 hours, at least about 4 hours to 12 hours, at least about 6 hours to 12 hours, at least about 8 hours to 12 hours, or at least about 10 hours to 12 hours upon standing at a temperature of about 2° C. to about 30° C.

In some embodiments, the elastic modulus of an aqueous mixture of one or more food components and one or more release modulators increases by less than 4-fold over a period of at least about 30 minutes to 12 hours, at least about 1 hour to 12 hours, at least about 2 hours to 12 hours, at least about 4 hours to 12 hours, at least about 6 hours to 12 hours, at least about 8 hours to 12 hours, or at least about 10 hours to 12 hours upon standing at a temperature of about 2° C. to about 30° C.

In some embodiments, the elastic modulus of an aqueous mixture of one or more food components and one or more release modulators increases by less than 8-fold over a period of at least about 30 minutes to 12 hours, at least about 1 hour to 12 hours, at least about 2 hours to 12 hours, at least about 4 hours to 12 hours, at least about 6 hours to 12 hours, at least about 8 hours to 12 hours, or at least about 10 hours to 12 hours upon standing at a temperature of about 2° C. to about 30° C.

In some embodiments, the elastic modulus of an aqueous mixture of one or more food components and one or more release modulators increases by at least 2-fold over a period of at least about 30 minutes to 12 hours, at least about 1 hour to 12 hours, at least about 2 hours to 12 hours, at least about 4 hours to 12 hours, at least about 6 hours to 12 hours, at least about 8 hours to 12 hours, or at least about 10 hours to 12 hours upon standing at a temperature of about 2° C. to about 30° C.

In some embodiments, the elastic modulus of an aqueous mixture of one or more food components and one or more release modulators increases by at least 4-fold over a period of at least about 30 minutes to 12 hours, at least about 1 hour to 12 hours, at least about 2 hours to 12 hours, at least about 4 hours to 12 hours, at least about 6 hours to 12 hours, at least about 8 hours to 12 hours, or at least about 10 hours to 12 hours upon standing at a temperature of about 2° C. to about 30° C.

In some embodiments, the elastic modulus of an aqueous mixture of one or more food components and one or more release modulators increases by at least 8-fold over a period of at least about 30 minutes to 12 hours, at least about 1 hour to 12 hours, at least about 2 hours to 12 hours, at least about 4 hours to 12 hours, at least about 6 hours to 12 hours, at least about 8 hours to 12 hours, or at least about 10 hours to 12 hours upon standing at a temperature of about 2° C. to about 30° C.

In some embodiments, the elastic modulus of an aqueous mixture of one or more food components and one or more release modulators increases by at least 10-fold over a period of at least about 30 minutes to 12 hours, at least about 1 hour to 12 hours, at least about 2 hours to 12 hours, at least about 4 hours to 12 hours, at least about 6 hours to 12 hours, at least about 8 hours to 12 hours, or at least about 10 hours to 12 hours upon standing at a temperature of about 2° C. to about 30° C.

Without wishing to be bound by any particular theory, it is contemplated that the longer it takes for a mixture of one or more food components and one or more release modulators to increase its elastic modulus (i.e., gel), the more homogeneous the resulting interpenetrating polymer network.

In some embodiments, one or more food components and one or more release modulators are heat-treated together to form an interpenetrating polymer network. In some embodiments, a mixture of one or more food components and one or more release modulators is heated to about 85° C. to 105° C., 90° C. to 105° C., 95° C. to 105° C., 100° C. to 105° C., 85° C. to 100° C., 90° C. to 100° C., 95° C. to 100° C., 85° C. to 95° C., 90° C. to 95° C., or 85° C. to 90° C. For example, in some embodiments a mixture of one or more food components and one or more release modulators is heated to about 90° C., 91° C., 92° C., 93° C., 94° C., 95° C., 96° C., 97° C., 98° C., 99° C., or 100° C.

In some embodiments, a mixture of one or more food components and one or more release modulators is heated for about 1 hour to 3 hours, 1.5 hours to 3 hours, 2 hours to 3 hours, 2.5 hours to 3 hours, 1 hour to 2.5 hours, 1.5 hours to 2.5 hours, 2 hours to 2.5 hours, 1 hour to 2 hours, 1.5 hours to 2 hours, or 1 hour to 1.5 hours. For example, in some embodiments a mixture of one or more food components and one or more release modulators is heated for about 1 hours, 1.5 hours, 2 hours, 2.5 hours, or 3 hours.

In some embodiments, a mixture of one or more food components and one or more release modulators is heated without agitation. In some embodiments, a mixture of one or more food components and one or more release modulators is heated with agitation. In some embodiments, a mixture of one or more food components and one or more release modulators is heated in one or more batch processes. In some embodiments, a mixture of one or more food components and one or more release modulators is heated in a continuous process.

In some embodiments, a heat-treated protein (e.g., heat-treated whey protein isolated) is mixed with one or more release modulators to form a crosslinked (e.g., calcium crosslinked) gelatinous polymer network. In some embodiments, a mixture of a protein (e.g., a whey protein isolate) and one or more release modulators is heated to about 95° C. for about 2 hours to form an interpenetrating polymer network.

In some embodiments, a release modulator or release modulator component comprises a hydrocolloid. In some embodiments, a hydrocolloid comprises pectin, alginate, carrageenan, or a combination thereof.

In some embodiments, a formulated composition comprises about 5% (w/w) to about 50% (w/w), about 10% (w/w) to about 50% (w/w), about 15% (w/w) to about 50% (w/w), about 20% (w/w) to about 50% (w/w), about 25% (w/w) to about 50% (w/w), about 30% (w/w) to about 50% (w/w), about 35% (w/w) to about 50% (w/w), about 40% (w/w) to about 50% (w/w), about 45% (w/w) to about 50% (w/w), about 5% (w/w) to about 45% (w/w), about 10% (w/w) to about 45% (w/w), about 15% (w/w) to about 45% (w/w), about 20% (w/w) to about 45% (w/w), about 25% (w/w) to about 45% (w/w), about 30% (w/w) to about 45% (w/w), about 35% (w/w) to about 45% (w/w), about 40% (w/w) to about 45% (w/w), about 5% (w/w) to about 40% (w/w), about 10% (w/w) to about 40% (w/w), about 15% (w/w) to about 40% (w/w), about 20% (w/w) to about 40% (w/w), about 25% (w/w) to about 40% (w/w), about 30% (w/w) to about 40% (w/w), about 35% (w/w) to about 40% (w/w), about 5% (w/w) to about 35% (w/w), about 10% (w/w) to about 35% (w/w), about 15% (w/w) to about 35% (w/w), about 20% (w/w) to about 35% (w/w), about 25% (w/w) to about 35% (w/w), about 30% (w/w) to about 35% (w/w), about 5% (w/w) to about 30% (w/w), about 10% (w/w) to about 30% (w/w), about 15% (w/w) to about 30% (w/w), about 20% (w/w) to about 30% (w/w), about 25% (w/w) to about 30% (w/w), about 5% (w/w) to about 25% (w/w), about 10% (w/w) to about 25% (w/w), about 15% (w/w) to about 25% (w/w), about 20% (w/w) to about 25% (w/w), about 5% (w/w) to about 20% (w/w), about 10% (w/w) to about 20% (w/w), about 15% (w/w) to about 20% (w/w), about 5% (w/w) to about 15% (w/w), about 10% (w/w) to about 15% (w/w), about 5% (w/w) to about 10% (w/w), or about 5% (w/w) to about 10% (w/w) hydrocolloid. In some embodiments, a formulated composition comprises about 5% (w/w), about 10% (w/w), about 15% (w/w), about 20% (w/w), about 25% (w/w), about 30% (w/w), about 35% (w/w), about 40% (w/w), about 45% (w/w), or about 50% (w/w) hydrocolloid.

In some embodiments, a release modulator or release modulator component comprises a gelator. In some embodiments, a gelator comprises dicalcium phosphate, gluconolactone, calcium carbonate, or a combination thereof.

In some embodiments, a formulated composition comprises about 1% (w/w) to about 20% (w/w), about 2% (w/w) to about 20% (w/w), about 4% (w/w) to about 20% (w/w), about 6% (w/w) to about 20% (w/w), about 8% (w/w) to about 20% (w/w), about 10% (w/w) to about 20% (w/w), about 12% (w/w) to about 20% (w/w), about 14% (w/w) to about 20% (w/w), about 16% (w/w) to about 20% (w/w), about 18% (w/w) to about 20% (w/w), 1% (w/w) to about 15% (w/w), about 2% (w/w) to about 15% (w/w), about 4% (w/w) to about 15% (w/w), about 6% (w/w) to about 15% (w/w), about 8% (w/w) to about 15% (w/w), about 10% (w/w) to about 15% (w/w), about 12% (w/w) to about 15% (w/w), about 14% (w/w) to about 15% (w/w), about 1% (w/w) to about 10% (w/w), about 2% (w/w) to about 10% (w/w), about 4% (w/w) to about 10% (w/w), about 6% (w/w) to about 10% (w/w), about 8% (w/w) to about 10% (w/w), about 1% (w/w) to about 5% (w/w), about 2% (w/w) to about 5% (w/w), about 4% (w/w) to about 5%, or about 1% (w/w) to about 2% (w/w) gelator. In some embodiments, a formulated composition comprises about 1% (w/w), about 2% (w/w), about 4% (w/w), about 6% (w/w), about 8% (w/w), about 10% (w/w), about 12% (w/w), about 14% (w/w), about 16% (w/w), about 18% (w/w), or about 20% (w/w) gelator.

In some embodiments, a release modulator comprises about 7.5 parts (w/w) or less of hydrocolloid to about 1 part (w/w) of gelator (e.g., about 7.5 parts (w/w) of hydrocolloid to about 1 part (w/w) of gelator, about 7 parts (w/w) of hydrocolloid to about 1 part (w/w) of gelator, about 6.5 parts (w/w) of hydrocolloid to about 1 part (w/w) of gelator, about 6 parts (w/w) of hydrocolloid to about 1 part (w/w) of gelator, about 5.5 parts (w/w) of hydrocolloid to about 1 part (w/w) of gelator, about 5 parts (w/w) of hydrocolloid to about 1 part (w/w) of gelator, about 4.5 parts (w/w) of hydrocolloid to about 1 part (w/w) of gelator, about 4 parts (w/w) of hydrocolloid to about 1 part (w/w) of gelator, about 3.5 parts (w/w) of hydrocolloid to about 1 part (w/w) of gelator, about 3 parts (w/w) of hydrocolloid to about 1 part (w/w) of gelator, about 2.5 parts (w/w) of hydrocolloid to about 1 part (w/w) of gelator, about 2 parts (w/w) of hydrocolloid to about 1 part (w/w) of gelator, about 1.5 parts (w/w) of hydrocolloid to about 1 part (w/w) of gelator, or about 1 part (w/w) of hydrocolloid to about 1 part (w/w) of gelator).

In some embodiments, a formulated composition comprises about 5% (w/w) to about 50% (w/w), about 10% (w/w) to about 50% (w/w), about 15% (w/w) to about 50% (w/w), about 20% (w/w) to about 50% (w/w), about 25% (w/w) to about 50% (w/w), about 30% (w/w) to about 50% (w/w), about 35% (w/w) to about 50% (w/w), about 40% (w/w) to about 50% (w/w), about 45% (w/w) to about 50% (w/w), about 5% (w/w) to about 45% (w/w), about 10% (w/w) to about 45% (w/w), about 15% (w/w) to about 45% (w/w), about 20% (w/w) to about 45% (w/w), about 25% (w/w) to about 45% (w/w), about 30% (w/w) to about 45% (w/w), about 35% (w/w) to about 45% (w/w), about 40% (w/w) to about 45% (w/w), about 5% (w/w) to about 40% (w/w), about 10% (w/w) to about 40% (w/w), about 15% (w/w) to about 40% (w/w), about 20% (w/w) to about 40% (w/w), about 25% (w/w) to about 40% (w/w), about 30% (w/w) to about 40% (w/w), about 35% (w/w) to about 40% (w/w), about 5% (w/w) to about 35% (w/w), about 10% (w/w) to about 35% (w/w), about 15% (w/w) to about 35% (w/w), about 20% (w/w) to about 35% (w/w), about 25% (w/w) to about 35% (w/w), about 30% (w/w) to about 35% (w/w), about 5% (w/w) to about 30% (w/w), about 10% (w/w) to about 30% (w/w), about 15% (w/w) to about 30% (w/w), about 20% (w/w) to about 30% (w/w), about 25% (w/w) to about 30% (w/w), about 5% (w/w) to about 25% (w/w), about 10% (w/w) to about 25% (w/w), about 15% (w/w) to about 25% (w/w), about 20% (w/w) to about 25% (w/w), about 5% (w/w) to about 20% (w/w), about 10% (w/w) to about 20% (w/w), about 15% (w/w) to about 20% (w/w), about 5% (w/w) to about 15% (w/w), about 10% (w/w) to about 15% (w/w), about 5% (w/w) to about 10% (w/w), or about 5% (w/w) to about 10% (w/w) alginate. In some embodiments, a formulated composition comprises about 5% (w/w), about 10% (w/w), about 15% (w/w), about 20% (w/w), about 25% (w/w), about 30% (w/w), about 35% (w/w), about 40% (w/w), about 45% (w/w), or about 50% (w/w) alginate.

In some embodiments, a formulated composition comprises about 0.5% (w/w) to about 1.5% (w/w), about 0.75% (w/w) to about 1.5% (w/w), about 1% (w/w) to about 1.5% (w/w), about 1.25% (w/w) to about 1.5% (w/w), about 0.5% (w/w) to about 1.25% (w/w), about 0.75% (w/w) to about 1.25% (w/w), about 1% (w/w) to about 1.25% (w/w), about 0.5% (w/w) to about 1% (w/w), about 0.75% (w/w) to about 1% (w/w), or about 0.5% (w/w) to about 0.75% (w/w) alginate. In some embodiments, a formulated composition comprises about 0.5% (w/w), about 0.75% (w/w), about 1% (w/w), about 1.25% (w/w), or about 1.5% (w/w) alginate.

In some embodiments, a formulated composition comprises about 5% (w/w) to about 50% (w/w), about 10% (w/w) to about 50% (w/w), about 15% (w/w) to about 50% (w/w), about 20% (w/w) to about 50% (w/w), about 25% (w/w) to about 50% (w/w), about 30% (w/w) to about 50% (w/w), about 35% (w/w) to about 50% (w/w), about 40% (w/w) to about 50% (w/w), about 45% (w/w) to about 50% (w/w), about 5% (w/w) to about 45% (w/w), about 10% (w/w) to about 45% (w/w), about 15% (w/w) to about 45% (w/w), about 20% (w/w) to about 45% (w/w), about 25% (w/w) to about 45% (w/w), about 30% (w/w) to about 45% (w/w), about 35% (w/w) to about 45% (w/w), about 40% (w/w) to about 45% (w/w), about 5% (w/w) to about 40% (w/w), about 10% (w/w) to about 40% (w/w), about 15% (w/w) to about 40% (w/w), about 20% (w/w) to about 40% (w/w), about 25% (w/w) to about 40% (w/w), about 30% (w/w) to about 40% (w/w), about 35% (w/w) to about 40% (w/w), about 5% (w/w) to about 35% (w/w), about 10% (w/w) to about 35% (w/w), about 15% (w/w) to about 35% (w/w), about 20% (w/w) to about 35% (w/w), about 25% (w/w) to about 35% (w/w), about 30% (w/w) to about 35% (w/w), about 5% (w/w) to about 30% (w/w), about 10% (w/w) to about 30% (w/w), about 15% (w/w) to about 30% (w/w), about 20% (w/w) to about 30% (w/w), about 25% (w/w) to about 30% (w/w), about 5% (w/w) to about 25% (w/w), about 10% (w/w) to about 25% (w/w), about 15% (w/w) to about 25% (w/w), about 20% (w/w) to about 25% (w/w), about 5% (w/w) to about 20% (w/w), about 10% (w/w) to about 20% (w/w), about 15% (w/w) to about 20% (w/w), about 5% (w/w) to about 15% (w/w), about 10% (w/w) to about 15% (w/w), about 5% (w/w) to about 10% (w/w), or about 5% (w/w) to about 10% (w/w) pectin. In some embodiments, a formulated composition comprises about 5% (w/w), about 10% (w/w), about 15% (w/w), about 20% (w/w), about 25% (w/w), about 30% (w/w), about 35% (w/w), about 40% (w/w), about 45% (w/w), or about 50% (w/w) pectin.

In some embodiments, a formulated composition comprises about 5% (w/w) to about 50% (w/w), about 10% (w/w) to about 50% (w/w), about 15% (w/w) to about 50% (w/w), about 20% (w/w) to about 50% (w/w), about 25% (w/w) to about 50% (w/w), about 30% (w/w) to about 50% (w/w), about 35% (w/w) to about 50% (w/w), about 40% (w/w) to about 50% (w/w), about 45% (w/w) to about 50% (w/w), about 5% (w/w) to about 45% (w/w), about 10% (w/w) to about 45% (w/w), about 15% (w/w) to about 45% (w/w), about 20% (w/w) to about 45% (w/w), about 25% (w/w) to about 45% (w/w), about 30% (w/w) to about 45% (w/w), about 35% (w/w) to about 45% (w/w), about 40% (w/w) to about 45% (w/w), about 5% (w/w) to about 40% (w/w), about 10% (w/w) to about 40% (w/w), about 15% (w/w) to about 40% (w/w), about 20% (w/w) to about 40% (w/w), about 25% (w/w) to about 40% (w/w), about 30% (w/w) to about 40% (w/w), about 35% (w/w) to about 40% (w/w), about 5% (w/w) to about 35% (w/w), about 10% (w/w) to about 35% (w/w), about 15% (w/w) to about 35% (w/w), about 20% (w/w) to about 35% (w/w), about 25% (w/w) to about 35% (w/w), about 30% (w/w) to about 35% (w/w), about 5% (w/w) to about 30% (w/w), about 10% (w/w) to about 30% (w/w), about 15% (w/w) to about 30% (w/w), about 20% (w/w) to about 30% (w/w), about 25% (w/w) to about 30% (w/w), about 5% (w/w) to about 25% (w/w), about 10% (w/w) to about 25% (w/w), about 15% (w/w) to about 25% (w/w), about 20% (w/w) to about 25% (w/w), about 5% (w/w) to about 20% (w/w), about 10% (w/w) to about 20% (w/w), about 15% (w/w) to about 20% (w/w), about 5% (w/w) to about 15% (w/w), about 10% (w/w) to about 15% (w/w), about 5% (w/w) to about 10% (w/w), or about 5% (w/w) to about 10% (w/w) carrageenan. In some embodiments, a formulated composition comprises about 5% (w/w), about 10% (w/w), about 15% (w/w), about 20% (w/w), about 25% (w/w), about 30% (w/w), about 35% (w/w), about 40% (w/w), about 45% (w/w), or about 50% (w/w) carrageenan.

In some embodiments, a formulated composition comprises about 0.25% (w/w) to about 2% (w/w), about 0.5% (w/w) to about 2% (w/w), about 0.75% (w/w) to about 2% (w/w), about 1% (w/w) to about 2% (w/w), about 1.25% (w/w) to about 2% (w/w), about 1.5% (w/w) to about 2% (w/w), about 1.75% (w/w) to about 2% (w/w), about 0.25% (w/w) to about 1.75 (w/w), about 0.5% (w/w) to about 1.75 (w/w), about 0.75% (w/w) to about 1.75 (w/w), about 1% (w/w) to about 1.75 (w/w), about 1.25% (w/w) to about 1.75 (w/w), about 1.5% (w/w) to about 1.75 (w/w), about 0.25% (w/w) to about 1.5 (w/w), about 0.5% (w/w) to about 1.5 (w/w), about 0.75% (w/w) to about 1.5 (w/w), about 1% (w/w) to about 1.5 (w/w), about 1.25% (w/w) to about 1.5 (w/w), about 0.25% (w/w) to about 1 (w/w), about 0.5% (w/w) to about 1 (w/w), about 0.75% (w/w) to about 1 (w/w), about 0.25% (w/w) to about 0.75 (w/w), about 0.5% (w/w) to about 0.75 (w/w), or about 0.25% (w/w) to about 0.5% (w/w) gluconolactone. In some embodiments, a formulated composition comprises about 0.25% (w/w), about 0.5% (w/w), about 0.75% (w/w), about 1% (w/w), about 1.25% (w/w), about 1.5% (w/w), about 1.75% (w/w), or about 2% (w/w) gluconolactone.

In some embodiments, a formulated composition comprises about 0.25% (w/w) to about 2% (w/w), about 0.5% (w/w) to about 2% (w/w), about 0.75% (w/w) to about 2% (w/w), about 1% (w/w) to about 2% (w/w), about 1.25% (w/w) to about 2% (w/w), about 1.5% (w/w) to about 2% (w/w), about 1.75% (w/w) to about 2% (w/w), about 0.25% (w/w) to about 1.75 (w/w), about 0.5% (w/w) to about 1.75 (w/w), about 0.75% (w/w) to about 1.75 (w/w), about 1% (w/w) to about 1.75 (w/w), about 1.25% (w/w) to about 1.75 (w/w), about 1.5% (w/w) to about 1.75 (w/w), about 0.25% (w/w) to about 1.5 (w/w), about 0.5% (w/w) to about 1.5 (w/w), about 0.75% (w/w) to about 1.5 (w/w), about 1% (w/w) to about 1.5 (w/w), about 1.25% (w/w) to about 1.5 (w/w), about 0.25% (w/w) to about 1 (w/w), about 0.5% (w/w) to about 1 (w/w), about 0.75% (w/w) to about 1 (w/w), about 0.25% (w/w) to about 0.75 (w/w), about 0.5% (w/w) to about 0.75 (w/w), or about 0.25% (w/w) to about 0.5% (w/w) dicalcium phosphate. In some embodiments, a formulated composition comprises about 0.25% (w/w), about 0.5% (w/w), about 0.75% (w/w), about 1% (w/w), about 1.25% (w/w), about 1.5% (w/w), about 1.75% (w/w), or about 2% (w/w) dicalcium phosphate.

### C. Additional Components

In some embodiments, a formulated composition of the present disclosure comprises 1 or more components in addition to one or more food component and one or more release modulators. In some embodiments, a formulated composition of the present disclosure comprises 1 to 25, 5 to 25, 10 to 25, 15 to 25, 20 to 25, 1 to 20, 5 to 20, 10 to 20, 15 to 20, 1 to 15, 5 to 15, 10 to 15, 1 to 10, 5 to 10, or 1 to 5 components in addition to one or more food component and one or more release modulators. For example, in some embodiments, a formulated composition of the present disclosure comprises 1, 2, 3, 4, 5, 6, 7, 8, 9, 10, 11, 12, 13, 14, 15, 16, 17, 18, 19, 20, 21, 22, 23, 24, or 25 components in addition to one or more food component and one or more release modulators.

### 1. Coatings

In some embodiments, a formulated composition of the present disclosure comprises one or more coatings that encapsulates one or more food components and one or more release modulators. In some embodiments, a coating comprises one or more hydrophobic materials, one or more hydrophilic materials, or one or more amphiphilic materials.

### D. Core-Shell Preparations

In some embodiments, the present disclosure provides core-shell preparations as a means of controlling the release of one or more food components from a formulated composition. For example, in some embodiments, formulated compositions are or comprise core-shell preparations. For example, core-shell preparations may comprise a core component (e.g., interior component) and/or a shell component (e.g., coating, exterior component), each of which are essentially comprised of one or more food components, as described herein. In some embodiments, a core component is comprised of one or more food components and/or one or more release modulators. In some embodiments, a shell component is comprised of one or more food components and/or one or more release modulators.

In some embodiments, a core-shell preparation may comprise a formulated composition comprising one or more food components and one or more release modulators arranged as a core component and one or more food components and/or one or more release modulators arranged as a shell component. In some embodiments, the one or more food components and one or more release modulators comprising a core component are additionally the one or more food components and/or one or more release modulators comprising a shell component. In some embodiments, one or more food components and one or more release modulators comprising a core component are different from the one or more food components and/or one or more release modulators comprising a shell component.

In some embodiments, a core-shell preparation may comprise a formulated composition comprising one or more food components, one or more release modulators, and one or more additional components (e.g., one or more excipients, and/or one or more probiotics, prebiotics, or postbiotics) arranged as a core component and one or more food components, one or more release modulators, and/or one or more additional components (e.g., one or more excipients, and/or one or more probiotics, prebiotics, or postbiotics) arranged as a shell component.

In some embodiments, one or more food components and one or more release modulators may be described as being dispersed within (e.g., embedded within) at least one shell component. In some embodiments, at least one shell component may be described as encapsulating a core component comprising one or more food components and one or more release modulators.

In some embodiments, a core component and/or one or more shell components may be further characterized as a matrix preparation. In some embodiments, a core component and/or one or more shell components comprise a matrix preparation.

In some embodiments, a core-shell preparation may comprise a formulated composition comprising multiple layers of one or more food components and one or more release modulators arranged as a core component and one or more food components and/or one or more release modulators arranged as at least one shell component.

In some embodiments, a core-shell preparation may be described as being dispersed within (e.g., embedded within) one or more additional shell component. In some embodiments, one or more shell components may be described as encapsulating a core-shell preparation and/or matrix preparation.

In some embodiments, a core component is encapsulated in 1 to 15, 3 to 15, 5 to 15, 7 to 15, 9 to 15, 11 to 15, 13 to 15, 1 to 13, 3 to 13, 5 to 13, 7 to 13, 9 to 13, 11 to 13, 1 to 11, 3 to 11, 5 to 11, 7 to 11, 9 to 11, 1 to 9, 3 to 9, 5 to 9, 7 to 9, 1 to 7, 3 to 7, 5 to 7, 1 to 5, 3 to 5, or 1 to 3 shell components. In some embodiments, one or more shell components are homogeneously blended.

In some embodiments, a core component is characterized as being a solid. In some embodiments, a core component is characterized as being a liquid. In some embodiments, one or more shell components are characterized as being solids. In some embodiments, one or more shell components are characterized as being liquids.

In some embodiments, a core-shell preparation may be characterized as a particle (e.g., particle preparation), an emulsion, a suspension, a powder (e.g., a protein powder), a bar, a gel, a capsule, a tablet, a fiber, an extrudate, a hard candy, a chip, and/or a mesh.

In some embodiments, a core-shell preparation may be further characterized as an emulsion. In some embodiments, one or more emulsions are characterized as having low solubility (e.g., miscibility) in water. Additionally, or alternatively, one or more emulsions comprise a core component, one or more shell components, or both a core component and one or more shell components characterized as being amphiphilic. In some embodiments, a core-shell preparation characterized as an emulsion may be described as having a core component having low solubility (e.g., miscibility) in water and one or more shell components characterized as being amphiphilic. In some embodiments, a core-shell preparation characterized as an emulsion may be described as having a core component having high solubility (e.g., miscibility) in water and one or more shell components characterized as being amphiphilic. In some embodiments, a core-shell preparation characterized as an emulsion is prepared prior to ingestion by a subject. In some embodiments, a core-shell preparation characterized as an emulsion spontaneously forms upon addition to one or more dissolution solvents. In some embodiments, a core-shell preparation characterized as an emulsion spontaneously forms upon exposure to one or more triggers. For example, in some embodiments, an emulsion spontaneously forms in response to one or more cross-linkers, pH, bile salts, surfactants, reducing and/or oxidizing agents, osmotic pressure, proteases, amylases, lipases, bacteria, yeast, transglutaminases, thrombin, temperature, time, mechanical forces, or combination thereof.

In some embodiments, one or more shell components controls the release of one or more food components from a core component. In some embodiments, a core component controls the release of one or more food components from a shell component. In some embodiments, control of the release of one more food components from a core component is achieved by one or more shell components providing a physical barrier to a dissolution solvent, a physical barrier to access of a digestive enzyme to one or more food components, controlling the diffusivity of one or more core components, controlling the chemical properties of one or more core components, controlling residence time of one or more core components in a dissolution medium, being responsive to one or more triggers described herein, or a combination thereof. In some embodiments, control of the release of one more food components from one or more shell components is achieved by a core component providing a physical barrier to a dissolution solvent, a physical barrier to access of a digestive enzyme to one or more food components, controlling the diffusivity of one or more shell components, controlling the chemical properties of one or more shell components, controlling residence time of one or more shell components in a dissolution medium, being responsive to one or more triggers described herein, or a combination thereof.

In some embodiments, one or more shell components provides a physical barrier between a dissolution solvent and a core component. For example, in some embodiments, one or more shell components may be characterized as insoluble in aqueous media (e.g., water, phosphate buffered saline solution, simulated intestinal fluid, simulated gastric fluid, simulated tear fluid, simulated urine, HEPES buffered saline solution, Dulbecco's Modified Eagle Medium, Hank's balanced salt solution, biological intestinal fluid, biological gastric fluid, plasma, saliva, urine, feces, sweat, tear fluid, and/or Kreb's buffer). For example, in some embodiments, one or more shell components may be characterized by slow and/or zero-order solubilization in aqueous media. Without wishing to be bound by any particular theory, it is contemplated that one or more shell components characterized by insolubility, slow, and/or zero-order solubilization in aqueous media prevent access of such aqueous media to a core component, thus preventing dissolution (e.g., release) of one or more food components from the core component.

In some embodiments, one or more shell components controls the chemical properties of a core component. In some embodiments, a core component controls the chemical properties of one or more shell components. For example, in some embodiments, one or more shell components may be ionically and/or covalently bonded (i.e., associated, complexed) to itself (e.g., a polymer and/or a crystal) and/or a core component. Additionally, or alternatively, a core component may be ionically and/or covalently bonded (i.e., associated, complexed) to itself (e.g., a polymer and/or a crystal) and/or one or more shell components. Without wishing to be bound by any particular theory, it is contemplated that ionic and/or covalent bonding of a core component with itself and/or one or more shell components may reduce preparation solubility, increase hydrophobicity, and/or increase molecular weight of the core component, thereby reducing diffusivity and release. Without wishing to be bound by any particular theory, it is contemplated that ionic and/or covalent bonding of one or more shell components and/or a core component may reduce preparation solubility, increase hydrophobicity, and/or increase molecular weight of the one or more shell components, thereby reducing diffusivity and release. In some embodiments, one or more shell components performs a chemical reaction on a core component to change its molecular weight and/or introduce a new chemical functionality. In some embodiments, a core component performs a chemical reaction on one or more shell components to change their molecular weight and/or introduce a new chemical functionality. Without wishing to be bound by any particular theory, it is contemplated that one or more components performing chemical reactions to reduce the molecular weight of a core component or one or more shell components may increase the release of one or more food components from a core-shell preparation.

In some embodiments, one or more shell components controls the chemical properties of a core component. In some embodiments, a core component controls the chemical properties of one or more shell components. For example, in some embodiments, one or more shell components may be non-covalently bonded (e.g., hydrogen bonding, Van der Waals forces, electrostatic interactions, hydrophobic interactions, etc.) to each other (e.g., a polymer and/or a crystal) and/or a core component. Additionally, or alternatively, a core component may be non-covalently bonded (i.e., associated, complexed) to itself (e.g., a polymer and/or a crystal) and/or one or more shell components. Without wishing to be bound by any particular theory, it is contemplated that non-covalent bonding of a core component with itself and/or one or more shell components may reduce its solubility, increase hydrophobicity, and/or increase molecular weight of the core component, thereby reducing diffusivity and release. Without wishing to be bound by any particular theory, it is contemplated that non-covalent bonding of one or more shell components with each other and/or a core component may reduce their solubility, increase hydrophobicity, and/or increase molecular weight of the one or more shell components, thereby reducing diffusivity and release. In some embodiments, one or more shell components perform a chemical reaction on a core component to change its molecular weight and/or introduce a new chemical functionality. In some embodiments, a core component performs a chemical reaction on one or more shell components to change their molecular weight and/or introduce a new chemical functionality. Without wishing to be bound by any particular theory, it is contemplated that chemical reactions to reduce the molecular weight of a core component or one or more shell components may increase the release of one or more food components from a core-shell preparation.

In some embodiments, one or more shell components control the residence time of a core component in a biological compartment. In some embodiments, a core-shell preparation, as provided herein, is characterized by retention in a biological compartment. In some embodiments, a core-shell preparation, as provided herein, is characterized by a lack of retention in a biological compartment. In some embodiments, one or more shell components is or are characterized as being mucoadhesive (e.g., having affinity and/or adhesion to one or more mucosal interfaces through interaction with the mucus, glycocalyx, extracellular matrix, cell membrane proteins, and/or cell membranes). In some embodiments, one or more shell components is or are characterized as being mucopenetrative (e.g., having a lack of interaction with the mucus, glycocalyx, extracellular matrix, cell membrane proteins, and/or cell membranes).

In some embodiments, a mucoadhesive shell component may comprise pH-responsive carbohydrates, as described herein. In some embodiments, pH-responsive carbohydrates are characterized by having water solubility at a predetermined pH. In some embodiments, pH-responsive carbohydrates are characterized as having water solubility at low pH (e.g., pH<about 5, pH<about 4, pH<about 3, pH<about 2, pH<about 1). In some embodiments, pH-responsive carbohydrates exhibit low water solubility at low pH and higher water solubility at moderate pH (e.g., pH of about 5.5, about 6, about 6.5, about 7, about 7.5, about 8) to high pH (e.g., pH>8, pH>9, pH>10, pH>11, pH>12). In some embodiments, pH-responsive carbohydrates exhibit higher water solubility at low pH and lower water solubility at moderate to high pH.

For example, in some embodiments, pH-responsive carbohydrates may comprise sodium alginate, potassium alginate, calcium alginate, magnesium alginate, zinc alginate, sodium pectinate, potassium pectinate, calcium pectinate, zinc pectinate, sodium hyaluronate, potassium hyaluronate, calcium hyaluronate, magnesium hyaluronate, zinc hyaluronate, cellulose acetate succinate, cellulose acetate butyrate, cellulose acetate phthalate, hydroxypropyl methylcellulose acetate succinate, heparin sodium, sodium carboxymethylcellulose, chitosan, or a combination thereof.

In some embodiments, a mucoadhesive shell component comprises a mucoadhesive carbohydrate. In some embodiments, a mucoadhesive carbohydrate is characterized by its ability to interact with the mucosal interface (e.g., mucus, mucins, glycocalyx, proteoglycans, cell membrane, phospholipids). Without wishing to be bound by any particular theory, mucoadhesive carbohydrates may utilize a combination of hydrogen bonding, charge-charge interaction, and hydrophobic effect to prolong residence time of core-shell preparations on a mucosal surface.

For example, in some embodiments, mucoadhesive carbohydrates may comprise sodium alginate, potassium alginate, calcium alginate, magnesium alginate, zinc alginate, sodium pectinate, potassium pectinate, calcium pectinate, zinc pectinate, sodium hyaluronate, potassium hyaluronate, calcium hyaluronate, magnesium hyaluronate, zinc hyaluronate, sodium carboxymethylcellulose, chitosan, or a combination thereof.

In some embodiments, a mucoadhesive shell component may comprise mucoadhesive proteins. In some embodiments, mucoadhesive proteins are characterized by their ability to interact with the mucosal interface (e.g., mucus, mucins, glycocalyx, proteoglycans, cell membrane, phospholipids). Without wishing to be bound by any particular theory, mucoadhesive proteins may utilize a combination of hydrogen bonding, charge-charge interaction, and hydrophobic effect to prolong residence time of formulations (e.g., particle preparations) on a mucosal surface.

For example, in some embodiments, mucoadhesive proteins may comprise Lycopersicon esculentum agglutinin, wheat germ agglutinin, Urtica dioica agglutinin, or a combination thereof.

In some embodiments, a mucoadhesive shell component comprises a catechol. In some embodiments, mucoadhesive catechols are polyphenols (e.g., as described herein) that are characterized by their ability to interact with the mucosal interface (e.g., mucus, mucins, glycocalyx, proteoglycans, cell membrane, phospholipids). Without wishing to be bound by any particular theory, mucoadhesive catechols may utilize a combination of hydrogen bonding, x-stacking, chemical cross-linking, and hydrophobic effect to prolong residence time of formulations (e.g., particle preparations) on a mucosal surface.

For example, in some embodiments, mucoadhesive catechols may comprise L-dopamine, poly(L-dopamine), hydroxytyrosol, catechol, caffeic acid, vanillin, veratraldehyde, eugenol, tannic acid, syringaldehyde, and/or protocatechuic aldehyde.

In some embodiments, a mucoadhesive shell component(s) may comprise charged polymers (e.g., polymers exhibiting charge depending on the pH of a dissolution solvent). In some embodiments, a charged polymer may be an anionic mucoadhesive polymer (e.g., polymers exhibiting a negative charge depending on pH of a dissolution solvent). In some embodiments, a charged polymer may be cationic mucoadhesive polymer (e.g., polymers exhibiting a positive charge depending on pH of a dissolution solvent). Without wishing to be bound by any particular theory, it is contemplated that charged polymers facilitate interaction with the mucosal interface (e.g., mucus, mucins, glycocalyx, proteoglycans, cell membrane, phospholipids) thereby enabling greater retention time of one or more core-shell preparation(s).

For example, in some embodiments, anionic mucoadhesive polymers may comprise poly(acrylic acid), poly(methacrylic acid), and/or poly(glycerol citrate). In alternative embodiments cationic mucoadhesive polymers may comprise poly(ethyleneimine), trimethylchitosan, and/or poly(L-arginine).

In some embodiments, a mucopenetrative shell component is characterized by a lack of interaction with the mucus, glycocalyx, extracellular matrix, cell membrane proteins, and/or cell membranes. Without wishing to be bound by any particular theory, one or more mucopenetrative shell components are contemplated to increase the diffusion of a core-shell preparation at a mucosal interface. For example, in some embodiments, a mucopenetrative shell component may comprise poly(ethylene glycol), poly(propylene glycol), poly(vinyl alcohol), and/or poly(ethylene oxide-co-propylene oxide).

In some embodiments, one or more shell components controls the release of one or more food components from a core component by responding to a trigger. In some embodiments, a core component controls the release of one or more food components from one or more shell components by responding to a trigger. In some embodiments, a trigger is characterized as a chemical trigger, enzymatic trigger, and/or a physical trigger. For example, a chemical trigger may be further characterized as a cross-linker, pH, bile salt, reducing and/or oxidizing agent, and/or osmotic pressure. For example, an enzymatic trigger may be further characterized as exposure to proteases, amylases, lipases, bacteria, transglutaminases, and/or thrombin. For example, a physical trigger may be further characterized as temperature, time, and/or mechanical forces. Without wishing to be bound by any particular theory, it is contemplated that one or more triggers may act to alter the physical and/or chemical properties of a core component and/or one or more shell components. It is further contemplated that alteration of one or more physical properties (e.g., increasing porosity, reducing molecular weight) acts to increase the release rate of one or more food components from a core-shell preparation. It is further contemplated that alteration of one or more chemical properties (e.g., increasing charge, increasing polarity) acts to increase the release rate of one or more food components from a core-shell preparation.

### E. Matrix Preparations

In some embodiments, the present disclosure provides matrix preparations as a means of controlling the release of one or more food components from a formulated composition. For example, in some embodiments, a formulated composition is or comprises a matrix preparation. For example, matrix preparations may comprise a solute component and/or a matrix component, each of which are essentially comprised of one or more food components and/or one or more release modulators. In some embodiments, a solute component comprises one or more food components and/or one or more release modulators. In some embodiments, a matrix component comprises one or more food components and/or one or more release modulators. In some embodiments, a solute component comprises one or more food components, one or more release modulators, and/or one or more excipients. In some embodiments, a matrix component comprises one or more food components, one or more release modulators, and/or one or more excipients.

In some embodiments, a matrix preparation comprises one or more food components and/or one release modulators arranged as a solute component and one or more food components and/or one or more release modulators arranged as a matrix component. In some embodiments, the one or more food components and/or one or more release modulators comprising a solute component are additionally the one or more food components and/or one or more release modulators components comprising a matrix component. In some embodiments, the one or more food components and/or one or more release modulators comprising a solute component are different than the one or more food components and/or one or more release modulators components comprising a matrix component. In some embodiments, one or more solute components are distributed homogeneously within a matrix preparation. In some embodiments, one or more solute components are distributed heterogeneously within a matrix preparation.

In some embodiments, a matrix component holds one or more food components and/or one or more release modulators in place at a pre-defined spatial distribution. In some embodiments, both a matrix component and a solute component are comprised of food components and release modulators. In some embodiments, a matrix preparation may also include an outer shell (e.g., coating).

In some embodiments, one or more food components and/or one or more release modulators may be described as being dispersed within (e.g., embedded within) one or more matrix components.

In some embodiments, one or more matrix components may be described as encapsulating one or more food components, and/or one or more release modulators.

In some embodiments, one or more food components, one or more release modulators, one or more matrix components, or a combination thereof, may be characterized as being dispersed within (e.g., encapsulated in) one or more matrix components (for example, radially contained within an outer shell).

In some embodiments, a matrix component and/or a solute component is characterized as being a liquid. In some embodiments, a matrix component and/or a solute component is characterized as being a solid.

In some embodiments, a provided matrix preparations may be characterized as a particle (e.g., particle preparation), a bar, a gel, a capsule, a tablet, a fiber, an extrudate, a hard candy, a chip, and/or a mesh.

In certain embodiments, a matrix preparation is characterized as having controlled release of one or more food components. In some embodiments, one or more matrix components controls the release of one or more food components from one or more solute components. In some embodiments, one or more solute components controls the release of one or more food components form one or more matrix components. In some embodiments, controlled release of one or more food components from one more solute components is achieved by one or more matrix components providing a physical barrier to a dissolution solvent, thereby controlling the diffusivity of one or more solute components, controlling the chemical properties of one or more solute components, controlling residence time of one or more solute components in a dissolution medium, and/or having responsiveness to one or more triggers, as described herein. In some embodiments, controlled release of one or more food components from one or more matrix components is achieved by one or more solute components controlling the chemical properties of one or more matrix components, and/or having responsiveness to one or more triggers, as described herein.

In some embodiments, one or more matrix components controls the diffusivity of one or more solute components. In some embodiments, one or more solute components controls the diffusivity of one or more matrix components. For example, in some embodiments, one or more matrix components and/or one or more solute components may be characterized by at least one of porosity, chain length, and/or electric charge. Without wishing to be bound by any particular theory, it is contemplated that one or more matrix components characterized as having reduced porosity and/or increased chain length and/or complementary charge to one or more solute components reduces free movement (e.g., diffusivity) of one or more solute components by presenting a physical obstruction and/or an electric field, thus preventing dissolution (e.g., release) of one or more food components from one or more solute components. Without wishing to be bound by any particular theory, it is contemplated that one or more solute components characterized as having increased chain length and/or complementary charge to one or more matrix components reduce free movement (e.g., diffusivity) of one or more matrix components by presenting a physical obstruction and/or an electric field, thus preventing dissolution (e.g., release) of one or more food components from one or more matrix components.

In certain embodiments, one or more matrix components controls the chemical properties of one or more solute components. In some embodiments, one or more solute components controls the chemical properties of one or more matrix components. For example, in some embodiments, one or more matrix components may be ionically and/or covalently bonded (i.e., associated, complexed) to itself (e.g., a polymer) and/or one or more solute components. Additionally, or alternatively, one or more solute components may be ionically and/or covalently bonded (i.e., associated, complexed) to itself (e.g., a polymer) and/or one or more matrix components. Without wishing to be bound by any particular theory, it is contemplated that ionic and/or covalent bonding of one or more solute components with itself and/or one or more matrix components may reduce its solubility, increase hydrophobicity, and/or increase molecular weight of the solute component, thereby reducing diffusivity and release of one or more food components. Without wishing to be bound by any particular theory, it is contemplated that ionic and/or covalent bonding of one or more matrix components with itself and/or one or more solute components may reduce its solubility, increase hydrophobicity, and/or increase molecular weight of the one or more matrix components, thereby reducing diffusivity and release of one of more food components. In some embodiments, one or more matrix components performs a chemical reaction on one or more solute components to change its molecular weight and/or introduce a new chemical functionality. In some embodiments, one or more solute components performs a chemical reaction on one or more matrix components to change its molecular weight and/or introduce a new chemical functionality. Without wishing to be bound by any particular theory, it is contemplated that one or more components performing chemical reactions to reduce the molecular weight of one or more solute components or one or more matrix components may increase the release of one or more food components from a matrix preparation.

In some embodiments, one or more matrix components controls the chemical properties of one or more solute components. In some embodiments, one or more solute components controls the chemical properties of one or more matrix components. For example, in some embodiments, one or more matrix components may be non-covalently bonded (e.g., hydrogen bonding, Van der Waals forces, electrostatic interactions, hydrophobic interactions, etc.) to itself (e.g., a polymer) and/or one or more solute components. Additionally, or alternatively, one or more solute components may be non-covalently bonded (e.g., hydrogen bonding, Van der Waals forces, electrostatic interactions, hydrophobic interactions, etc.) to itself (e.g., a polymer) and/or one or more matrix components. Without wishing to be bound by any particular theory, it is contemplated that non-covalent bonds (e.g., hydrogen bonding, Van der Waals forces, electrostatic interactions, hydrophobic interactions, etc.) of one or more solute components with itself and/or one or more matrix components may reduce its solubility, increase hydrophobicity, and/or increase molecular weight of the one or more solute components, thereby reducing diffusivity and release of one or more food components. Without wishing to be bound by any particular theory, it is contemplated that non-covalent bonds (e.g., hydrogen bonding, Van der Waals forces, electrostatic interactions, hydrophobic interactions, etc.) of one or more matrix components with itself and/or one or more solute components may reduce its solubility, increase hydrophobicity, and/or increase molecular weight of the matrix component, thereby reducing diffusivity and release of one or more food components. In some embodiments, one or more matrix components performs a chemical reaction on one or more solute components to change its molecular weight and/or introduce a new chemical functionality. In some embodiments, one or more solute components performs a chemical reaction on one or more matrix components to change its molecular weight and/or introduce a new chemical functionality. Without wishing to be bound by any particular theory, it is contemplated that one or more components performing chemical reactions to reduce the molecular weight of a solute component or a matrix component may increase the release of one or more food components from a matrix preparation.

In some embodiments, one or more matrix components controls the release of one or more food components from one or more solute components by responding to a trigger. In some embodiments, one or more solute components controls the release of one or more food components from one or more matrix components by responding to a trigger. In certain embodiments, a trigger is characterized as a chemical trigger, enzymatic trigger, and/or a physical trigger. For example, in some embodiments, a chemical trigger may be further characterized as a cross-linker, pH, bile salts, reducing and/or oxidizing agents, and/or osmotic pressure. For example, in some embodiments, an enzymatic trigger may be further characterized as exposure to proteases, amylases, lipases, bacteria, transglutaminases, and/or thrombin. For example, in some embodiments, a physical trigger may be further characterized as temperature, time, and/or mechanical forces. Without wishing to be bound by any particular theory, it is contemplated that one or more triggers may act to alter the physical and/or chemical properties of one or more solute components and/or one or more matrix components. It is further contemplated that alteration of one or more physical properties (e.g., increasing porosity, reducing molecular weight) acts to increase the release rate of one or more food components. It is further contemplated that alteration of one or more chemical properties (e.g., increasing charge, increasing polarity) acts to increase the release rate of one or more food components.

### II. Formulated Preparations

In some embodiments, the present disclosure provides a formulated preparation comprising two or more formulated compositions. In some embodiments, the two or more formulated compositions are the same or substantially the same. In some embodiments, a formulated preparation comprises two or more formulated compositions that are different.

### 1. Excipients

In some embodiments, a formulated preparation of the present disclosure comprises 1 or more excipients. In some embodiments, a formulated preparation of the present disclosure comprises 1 to 25, 5 to 25, 10 to 25, 15 to 25, 20 to 25, 1 to 20, 5 to 20, 10 to 20, 15 to 20, 1 to 15, 5 to 15, 10 to 15, 1 to 10, 5 to 10, or 1 to 5 components in addition to one or more food component and one or more release modulators. For example, in some embodiments, a formulated preparation of the present disclosure comprises 1, 2, 3, 4, 5, 6, 7, 8, 9, 10, 11, 12, 13, 14, 15, 16, 17, 18, 19, 20, 21, 22, 23, 24, or 25 components in addition to one or more food component and one or more release modulators.

In some embodiments, one or more excipients comprise processing aids in the production and/or manufacture of a formulated composition of the present disclosure. In some embodiments, one or more excipients comprise an anti-caking processing aid (e.g., anti-caking agent). In some embodiments, an anti-caking processing aid comprises one or more of SIPERNAT 22S, SIPERNAT 50S, SIPERNAT 380, Wacker HDK N20, bamboo fiber, carrot fiber, rice hull powder, and calcium carbonate. In some embodiments, one or more excipients comprise an anti-sticking processing aid (e.g., anti-sticking agent). In some embodiments, an anti-sticking processing aid (e.g., an anti-sticking agent) comprises one or more of sunflower lecithin, soybean lecithin, and aquafaba. In some embodiments, one or more excipients comprise a sequestering processing aid (e.g., a sequestrant). In some embodiments, a sequestrant comprises one or more of sodium hexametaphosphate, sodium tripolyphosphate, potassium hexametaphosphate, potassium tripolyphosphate, calcium hexametaphosphate, calcium tripolyphosphate, sodium polyglutamate, potassium polyglutamate, or chitosan. In some embodiments, one or more excipients comprise an anti-foaming processing aid (e.g., an anti-foaming agent). In some embodiments, an anti-foaming processing aid comprises one or more of monoglycerides, diglycerides, propylene glycol, and acetone. In some embodiments, one or more excipients comprise an anti-tack processing aid. In some embodiments, an anti-tack processing aid (e.g., an anti-tack agent) comprises one or more of talc, zinc oxide, magnesium oxide, and microcrystalline cellulose. In some embodiments, one or more excipients comprise an emulsion-stabilizing processing aid (e.g., a stabilizer). In some embodiments, an emulsion-stabilizing processing aid comprises one or more of xanthan gum, guar gum, modified cellulose gum, and locust bean gum. In some embodiments, one or more excipients comprise an anti-caking (e.g., anti-agglomerating, anti-clumping, anti-aggregating) component, a surfactant component, a plasticizing component, an acid scavenger (e.g., buffering agent), a moisture scavenger, a water scavenger, an oxygen scavenger, a desiccant, a polymer, a preservative, a colorant, a flavoring, an anti-oxidant, a humectant, a solvent, or a combination thereof.

In some embodiments, a formulated preparation of the present disclosure comprises about 0.01% to 10% (w/w), 0.1% to 10% (w/w), 0.2% to 10% (w/w), 0.3% to 10% (w/w), 0.4% to 10% (w/w), 0.5% to 10% (w/w), 0.7% to 10% (w/w), 0.8% to 10% (w/w), 0.9% to 10% (w/w), 1% to 10% (w/w), 1.5% to 10% (w/w), 2% to 10% (w/w), 2.5% to 10% (w/w), 3% to 10% (w/w), 3.5% to 10% (w/w), 4% to 10% (w/w), 4.5% to 10% (w/w), 5% to 10% (w/w), 5.5% to 10% (w/w), 6% to 10% (w/w), 6.5% to 10% (w/w), 7% to 10% (w/w), 7.5% to 10% (w/w), 8% to 10% (w/w), 8.5% to 10% (w/w), 9% to 10% (w/w), 9.5% to 10% (w/w), 0.01% to 5% (w/w), 0.1% to 5% (w/w), 0.2% to 5% (w/w), 0.3% to 5% (w/w), 0.4% to 5% (w/w), 0.5% to 5% (w/w), 0.7% to 5% (w/w), 0.8% to 5% (w/w), 0.9% to 5% (w/w), 1% to 5% (w/w), 1.5% to 5% (w/w), 2% to 5% (w/w), 2.5% to 5% (w/w), 3% to 5% (w/w), 3.5% to 5% (w/w), 4% to 5% (w/w), 4.5% to 5% (w/w), 0.01% to 1% (w/w), 0.1% to 1% (w/w), 0.2% to 1% (w/w), 0.3% to 1% (w/w), 0.4% to 1% (w/w), 0.5% to 1% (w/w), 0.7% to 1% (w/w), 0.8% to 1% (w/w), 0.9% to 1% (w/w), 0.01% to 0.5% (w/w), 0.1% to 0.5% (w/w), 0.2% to 0.5% (w/w), 0.3% to 0.5% (w/w), 0.4% to 0.5% (w/w), or 0.01% to 0.1% (w/w) of one or more excipients.

In some embodiments, a formulated preparation of the present disclosure comprises about 0.01% to 10% (w/w), 0.1% to 10% (w/w), 0.2% to 10% (w/w), 0.3% to 10% (w/w), 0.4% to 10% (w/w), 0.5% to 10% (w/w), 0.7% to 10% (w/w), 0.8% to 10% (w/w), 0.9% to 10% (w/w), 1% to 10% (w/w), 1.5% to 10% (w/w), 2% to 10% (w/w), 2.5% to 10% (w/w), 3% to 10% (w/w), 3.5% to 10% (w/w), 4% to 10% (w/w), 4.5% to 10% (w/w), 5% to 10% (w/w), 5.5% to 10% (w/w), 6% to 10% (w/w), 6.5% to 10% (w/w), 7% to 10% (w/w), 7.5% to 10% (w/w), 8% to 10% (w/w), 8.5% to 10% (w/w), 9% to 10% (w/w), 9.5% to 10% (w/w), 0.01% to 5% (w/w), 0.1% to 5% (w/w), 0.2% to 5% (w/w), 0.3% to 5% (w/w), 0.4% to 5% (w/w), 0.5% to 5% (w/w), 0.7% to 5% (w/w), 0.8% to 5% (w/w), 0.9% to 5% (w/w), 1% to 5% (w/w), 1.5% to 5% (w/w), 2% to 5% (w/w), 2.5% to 5% (w/w), 3% to 5% (w/w), 3.5% to 5% (w/w), 4% to 5% (w/w), 4.5% to 5% (w/w), 0.01% to 1% (w/w), 0.1% to 1% (w/w), 0.2% to 1% (w/w), 0.3% to 1% (w/w), 0.4% to 1% (w/w), 0.5% to 1% (w/w), 0.7% to 1% (w/w), 0.8% to 1% (w/w), 0.9% to 1% (w/w), 0.01% to 0.5% (w/w), 0.1% to 0.5% (w/w), 0.2% to 0.5% (w/w), 0.3% to 0.5% (w/w), 0.4% to 0.5% (w/w), or 0.01% to 0.1% (w/w) silica.

In some embodiments, a formulated preparation of the present disclosure comprises about 0.01% to 10% (w/w), 0.1% to 10% (w/w), 0.2% to 10% (w/w), 0.3% to 10% (w/w), 0.4% to 10% (w/w), 0.5% to 10% (w/w), 0.7% to 10% (w/w), 0.8% to 10% (w/w), 0.9% to 10% (w/w), 1% to 10% (w/w), 1.5% to 10% (w/w), 2% to 10% (w/w), 2.5% to 10% (w/w), 3% to 10% (w/w), 3.5% to 10% (w/w), 4% to 10% (w/w), 4.5% to 10% (w/w), 5% to 10% (w/w), 5.5% to 10% (w/w), 6% to 10% (w/w), 6.5% to 10% (w/w), 7% to 10% (w/w), 7.5% to 10% (w/w), 8% to 10% (w/w), 8.5% to 10% (w/w), 9% to 10% (w/w), 9.5% to 10% (w/w), 0.01% to 5% (w/w), 0.1% to 5% (w/w), 0.2% to 5% (w/w), 0.3% to 5% (w/w), 0.4% to 5% (w/w), 0.5% to 5% (w/w), 0.7% to 5% (w/w), 0.8% to 5% (w/w), 0.9% to 5% (w/w), 1% to 5% (w/w), 1.5% to 5% (w/w), 2% to 5% (w/w), 2.5% to 5% (w/w), 3% to 5% (w/w), 3.5% to 5% (w/w), 4% to 5% (w/w), 4.5% to 5% (w/w), 0.01% to 1% (w/w), 0.1% to 1% (w/w), 0.2% to 1% (w/w), 0.3% to 1% (w/w), 0.4% to 1% (w/w), 0.5% to 1% (w/w), 0.7% to 1% (w/w), 0.8% to 1% (w/w), 0.9% to 1% (w/w), 0.01% to 0.5% (w/w), 0.1% to 0.5% (w/w), 0.2% to 0.5% (w/w), 0.3% to 0.5% (w/w), 0.4% to 0.5% (w/w), or 0.01% to 0.1% (w/w) sodium hexametaphosphate.

In some embodiments, a formulated preparation of the present disclosure comprises about 0.01% to 10% (w/w), 0.1% to 10% (w/w), 0.2% to 10% (w/w), 0.3% to 10% (w/w), 0.4% to 10% (w/w), 0.5% to 10% (w/w), 0.7% to 10% (w/w), 0.8% to 10% (w/w), 0.9% to 10% (w/w), 1% to 10% (w/w), 1.5% to 10% (w/w), 2% to 10% (w/w), 2.5% to 10% (w/w), 3% to 10% (w/w), 3.5% to 10% (w/w), 4% to 10% (w/w), 4.5% to 10% (w/w), 5% to 10% (w/w), 5.5% to 10% (w/w), 6% to 10% (w/w), 6.5% to 10% (w/w), 7% to 10% (w/w), 7.5% to 10% (w/w), 8% to 10% (w/w), 8.5% to 10% (w/w), 9% to 10% (w/w), 9.5% to 10% (w/w), 0.01% to 5% (w/w), 0.1% to 5% (w/w), 0.2% to 5% (w/w), 0.3% to 5% (w/w), 0.4% to 5% (w/w), 0.5% to 5% (w/w), 0.7% to 5% (w/w), 0.8% to 5% (w/w), 0.9% to 5% (w/w), 1% to 5% (w/w), 1.5% to 5% (w/w), 2% to 5% (w/w), 2.5% to 5% (w/w), 3% to 5% (w/w), 3.5% to 5% (w/w), 4% to 5% (w/w), 4.5% to 5% (w/w), 0.01% to 1% (w/w), 0.1% to 1% (w/w), 0.2% to 1% (w/w), 0.3% to 1% (w/w), 0.4% to 1% (w/w), 0.5% to 1% (w/w), 0.7% to 1% (w/w), 0.8% to 1% (w/w), 0.9% to 1% (w/w), 0.01% to 0.5% (w/w), 0.1% to 0.5% (w/w), 0.2% to 0.5% (w/w), 0.3% to 0.5% (w/w), 0.4% to 0.5% (w/w), or 0.01% to 0.1% (w/w) chitosan.

In some embodiments, one or more excipients impart a benefit (e.g., reduced caking, increased stability, increased solubility, improved physical properties, improved taste, improved longevity, and/or increased biocompatibility) to a formulated composition and/or formulated preparation of the present disclosure. In some embodiments, one or more excipients impart a change to an environment of a formulated composition and/or formulated preparation. In some embodiments, one or more excipients impart a pH change, a color change, an oxygen concentration change, a water concentration change, or a combination thereof, within a formulated composition and/or formulated preparation. In some embodiments, one or more excipients impart a change (e.g., pH change, oxygen concentration change, flavor change, water concentration change, or combination thereof) to a local environment (e.g., gastrointestinal compartment, powder (e.g., protein powder) product, food product, beverage product) where a formulated composition and/or formulated preparation resides at a point in time. In some embodiments, one or more excipients increase and/or decrease the solubility of one or more food components in a formulated composition and/or formulated preparation of the present disclosure upon mixing in one or more dissolution solvents.

In some embodiments, one or more excipients in a formulated composition and/or formulated preparation of the present disclosure is identified by one or more governing bodies as safe for animal consumption (e.g., generally regarded as safe and/or food additives). Without wishing to be bound by any particular theory, those skilled in the art will appreciate that one or more excipient components are or may be selected from excipients recognized as Generally Regarded as Safe (i.e., GRAS) by the U.S. Food and Drug Administration. In some embodiments, those skilled in the art will appreciate that one or more excipient components are or may be selected from excipients enumerated in 21 C.F.R. 184. In some embodiments, those skilled in the art will appreciate that one or more excipient components are or may be selected from those excipients enumerated in GB2760-2014 by the National Health and Family Planning Commission of the People's Republic of China.

In some embodiments, one or more excipients can lower the water activity of a formulated composition and/or formulated preparation of the present disclosure.

In some embodiments, one or more excipients can lower the moisture content of a formulated composition and/or formulated preparation of the present disclosure.

In some embodiments, one or more excipients can lower the residual solvent content of a formulated composition and/or formulated preparation of the present disclosure.

In some embodiments, one or more excipients can reduce the friability of a formulated composition and/or formulated preparation of the present disclosure.

In some embodiments, one or more excipients can improve the flowability of a formulated composition and/or formulated preparation of the present disclosure.

In some embodiments, one or more excipients can reduce clumping upon storage of a formulated composition and/or formulated preparation of the present disclosure.

In some embodiments, one or more excipients can improve the solubility of one or more food components in a formulated composition and/or formulated preparation of the present disclosure.

In some embodiments, one or more excipients can improve the mixing of one or more food components with one or more dissolution solvents. In some embodiments, one or more excipients can improve the mixing a formulated composition and/or formulated preparation with one or more dissolution solvents.

In some embodiments, one or more excipients can improve the mixing of one or more food components in a powder (e.g., protein powder) product. In some embodiments, one or more excipients can improve the mixing of a formulated composition and/or formulated preparation in a powder product (e.g., protein powder).

In some embodiments, one or more excipients can improve the mixing of one or more food components in a food product. In some embodiments, one or more excipients can improve the mixing of a formulated composition and/or formulated preparation in a food product.

In some embodiments, one or more excipients can improve the mixing of one or more food components in a beverage product. In some embodiments, one or more excipients can improve the mixing of a formulated composition and/or formulated preparation in a beverage product.

In some embodiments, one or more excipients can improve the mixing of one or more food components in a supplement product. In some embodiments, one or more excipients can improve the mixing of a formulated composition and/or formulated preparation in a supplement product.

In some embodiments, one or more excipients can improve the mixing of one or more food components with one or more other food components in a formulated composition and/or formulated preparation of the present disclosure.

In some embodiments, one or more excipients can raise or lower the elastic modulus of a formulated composition and/or formulated preparation of the present disclosure. Without wishing to be bound by any particular theory, raising or lowering the elastic modulus may enable or facilitate methods of formulating or manufacturing a formulated composition and/or formulated preparation of the present disclosure.

In some embodiments, one or more excipients can raise or lower the crystallinity of a formulated composition and/or formulated preparation of the present disclosure.

In some embodiments, one or more excipients can alter and/or maintain the pH of a formulated composition of the present disclosure. In some embodiments, one or more excipients that maintain pH may additionally be used as a means of gas generation. Without wishing to be bound by any particular theory, generation of gas within a formulated composition and/or formulated preparation of the present disclosure may reduce the density of the formulation and increase buoyancy. Without wishing to be bound by any particular theory, increased buoyancy may contribute to increased residence time of a formulation and/or formulated preparation in one or more gastrointestinal compartments.

In some embodiments, one or more excipients can react with environmental molecular oxygen or introduce molecular oxygen in a formulated composition and/or formulated preparation of the present disclosure.

In some embodiments, one or more excipients triggers release of one or more food components from a formulated composition and/or formulated preparation of the present disclosure.

In some embodiments, one or more excipient component can alter the pH within a microenvironment (e.g., stomach, powder (e.g., protein powder) product, food product, beverage product) where a formulated composition and/or formulated preparation of the present disclosure resides.

In some embodiments, one or more excipients affects a response of a formulated composition and/or formulated preparation of the present disclosure to heat.

In some embodiments, one or more excipients affects a response of a formulated composition and/or formulated preparation of the present disclosure to shear forces.

In some embodiments, one or more excipients affects a response of a formulated composition and/or formulated preparation of the present disclosure to elevated pressure.

In some embodiments, one or more excipients prevents fouling (e.g., microbial growth) of a formulated composition and/or formulated preparation of the present disclosure over a period of at least 4 weeks, at least 12 weeks, at least 6 months, at least 1 year, at least 2 years, at least 5 years, and/or at least 10 years.

In some embodiments, one or more excipients improves the taste and/or fragrance of a formulated composition and/or formulated preparation of the present disclosure.

In some embodiments, one or more excipients maintain the water activity a formulated composition and/or formulated preparation of the present disclosure.

In some embodiments, one or more excipients provides a visually pleasing appearance to a formulated composition and/or formulated preparation of the present disclosure.

In some embodiments, one or more excipients affect the stability of a formulated composition and/or formulated preparation of the present disclosure in light, heat, pressure, shear, enzymes, bacteria, and/or one or more dissolution solvents.

In some embodiments, one or more excipients are added to one or more food components and/or one or more release modulators during a manufacturing process of a formulated composition and/or formulated preparation. In some embodiments, one or more excipients are added to one or more food components and/or one or more release modulators after a manufacturing process of a formulated composition and/or formulated preparation and/or before ingestion of said formulation and/or formulated preparation by a subject.

2. Probiotics, Prebiotics and/or Postbiotics

In some embodiments, a formulated preparation of the present disclosure comprises 1 or more probiotics. In some embodiments, a formulated preparation of the present disclosure comprises 1 to 25, 5 to 25, 10 to 25, 15 to 25, 20 to 25, 1 to 20, 5 to 20, 10 to 20, 15 to 20, 1 to 15, 5 to 15, 10 to 15, 1 to 10, 5 to 10, or 1 to 5 probiotics. For example, in some embodiments, a formulated preparation of the present disclosure comprises 1, 2, 3, 4, 5, 6, 7, 8, 9, 10, 11, 12, 13, 14, 15, 16, 17, 18, 19, 20, 21, 22, 23, 24, or 25 probiotics.

In some embodiments, a formulated preparation of the present disclosure comprises Bacillus coagulans, Bacillus licheniformis, Bacillus subtilis, Bifidobacterium angulatum, Bifidobacterium animalis, Bifidobacterium bifidum, Bifidobacterium breve, Bifidobacterium infantis, Bifidobacterium longum, Enterococcus faecium, Enterococcus faecalis, Lactobacillus acidophilus, Lactobacillus amylovorus, Lactobacillus alimentarius, Lactobacillus bulgaricus, Lactobacillus casei subsp., Lactobacillus casei shirota, Lactobacillus curvatus, Lactobacillus delbrueckii subsp lactis, Lactobacillus fermentum, Lactobacillus farciminis, Lactobacillus gasseri, Lactobacillus helveticus, Lactobacillus johnsonii, Lactobacillus lacti, Lactobacillus paracasei, Lactobacillus pentosaceus, Lactobacillus plantarum, Lactobacillus reuteri, Lactobacillus rhamnosus (Lactobacillus GG), Lactobacillus sake, Lactobacillus salivarius, Lactococcus lactis, Micrococcus varians, Pediococcus acidilactici Pediococcus pentosaceus, Pediococcus acidilactici, Pediococcus halophilus, Streptococcus faecalis, Streptococcus thermophilus, Staphylococcus carnosus, or Staphylococcus xylosus Lactobacillus acidophilus, Lactobacillus bulgaricus, Lactobacillus rhamnosus, Lactobacillus reuteri, Streptococcus thermophilus, Bifidobacterium animalis, Bifidobacterium bifidum, Bifidobacterium lactis, Bacillus subtilis, Bacteroides fragilis, Akkermansia muciniphila, Faecalibacterium prausnitzii, or a combination thereof.

In some embodiments, a formulated preparation of the present disclosure comprises about 1×10⁴ CFU to about 1×10¹¹ CFU, about 1×10⁵ CFU to about 1×10¹¹ CFU, about 1×10⁶ CFU to about 1×10¹¹ CFU, about 1×10⁷ CFU to about 1×10¹¹ CFU, about 1×10⁸ CFU to about 1×10¹¹ CFU, about 1×10⁹ CFU to about 1×10¹¹ CFU, about 1×10¹⁰ CFU to about 1×10¹¹ CFU, about 1×10⁴ CFU to about 1×10¹⁰ CFU, about 1×10⁵ CFU to about 1×10¹⁰ CFU, about 1×10⁶ CFU to about 1×10¹⁰ CFU, about 1×10⁷ CFU to about 1×10¹⁰ CFU, about 1×10⁸ CFU to about 1×10¹⁰ CFU, about 1×10⁹ CFU to about 1×10¹⁰ CFU, about 1×10⁴ CFU to about 1×10⁹ CFU, about 1×10⁵ CFU to about 1×10⁹ CFU, about 1×10⁶ CFU to about 1×10⁹ CFU, about 1×10⁷ CFU to about 1×10⁹ CFU, about 1×10⁸ CFU to about 1×10⁹ CFU, about 1×10⁴ CFU to about 1×10⁸ CFU, about 1×10⁵ CFU to about 1×10⁸ CFU, about 1×10⁶ CFU to about 1×10⁸ CFU, about 1×10⁷ CFU to about 1×10⁸ CFU, about 1×10⁴ CFU to about 1×10⁷ CFU, about 1×10⁵ CFU to about 1×10⁷ CFU, about 1×10⁶ CFU to about 1×10⁷ CFU, about 1×10⁴ CFU to about 1×10⁶ CFU, about 1×10⁵ CFU to about 1×10⁶ CFU, or about 1×10⁴ CFU to about 1×10⁵ CFU, of one or more probiotics per gram of the preparation.

In some embodiments, a formulated composition and/or formulated preparation of the present disclosure is characterized as having a water activity less than 0.2. Without wishing to be bound by any particular theory, a water activity less than 0.2 supports the stability and/or longevity of one or more probiotic in a formulated preparation of the present disclosure.

In some embodiments, a formulated preparation of the present disclosure comprises one or more prebiotic. In some embodiments, a formulated preparation of the present disclosure comprises galactooligosaccharides, inulin, fructan, β-glucan, xylan, pectin, and or a combination thereof.

In some embodiments, a formulated preparation of the present disclosure comprises one or more postbiotic. In some embodiments, a formulated preparation of the present disclosure comprises lactic acid, butyrate, propionate, acetate, or a combination thereof.

### 3. Dietary Fiber

In some embodiments, a formulated preparation of the present disclosure comprises 1 or more dietary fibers. In some embodiments, a formulated preparation of the present disclosure comprises 1 to 25, 5 to 25, 10 to 25, 15 to 25, 20 to 25, 1 to 20, 5 to 20, 10 to 20, 15 to 20, 1 to 15, 5 to 15, 10 to 15, 1 to 10, 5 to 10, or 1 to 5 dietary fibers. For example, in some embodiments, a formulated preparation of the present disclosure comprises 1, 2, 3, 4, 5, 6, 7, 8, 9, 10, 11, 12, 13, 14, 15, 16, 17, 18, 19, 20, 21, 22, 23, 24, or 25 fibers.

In some embodiments, a formulated preparation of the present disclosure comprises at least 0.1 parts of dietary fiber to at least 1 part of food component (e.g., protein) by dry weight, at least 0.15 parts of dietary fiber to at least 1 part of food component (e.g., protein) by dry weight, at least 0.2 parts of dietary fiber to at least 1 part of food component (e.g., protein) by dry weight, or at least 0.25 parts of dietary fiber to at least 1 part of food component (e.g., protein) by dry weight.

In some embodiments, a formulated preparation of the present disclosure may comprise cellulose, dextrin, amylose, amylopectin, pectin, inulin, lignin, chitin, xanthan gum, sodium alginate, potassium alginate, calcium alginate, ammonium alginate, propylene glycol alginate, sodium hyaluronate, potassium hyaluronate, calcium hyaluronate, agar, agarose, carrageenan, raffinose, cellulose acetate, methyl cellulose, ethyl cellulose, cellulose acetate propionate, cellulose acetate butyrate, cellulose acetate succinate, hydroxypropyl methylcellulose, hydroxypropyl cellulose, sodium carboxymethylcellulose, naturally occurring fiber (e.g., vegetable fiber, whole grain fiber, fruit fiber, cereal bran fiber, flaked cereal fiber, flour fiber), β-glucan soluble fiber, psyllium husk, guar gum, locust bean gum, mixed plant cell wall fibers (e.g., sugar cane fiber, apple fiber), arabinoxylan, alginate, inulin-type fructans, high amylose starch, galactooligosaccharide, polydextrose, maltodextrin, cross-linked phosphorylated RS4, glucomannan, acacia (gum arabic), or a combination thereof.

### B. Controlled/Extended-Release

In some embodiments, the present disclosure provides a formulated composition and/or formulated preparation that is characterized as providing controlled release of one or more food components as compared to one or more food components that are not formulated (e.g., do not comprise one or more release modulators). Without wishing to be bound by any particular theory, controlled release may be characterized as the amount of one or more food components released from a formulated composition and/or formulated preparation in the presence of one or more dissolution solvents. In some embodiments, controlled release may be characterized as a predetermined release rate of one or more food components from a formulated composition and/or formulated preparation. In some embodiments, controlled release is characterized as a combination of the amount of one or more food components released from a formulated composition and/or formulated preparation and the rate at which one or more food components is released from the formulated composition.

In some embodiments, controlled release of one or more food components from a formulated composition and/or formulated preparation is characterized by at least one of: a total amount (e.g., mass and/or weight) of one or more food components released; a total amount (e.g., mass and/or weight) of one or more food components released relative to initial loading (e.g., percent release); a total amount of energy provided (e.g., calories released); the location of release in a given incubation period in one or more dissolution solvents; or a combination thereof.

Without wishing to be bound by any particular theory, those skilled in the art will appreciate that one or more food components may be characterized by solubility. Those skilled in the art will further appreciate that one or more food components may be characterized as slightly soluble, partially soluble, and/or completely soluble in one or more dissolution solvents. Those skilled in the art will further appreciate that solubility of one or more food components may be achieved by physical and/or chemical dispersal of one more food components within one or more dissolution solvents. Those skilled in the art will further appreciate that the dispersal (e.g., dissolution) of one or more food components in one or more dissolution solvents may be characterized as release of said one or more food components from a formulated composition and/or formulated preparation.

Without wishing to be bound by any particular theory, it is contemplated that the solubility of one more food components is an important factor determining the gastrointestinal absorption of said one or more food components. Without wishing to be bound by any particular theory, controlling the solubility of one or more food components influences the gastrointestinal absorption and/or bioavailability of the one or more food components in a subject that has ingested a formulation. In some embodiments, controlling the solubility of one or more food components may be characterized as a means of controlling the release of one or more food components.

In some embodiments, controlled release of one or more food components from a formulated composition and/or formulated preparation comprises regulating access of one or more dissolution solvents to the one or more food components, regulating access of one or more enzymes (e.g., digestive enzymes) to the one or more food components, regulating the diffusivity of the one or more food components, or a combination thereof.

In some embodiments, the release of one or more food components is characterized by the amount (e.g., concentration) of the one or more food components solubilized in one or more dissolution solvents over a predetermined period of time (e.g., incubation period).

In some embodiments, the present disclosure provides a formulated composition and/or formulated preparation that is characterized as providing a delayed release of one or more food components as compared to one or more food components that are not formulated (e.g., do not comprise one or more release modulators). In some embodiments, two or more food components in a formulated composition and/or formulated preparation are characterized as having the same delayed release profile. In some embodiments, two or more food components in a formulated composition and/or formulated preparation are characterized as having different delayed release profiles.

In some embodiments, the present disclosure provides a formulated composition and/or formulated preparation that is characterized as providing an extended duration of release of one or more food components as compared to one or more food components that are not formulated (e.g., do not comprise one or more release modulators). In some embodiments, two or more food components in a formulated composition and/or formulated preparation are characterized as having the same extended durations of release. In some embodiments, two or more food components in a formulated composition and/or formulated preparation are characterized as having different extended durations of release.

In some embodiments, a formulated composition and/or formulated preparation is characterized as providing controlled release of one or more food components in a gastrointestinal compartment (e.g., stomach, small intestine, large intestine) of a subject that has ingested said formulation. In some embodiments, a formulated composition and/or formulated preparation is characterized as providing a delayed release of one or more food components in a gastrointestinal compartment (e.g., stomach, small intestine, large intestine) of a subject that has ingested said formulation. In some embodiments, a formulated composition and/or formulated preparation is characterized as providing an extended duration of release of one or more food components in a gastrointestinal compartment (e.g., stomach, small intestine, large intestine) of a subject that has ingested said formulation.

In some embodiments, a formulated composition is characterized as providing controlled release of one or more food components at a gastrointestinal epithelial surface of a subject that has ingested said formulation. In some embodiments, a formulated composition is characterized as providing a delayed release of one or more food components at a gastrointestinal epithelial surface of a subject that has ingested said formulation. In some embodiments, a formulated composition is characterized as providing an extended duration of release of one or more food components at a gastrointestinal epithelial surface of a subject that has ingested said formulation.

In some embodiments, a formulated composition is characterized as providing controlled release of one or more food components at a gastrointestinal mucosal surface of a subject that has ingested said formulation. In some embodiments, a formulated composition is characterized as providing a delayed release of one or more food components at a gastrointestinal mucosal surface of a subject that has ingested said formulation. In some embodiments, a formulated composition is characterized as providing an extended duration of release of one or more food components at a gastrointestinal mucosal surface of a subject that has ingested said formulation.

In some embodiments, a formulated composition of the present disclosure is characterized as providing a controlled time of residence of one or more released food components in one or more gastrointestinal compartments (e.g., stomach, small intestine, large intestine) of a subject that has ingested said formulation. In some embodiments, a formulated composition of the present disclosure is characterized as providing a delayed residence of one or more released food components in one or more gastrointestinal compartments of a subject that has ingested said formulation. In some embodiments, a formulated composition of the present disclosure is characterized as providing an extended duration of residence of one or more released food components in one or more gastrointestinal compartments of a subject that has ingested said formulation.

In some embodiments, controlled release of one or more food components is characterized as being targeted to a gastrointestinal location. In some embodiments, a formulated composition releases one or more food components in the buccal cavity, stomach, duodenum, jejunum, ileum, colon, rectum, or combination thereof of a subject ingesting said formulation.

In some embodiments, release of one or more food components from a formulated composition and/or formulated preparation into the buccal cavity, stomach, duodenum, jejunum, ileum, colon, rectum, or combination thereof of a subject ingesting said formulation is triggered by a change in pH. In some embodiments, a pH of about 1-2, about 2-3, about 3-4, about 4-5, about 5-6, about 6-7, about 7-8, about 8-9, or about 9-10, triggers release of one of more food components from a formulated composition and/or formulated preparation.

In some embodiments, release of one or more food components from a formulated composition and/or formulated preparation into the buccal cavity, stomach, duodenum, jejunum, ileum, colon, rectum, or combination thereof of a subject ingesting said formulation is triggered by the presence of one or more enzymes. In some embodiments, the presence of one or more amylases, one or more lipases, one or more proteases, one or more bacteria, or combination thereof, triggers release of one of more food components from a formulated composition and/or formulated preparation.

In some embodiments, release of one or more food components from a formulated composition and/or formulated preparation into the buccal cavity, stomach, duodenum, jejunum, ileum, colon, rectum, or combination thereof of a subject ingesting said formulation is triggered by the application of a physical force to said formulation. In some embodiments, the application of physical pressure and/or shear forces, triggers the release of one of more food components from a formulated composition and/or formulated preparation.

In some embodiments, release of one or more food components from a formulated composition and/or formulated preparation into the buccal cavity, stomach, duodenum, jejunum, ileum, colon, rectum, or combination thereof of a subject ingesting said formulation is triggered by a change in temperature. In some embodiments, a temperature of about 20° C., about 25° C., about 28° C., about 30° C., about 35° C., about 37° C., about 40° C., about 45° C., or about 50° C. triggers the release of one of more food components from a formulated composition and/or formulated preparation.

In some embodiments, a formulated composition and/or formulated preparation of the present disclosure is characterized as providing a controlled time of absorption of one or more released food components in one or more gastrointestinal compartments (e.g., stomach, small intestine, large intestine) of a subject that has ingested said formulation. In some embodiments, a formulated composition and/or formulated preparation of the present disclosure is characterized as providing delayed absorption of one or more released food components in one or more gastrointestinal compartments of a subject that has ingested said formulation. In some embodiments, a formulated composition and/or formulated preparation of the present disclosure is characterized as providing an extended duration of absorption of one or more released food components in one or more gastrointestinal compartments of a subject that has ingested said formulation.

Without wishing to be bound by any particular theory, it is contemplated that controlled and/or an extended duration of release of one or more food components in one or more gastrointestinal compartments (e.g., stomach, small intestine, large intestine) of a subject that has ingested said formulation increases the bioavailability of the one or more food components to the subject.

In some embodiments, a formulated composition and/or formulated preparation is characterized as providing controlled energy (e.g., caloric) release to a subject that has ingested said formulation. In some embodiments, a formulated composition and/or formulated preparation is characterized as providing delayed energy (e.g., caloric) release to a subject that has ingested said formulation. In some embodiments, a formulated composition and/or formulated preparation is characterized as providing an extended duration of energy (e.g., caloric) release to a subject that has ingested said formulation.

In some embodiments, the release of one or more food components from a formulated composition and/or formulated preparation of the present disclosure may be held at a constant rate for about 0.5 hours to 72 hours, 1 hour to 72 hours, 2 hours to 72 hours, 4 hours to 72 hours, 6 hours to 72 hours, 8 hours to 72 hours, 10 hours to 72 hours, 12 hours to 72 hours, 18 hours to 72 hours, 24 hours to 72 hours, 30 hours to 72 hours, 36 hours to 72 hours, 42 hours to 72 hours, 48 hours to 72 hours, 54 hours to 72 hours, 60 hours to 72 hours, 66 hours to 72 hours, 0.5 hours to 66 hours, 1 hour to 66 hours, 2 hours to 66 hours, 4 hours to 66 hours, 6 hours to 66 hours, 8 hours to 66 hours, 10 hours to 66 hours, 12 hours to 66 hours, 18 hours to 66 hours, 24 hours to 66 hours, 30 hours to 66 hours, 36 hours to 66 hours, 42 hours to 66 hours, 48 hours to 66 hours, 54 hours to 66 hours, 60 hours to 66 hours, 0.5 hours to 60 hours, 1 hour to 60 hours, 2 hours to 60 hours, 4 hours to 60 hours, 6 hours to 60 hours, 8 hours to 60 hours, 10 hours to 60 hours, 12 hours to 60 hours, 18 hours to 60 hours, 24 hours to 60 hours, 30 hours to 60 hours, 36 hours to 60 hours, 42 hours to 60 hours, 48 hours to 60 hours, 54 hours to 60 hours, 0.5 hours to 54 hours, 1 hour to 54 hours, 2 hours to 54 hours, 4 hours to 54 hours, 6 hours to 54 hours, 8 hours to 54 hours, 10 hours to 54 hours, 12 hours to 54 hours, 18 hours to 54 hours, 24 hours to 54 hours, 30 hours to 54 hours, 36 hours to 54 hours, 42 hours to 54 hours, 48 hours to 54 hours, 0.5 hours to 48 hours, 1 hour to 48 hours, 2 hours to 48 hours, 4 hours to 48 hours, 6 hours to 48 hours, 8 hours to 48 hours, 10 hours to 48 hours, 12 hours to 48 hours, 18 hours to 48 hours, 24 hours to 48 hours, 30 hours to 48 hours, 36 hours to 48 hours, 42 hours to 48 hours, 0.5 hours to 42 hours, 1 hour to 42 hours, 2 hours to 42 hours, 4 hours to 42 hours, 6 hours to 42 hours, 8 hours to 42 hours, 10 hours to 42 hours, 12 hours to 42 hours, 18 hours to 42 hours, 24 hours to 42 hours, 30 hours to 42 hours, 36 hours to 42 hours, 0.5 hours to 36 hours, 1 hour to 36 hours, 2 hours to 36 hours, 4 hours to 36 hours, 6 hours to 36 hours, 8 hours to 36 hours, 10 hours to 36 hours, 12 hours to 36 hours, 18 hours to 36 hours, 24 hours to 36 hours, 30 hours to 36 hours, 0.5 hours to 30 hours, 1 hour to 30 hours, 2 hours to 30 hours, 4 hours to 30 hours, 6 hours to 30 hours, 8 hours to 30 hours, 10 hours to 30 hours, 12 hours to 30 hours, 18 hours to 30 hours, 24 hours to 30 hours, 0.5 hours to 24 hours, 1 hour to 24 hours, 2 hours to 24 hours, 4 hours to 24 hours, 6 hours to 24 hours, 8 hours to 24 hours, 10 hours to 24 hours, 12 hours to 24 hours, 18 hours to 24 hours, 0.5 hours to 18 hours, 1 hour to 18 hours, 2 hours to 18 hours, 4 hours to 18 hours, 6 hours to 18 hours, 8 hours to 18 hours, 10 hours to 18 hours, 12 hours to 18 hours, 0.5 hours to 12 hours, 1 hour to 12 hours, 2 hours to 12 hours, 4 hours to 12 hours, 6 hours to 12 hours, 8 hours to 12 hours, 10 hours to 12 hours, 0.5 hours to 10 hours, 1 hour to 10 hours, 2 hours to 10 hours, 4 hours to 10 hours, 6 hours to 10 hours, 8 hours to 10 hours, 0.5 hours to 8 hours, 1 hour to 8 hours, 2 hours to 8 hours, 4 hours to 8 hours, 6 hours to 8 hours, 0.5 hours to 6 hours, 1 hour to 6 hours, 2 hours to 6 hours, 4 hours to 6 hours, 0.5 hours to 4 hours, 1 hour to 4 hours, 2 hours to 4 hours, 0.5 hours to 2 hours, 1 hour to 2 hours, or 0.5 hours to 1.5 hours.

In some embodiments, controlled release of one or more food components from a formulated composition and/or formulated preparation is characterized by the percent release of the one or more food components (based on dry weight) in a given time period (e.g., incubation period) in one or more dissolution solvents.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 1 hour.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 1.5 hours.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 2 hours.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 2.5 hours.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 3 hours.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 4 hours.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 6 hours.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 12 hours.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 24 hours.

In some embodiments, about 5% to 80% (w/w), 10% to 80% (w/w), 15% to 80% (w/w), 20% to 80% (w/w), 25% to 80% (w/w), 30% to 80% (w/w), 35% to 80% (w/w), 40% to 80% (w/w), 45% to 80% (w/w), 50% to 80% (w/w), 55% to 80% (w/w), 60% to 80% (w/w), 65% to 80% (w/w), 70% to 80% (w/w), 75% to 80% (w/w), 5% to 70% (w/w), 10% to 70% (w/w), 15% to 70% (w/w), 20% to 70% (w/w), 25% to 70% (w/w), 30% to 70% (w/w), 35% to 70% (w/w), 40% to 70% (w/w), 45% to 70% (w/w), 50% to 70% (w/w), 55% to 70% (w/w), 60% to 70% (w/w), 65% to 70% (w/w), 5% to 60% (w/w), 10% to 60% (w/w), 15% to 60% (w/w), 20% to 60% (w/w), 25% to 60% (w/w), 30% to 60% (w/w), 35% to 60% (w/w), 40% to 60% (w/w), 45% to 60% (w/w), 50% to 60% (w/w), 55% to 60% (w/w), 5% to 50% (w/w), 10% to 50% (w/w), 15% to 50% (w/w), 20% to 50% (w/w), 25% to 50% (w/w), 30% to 50% (w/w), 35% to 50% (w/w), 40% to 50% (w/w), 45% to 50% (w/w), 5% to 40% (w/w), 10% to 40% (w/w), 15% to 40% (w/w), 20% to 40% (w/w), 25% to 40% (w/w), 30% to 40% (w/w), 35% to 40% (w/w), 5% to 30% (w/w), 10% to 30% (w/w), 15% to 30% (w/w), 20% to 30% (w/w), 25% to 30% (w/w), 5% to 20% (w/w), 10% to 20% (w/w), 15% to 20% (w/w), or 5% to 10% (w/w) of one or more food component is released from a formulated composition and/or formulated preparation into one or more dissolution solvents over a duration of about 48 hours.

In some embodiments, the release of one or more food components from a formulated composition and/or formulated preparation of the present disclosure may occur in intervals (e.g., bolus doses) once every about 30 minutes to 24 hours, 60 minutes to 24 hours, 1.5 hours to 24 hours, 2 hours to 24 hours, 4 hours to 24 hours, 6 hours to 24 hours, 8 hours to 24 hours, 12 hours to 24 hours, 18 hours to 24 hours, 30 minutes to 18 hours, 60 minutes to 18 hours, 1.5 hours to 18 hours, 2 hours to 18 hours, 4 hours to 18 hours, 6 hours to 18 hours, 8 hours to 18 hours, 12 hours to 18 hours, 30 minutes to 12 hours, 60 minutes to 12 hours, 1.5 hours to 12 hours, 2 hours to 12 hours, 4 hours to 12 hours, 6 hours to 12 hours, 8 hours to 12 hours, 30 minutes to 6 hours, 60 minutes to 6 hours, 1.5 hours to 6 hours, 2 hours to 6 hours, 4 hours to 6 hours, 30 minutes to 4 hours, 60 minutes to 4 hours, 1.5 hours to 4 hours, 2 hours to 4 hours, 30 minutes to 2 hours, 60 minutes to 2 hours, 1.5 hours to 2 hours, 30 minutes to 1.5 hours, 60 minutes to 1.5 hours, or 30 minutes to 1 hour.

In some embodiments, the release rate of one or more food components from a formulated composition and/or formulated preparation of the present disclosure may increase one or more times after about 1 hour to 48 hours, 2 hours to 48 hours, 4 hours to 48 hours, 6 hours to 48 hours, 8 hours to 48 hours, 12 hours to 48 hours, 18 hours to 48 hours, 24 hours to 48 hours, 30 hours to 48 hours, 36 hours to 48 hours, 42 hours to 48 hours, 1 hour to 42 hours, 2 hours to 42 hours, 4 hours to 42 hours, 6 hours to 42 hours, 8 hours to 42 hours, 12 hours to 42 hours, 18 hours to 42 hours, 24 hours to 42 hours, 30 hours to 42 hours, 36 hours to 42 hours, 1 hour to 36 hours, 2 hours to 36 hours, 4 hours to 36 hours, 6 hours to 36 hours, 8 hours to 36 hours, 12 hours to 36 hours, 18 hours to 36 hours, 24 hours to 36 hours, 30 hours to 36 hours, 1 hour to 30 hours, 2 hours to 30 hours, 4 hours to 30 hours, 6 hours to 30 hours, 8 hours to 30 hours, 12 hours to 30 hours, 18 hours to 30 hours, 24 hours to 30 hours, 1 hour to 24 hours, 2 hours to 24 hours, 4 hours to 24 hours, 6 hours to 24 hours, 8 hours to 24 hours, 12 hours to 24 hours, 18 hours to 24 hours, 1 hour to 18 hours, 2 hours to 18 hours, 4 hours to 18 hours, 6 hours to 18 hours, 8 hours to 18 hours, 12 hours to 18 hours, 1 hour to 12 hours, 2 hours to 12 hours, 4 hours to 12 hours, 6 hours to 12 hours, 8 hours to 12 hours, 1 hour to 8 hours, 2 hours to 8 hours, 4 hours to 8 hours, 6 hours to 8 hours, 1 hour to 6 hours, 2 hours to 6 hours, 4 hours to 6 hours, 1 hour to 4 hours, 2 hours to 4 hours, or 1 hour to 2 hours, following ingestion of said formulation by a subject.

In some embodiments, the release rate of one or more food components from a formulated composition and/or formulated preparation of the present disclosure may increase one or more times after about 1 hour to 48 hours, 2 hours to 48 hours, 4 hours to 48 hours, 6 hours to 48 hours, 8 hours to 48 hours, 12 hours to 48 hours, 18 hours to 48 hours, 24 hours to 48 hours, 30 hours to 48 hours, 36 hours to 48 hours, 42 hours to 48 hours, 1 hour to 42 hours, 2 hours to 42 hours, 4 hours to 42 hours, 6 hours to 42 hours, 8 hours to 42 hours, 12 hours to 42 hours, 18 hours to 42 hours, 24 hours to 42 hours, 30 hours to 42 hours, 36 hours to 42 hours, 1 hour to 36 hours, 2 hours to 36 hours, 4 hours to 36 hours, 6 hours to 36 hours, 8 hours to 36 hours, 12 hours to 36 hours, 18 hours to 36 hours, 24 hours to 36 hours, 30 hours to 36 hours, 1 hour to 30 hours, 2 hours to 30 hours, 4 hours to 30 hours, 6 hours to 30 hours, 8 hours to 30 hours, 12 hours to 30 hours, 18 hours to 30 hours, 24 hours to 30 hours, 1 hour to 24 hours, 2 hours to 24 hours, 4 hours to 24 hours, 6 hours to 24 hours, 8 hours to 24 hours, 12 hours to 24 hours, 18 hours to 24 hours, 1 hour to 18 hours, 2 hours to 18 hours, 4 hours to 18 hours, 6 hours to 18 hours, 8 hours to 18 hours, 12 hours to 18 hours, 1 hour to 12 hours, 2 hours to 12 hours, 4 hours to 12 hours, 6 hours to 12 hours, 8 hours to 12 hours, 1 hour to 8 hours, 2 hours to 8 hours, 4 hours to 8 hours, 6 hours to 8 hours, 1 hour to 6 hours, 2 hours to 6 hours, 4 hours to 6 hours, 1 hour to 4 hours, 2 hours to 4 hours, or 1 hour to 2 hours, following placement of said formulation in one or more dissolution solvents.

In some embodiments, the release rate of one or more food components from a formulated composition and/or formulated preparation of the present disclosure may decrease one or more times after about 1 hour to 48 hours, 2 hours to 48 hours, 4 hours to 48 hours, 6 hours to 48 hours, 8 hours to 48 hours, 12 hours to 48 hours, 18 hours to 48 hours, 24 hours to 48 hours, 30 hours to 48 hours, 36 hours to 48 hours, 42 hours to 48 hours, 1 hour to 42 hours, 2 hours to 42 hours, 4 hours to 42 hours, 6 hours to 42 hours, 8 hours to 42 hours, 12 hours to 42 hours, 18 hours to 42 hours, 24 hours to 42 hours, 30 hours to 42 hours, 36 hours to 42 hours, 1 hour to 36 hours, 2 hours to 36 hours, 4 hours to 36 hours, 6 hours to 36 hours, 8 hours to 36 hours, 12 hours to 36 hours, 18 hours to 36 hours, 24 hours to 36 hours, 30 hours to 36 hours, 1 hour to 30 hours, 2 hours to 30 hours, 4 hours to 30 hours, 6 hours to 30 hours, 8 hours to 30 hours, 12 hours to 30 hours, 18 hours to 30 hours, 24 hours to 30 hours, 1 hour to 24 hours, 2 hours to 24 hours, 4 hours to 24 hours, 6 hours to 24 hours, 8 hours to 24 hours, 12 hours to 24 hours, 18 hours to 24 hours, 1 hour to 18 hours, 2 hours to 18 hours, 4 hours to 18 hours, 6 hours to 18 hours, 8 hours to 18 hours, 12 hours to 18 hours, 1 hour to 12 hours, 2 hours to 12 hours, 4 hours to 12 hours, 6 hours to 12 hours, 8 hours to 12 hours, 1 hour to 8 hours, 2 hours to 8 hours, 4 hours to 8 hours, 6 hours to 8 hours, 1 hour to 6 hours, 2 hours to 6 hours, 4 hours to 6 hours, 1 hour to 4 hours, 2 hours to 4 hours, or 1 hour to 2 hours, following ingestion of said formulation by a subject.

In some embodiments, the release rate of one or more food components from a formulated composition and/or formulated preparation of the present disclosure may decrease one or more times after about 1 hour to 48 hours, 2 hours to 48 hours, 4 hours to 48 hours, 6 hours to 48 hours, 8 hours to 48 hours, 12 hours to 48 hours, 18 hours to 48 hours, 24 hours to 48 hours, 30 hours to 48 hours, 36 hours to 48 hours, 42 hours to 48 hours, 1 hour to 42 hours, 2 hours to 42 hours, 4 hours to 42 hours, 6 hours to 42 hours, 8 hours to 42 hours, 12 hours to 42 hours, 18 hours to 42 hours, 24 hours to 42 hours, 30 hours to 42 hours, 36 hours to 42 hours, 1 hour to 36 hours, 2 hours to 36 hours, 4 hours to 36 hours, 6 hours to 36 hours, 8 hours to 36 hours, 12 hours to 36 hours, 18 hours to 36 hours, 24 hours to 36 hours, 30 hours to 36 hours, 1 hour to 30 hours, 2 hours to 30 hours, 4 hours to 30 hours, 6 hours to 30 hours, 8 hours to 30 hours, 12 hours to 30 hours, 18 hours to 30 hours, 24 hours to 30 hours, 1 hour to 24 hours, 2 hours to 24 hours, 4 hours to 24 hours, 6 hours to 24 hours, 8 hours to 24 hours, 12 hours to 24 hours, 18 hours to 24 hours, 1 hour to 18 hours, 2 hours to 18 hours, 4 hours to 18 hours, 6 hours to 18 hours, 8 hours to 18 hours, 12 hours to 18 hours, 1 hour to 12 hours, 2 hours to 12 hours, 4 hours to 12 hours, 6 hours to 12 hours, 8 hours to 12 hours, 1 hour to 8 hours, 2 hours to 8 hours, 4 hours to 8 hours, 6 hours to 8 hours, 1 hour to 6 hours, 2 hours to 6 hours, 4 hours to 6 hours, 1 hour to 4 hours, 2 hours to 4 hours, or 1 hour to 2 hours, following placement of said formulation in one or more dissolution solvents.

### 1. Measuring Release

In some embodiments, the release of one or more food components from a formulated composition and/or formulated preparation is characterized by quantification of solubilization of the one or more food components in one or more dissolution solvents.

In some embodiments, a dissolution solvent may be characterized by its miscibility with water. In some embodiments, one or more dissolution solvents is characterized as being miscible with water. In some embodiments, a dissolution solvent may comprise water, phosphate buffered saline (PBS), simulated gastric fluid (SGF), fasted state simulated gastric fluid (FaSSGF), simulated intestinal fluid, simulated tear fluid, simulated urine, HEPES buffered saline, Dulbecco's Modified Eagle Medium (DMEM), Hank's balanced salt solution, cyrene, glycofurol, furfural, Kreb's buffer, acetone, tetrahydrofuran, ethanol, methanol, dimethylformamide, dimethyl sulfoxide, or a combination thereof.

In some embodiments, one or more dissolution solvents is characterized as being immiscible with water. In some embodiments, a dissolution solvent may comprise n-octanol, n-nonanol, n-decanol, n-dodecanol, n-tetradecanol, n-hexadecanol, n-octadecanol, n-icosanol, fatty alcohol monoglyceride ethers, fatty acid monoglyceride esters, fatty alcohol diglyceride ethers, fatty acid diglyceride esters, fatty alcohol triglyceride ethers, fatty acid triglyceride esters, fatty alcohol glycol monoether, fatty acid glycol monoesters, fatty alcohol glycol diethers, fatty acid glycol diesters, coconut oil, corn oil, cottonseed oil, olive oil, palm oil, peanut oil, rapeseed oil, safflower oil, sesame oil, soybean oil, sunflower oil, almond oil, pine nut oil, cashew oil, fully hydrogenated palm oil, partially hydrogenated palm oil, fully hydrogenated sunflower oil, partially hydrogenated sunflower oil, fully hydrogenated soybean oil, partially hydrogenated soybean oil, fully hydrogenated vegetable oil, partially hydrogenated vegetable oil, fully hydrogenated cottonseed oil, partially hydrogenated cottonseed oil, dichloromethane, hexanes, toluene, 2-methyltetrahydrofuran, ethyl acetate, or a combination thereof.

In some embodiments, a dissolution solvent may be characterized by its salinity. In some embodiments, a dissolution solvent may comprise water, PBS, simulated intestinal fluid, SGF, FaSSGF, simulated tear fluid, simulated urine, HEPES buffered saline, DMEM, Hank's balanced salt solution, biological intestinal fluid, biological gastric fluid, plasma, saliva, urine, feces, sweat, tear fluid, Kreb's buffer, or a combination thereof.

In some embodiments, a dissolution solvent may be characterized by its pH. In some embodiments, a dissolution solvent may comprise water, PBS, simulated intestinal fluid, SGF, FaSSGF, simulated tear fluid, simulated urine, HEPES buffered saline, DMEM, Hank's balanced salt solution, biological intestinal fluid, biological gastric fluid, plasma, saliva, urine, feces, sweat, tear fluid, and/or Kreb's buffer.

In some embodiments, a dissolution solvent may be characterized as a native biological fluid (i.e., a fluid characterized as essential to a living organism). In some embodiments, a dissolution solvent may comprise water, biological intestinal fluid, biological gastric fluid, plasma, saliva, urine, feces, sweat, oral fluid, cecum fluid, bile, and/or tear fluid.

In some embodiments, a dissolution solvent may be characterized as a surrogate of a native biological fluid (i.e., a surrogate of a fluid characterized as essential to a living organism). In some embodiments, a dissolution solvent may comprise water, PBS, simulated intestinal fluid, SGF, FaSSGF, simulated tear fluid, simulated urine, HEPES buffered saline, DMEM, Hank's balanced salt solution, and/or Kreb's buffer.

In some embodiments, a dissolution solvent may be characterized by its miscibility with water, by its pH, by its salinity, as being a native biological fluid, as being a surrogate of a native biological fluid, or a combination thereof.

In some embodiments, to determine release of one or more food components from a formulated composition and/or formulated preparation, a preparation of a formulated composition and/or formulated preparation is added to an excess quantity, by weight, of one or more dissolution solvents. In some embodiments, a preparation of a formulated composition and/or formulated preparation is added to at least about 5 fold, at least about 10 fold, at least about 20 fold, at least about 50 fold, at least about 100 fold, at least about 200 fold, at least about 1000 fold, at least about 5000 fold, or at least about 10000 fold, by weight, excess of one or more dissolution solvents.

In some embodiments, to determine release of one or more food components from a formulated composition and/or formulated preparation, a preparation of a formulated composition and/or formulated preparation is added to one or more dissolution solvents at a controlled temperature. In some embodiments, a preparation of a formulated composition and/or formulated preparation is added to one or more dissolution solvents held at least at about −20° C., at least at about 0° C., at least at about 4° C., at least at about 20° C., at least at about 37° C., and/or at least at about 50° C.

In some embodiments, to determine release of one or more food components from a formulated composition and/or formulated preparation, a preparation of a formulated composition and/or formulated preparation is added to one or more dissolution solvents for a predetermined period of time (e.g., incubation period). In some embodiments, a preparation of a formulated composition and/or formulated preparation is added to one or more dissolution solvents and incubated for about 0.5 min. to 1500 min., 1 min. to 1500 min., 5 min. to 1500 min., 10 min. to 1500 min., 15 min. to 1500 min., 30 min. to 1500 min., 45 min. to 1500 min., 60 min. to 1500 min., 90 min. to 1500 min., 120 min. to 1500 min., 150 min. to 1500 min., 200 min. to 1500 min., 250 min. to 1500 min., 300 min. to 1500 min., 400 min. to 1500 min., 500 min. to 1500 min., 600 min. to 1500 min., 700 min. to 1500 min., 800 min. to 1500 min., 900 min. to 1500 min., 1000 min. to 1500 min., 1100 min. to 1500 min., 1200 min. to 1500 min., 1300 min. to 1500 min., 1400 min. to 1500 min., 0.5 min. to 1250 min., 1 min. to 1250 min., 5 min. to 1250 min., 10 min. to 1250 min., 15 min. to 1250 min., 30 min. to 1250 min., 45 min. to 1250 min., 60 min. to 1250 min., 90 min. to 1250 min., 120 min. to 1250 min., 150 min. to 1250 min., 200 min. to 1250 min., 250 min. to 1250 min., 300 min. to 1250 min., 400 min. to 1250 min., 500 min. to 1250 min., 600 min. to 1250 min., 700 min. to 1250 min., 800 min. to 1250 min., 900 min. to 1250 min., 1000 min. to 1250 min., 1100 min. to 1250 min., 0.5 min. to 1000 min., 1 min. to 1000 min., 5 min. to 1000 min., 10 min. to 1000 min., 15 min. to 1000 min., 30 min. to 1000 min., 45 min. to 1000 min., 60 min. to 1000 min., 90 min. to 1000 min., 120 min. to 1000 min., 150 min. to 1000 min., 200 min. to 1000 min., 250 min. to 1000 min., 300 min. to 1000 min., 400 min. to 1000 min., 500 min. to 1000 min., 600 min. to 1000 min., 700 min. to 1000 min., 800 min. to 1000 min., 900 min. to 1000 min., 0.5 min. to 750 min., 1 min. to 750 min., 5 min. to 750 min., 10 min. to 750 min., 15 min. to 750 min., 30 min. to 750 min., 45 min. to 750 min., 60 min. to 750 min., 90 min. to 750 min., 120 min. to 750 min., 150 min. to 750 min., 200 min. to 750 min., 250 min. to 750 min., 300 min. to 750 min., 400 min. to 750 min., 500 min. to 750 min., 600 min. to 750 min., 700 min. to 750 min., 0.5 min. to 500 min., 1 min. to 500 min., 5 min. to 500 min., 10 min. to 500 min., 15 min. to 500 min., 30 min. to 500 min., 45 min. to 500 min., 60 min. to 500 min., 90 min. to 500 min., 120 min. to 500 min., 150 min. to 500 min., 200 min. to 500 min., 250 min. to 500 min., 300 min. to 500 min., 400 min. to 500 min., 0.5 min. to 250 min., 1 min. to 250 min., 5 min. to 250 min., 10 min. to 250 min., 15 min. to 250 min., 30 min. to 250 min., 45 min. to 250 min., 60 min. to 250 min., 90 min. to 250 min., 120 min. to 250 min., 150 min. to 250 min., 200 min. to 250 min., 0.5 min. to 100 min., 1 min. to 100 min., 5 min. to 100 min., 10 min. to 100 min., 15 min. to 100 min., 30 min. to 100 min., 45 min. to 100 min., 60 min. to 100 min., 90 min. to 100 min., 0.5 min. to 60 min., 1 min. to 60 min., 5 min. to 60 min., 10 min. to 60 min., 15 min. to 60 min., 30 min. to 60 min., 45 min. to 60 min., 0.5 min. to 30 min., 1 min. to 30 min., 5 min. to 30 min., 10 min. to 30 min., 15 min. to 30 min., 0.5 min. to 15 min., 1 min. to 15 min., 5 min. to 15 min., 10 min. to 15 min., 0.5 min. to 10 min., 1 min. to 10 min., 5 min. to 10 min., 0.5 min. to 5 min., 1 min. to 5 min., or 0.5 min to 1 min.

For example, in some embodiments, to determine release of one or more food components from a formulated composition and/or formulated preparation, a preparation of a formulated composition and/or formulated preparation is added to one or more dissolution solvents and incubated for at least about 30 seconds, at least about 1 minute, at least about 5 minutes, at least about 10 minutes, at least about 30 minutes, at least about 60 minutes, at least about 120 minutes, at least about 6 hours, at least about 12 hours, or at least about 24 hours.

### C. Additional Features

### 1. Low Water Content/Water Activity

In some embodiments, a formulated composition and/or formulated preparation of the present disclosure is characterized as having low moisture content. Without wishing to be bound by any particular theory, formulated compositions having low moisture content are characterized as having improved stability (e.g., reduced degradation, delayed degradation, etc.) of one or more food components as compared to food components and/or formulations having higher moisture content.

In some embodiments, a formulated composition and/or formulated preparation is characterized as having a moisture content of less than about 0.5 wt % to 10 wt %, 1 wt % to 10 wt %, 2 wt % to 10 wt %, 4 wt % to 10 wt %, 6 wt % to 10 wt %, 8 wt % to 10 wt %, 0.5 wt % to 8 wt %, 1 wt % to 8 wt %, 2 wt % to 8 wt %, 4 wt % to 8 wt %, 6 wt % to 8 wt %, 0.5 wt % to 6 wt %, 1 wt % to 6 wt %, 2 wt % to 6 wt %, 4 wt % to 6 wt %, 0.5 wt % to 4 wt %, 1 wt % to 4 wt %, 2 wt % to 4 wt %, 0.5 wt % to 2 wt %, or 0.5% wt % to 1 wt %. For example, in some embodiments a formulated composition and/or formulated preparation is characterized as having a moisture content of less than about 0.5 wt %, 1 wt %, 2 wt %, 4 wt %, 6 wt %, 8 wt %, or 10 wt %.

In some embodiments, a formulated composition and/or formulated preparation is characterized by resistance or mitigation of water/moisture absorption when exposed to high humidity or moisture content. In some embodiments, a formulated composition and/or formulated preparation does not absorb more than about 0.25% (w/w) to 5% (w/w), 0.5% (w/w) to 5% (w/w), 0.75% (w/w) to 5% (w/w), 1% (w/w) to 5% (w/w), 1.5% (w/w) to 5% (w/w), 2% (w/w) to 5% (w/w), 2.5% (w/w) to 5% (w/w), 3% (w/w) to 5% (w/w), 3.5% (w/w) to 5% (w/w), 4% (w/w) to 5% (w/w), 4.5% (w/w) to 5% (w/w), 0.25% (w/w) to 4% (w/w), 0.5% (w/w) to 4% (w/w), 0.75% (w/w) to 4% (w/w), 1% (w/w) to 4% (w/w), 1.5% (w/w) to 4% (w/w), 2% (w/w) to 4% (w/w), 2.5% (w/w) to 4% (w/w), 3% (w/w) to 4% (w/w), 3.5% (w/w) to 4% (w/w), 0.25% (w/w) to 3% (w/w), 0.5% (w/w) to 3% (w/w), 0.75% (w/w) to 3% (w/w), 1% (w/w) to 3% (w/w), 1.5% (w/w) to 3% (w/w), 2% (w/w) to 3% (w/w), 2.5% (w/w) to 3% (w/w), 0.25% (w/w) to 2% (w/w), 0.5% (w/w) to 2% (w/w), 0.75% (w/w) to 2% (w/w), 1% (w/w) to 2% (w/w), 1.5% (w/w) to 2% (w/w), 0.25% (w/w) to 1% (w/w), 0.5% (w/w) to 1% (w/w), 0.75% (w/w) to 1% (w/w), or 0.25% (w/w) to 0.5% (w/w) when exposed to an environment having a relative humidity of about 33% to 75%, 53% to 75%, or 33% to 53%.

In some embodiments, a formulated composition and/or formulated preparation of the present disclosure is characterized as having low water activity. Without wishing to be bound by any particular theory, formulated compositions having low water activity are characterized as having improved stability (e.g., reduced degradation, delayed degradation, etc.) of one or more food components as compared to food components and/or formulations having higher water activity. In some embodiments, a formulated composition and/or formulated preparation of the present disclosure is characterized as having a water of activity of less than 0.1 to 0.5, 0.15 to 0.5, 0.2 to 0.5, 0.25 to 0.5, 0.3 to 0.5, 0.35 to 0.5, 0.4 to 0.5, 0.45 to 0.5, 0.1 to 0.4, 0.15 to 0.4, 0.2 to 0.4, 0.25 to 0.4, 0.3 to 0.4, 0.35 to 0.4, 0.1 to 0.3, 0.15 to 0.3, 0.2 to 0.3, 0.25 to 0.3, 0.1 to 0.2, 0.15 to 0.2, or 0.1 to 0.15. For example, in some embodiments, a formulated composition and/or formulated preparation of the present disclosure is characterized as having a water of activity of less than 0.1. In some embodiments, a formulated composition and/or formulated preparation of the present disclosure is characterized as having a water of activity of less than 0.2. In some embodiments, a formulated composition and/or formulated preparation of the present disclosure is characterized as having a water of activity of less than 0.3. In some embodiments, a formulated composition and/or formulated preparation of the present disclosure is characterized as having a water of activity of less than 0.4. In some embodiments, a formulated composition and/or formulated preparation of the present disclosure is characterized as having a water of activity of less than 0.5.

In some embodiments, a formulated composition and/or formulated preparation of the present disclosure is characterized as having a water activity less than 0.2. Without wishing to be bound by any particular theory, a water activity less than 0.2 supports the stability and/or longevity of one or more probiotic in a formulated preparation of the present disclosure.

### 2. Particle Size

In some embodiments, a preparation of a formulated composition and/or formulated preparation of the present disclosure comprises one or more particles of the formulated composition. In some embodiments, a preparation comprising one or more particles of a formulated composition and/or formulated preparation has a particular shape or form. In some embodiments, a preparation comprising one or more particles of a formulated composition and/or formulated preparation has an irregular shape or a cross-sectional shape of a circle, an oval, a triangle, a square, or a hexagon. In some embodiments, a preparation comprising one or more particles of a formulated composition and/or formulated preparation includes two or more particles having different shapes or forms. In some embodiments, a preparation comprises particles where substantially all or all the particles have a common shape.

In some embodiments, a preparation of a formulated composition and/or formulated preparation of the present disclosure is characterized as comprising particles having a distribution of diameters (e.g., Dv(10), Dv(20), Dv(30), Dv(40), Dv(50), Dv(60), Dv(70), Dv(80), Dv(90), Dv(99), etc.). In some embodiments, a preparation of a formulated composition and/or formulated preparation of the present disclosure is characterized as comprising particles having an average diameter (e.g., D[3,2], D[4,3], etc.). Regardless of the shape of a particle, the “diameter” (i.e., size) of a particle is the longest distance from one end of the particle to another end of the particle.

In some embodiments, a preparation of a formulated composition and/or formulated preparation of the present disclosure is characterized as comprising particles having a distribution of diameters (e.g., Dv(10), Dv(20), Dv(30), Dv(40), Dv(50), Dv(60), Dv(70), Dv(80), Dv(90), Dv(99), etc.) of about 5 μm to 10,000 μm, 10 μm to 10,000 μm, 20 μm to 10,000 μm, 30 μm to 10,000 μm, 40 μm to 10,000 μm, 50 μm to 10,000 μm, 100 μm to 10,000 μm, 200 μm to 10,000 μm, 400 μm to 10,000 μm, 800 μm to 10,000 μm, 1000 μm to 10,000 μm, 1250 μm to 10,000 μm, 1500 μm to 10,000 μm, 2000 μm to 10,000 μm, 2500 μm to 10,000 μm, 5000 μm to 10,000 μm, 7500 μm to 10,000 μm, 5 μm to 5000 μm, 10 μm to 5000 μm, 20 μm to 5000 μm, 30 μm to 5000 μm, 40 μm to 5000 μm, 50 μm to 5000 μm, 100 μm to 5000 μm, 200 μm to 5000 μm, 400 μm to 5000 μm, 800 μm to 5000 μm, 1000 μm to 5000 μm, 1250 μm to 5000 μm, 1500 μm to 5000 μm, 2000 μm to 5000 μm, 2500 μm to 5000 μm, 5 μm to 2500 μm, 10 μm to 2500 μm, 20 μm to 2500 μm, 30 μm to 2500 μm, 40 μm to 2500 μm, 50 μm to 2500 μm, 100 μm to 2500 μm, 200 μm to 2500 μm, 400 μm to 2500 μm, 800 μm to 2500 μm, 1000 μm to 2500 μm, 1250 μm to 2500 μm, 1500 μm to 2500 μm, 2000 μm to 2500 μm, 5 μm to 1250 μm, 10 μm to 1250 μm, 20 μm to 1250 μm, 30 μm to 1250 μm, 40 μm to 1250 μm, 50 μm to 1250 μm, 100 μm to 1250 μm, 200 μm to 1250 μm, 400 μm to 1250 μm, 800 μm to 1250 μm, 1000 μm to 1250 μm, 5 μm to 625 μm, 10 μm to 625 μm, 20 μm to 625 μm, 30 μm to 625 μm, 40 μm to 625 μm, 50 μm to 625 μm, 100 μm to 625 μm, 200 μm to 625 μm, 400 μm to 625 μm, 5 μm to 300 μm, 10 μm to 300 μm, 20 μm to 300 μm, 30 μm to 300 μm, 40 μm to 300 μm, 50 μm to 300 μm, 100 μm to 300 μm, 200 μm to 300 μm, 5 μm to 150 μm, 10 μm to 150 μm, 20 μm to 150 μm, 30 μm to 150 μm, 40 μm to 150 μm, 50 μm to 150 μm, 100 μm to 150 μm, 5 μm to 100 μm, 10 μm to 100 μm, 20 μm to 100 μm, 30 μm to 100 μm, 40 μm to 100 μm, 50 μm to 100 μm, 5 μm to 50 μm, 10 μm to 50 μm, 20 μm to 50 μm, 30 μm to 50 μm, 40 μm to 50 μm, 5 μm to 20 μm, 10 μm to 20 μm, or 5 μm to 10 μm. For example, in some embodiments, a preparation of a formulated composition and/or formulated preparation of the present disclosure is characterized as comprising particles having a distribution of diameters of up to about 5 μm, 10 μm, 20 μm, 30 μm, 40 μm, 50 μm, 100 μm, 200 μm, 400 μm, 800 μm, 1250 μm, 2500 μm, or 5000 μm.

Without wishing to be bound to any particular theory, a preparation of a formulated composition and/or formulated preparation of the present disclosure comprising particles having substantially uniform shapes and small particle diameters may be characterized as being amenable for incorporating into a food and or beverage product (e.g., a powder (e.g., protein powder), bar, food, beverage, or supplement). In some embodiments, a preparation of a formulated composition and/or formulated preparation of the present disclosure comprising particles having substantially uniform shapes and small particle diameters may be characterized as having an appealing mouth-feel when ingested by a subject.

### 3. Density

In some embodiments, a formulated composition and/or formulated preparation of the present disclosure is characterized as having a bulk density of about 0.15 g/mL, about 0.2 g/mL, about 0.25 g/mL, about 0.3 g/mL, about 0.35 g/mL, about 0.4 g/mL, about 0.45 g/mL, about 0.5 g/mL, about 0.55 g/mL, about 0.6 g/mL, about 0.65 g/mL, about 0.7 g/mL, about 0.75 g/mL, about 0.8 g/mL, about 0.85 g/mL, about 0.9 g/mL, about 0.95 g/mL, about 1.0 g/mL, about 1.05 g/mL, about 1.1 g/mL, about 1.15 g/mL, about 1.2 g/mL, about 1.25 g/mL, about 1.3 g/mL, about 1.35 g/mL, about 1.4 g/mL, about 1.45 g/mL, about 1.5 g/mL, about 1.55 g/mL, about 1.6 g/mL, about 1.65 g/mL, about 1.7 g/mL, about 1.75 g/mL, about 1.8 g/mL, about 1.85 g/mL, about 1.9 g/mL, about 1.95 g/mL, or about 2 g/mL. In some embodiments, a formulated composition and/or formulated preparation of the present disclosure is characterized as having a tap density of about 0.15 g/mL, about 0.2 g/mL, about 0.25 g/mL, about 0.3 g/mL, about 0.35 g/mL, about 0.4 g/mL, about 0.45 g/mL, about 0.5 g/mL, about 0.55 g/mL, about 0.6 g/mL, about 0.65 g/mL, about 0.7 g/mL, about 0.75 g/mL, about 0.8 g/mL, about 0.85 g/mL, about 0.9 g/mL, about 0.95 g/mL, about 1.0 g/mL, about 1.05 g/mL, about 1.1 g/mL, about 1.15 g/mL, about 1.2 g/mL, about 1.25 g/mL, about 1.3 g/mL, about 1.35 g/mL, about 1.4 g/mL, about 1.45 g/mL, about 1.5 g/mL, about 1.55 g/mL, about 1.6 g/mL, about 1.65 g/mL, about 1.7 g/mL, about 1.75 g/mL, about 1.8 g/mL, about 1.85 g/mL, about 1.9 g/mL, about 1.95 g/mL, or about 2 g/mL. In some embodiments, a formulated composition and/or formulated preparation of the present disclosure is characterized by a Hausner ratio of less than about 0.7, less than about 0.8, less than about 0.9, less than about 1.0, less than about 1.1, less than about 1.2, less than about 1.3, less than about 1.4, less than about 1.5, less than about 1.6, less than about 1.7, less than about 1.8, less than about 1.9, less than about 2.0, less than about 2.1, less than about 2.2, less than about 2.3, or less than about 2.4,

### 4. Low Viscosity

In some embodiments, a formulated composition and/or formulated preparation of the present disclosure is characterized as having a viscosity of about 1 cP to about 150 cP, about 25 cP to about 150 cP, about 50 cP to about 150 cP, about 75 cP to about 150 cP, about 100 cP to about 150 cP, about 125 cP to about 150 cP, about 1 cP to about 125 cP, about 25 cP to about 125 cP, about 50 cP to about 125 cP, about 75 cP to about 125 cP, about 100 cP to about 125 cP, about 1 cP to about 100 cP, about 25 cP to about 100 cP, about 50 cP to about 100 cP, about 75 cP to about 100 cP, about 1 cP to about 75 cP, about 25 cP to about 75 cP, about 50 cP to about 75 cP, about 1 cP to about 50 cP, about 25 cP to about 50 cP, or about 1 cP to about 25 cP. For example, in some embodiments, a formulated composition and/or formulated preparation of the present disclosure is characterized as having a viscosity of about 1 cP, 25 cP, about 50 cP, about 75 cP, about 100 cP, about 125 cP, or about 150 cP.

In some embodiments, viscosity of a formulated preparation may be measured using a rotating disk (e.g., 20 mm).

In some embodiments, the presence of one or more excipients (e.g., 1, 2, 3, 4, 5, 6, 7, 8, 9, or 10 excipients) in a formulated preparation reduces the viscosity of the preparation.

Without wishing to be bound by any particular theory, it is contemplated that formulated preparations characterized as having low viscosities (e.g., about 25 cP to about 150 cP) can be more homogeneously incorporated into food and/or beverage products. In some embodiments, formulated preparations characterized as having low viscosities (e.g., about 25 cP to about 150 cP) may improve mouth-feel when said preparation is incorporated into food and/or beverage products.

### D. Additional Benefits

1. Incorporation into Foods/Beverage Products

In some embodiments, the present disclosure provides for the incorporation of a formulated composition and/or formulated preparation into a food and/or beverage product.

In some embodiments, a formulated composition and/or formulation preparation may be incorporated into a food and/or beverage product including, but not limited to, protein powder, whey powder, baby formula, dry food powder, protein bar, snack bar, yogurt, supplements, bread, candy, cake, cereal, chip, chocolate, confectionary, cookie, food additive, gummy, ice cream, kefir, rice, pasta, dry food, nutrition supplement, packaged food, pet feed, pet food, protein powder, sachet, salad dressing, salty snack, seed, smoothie, spice, Meal Ready-to-Eat (MRE), frozen food, medical food, fermented food, protein shakes, drink mix powders, dairy milk, oat milk, almond milk, soy milk, coffee grinds, Meal Ready-to-Drink (RTD), ready-to-drink low phenylalanine medical food, electrolyte beverages, sports beverages, water, hard seltzers, alcoholic seltzers, beer, wine, soda, coffee, tea, fermented beverages, carbonated beverage, Gatorade™, Truly™, Ensure™, PKU Sphere™ Liquid.

In some embodiments, a formulated composition and/or formulated preparation is characterized as having physical and/or chemical properties that allow said formulation to have sufficient mixability to incorporate with a food and/or beverage product. In some embodiments, a formulated composition and/or formulated preparation is characterized as having physical and/or chemical properties that allow said formulation to have sufficient flowability to incorporate with a food and/or beverage product.

In some embodiments, a formulated composition and/or formulated preparation, may be incorporated into a food and/or beverage product during the manufacturing process of said food and/or beverage product. In some embodiments, a formulated composition and/or formulated preparation, may be incorporated into a food and/or beverage product during the packaging of said food and/or beverage product. In some embodiments, a formulated composition and/or formulated preparation, may be incorporated into a food and/or beverage product during the pasteurization of said food and/or beverage product. In some embodiments, a formulated composition and/or formulated preparation, may be incorporated into a food and/or beverage product prior to mixing of said food and/or beverage product. In some embodiments, a formulated composition and/or formulated preparation, may be incorporated into a finished food and/or beverage product. For example, in some embodiments, a formulated composition and/or formulated preparation may be provided as a ready-to-mix powder for incorporation into a finished food and/or beverage product.

In some embodiments, a formulated composition and/or formulated preparation, may be incorporated into a food and/or beverage product immediately before ingestion of said food and/or beverage product. In some embodiments, a formulated composition and/or formulated preparation, may be incorporated into a food and/or beverage product before sterilization of said food and/or beverage product. In some embodiments, a formulated composition and/or formulated preparation, may be incorporated into a food and/or beverage product after sterilization of said food and/or beverage product.

In some embodiments, incorporation of a formulated composition and/or formulated preparation into a food and/or beverage product comprises one or more size reduction steps. In some embodiments, a size reduction step may be performed on a formulated composition and/or formulated preparation prior to incorporation into a food and/or beverage product. In some embodiments, a size reduction step may be performed concurrently with the incorporation of a formulated composition and/or formulated preparation with a food and/or beverage product. In some embodiments, a size reduction step may be performed after the incorporation of a formulated composition and/or formulated preparation with a food and/or beverage product.

In some embodiments, a size reduction step may comprise planetary milling, ball milling, burr milling, roller milling, media milling, impact milling, jet milling, high-pressure homogenization, cryo-milling, hammer milling, conical milling, hand screening, granulation/extrusion, extrusion, spray drying, lyophilization/milling, fluid bed agglomeration, spray congealing, high-shear granulation, tableting, pouring, roller compaction, crosslinking, prilling, spinning disc atomization, or a combination thereof.

In some embodiments, incorporation of a formulated composition and/or formulated preparation into a food and/or beverage product comprises one or more homogenization steps.

In some embodiments, a homogenization step is applied to a formulated composition and/or formulated preparation after incorporation of said formulation into a food and/or beverage product. In some embodiments, a homogenization step may comprise use of an overhead stirrer, manual stirring, stir bar, high pressure homogenization, low pressure homogenization, sonication, ultrasonication, vortexing, or a combination thereof.

In some embodiments, incorporation of a formulated composition and/or formulated preparation into a food and/or beverage product significantly affects the visual appearance of said food and/or beverage product. In some embodiments, incorporation of a formulated composition and/or formulated preparation into a food and/or beverage product minimally affects the visual appearance of said food and/or beverage product. In some embodiments, incorporation of a formulated composition and/or formulated preparation into a food and/or beverage product improves the visual appearance of said food and/or beverage product.

In some embodiments, incorporation of a formulated composition and/or formulated preparation into a food and/or beverage product significantly affects the taste of said food and/or beverage product. In some embodiments, incorporation of a formulated composition and/or formulated preparation into a food and/or beverage product minimally affects the taste of said food and/or beverage product. In some embodiments, incorporation of a formulated composition and/or formulated preparation into a food and/or beverage product improves the taste of said food and/or beverage product.

In some embodiments, a formulated composition and/or formulated preparation is incorporated into a food product including, but not limited to, protein powder, whey powder, baby formula, dry food powder, protein bar, snack bar, yogurt, supplements, bread, candy, cake, cereal, chip, chocolate, confectionary, cookie, food additive, gummy, ice cream, kefir, rice, pasta, dry food, nutrition supplement, packaged food, pet feed, pet food, protein powder, sachet, salad dressing, salty snack, seed, smoothie, spice, Meal Ready-to-Eat (MRE), frozen food, medical food, or fermented food.

In some embodiments, a formulated composition and/or formulated preparation is incorporated into a beverage product characterized as having high water activity. In some embodiments, a formulated composition and/or formulated preparation is incorporated into a beverage product including, but not limited to, protein shakes, drink mix powders, dairy milk, oat milk, almond milk, soy milk, coffee grinds, Meal Ready-to-Drink (RTD), ready-to-drink low phenylalanine medical food, electrolyte beverages, sports beverages, water, hard seltzers, alcoholic seltzers, beer, wine, soda, coffee, tea, fermented beverages, carbonated beverage, Gatorade™, Truly™, Ensure™, PKU Sphere™ Liquid.

In some embodiments, a formulated composition and/or formulated preparation is incorporated into a powder-based supplement product, powder-based food product, and/or powder-based beverage-mix product. In some embodiments, a powder-based supplement product, powder-based food product, and/or powder-based beverage-mix product includes, but is not limited to, a pre-workout powder, a post-workout powder or pill, a pre-workout capsule/pill, a protein powder, a whey powder, a baby formula, a milk powder, or a drink powder (e.g., Kool-Aid type mix).

In some embodiments, a formulated composition and/or formulated preparation incorporated into a powder-based supplement product, powder-based food product, and/or powder-based beverage product (e.g., beverage mix) exhibits visual homogeneity when the formulated food product, formulated beverage product, or formulated supplement product is mixed into a beverage including, but not limited to, protein shakes, drink mix powders, dairy milk, oat milk, almond milk, soy milk, coffee grinds, Meal Ready-to-Drink (RTD), ready-to-drink low phenylalanine medical food, electrolyte beverages, sports beverages, water, hard seltzers, alcoholic seltzers, beer, wine, soda, coffee, tea, fermented beverages, carbonated beverage, Gatorade™, Truly™, Ensure™, PKU Sphere™ Liquid at a concentration of at least about 1% (w/v), 2% (w/v), 5% (w/v), 8% (w/v), 10% (w/v), 14% (w/v), 18% (w/v), or 20% (w/v). In some embodiments, a formulated composition and/or formulated preparation incorporated into a powder-based supplement product, powder-based food product (e.g., protein powder), and/or powder-based beverage-mix product exhibits no flocculation over a period of at least about 1 minute, at least about 2 minutes, at least about 5 minutes, at least about 10 minutes, at least about 20 minutes, at least about 30 minutes, at least about 45 minutes, or at least about 60 minutes when the formulated food product, formulated beverage product, or formulated supplement product is mixed into a beverage including, but not limited to, protein shakes, drink mix powders, dairy milk, oat milk, almond milk, soy milk, coffee grinds, Meal Ready-to-Drink (RTD), ready-to-drink low phenylalanine medical food, electrolyte beverages, sports beverages, water, hard seltzers, alcoholic seltzers, beer, wine, soda, coffee, tea, fermented beverages, carbonated beverage, Gatorade™, Truly™, Ensure™, PKU Sphere™ Liquid, at a concentration of at least about 1% (w/v), 2% (w/v), 5% (w/v), 8% (w/v), 10% (w/v), 14% (w/v), 18% (w/v), or 20% (w/v). In some embodiments, a formulated composition and/or formulated preparation incorporated into a powder-based supplement product, powder-based food product (e.g., protein powder), and/or powder-based beverage-mix, affords a viscosity change of less than about 1.2-fold, less than about 1.6-fold, less than about 2-fold, less than about 3-fold, less than about 5-fold, less than about 10-fold, less than about 20-fold, less than about 50-fold, less than about 100-fold, less than about 200-fold, less than about 500-fold, or less than about 1000-fold when the formulated food product, formulated beverage product, formulated supplement product is mixed into a beverage including, but not limited to, protein shakes, drink mix powders, dairy milk, oat milk, almond milk, soy milk, coffee grinds, Meal Ready-to-Drink (RTD), ready-to-drink low phenylalanine medical food, electrolyte beverages, sports beverages, water, hard seltzers, alcoholic seltzers, beer, wine, soda, coffee, tea, fermented beverages, carbonated beverage, Gatorade™, Truly™, Ensure™, PKU Sphere™ Liquid at a concentration of at least about 1% (w/v), 2% (w/v), 5% (w/v), 8% (w/v), 10% (w/v), 14% (w/v), 18% (w/v), or 20% (w/v).

### 2. Stability of Food Components

Without wishing to be bound by any particular theory, those skilled in the art will recognize that incorporation of a formulated composition and/or formulated preparation into a food and/or beverage product may be associated with a significant reduction in the stability of said preparation, composition, and/or one or more food components therein. In some embodiments, stability of one or more food components may be characterized as the chemical stability, physical stability, stability of function, stability of benefit (e.g., nutritional benefit), or a combination thereof, of the one or more food components.

In some embodiments, stability of one or more food components in a formulated composition, formulated preparation, and/or formulated composition incorporated into a food and/or beverage product is characterized upon mixing of said formulation, preparation, or food and/or beverage product with one or more dissolution solvents at a predetermined temperature, humidity, period of time (e.g., incubation period), or a combination thereof. In some embodiments, a formulated composition and/or formulated preparation provides improved stability of one or more food components in one or more dissolution solvents (e.g., water, simulated gastric fluid, simulated intestinal fluid) as compared to one or more unformulated food component. In some embodiments, a formulated composition and/or food preparation provides improved stability of one or more food components when incorporated into a food and/or beverage product as compared to one or more unformulated food component. In some embodiments, a formulated composition and/or food preparation provides improved stability of one or more food components in high relative humidity environments as compared to one or more unformulated food component. In some embodiments, a formulated composition and/or food preparation provides improved stability of one or more food components in high temperature environments as compared to one or more unformulated food component.

In some embodiments, a formulated composition and/or formulated preparation of the present disclosure may be characterized as being able to protect one or more food components against a physical change, chemical change, functional change, change in benefit, or a combination thereof. In some embodiments, a physical change, chemical change, functional change or change in benefit may be induced by heat, light, shear, water, acid, enzymes, bacteria, or a combination thereof.

In some embodiments, stability of one or more food components in a formulated composition, a formulated preparation, or a food and/or beverage product incorporating a formulated composition, may be characterized as the percentage of change of a measured stability characteristic (e.g., stability property) after a period of storage as compared to the measured stability characteristic immediately after production of the formulated composition and/or formulated preparation.

In some embodiments, stability of one or more food components in a formulated composition, formulated preparation, or a food and/or beverage product incorporating a formulated composition, may be characterized as a percent change in the quantity (e.g., mol., g., lbs.) of one or more food components in a formulated composition and/or formulated preparation after a period of storage as compared to the quantity of one or more food components immediately after production of the formulated composition and/or formulated preparation.

In some embodiments, stability of one or more food components in a formulated composition, formulated preparation, or a food and/or beverage product incorporating a formulated composition, may be characterized as a percent change in a physical parameter (e.g., diameter, morphology, porosity) of a formulated composition and/or formulated preparation after a period of storage as compared to the physical parameter of the formulated composition and/or formulated preparation immediately after production.

In some embodiments, stability of one or more food components in a formulated composition, formulated preparation, or a food and/or beverage product incorporating a formulated composition, may be characterized as a change in a release profile of one or more food components from a formulated composition and/or formulated preparation after a period of storage as compared to the release profile of the one or more food components from the formulated composition and/or formulated preparation immediately after production.

In some embodiments, stability of one or more food components in a formulated composition, formulated preparation, or a food and/or beverage product incorporating a formulated composition, may be characterized as a change in a health benefit provided by one or more food components from a formulated composition and/or formulated preparation in a subject ingesting said formulation after a period of storage as compared to the health benefit provided by the one or more food components from the formulated composition and/or formulated preparation immediately after production.

In some embodiments, stability of one or more food components in a formulated composition, formulated preparation, or a food and/or beverage product incorporating a formulated composition, may be characterized as a change in microbiome health benefit provided by one or more food components from a formulated composition and/or formulated preparation in a subject ingesting said formulated composition and/or formulated preparation after a period of storage as compared to the microbiome health benefit provided by the one or more food components from the formulated composition and/or formulated preparation immediately after production.

In some embodiments, stability of one or more food components in a formulated composition, formulated preparation, or a food and/or beverage product incorporating a formulated composition, may be characterized as a change in energy (e.g., calories) provided by one or more food components from a formulated composition and/or formulated preparation after a period of storage as compared to the energy (e.g., calories) provided by the one or more food components from the formulated composition and/or formulated preparation immediately after production.

In some embodiments, stability of one or more food components in a formulated composition, formulated preparation, or a food and/or beverage product incorporating a formulated composition, may be characterized as a change in metabolic intermediates provided by one or more food components from a formulated composition after a period of storage as compared to the metabolic intermediates provided by the one or more food components from the formulated composition and/or formulated preparation immediately after production.

In some embodiments, stability of one or more food components in a formulated composition, formulated preparation, or a food and/or beverage product incorporating a formulated composition, may be characterized as a change in osmotic stability provided by one or more food components from a formulated composition after a period of storage as compared to the osmotic stability provided by the one or more food components from the formulated composition and/or formulated preparation immediately after production.

In some embodiments, stability of one or more food components in a formulated composition, formulated preparation, or a food and/or beverage product incorporating a formulated composition, changes by less than about 40%, less than about 35%, less than about 30%, less than about 25%, less than about 20%, less than about 10%, less than about 9%, less than about 8%, less than about 7%, less than about 6%, less than about 5%, less than about 4%, less than about 3%, less than about 2%, less than about 1%, or less than about 0.5% after a specified period of time (e.g., storage period, or incubation period) in specified environmental conditions.

In some embodiments, a specified period of time (e.g., storage period, or incubation period) for characterizing stability of one or more food components is about 1 week, about 2 weeks, about 3 weeks, about 4 weeks, about 5 weeks, about 6 weeks, about 7 weeks, about 8 weeks, about 9 weeks, about 10 weeks, about 12 weeks, about 16 weeks, about 24 weeks, or about 52 weeks, about 1.5 years, about 2 years, about 2.5 years, about 3 years, about 4 years, or about 5 years.

In some embodiments, specified environmental conditions for characterizing stability of one or more food components includes, but are not limited to, ambient temperature, body temperature (e.g., 37 C), aqueous environment, exposure to light, incorporation into a food and/or beverage product, high relative humidity, high water content, or a combination thereof.

In some embodiments, stability of one or more food components in a formulated composition, formulated preparation, or a food and/or beverage product incorporating a formulated composition, is maintained (<about 20% change in one or more stability properties) after storage in a solid food (e.g., bread, rice, baked goods, etc.) at an ambient temperature for about 1 week, about 2 weeks, about 3 weeks, about 4 weeks, about 5 weeks, about 6 weeks, about 7 weeks, about 8 weeks, about 9 weeks, about 10 weeks, about 12 weeks, about 16 weeks, about 24 weeks, or about 52 weeks, about 1.5 years, about 2 years, about 2.5 years, about 3 years, about 4 years, or about 5 years.

In some embodiments, stability of one or more food components in a formulated composition, formulated preparation, or a food and/or beverage product incorporating a formulated composition, is maintained (<about 20% change in one or more stability properties) after storage in a dry powder (e.g., protein powder, milk powder, baby formula, supplement powder, flour, etc.) at an ambient temperature for about 1 week, about 2 weeks, about 3 weeks, about 4 weeks, about 5 weeks, about 6 weeks, about 7 weeks, about 8 weeks, about 9 weeks, about 10 weeks, about 12 weeks, about 16 weeks, about 24 weeks, or about 52 weeks, about 1.5 years, about 2 years, about 2.5 years, about 3 years, about 4 years, or about 5 years.

In some embodiments, stability of one or more food components in a formulated composition, a preparation of a formulated composition, or a food and/or beverage product incorporating a formulated composition, is maintained (<about 20% change in one or more stability properties) after storage in a liquid beverage (e.g., water, coffee, drinkable yogurt, protein beverage, water, soda, Gatorade, sports drinks, etc.) at an ambient temperature for about 1 week, about 2 weeks, about 3 weeks, about 4 weeks, about 5 weeks, about 6 weeks, about 7 weeks, about 8 weeks, about 9 weeks, about 10 weeks, about 12 weeks, about 16 weeks, about 24 weeks, or about 52 weeks, about 1.5 years, about 2 years, about 2.5 years, about 3 years, about 4 years, or about 5 years.

In some embodiments, stability of one or more food components in a formulated composition, formulated preparation, or a food and/or beverage product incorporating a formulated composition, is maintained (<about 20% change in one or more stability properties) after storage in yogurt at ambient temperature for up to 2 weeks.

In some embodiments, stability of one or more food components in a formulated composition, formulated preparation, or a food and/or beverage product incorporating a formulated composition, is maintained (<about 20% change in one or more stability properties) after storage in milk powder at ambient temperature for up to 2 weeks.

In some embodiments, stability of one or more food components in a formulated composition, formulated preparation, or a food and/or beverage product incorporating a formulated composition, is maintained (<about 20% change in one or more stability properties) after storage in baby formula at ambient temperature for up to 2 weeks.

In some embodiments, stability of one or more food components in a formulated composition, formulated preparation, or a food and/or beverage product incorporating a formulated composition, is maintained (<about 20% change in one or more stability properties) after storage in high dry powder at ambient temperature for up to 2 weeks.

In some embodiments, stability of one or more food components in a formulated composition, formulated preparation, or a food and/or beverage product incorporating a formulated composition, is maintained (<about 20% change in one or more stability properties) after storage in a sachet at ambient temperature for up to 2 weeks.

In some embodiments, stability of one or more food components in a formulated composition, formulated preparation, or a food and/or beverage product incorporating a formulated composition, is maintained (<about 20% change in one or more stability properties) after combination and storage in an animal feed at ambient temperature for up to 2 weeks.

In some embodiments, stability of one or more food components in a formulated composition, formulated preparation, or a food and/or beverage product incorporating a formulated composition, is maintained (<about 20% change in one or more stability properties) after storage at ambient temperature, about 30% relative humidity, for up to 6 weeks.

In some embodiments, stability of one or more food components in a formulated composition, formulated preparation, or a food and/or beverage product incorporating a formulated composition, is maintained (<about 20% change in one or more stability properties) after storage at greater than about 15% relative humidity, greater than about 20% relative humidity, greater than about 25% relative humidity, greater than about 30% relative humidity, greater than about 35% relative humidity, at greater than about −20° C., greater than about 4° C., greater than about 25° C., greater than about 30° C., greater than about 35° C., or greater than about 37° C., for greater than about 1 week, greater than about 2 weeks, greater than about 3 weeks, greater than about 4 weeks, greater than about 6 weeks, or greater than about 8 weeks.

In some embodiments, stability of one or more food components in a formulated composition, formulated preparation, or a food and/or beverage product incorporating a formulated composition, is maintained (<about 20% change in one or more stability properties) after storage in a freezer (−85 C to 0° C.), a refrigerator (1-10° C.), or atmospheric temperature (−10° C.-40° C.) for time periods about 1 week, about 2 weeks, about 3 weeks, about 4 weeks, about 5 weeks, about 6 weeks, about 7 weeks, about 8 weeks, about 9 weeks, about 10 weeks, about 12 weeks, about 16 weeks, about 24 weeks, or about 52 weeks, about 1.5 years, about 2 years, about 2.5 years, about 3 years, about 4 years, or about 5 years.

In some embodiments, stability of one or more food components in a formulated composition, formulated preparation, or a food and/or beverage product incorporating a formulated composition, is maintained (<about 20% change in one or more stability properties) after storage in a high water activity environment at ambient temperature for about 1 week, about 2 weeks, about 3 weeks, about 4 weeks, about 5 weeks, about 6 weeks, about 7 weeks, about 8 weeks, about 9 weeks, about 10 weeks, about 12 weeks, about 16 weeks, about 24 weeks, or about 52 weeks, about 1.5 years, about 2 years, about 2.5 years, about 3 years, about 4 years, or about 5 years.

### 3. Reduced Meal Frequency

Among other things, the present disclosure provides a means for reducing daily meal frequency. As used herein, a daily meal refers one or more food components ingested by a subject in a singular portion at regular intervals (e.g., meal frequency) over a 24-hour period (e.g., daily). A daily meal may be characterized as providing an effective dose of one or more food components to achieve satisfaction and/or satiety (e.g., a pleasurable taste, a pleasurable fragrance, comfort, mitigate hunger) in a subject consuming said meal. A daily meal may be further characterized as providing an effective dose of one or more nutritional benefits (e.g., providing energy (e.g., caloric content), maintaining health, maintaining microbiome health, providing metabolic intermediates, providing osmotic stabilizers, or a combination thereof) to a subject consuming said meal. An effective dose may be achieved in a subject by consuming one daily meal (e.g., in a 24-hour period). Alternatively, an effective dose may be achieved by consuming two or more (e.g., 2, 3, 4) daily meals (e.g., in a 24-hour period).

Those skilled in the art will appreciate that a subject may consume one or more meals at regular intervals (e.g., meal frequency) over a 24-hour period (e.g., daily). For example, a subject may consume at least 1, 2, 3, 4, 5, 6, 7, 8, 9, or at least 10 meals in a 24-hour period (e.g., to 5 meals daily). Without wishing to be bound by any particular theory, it is contemplated that the consumption of one or more meals by a subject may be primarily driven by a need for satisfaction and/or satiety and secondarily driven by a need for a nutritional benefit.

Without wishing to be bound by any particular theory, it is contemplated that a meal characterized as providing satisfaction and/or satiety is not necessarily characterized as providing a nutritional benefit to a subject consuming said meal. Similarly, without wishing to be bound by any particular theory, it is contemplated that a meal characterized as providing a nutritional benefit is not necessarily satisfying and/or satiating to a subject consuming said meal. It is similarly contemplated that an effective daily dose of one or more food components such as to provide for satisfaction and/or satiety, may be an insufficient daily dose of one or more food components such as to provide for a nutritional benefit. It is similarly contemplated that an effective daily dose of one or more food components such as to provide for satisfaction and/or satiety, may be an excessive daily dose of one or more food components such as to provide for a nutritional benefit.

Without wishing to be bound by any particular theory, it is contemplated that the consumption of several meals in a 24-hour period facilitates provision of satisfaction, satiety, and/or a nutritional benefit at the cost of meal preparation time and meal consumption time. Moreover, it is contemplated that consumption of several meals in a 24-hour period is associated with an increased risk of an error in selection of one or more food components, such as to provide an insufficient and/or excessive daily dose of one or more food components. Without wishing to be bound by any particular theory, reduction of meal frequency mitigates meal preparation time and meal consumption time at the cost of satisfaction, satiety, and/or nutritional benefit.

The present disclosure provides a method of reducing daily meal frequency of a subject. In some embodiments, a method of the present disclosure provides satisfaction to a subject. In some embodiments, a method of the present disclosure provides satiety to a subject. In some embodiments, a method of the present disclosure provides a nutritional benefit to a subject. In some embodiments, a method of the present disclosure provides an effective daily dose of one or more food components to a subject.

In some embodiments, the present disclosure provides a method for reducing a daily meal frequency of a subject comprising administering a formulated composition (and/or formulated preparation) to the subject. In some embodiments, the present disclosure provides a method for providing satisfaction to a subject comprising administering a formulated composition (and/or formulated preparation) to the subject. In some embodiments, the present disclosure provides a method for providing satiety to a subject comprising administering a formulated composition (and/or formulated preparation) to the subject. In some embodiments, the present disclosure provides a method for providing a nutritional benefit to a subject comprising administering a formulated composition (and/or formulated preparation) to the subject.

In some embodiments, one or more food components of a formulated composition (and/or formulated preparation) provides satisfaction, satiety, and/or a nutritional benefit to a subject ingesting said formulation (and/or preparation of the formulated composition). In some embodiments, one or more excipient components of a formulated composition (and/or formulated preparation) provides satisfaction, satiety, and/or a nutritional benefit to a subject ingesting said formulation (and/or preparation of the formulated composition). In some embodiments, the physical arrangement of a formulated composition (and/or formulated preparation) provides satisfaction, satiety, and/or a nutritional benefit to a subject ingesting said formulation (and/or preparation of the formulated composition).

In some embodiments, a method of providing satiety to a subject of the present disclosure, also provides satisfaction and/or nutritional benefit. In some embodiments, a method of providing satisfaction to a subject of the present disclosure, also provides satiety and/or a nutritional benefit. In some embodiments, a method of providing a nutritional benefit to a subject of the present disclosure, also provides satisfaction and/or satiety.

### 4. Satisfaction and Satiety

As provided herein, among other things, the present disclosure provides a method for providing satisfaction and/or satiety to a subject comprising administering a formulated composition (and/or formulated preparation) to the subject. Without wishing to be bound by any particular theory, satisfaction and/or satiety in a subject is achieved by: providing a pleasing taste; providing a pleasing texture; controlling release (e.g., extending the duration of release) of one or more food components; controlling the physiological response of a subject ingesting a formulated composition; or a combination thereof.

In some embodiments, a formulated composition of the present disclosure (and/or formulated preparation) provides satisfaction and/or satiety to a subject. In some embodiments, one or more food components of an ingestible preparation of the present disclosure (and/or formulated preparation) provides satisfaction and/or satiety to a subject. In some embodiments, one or more release modulators of an ingestible preparation of the present disclosure (and/or formulated preparation) provides satisfaction and/or satiety to a subject. In some embodiments, one or more excipients of an ingestible preparation of the present disclosure (and/or formulated preparation) provides satisfaction and/or satiety to a subject.

In some embodiments of the present disclosure, a formulated composition (and/or formulated preparation) characterized as providing controlled, extended and/or delayed release of one or more food components satisfies a craving a subject that has ingested said formulated composition (and/or formulated preparation).

### (i) Taste

In some embodiments, a formulated composition of the present disclosure is characterized as having a pleasing taste. In some embodiments, one or more food components are characterized as taste modifiers, wherein the one of more food components confer a pleasing taste to a formulated composition (e.g., as experienced by a subject ingesting said formulation). In some embodiments, one or more release modulators are characterized as taste modifiers, wherein the one of more release modulators confer a pleasing taste to a formulated composition (e.g., as experienced by a subject ingesting said formulation). In some embodiments, one or more excipients are characterized as taste modifiers, wherein the one of more excipients confer a pleasing taste to a formulated composition (e.g., as experienced by a subject ingesting said formulation). In some embodiments, a combination of one or more food components, one or more release modulators, and/or one or more excipients is characterized as a taste modifier, wherein the combination confers a pleasing taste to a formulated composition (e.g., as experienced by a subject ingesting said formulation).

Without wishing to be bound by any particular theory, a taste modifier may be characterized as providing a sweet taste, sour taste, salty taste, bitter taste, savory taste, or a combination thereof. In some embodiments, a taste modifier may directly provide a pleasing taste to a formulated composition (e.g., as experienced by a subject ingesting said formulation). In some embodiments, a taste modifier may modulate the taste of one or more other food components.

In some embodiments, a formulated composition of the present disclosure may further comprise one or more additional taste modifiers. In some embodiments, one or more additional taste modifiers may include, but are not limited to, glucose, vanillin, acetic acid, chlorogenic acid, cafestol, sodium chloride, or a combination thereof.

Without wishing to be bound by any particular theory, one skilled in the art will appreciate that one or more additional taste modifiers may further include, but are not limited to, substances identified by one or more governing bodies as safe (e.g., generally regarded as safe and/or food additives). In some embodiments, those skilled in the art will appreciate that one or more taste modifiers may be selected from those substances recognized as Generally Regarded as Safe (i.e., GRAS) by the U.S. Food and Drug Administration. In some embodiments, those skilled in the art will appreciate that one or more taste modifiers may be selected from those substances recognized in 21 C.F.R. 184. In some embodiments, those skilled in the art will appreciate that one or more taste modifiers may be selected from those substances recognized in GB2760-2014 by the National Health and Family Planning Commission of the People's Republic of China.

### (ii) Texture

In some embodiments, a formulated composition of the present disclosure is characterized as having a pleasing texture. In some embodiments, one or more food components are characterized as texture modifiers, wherein the one of more food components confer a pleasing texture to a formulated composition (e.g., as experienced by a subject ingesting said formulation). In some embodiments, one or more release modulators are characterized as texture modifiers, wherein the one of more release modulators confer a pleasing texture to a formulated composition (e.g., as experienced by a subject ingesting said formulation). In some embodiments, one or more excipients are characterized as texture modifiers, wherein the one of more excipients confer a pleasing texture to a formulated composition (e.g., as experienced by a subject ingesting said formulation). In some embodiments, a combination of one or more food components, one or more release modulators, and/or one or more excipients is characterized as a texture modifier, wherein the combination confers a pleasing texture to a formulated composition (e.g., as experienced by a subject ingesting said formulation).

Without wishing to be bound by any particular theory, a texture modifier may be characterized as providing a mechanically, geometrically, and/or chemically pleasing texture. In some embodiments, a texture modifier may directly provide a pleasing texture to a formulated composition (e.g., as experienced by a subject ingesting said formulation). In some embodiments, a texture modifier may modulate the texture of one or more other food components.

In some embodiments, a texture modifier may contribute to the hardness, cohesiveness, viscosity, elasticity, brittleness, chewiness, gumminess, or a combination thereof, of a formulated composition. For example, in some embodiments, a texture modifier may contribute to one or more geometric properties of a formulated composition that influences the particle size and/or morphology of the formulated composition. In some embodiments, a texture modifier may contribute to a formulated composition's adhesiveness, oiliness, and/or greasiness (e.g., as experienced by a subject ingesting said formulation).

Without wishing to be bound by any particular theory, one skilled in the art will appreciate that one or more additional texture modifiers may further include, but are not limited to, substances identified by one or more governing bodies as safe (e.g., generally regarded as safe and/or food additives). In some embodiments, those skilled in the art will appreciate that one or more taste modifiers may be selected from those substances recognized as Generally Regarded as Safe (i.e., GRAS) by the U.S. Food and Drug Administration. In some embodiments, those skilled in the art will appreciate that one or more taste modifiers may be selected from those substances recognized in 21 C.F.R. 184. In some embodiments, those skilled in the art will appreciate that one or more taste modifiers may be selected from those substances recognized in GB2760-2014 by the National Health and Family Planning Commission of the People's Republic of China.

(iii) Satiety Profiles

Without wishing to be bound by any particular theory, one or more food components of a formulated composition provides satiety to a subject who has ingested said formulation. In some embodiments, the controlled, delayed, and/or extended duration of release of one or more food components from a formulated composition is characterized as having a satiety profile.

In some embodiments, a satiety profile is characterized by the controlled, delayed, and/or extended duration of release of one or more carbohydrates from a formulated composition of the present disclosure. For example, a satiety profile is characterized by the controlled, delayed, and/or extended duration of release of glucose, amylose, allulose, xylitol, cellulose, or a combination thereof, from a formulated composition.

In some embodiments, a satiety profile is characterized by the controlled, delayed, and/or extended duration of release of one or more proteins from a formulated composition of the present disclosure. For example, a satiety profile is characterized by the controlled, delayed, and/or extended duration of release of whey protein (e.g., heat-treated whey protein isolate), casein, soy protein, pea protein, corn protein, zein, gliadin, gelatin, collagen, or a combination thereof, from a formulated composition.

In some embodiments, a satiety profile is characterized by the controlled, delayed, and/or extended duration of release of one or more fats from a formulated composition of the present disclosure. For example, a satiety profile is characterized by the controlled, delayed, and/or extended duration of release of soybean oil, fish oil, palm oil, carnauba wax, lecithin, or combination thereof, from a formulated composition.

In some embodiments, a method of the present disclosure provides satisfaction and/or satiety to a subject comprising administering to the subject a formulated composition characterized as having a satiety profile. For example, in some embodiments, a method comprises administering a multi-layered core-shell preparation that sequentially releases a bolus dose of sucrose in 8-hour intervals, such that 15 g of sucrose is released after 8 hours, 30 g of sucrose is released after 16 hours, and 60 g of sucrose is released after 24 hours. In another other embodiments, a mucoadhesive core-shell preparation extends the duration of jejunal residence time of a formulated composition thereby achieving extended-release of one or more food components (e.g., sucrose), such that 60 g of the one or more food components (e.g., sucrose) is released over a 24-hour period. Without wishing to be bound by any particular theory, it is contemplated that release of one or more food components from a formulated composition as a bolus in 8-hour intervals improves satiety experienced by a subject ingesting said formulation relative to unformulated food components. Without wishing to be bound by any particular theory, it is contemplated that an extended duration of release of one or more food components from a formulated composition over a 24-hour period improves satiety experienced by a subject ingesting said formulation relative to unformulated food components.

### (iv) Modulating Physiological Responses

In some embodiments, a formulated composition of the present disclosure may be characterized as capable of modulating a physiological response in a subject ingesting said formulation. In some embodiments, modulating a physiological response includes eliciting a sensation of satisfaction and/or satiety (as experienced by a subject ingesting a formulation of the present disclosure).

In some embodiments, a formulated composition of the present disclosure may elicit an endogenous physiological satiety response in a subject ingesting said formulation. In some embodiments, an endogenous physiological satiety response may include, but is not limited to, a feeling of fullness, a reduced desire to eat, reduced serum ghrelin, and/or increased serum leptin.

In some embodiments, a physiological response may be physical. For example, in some embodiments, a physical physiological response may include, but is not limited to, application of pressure by a formulated composition of the present disclosure or interaction between the formulated composition with a surface area of one or more gastrointestinal compartments of a subject ingesting said formulation.

In some embodiments, a formulated composition of the present disclosure modulates a physical physiological response by increasing its size, and/or increasing its surface area. For example, in some embodiments, a formulated composition of the present disclosure increases its size by swelling and uptake of one or more dissolution solvents, self-assembly in response to a trigger, or a combination thereof, thereby eliciting a physical physiological response. In some embodiments, a formulated composition increases its surface area by unfolding (e.g., denaturing) in response to a trigger, increases porosity in response to a trigger, or a combination thereof, thereby eliciting a physical physiological response.

In some embodiments, a physiological response may be chemical. For example, in some embodiments, a chemical physiological response may include, but is not limited to chemical-mediated activation of one or more receptors in a gastrointestinal compartment of a subject. In some embodiments, one or more food components characterized as chemical physiological response modulators include, but are not limited to, micronutrients, macronutrients, or a combination thereof. In some embodiments, a micronutrient includes, but is not limited to, tannic acid, ellagitannin, apigenin, luteolin, tangeritin, isorhamnetin, kaempferol, myricetin, quercetin, genipin, rutin, eriodictyol, hesperetin, naringenin, catechin, gallocatechin, epicatechin, epigallocatechin, theaflavin, daidzein, genistein, glycitein, resveratrol, pterostilbene, hydroxytyrosol, cyanidin, delphinidin, malvidin, pelargonidin, peonidin, petunidin, chicoric acid, chlorogenic acid, cinnamic acid, ellagic acid, gallic acid, sinapic acid, rosmarinic acid, salicylic acid, curcumin, piperine, silymarin, silybin, eugenol, melatonin, methylcobalamin, adrafinil, cathine, cathinone, dextroamphetamine, ephedrine, epinephrine, armodafinil, modafinil, phenylethylamine, synephrine, theanine, 5-hydroxytryptophan, caffeine, theobromine, or taurine. In some embodiments, a macronutrient includes, but is not limited to, aspartame, GLP-1, GLP-2, collagen, sermorelin, tesamorelin, lenomorelin, anamorelin, ipamorelin, macimorelin, ghrelin, leptin, tabimorelin, alexamorelin, GHRP-1, GHRP-2, GHRP-3, GHRP-4, GHRP-5, GHRP-6, hexarelin, cellulose, dextrins, amylose, amylopectin, pectin, inulin, lignin, chitin, xanthan gum, sodium alginate, potassium alginate, calcium alginate, ammonium alginate, propylene glycol alginate, sodium hyaluronate, potassium hyaluronate, calcium hyaluronate, agar, agarose, carrageenan, raffinose, cellulose acetate, methyl cellulose, ethyl cellulose, cellulose acetate propionate, cellulose acetate butyrate, cellulose acetate succinate, hydroxypropyl methylcellulose, hydroxypropyl cellulose, whey protein isolate, whey protein concentrate, pea protein isolate, pea protein concentrate, oat protein isolate, oat protein concentrate, soy protein isolate, soy protein concentrate, wheat protein isolate, wheat protein concentrate, egg protein isolate, egg protein concentrate, dairy protein, plant-based protein, casein, bovine serum albumin, ovalbumin, α-lactalbumin, β-lactoglobulin, collagen, glutanin, gliadin, kefirin, avenin, zein, silk, gelatin, hordein, or sodium carboxymethylcellulose.

In some embodiments, the controlled, delayed, and/or extended duration of release of one or more food components from a formulated composition of the present disclosure modulates a physiological response that elicits a sensation of satisfaction and/or satiety (as experienced by a subject ingesting said formulation). In some embodiments, the release of one or more food components (e.g., proteins, carbohydrates, fats) is controlled in order to maximize a sensation of satisfaction and/or satiety (as experienced by a subject ingesting a formulation of the present disclosure). For example, without wishing to be bound by any particular theory, in some embodiments, the release of one or more food components may be characterized as a slow, constant release to provide a continuous source of energy and/or nutrition. For example, in some embodiments, the release of one or more proteins (e.g., whey protein, soy protein) form a formulated composition of the present disclosure as bolus doses in 8 hour intervals stimulates growth hormone secretion in a subject that has ingested said formulation of the present disclosure.

In some embodiments, the extended duration of release of one or more food components from a formulated composition of the present disclosure provides a corresponding extended duration of release of one or more hormones from one or more cells of a subject that has ingested said formulation, where the extended duration of release of one or more hormones elicits a sensation of satisfaction and/or satiety (as experienced by the subject). For example, in some embodiments, the one or more hormones include, but are not limited to, GLP-1, leptin, ghrelin, cholecystokinin, peptide YY, melanocyte-stimulating hormone, or a combination thereof.

In some embodiments, a formulated composition of the present disclosure modulates a physiological response that elicits a sensation of satisfaction and/or satiety (as experienced by a subject ingesting said formulation) that persists for at least about 2 hours to 72 hours, at least about 4 hours to 72 hours, at least about 6 hours to 72 hours, at least about 12 hours to 72 hours, at least about 18 hours to 72 hours, at least about 24 hours to 72 hours, at least about 36 hours to 72 hours, at least about 48 hours to 72 hours, at least about 2 hours to 48 hours, at least about 4 hours to 48 hours, at least about 6 hours to 48 hours, at least about 12 hours to 48 hours, at least about 18 hours to 48 hours, at least about 24 hours to 48 hours, at least about 36 hours to 48 hours, at least about 2 hours to 36 hours, at least about 4 hours to 36 hours, at least about 6 hours to 36 hours, at least about 12 hours to 36 hours, at least about 18 hours to 36 hours, at least about 24 hours to 36 hours, at least about 2 hours to 24 hours, at least about 4 hours to 24 hours, at least about 6 hours to 24 hours, at least about 12 hours to 24 hours, at least about 18 hours to 24 hours, at least about 2 hours to 18 hours, at least about 4 hours to 18 hours, at least about 6 hours to 18 hours, at least about 12 hours to 18 hours, at least about 2 hours to 12 hours, at least about 4 hours to 12 hours, at least about 6 hours to 12 hours, at least about 2 hours to 6 hours, at least about 4 hours to 6 hours, or at least about 2 hours to 4 hours. For example, in some embodiments, a formulated composition of the present disclosure modulates a physiological response that elicits a sensation of satisfaction and/or satiety (as experienced by a subject ingesting said formulation) that persists for at least about 2 hours, at least about 4 hours, at least about 6 hours, at least about 8 hours, at least about 10 hours, at least about 12 hours, at least about 16 hours, at least about 20 hours, at least about 24 hours, at least about 36 hours, or at least about 72 hours.

### 5. Sufficient Nutrition

### (i) Sufficient Daily Nutritional Requirement

In some embodiments, a formulated composition of the present disclosure provides a sufficient daily nutritional requirement (e.g., calories, grams) of one or more food components to a subject ingesting said formulation. In some embodiments, ingestion of a sufficient daily nutritional requirement of one or more food components elicits a sensation of satisfaction and/or satiety (as experienced by a subject ingesting a formulation of the present disclosure).

In some embodiments, a sufficient daily nutritional requirement of one or more food components refers to the amount present in a formulated composition (or preparation of a formulated composition) upon manufacture. In some embodiments, a sufficient daily nutritional requirement of one or more food components refers to the amount released over a given period of time in one or more dissolution solvents. In some embodiments, a sufficient daily nutritional requirement of one or more food components refers to the amount absorbed by a subject that has ingested a formulation of the present disclosure.

In some embodiments, a formulated composition (and/or formulated preparation) of the present disclosure provides a total mass of at least about 50 g, about 125 g, about 250 g, about 500 g, about 750 g, about 1000 g, about 1250 g about 1500 g, about 1750 g, about 2000 g, about 2500 g, or about 3000 g of one or more food components, wherein the total mass is a sufficient daily nutritional amount for a subject ingesting said formulation. In some embodiments, a formulated composition (and/or formulated preparation) of the present disclosure provides a total caloric amount of at least about 250 kcal, at least about 500 kcal, at least about 1000 kcal, at least about 1500 kcal, at least about 2500 kcal, at least about 3500 kcal, or at least about 4500 kcal of one or more food components, wherein the total caloric amount is a sufficient daily nutritional amount for a subject ingesting said formulation.

### (ii) Food Components Balance

In some embodiments, a formulated composition of the present disclosure provides a sufficient balance of two or more food components to a subject ingesting said formulation. In some embodiments, ingestion of a sufficient balance of two or more food components elicits a sensation of satisfaction and/or satiety (as experienced by a subject ingesting a formulation of the present disclosure).

In some embodiments, a balance of two or more food components refers to the relative amount of two or more food components present in a formulated composition (or preparation of a formulated composition) upon manufacture. In some embodiments, a sufficient daily nutritional requirement of one or more food components refers to the amount released over a given period of time in one or more dissolution solvents. In some embodiments, a sufficient daily nutritional requirement of one or more food components refers to the amount absorbed by a subject that has ingested a formulation of the present disclosure.

In some embodiments, a formulated composition (and/or formulated preparation) of the present disclosure provides a total carbohydrate amount of at least about 25 g, at least about 50 g, at least about 100 g, at least about 200 g, at least about 300 g, at least about 400 g, or at least about 500 g. In some embodiments, a formulated composition (and/or formulated preparation) of the present disclosure provides a total protein amount of at least about 100 g, at least about 200 g, at least about 300 g, at least about 400 g, at least about 500 g, at least about 600 g, or at least about 700 g. In some embodiments, a formulated composition (and/or formulated preparation) of the present disclosure provides a total fat content of at least about 12 g, at least about 25 g, at least about 50 g, at least about 100 g, at least about 200 g, at least about 300 g, or at least about 400 g.

In some embodiments, a formulated composition (or preparation of a formulated composition) of the present disclosure provides a ratio of carbohydrates to fats, by dry weight, between about 3 and about 12. In some embodiments, a formulated composition (or preparation of a formulated composition) of the present disclosure provides a ratio of proteins to fats, by dry weight, between about 0.5 and about 5. In some embodiments, a formulated composition (or preparation of a formulated composition) of the present disclosure provides a ratio of carbohydrates to proteins, by dry weight, between about 0.5 and about 10.

(iii) Enzyme Inhibitors

In some embodiments, a formulated composition (or preparation of the formulated composition) comprises an effective dose of one or more enzyme inhibitors. Without wishing to be bound by any particular theory, one or more enzyme inhibitors are characterized as reducing the activity of one or more gastrointestinal enzymes, thereby prolonging the digestion and/or absorption of one or more food components released from a formulated composition. In some embodiments, prolonged digestion and/or absorption of one or more food components elicits a sensation of satisfaction and/or satiety (as experienced by a subject ingesting a formulation of the present disclosure).

In some embodiments, an effective dose of one or more enzyme inhibitors prolongs the half-life of one or more food components by at least about 10%, about 20%, about 30%, about 40%, about 50%, about 60%, about 75%, about 90%, or about 100% in one or more dissolution solvents (e.g., simulated intestinal fluid comprising pancreatin). For example, in some embodiments, an effective dose of one or more enzyme inhibitors prolongs the half-life of one or more food component(s) by at least about 1-fold, about 2-fold, about 3-fold, about 5-fold, about 10-fold, about 20-fold, or about 50-fold in one or more dissolution solvents (e.g., simulated intestinal fluid comprising pancreatin).

In some embodiments, one or more enzyme inhibitors may include, but are not limited to amylostatin Y, amylostatin S-AI, adiposin, gurmarin, zeamatin, helianthamide, lipstatin, orlistat, esterastin, valilactone, panclicin D, ebelactone, vibralactone, soybean trypsin inhibitor, serpin A1, serpin A3, serpin A4, serpin E1, serpin C1, Elafin, TIMP-1, TIMP-2, TIMP-3, TIMP-4, an antioxidant, or a combination thereof.

Without wishing to be bound by any particular theory, one skilled in the art will appreciate that one or more enzyme inhibitors may further include, but are not limited to, substances identified by one or more governing bodies as safe (e.g., generally regarded as safe and/or food additives). In some embodiments, those skilled in the art will appreciate that one or more taste modifiers may be selected from those substances recognized as Generally Regarded as Safe (i.e., GRAS) by the U.S. Food and Drug Administration. In some embodiments, those skilled in the art will appreciate that one or more taste modifiers may be selected from those substances recognized in 21 C.F.R. 184. In some embodiments, those skilled in the art will appreciate that one or more taste modifiers may be selected from those substances recognized in GB2760-2014 by the National Health and Family Planning Commission of the People's Republic of China.

### (iv) Absorption Enhancers

In some embodiments, a formulated composition (or preparation of the formulated composition) comprises an effective dose of one or more absorption enhancers. Without wishing to be bound by any particular theory, one or more absorption enhancers are characterized as increasing the gastrointestinal absorption of one or more food components. In some embodiments, increased absorption of one or more food components elicits a sensation of satisfaction and/or satiety (as experienced by a subject ingesting a formulation of the present disclosure).

In some embodiments, an effective dose of one or more absorption enhancers increases the bioavailability of one or more food components by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. For example, in some embodiments, an effective dose of one or more absorption enhancers increases the bioavailability of one or more food components by at least about 1-fold, about 2-fold, about 3-fold, about 5-fold, about 10-fold, about 20-fold, or about 50-fold.

In some embodiments, one or more absorption enhancers may include, but are not limited to sodium caprylate, sodium caprate, sodium laurate, sodium oleate, sodium linoleate, propyl gallate, propyl syringate, propyl shikimate, octyl gallate, octyl syringate, octyl shikimate, ammoniated glycyrrhizin, quillaia extract, tocopherol PEG succinate, lauroyl polyoxylglycerides, polysorbate 80, ethanol, propylene glycol, poly(ethylene glycol), diethylene glycol monoethyl ether, sodium citrate, medium chain triglycerides, lipase, sodium lauryl sulfate, ascorbyl palmitate, or a combination thereof.

Without wishing to be bound by any particular theory, one skilled in the art will appreciate that one or more absorption enhancers may further include, but are not limited to, substances identified by one or more governing bodies as safe (e.g., generally regarded as safe and/or food additives). In some embodiments, those skilled in the art will appreciate that one or more taste modifiers may be selected from those substances recognized as Generally Regarded as Safe (i.e., GRAS) by the U.S. Food and Drug Administration. In some embodiments, those skilled in the art will appreciate that one or more taste modifiers may be selected from those substances recognized in 21 C.F.R. 184. In some embodiments, those skilled in the art will appreciate that one or more taste modifiers may be selected from those substances recognized in GB2760-2014 by the National Health and Family Planning Commission of the People's Republic of China.

### 6. Other Benefits

In some embodiments, a formulated composition (and/or formulated preparation) of the present disclosure comprises one or more food components in an amount sufficient to stimulate myofibrillar protein synthesis when said formulated composition (and/or formulated preparation). Without wishing to be bound by any particular theory, stimulating myofibrillar synthesis includes promoting skeletal muscle growth, promoting strength, and/or improving oral glucose tolerance. In some embodiments, a formulated composition of the present disclosure comprises one or more food components in an amount sufficient to promote activation of the mTORC1 pathway when released into an anatomical compartment of a subject ingesting said formulation.

Without wishing to be bound by any particular theory, it is contemplated that elevated concentration(s) of one or more food components solubilized (e.g., released) in one or more gastrointestinal compartments (e.g., stomach, small intestine, large intestine) at any one period of time increases the gastrointestinal discomfort experienced by a consuming subject, reflexively minimizing intake of said one or more food components. Without wishing to be bound by any particular theory, gastrointestinal discomfort includes upper abdominal pain, bloating, gastric reflux, gastroparesis, lower abdominal pain, intestinal inflammation, flatulence, diarrhea, and/or constipation.

Without wishing to be bound by any particular theory, it is further contemplated that gastrointestinal discomfort in a subject derives from factors including but not limited to incomplete trituration, incomplete digestion, incomplete absorption, modification of gut microbiota, and/or inappropriate stool consistency that each arise due to elevated concentrations of one or more food components solubilized in one or more gastrointestinal compartments (e.g., released) at any one period of time.

In some embodiments of the present disclosure, formulated compositions (and/or formulated preparations) providing for controlled release of one or more food components reduce the maximum release of one or more food components at any one period of time as compared to one or more food components that are not formulated (e.g., do not comprise one or more release modulators). In some embodiments of the present disclosure, formulated compositions (and/or formulated preparations) providing for delayed release reduce the maximum release of one or more food components at any one period of time as compared to one or more food components that are not formulated (e.g., do not comprise one or more release modulators). In some embodiments of the present disclosure, formulated compositions (and/or formulated preparations) providing for an extended duration of release reduce the maximum release of one or more food components at any one period of time as compared to one or more food components that are not formulated (e.g., do not comprise one or more release modulators).

Without wishing to be bound by any particular theory, it is contemplated that formulated compositions (and/or formulated preparations) of the present disclosure, by reducing the maximum release of one or more food components at any one period of time, minimize gastrointestinal discomfort by factors including, but not limited to, promoting complete trituration, complete digestion, complete absorption, maintenance of gut microbiota, and/or appropriate stool consistency.

In some embodiments of the present disclosure, formulated compositions (and/or formulated preparations) characterized as providing controlled release of one or more food components in a gastrointestinal compartment (e.g., stomach, small intestine, large intestine) of a subject that has ingested said formulated composition (and/or formulated preparation) reduces the gastrointestinal discomfort of the subject as compared to consumption of an equivalent amount of one or more food components that are not formulated (e.g., do not comprise one or more release modulators). In some embodiments of the present disclosure, formulated compositions (and/or formulated preparations) characterized as providing controlled release of one or more food components in a gastrointestinal compartment (e.g., stomach, small intestine, large intestine) of a subject that has ingested said formulation reduce malnutrition of the subject as compared to consumption of an equivalent amount of one or more food components that are not formulated (e.g., do not comprise one or more release modulators). In some embodiments of the present disclosure, formulated compositions (and/or formulated preparations) characterized as providing controlled release of one or more food components in a gastrointestinal compartment (e.g., stomach, small intestine, large intestine) of a subject that has ingested said formulation improve the digestive processes of the subject as compared to ingestion of an equivalent amount of one or more food components that are not formulated (e.g., do not comprise one or more release modulators). In some embodiments of the present disclosure, formulated compositions (and/or formulated preparations) characterized as providing controlled release of one or more food components in a gastrointestinal compartment (e.g., stomach, small intestine, large intestine) of a subject that has ingested said formulation reduce gastrointestinal inflammation of the subject as compared to ingestion of an equivalent amount of one or more food components that are not formulated (e.g., do not comprise one or more release modulators).

In some embodiments, a formulated composition and/or formulated preparation of the present disclosure reduces the bodyweight of a subject ingesting said composition and/or preparation when consumed at least once daily for a period of 12 weeks by at least about 1% to 20%, at least about 3% to 20%, at least about 5% to 20%, at least about 8% to 20%, at least about 11% to 20%, at least about 14% to 20%, at least about 17% to 20%, at least about 1% to 18%, at least about 3% to 18%, at least about 5% to 18%, at least about 8% to 18%, at least about 11% to 18%, at least about 14% to 18%, at least about 17% to 18%, at least about 1% to 15%, at least about 3% to 15%, at least about 5% to 15%, at least about 8% to 15%, at least about 11% to 15%, at least about 14% to 15%, at least about 1% to 12%, at least about 3% to 12%, at least about 5% to 12%, at least about 8% to 12%, at least about 11% to 12%, at least about 1% to 9%, at least about 3% to 9%, at least about 5% to 9%, at least about 8% to 9%, at least about 1% to 6%, at least about 3% to 6%, at least about 5% to 6%, at least about 1% to 4%, at least about 3% to 4%, or at least about 1% to 2% as compared to the initial bodyweight of the subject prior to consumption of the formulated composition and/or formulated preparation of the present disclosure. In some embodiments, a formulated composition and/or formulated preparation of the present disclosure reduces the bodyweight of a subject ingesting said composition and/or preparation when consumed at least twice daily for a period of 12 weeks by at least about 1% to 20%, at least about 3% to 20%, at least about 5% to 20%, at least about 8% to 20%, at least about 11% to 20%, at least about 14% to 20%, at least about 17% to 20%, at least about 1% to 18%, at least about 3% to 18%, at least about 5% to 18%, at least about 8% to 18%, at least about 11% to 18%, at least about 14% to 18%, at least about 17% to 18%, at least about 1% to 15%, at least about 3% to 15%, at least about 5% to 15%, at least about 8% to 15%, at least about 11% to 15%, at least about 14% to 15%, at least about 1% to 12%, at least about 3% to 12%, at least about 5% to 12%, at least about 8% to 12%, at least about 11% to 12%, at least about 1% to 9%, at least about 3% to 9%, at least about 5% to 9%, at least about 8% to 9%, at least about 1% to 6%, at least about 3% to 6%, at least about 5% to 6%, at least about 1% to 4%, at least about 3% to 4%, or at least about 1% to 2% as compared to the initial bodyweight of the subject prior to consumption of formulated compositions and/or preparation of the present disclosure.

In some embodiments, a formulated composition and/or formulated preparation of the present disclosure reduces the bodyweight of a subject ingesting said composition when consumed at least once daily for a period of 24 weeks by at least about 1% to 20%, at least about 3% to 20%, at least about 5% to 20%, at least about 8% to 20%, at least about 11% to 20%, at least about 14% to 20%, at least about 17% to 20%, at least about 1% to 18%, at least about 3% to 18%, at least about 5% to 18%, at least about 8% to 18%, at least about 11% to 18%, at least about 14% to 18%, at least about 17% to 18%, at least about 1% to 15%, at least about 3% to 15%, at least about 5% to 15%, at least about 8% to 15%, at least about 11% to 15%, at least about 14% to 15%, at least about 1% to 12%, at least about 3% to 12%, at least about 5% to 12%, at least about 8% to 12%, at least about 11% to 12%, at least about 1% to 9%, at least about 3% to 9%, at least about 5% to 9%, at least about 8% to 9%, at least about 1% to 6%, at least about 3% to 6%, at least about 5% to 6%, at least about 1% to 4%, at least about 3% to 4%, or at least about 1% to 2% as compared to the initial bodyweight of the subject prior to consumption of formulated compositions and/or formulated preparations of the present disclosure. In some embodiments, a formulated composition and/or formulated preparation of the present disclosure reduces the bodyweight of a subject ingesting said composition or preparation when consumed at least twice daily for a period of 24 weeks by at least about 1% to 20%, at least about 3% to 20%, at least about 5% to 20%, at least about 8% to 20%, at least about 11% to 20%, at least about 14% to 20%, at least about 17% to 20%, at least about 1% to 18%, at least about 3% to 18%, at least about 5% to 18%, at least about 8% to 18%, at least about 11% to 18%, at least about 14% to 18%, at least about 17% to 18%, at least about 1% to 15%, at least about 3% to 15%, at least about 5% to 15%, at least about 8% to 15%, at least about 11% to 15%, at least about 14% to 15%, at least about 1% to 12%, at least about 3% to 12%, at least about 5% to 12%, at least about 8% to 12%, at least about 11% to 12%, at least about 1% to 9%, at least about 3% to 9%, at least about 5% to 9%, at least about 8% to 9%, at least about 1% to 6%, at least about 3% to 6%, at least about 5% to 6%, at least about 1% to 4%, at least about 3% to 4%, or at least about 1% to 2% as compared to the initial bodyweight of the subject prior to consumption of formulated compositions and/or preparations of the present disclosure.

In some embodiments, a formulated composition and/or formulated preparation of the present disclosure reduces the bodyweight of a subject ingesting said composition when consumed at least once daily for a period of 36 weeks by at least about 1% to 20%, at least about 3% to 20%, at least about 5% to 20%, at least about 8% to 20%, at least about 11% to 20%, at least about 14% to 20%, at least about 17% to 20%, at least about 1% to 18%, at least about 3% to 18%, at least about 5% to 18%, at least about 8% to 18%, at least about 11% to 18%, at least about 14% to 18%, at least about 17% to 18%, at least about 1% to 15%, at least about 3% to 15%, at least about 5% to 15%, at least about 8% to 15%, at least about 11% to 15%, at least about 14% to 15%, at least about 1% to 12%, at least about 3% to 12%, at least about 5% to 12%, at least about 8% to 12%, at least about 11% to 12%, at least about 1% to 9%, at least about 3% to 9%, at least about 5% to 9%, at least about 8% to 9%, at least about 1% to 6%, at least about 3% to 6%, at least about 5% to 6%, at least about 1% to 4%, at least about 3% to 4%, or at least about 1% to 2% as compared to the initial bodyweight of the subject prior to consumption of formulated compositions and/or preparations of the present disclosure. In some embodiments, a formulated composition and/or formulated preparation of the present disclosure reduces the bodyweight of a subject ingesting said composition when consumed at least twice daily for a period of 36 weeks by at least about 1% to 20%, at least about 3% to 20%, at least about 5% to 20%, at least about 8% to 20%, at least about 11% to 20%, at least about 14% to 20%, at least about 17% to 20%, at least about 1% to 18%, at least about 3% to 18%, at least about 5% to 18%, at least about 8% to 18%, at least about 11% to 18%, at least about 14% to 18%, at least about 17% to 18%, at least about 1% to 15%, at least about 3% to 15%, at least about 5% to 15%, at least about 8% to 15%, at least about 11% to 15%, at least about 14% to 15%, at least about 1% to 12%, at least about 3% to 12%, at least about 5% to 12%, at least about 8% to 12%, at least about 11% to 12%, at least about 1% to 9%, at least about 3% to 9%, at least about 5% to 9%, at least about 8% to 9%, at least about 1% to 6%, at least about 3% to 6%, at least about 5% to 6%, at least about 1% to 4%, at least about 3% to 4%, or at least about 1% to 2% as compared to the initial bodyweight of the subject prior to consumption of formulated compositions and/or preparations of the present disclosure.

### E. Exemplary Formulations

TABLE 1 provides exemplary formulated compositions (e.g., one or more food components, and one or more release modulators) of the present disclosure.

TABLE 1
Exemplary Formulated compositions

| | Food | |
|---|---|---|
| Formulation | Component(s) | Release Modulator(s) |
| 1 | 91% (w/w) | 9% (w/w) Methocel ™ 15LV |
| | Heat-Treated | |
| | Whey Protein | |
| | Isolate | |
| 2 | 91% (w/w) | 9% (w/w) Methocel ™ 4C |
| | Heat-Treated | |
| | Whey Protein | |
| | Isolate | |
| 3 | 91% (w/w) | 9% (w/w) Methocel ™ 15C |
| | Heat-Treated | |
| | Whey Protein | |
| | Isolate | |
| 4 | 91% (w/w) | 9% (w/w) Methocel ™ 4M |
| | Heat-Treated | |
| | Whey Protein | |
| | Isolate | |
| 5 | 91% (w/w) | 9% (w/w) Glycine |
| | Heat-Treated | |
| | Whey Protein | |
| | Isolate | |
| 6 | 91% (w/w) | 9% (w/w) Lysine |
| | Heat-Treated | |
| | Whey Protein | |
| | Isolate | |
| 7 | 76% (w/w) | 6% (w/w) Gluconolactone |
| | Heat-Treated | 3% (w/w) Calcium Carbonate |
| | Whey Protein | 15% (w/w) VIVAPHARM ™ PH R5 |
| | Isolate | |
| 8 | 76% (w/w) | 6% (w/w) Gluconolactone |
| | Heat-Treated | 3% (w/w) Calcium Carbonate |
| | Whey Protein | 15% (w/w) VIVAPHARM ™ PH 155 |
| | Isolate | |
| 9 | 76% (w/w) | 6% (w/w) Gluconolactone |
| | Heat-Treated | 3% (w/w) Calcium Carbonate |
| | Whey Protein | 15% (w/w) VIVAPHARM ™ PH 176 |
| | Isolate | |
| 10 | 76% (w/w) | 6% (w/w) Gluconolactone |
| | Heat-Treated | 3% (w/w) Dicalcium phosphate |
| | Whey Protein | 15% (w/w) VIVAPHARM ™ PH 155 |
| | Isolate | |
| 11 | 76% (w/w) | 6% (w/w) Gluconolactone |
| | Heat-Treated | 3% (w/w) Calcium Carbonate |
| | Whey Protein | 15% (w/w) PECTNER ™ APC210 |
| | Isolate | |
| 12 | 66% (w/w) | 5% (w/w) Gluconolactone |
| | Heat-Treated | 3% (w/w) Calcium Carbonate |
| | Whey Protein | 26% (w/w) PECTNER ™ APC220 |
| | Isolate | |
| 13 | 91% (w/w) | 9% (w/w) Iota Carrageenan |
| | Heat-Treated | |
| | Whey Protein | |
| | Isolate | |
| 14 | 91% (w/w) | 9% (w/w) Lambda Carrageenan |
| | Heat-Treated | |
| | Whey Protein | |
| | Isolate | |
| 15 | 76% (w/w) | 6% (w/w) Gluconolactone |
| | Heat-Treated | 3% (w/w) Calcium Carbonate |
| | Whey Protein | 15% (w/w) PECTNER ™ 169B |
| | Isolate | |
| 16 | 95% (w/w) | 3% (w/w) Ammoniated Shellac |
| | Heat-Treated | 1.5% (w/w) Tween ™ 80 |
| | Whey Protein | 0.5% (w/w) SPAN ™ 20 |
| | Isolate | |
| 17 | 71% (w/w) | 27% (w/w) Nutritional Yeast |
| | Heat-Treated | 2% (w/w) Transglutaminase |
| | Whey Protein | |
| | Isolate | |
| 18 | 91% (w/w) | 9% (w/w) Hypromellose Acetate |
| | Heat-Treated | Succinate |
| | Whey Protein | |
| | Isolate | |
| 19 | 71% (w/w) | 14.3% (w/w) Maltodextrin DE16 |
| | Heat-Treated | 14.3% (w/w) Functional Rice Starch |
| | Whey Protein | |
| | Isolate | |
| 20 | 71% (w/w) | 14.3% (w/w) Maltodextrin DE16 |
| | Heat-Treated | 14.3% (w/w) Corn Starch |
| | Whey Protein | |
| | Isolate | |
| 21 | 91% (w/w) | 9% (w/w) ORAFT Inulin GR |
| | Heat-Treated | |
| | Whey Protein | |
| | Isolate | |
| 22 | 91% (w/w) | 9% (w/w) PromOat ™ Fiber |
| | Heat-Treated | |
| | Whey Protein | |
| | Isolate | |
| 23 | 91% (w/w) | 9% (w/w) Glucose |
| | Heat-Treated | |
| | Whey Protein | |
| | Isolate | |
| 24 | 91% (w/w) | 9% (w/w) Functional Rice Starch |
| | Heat-Treated | |
| | Whey Protein | |
| | Isolate | |
| 25 | 91% (w/w) | 9% (w/w) Tapioca Polydextrose |
| | Heat-Treated | |
| | Whey Protein | |
| | Isolate | |
| 26 | 83% (w/w) | 8% (w/w) Sodium silicate |
| | Heat-Treated | 9% (w/w) Calcium Carbonate |
| | Whey Protein | |
| | Isolate | |
| 27 | 82.6% (w/w) | 15.1% (w/w) Vivapur ™ Alginate FD176 |
| | Heat-Treated | 1.5% (w/w) Gluconolactone |
| | Whey Protein | 0.8% (w/w) Dicalcium phosphate |
| | Isolate | |
| 28 | 80% (w/w) | 13.4% (w/w) Swanlac ASL10 |
| | Heat-Treated | 0.9% (w/w) Vivapur ™ Alginate FD176 |
| | Whey Protein | 2.9% (w/w) Chitosan |
| | Isolate | 1.4% (w/w) Gluconolactone |
| | | 1.4% (w/w) Dicalcium phosphate |
| 29 | 81.2% (w/w) | 16.2% (w/w) Rice Starch |
| | Heat-Treated | 1% (w/w) Chitosan |
| | Whey Protein | 1% (w/w) Tannic Acid |
| | Isolate | 0.6% (w/w) Laccase |
| 30 | 71% (w/w) | 27% (w/w) Nutritional Yeast |
| | Heat-Treated | 2% (w/w) Transglutaminase |
| | Whey Protein | |
| | Isolate | |
| 31 | 82.4% (w/w) | 13.8% (w/w) Pectner ™ APC169B |
| | Heat-Treated | 0.9% (w/w) Vivapharm ™ sodium |
| | Whey Protein | alginate PH176 |
| | Isolate | 1.45% (w/w) Gluconolactone |
| | | 1.45% (w/w) Calcium hydrogen |
| | | phosphate anhydrous |
| 32 | 80% (w/w) | 13.4% (w/w) Pectner ™ APC 210 |
| | Heat-Treated | 0.86% (w/w) Vivapur ™ Alginate FD176 |
| | Whey Protein | 2.9% (w/w) Chitosan |
| | Isolate | 1.4% (w/w) Gluconolactone |
| | (Glanbia ™ | 1.4% (w/w) Dicalcium phosphate |
| | Provon 190) | |
| 33 | 70% (w/w) | 23.5% (w/w) Pectner APC210 |
| | Heat-Treated | 1.6% (w/w) Vivapur ™ Alginate FD176 |
| | Whey Protein | 2.4% (w/w) Gluconolactone |
| | Isolate | 2.4% (w/w) Dicalcium phosphate |
| 34 | 75% (w/w) | 19.6% (w/w) Pectner APC210 |
| | Heat-Treated | 1.3% (w/w) Vivapur ™ Alginate FD176 |
| | Whey Protein | 2% (w/w) Gluconolactone |
| | Isolate | 2% (w/w) Dicalcium phosphate |
| 35 | 80% (w/w) | 15.7% (w/w) Pectner APC210 |
| | Heat-Treated | 1% (w/w) Vivapur ™ Alginate FD176 |
| | Whey Protein | 1.6% (w/w) Gluconolactone |
| | Isolate | 1.6% (w/w) Dicalcium phosphate |
| 36 | 85% (w/w) | 11.7% (w/w) Pectner APC210 |
| | Heat-Treated | 0.8% (w/w) Vivapur ™ Alginate FD176 |
| | Whey Protein | 1.2% (w/w) Gluconolactone |
| | Isolate | 1.2% (w/w) Dicalcium phosphate |
| 37 | 90% (w/w) | 7.8% (w/w) Pectner APC210 |
| | Heat-Treated | 0.5% (w/w) Vivapur ™ Alginate FD176 |
| | Whey Protein | 0.8% (w/w) Gluconolactone |
| | Isolate | 0.8% (w/w) Dicalcium phosphate |
| 38 | 90.8% (w/w) | 5% (w/w) Pectner APC210 |
| | Heat-Treated | 1% (w/w) Vivapur ™ Alginate FD176 |
| | Whey Protein | 1.6% (w/w) Gluconolactone |
| | Isolate | 1.6% (w/w) Dicalcium phosphate |
| 39 | 86% (w/w) | 10% (w/w) Pectner APC210 |
| | Heat-Treated | 1% (w/w) Vivapur ™ Alginate FD176 |
| | Whey Protein | 1.5% (w/w) Gluconolactone |
| | Isolate | 1.5% (w/w) Dicalcium phosphate |
| 40 | 81.2% (w/w) | 15% (w/w) Pectner APC210 |
| | Heat-Treated | 0.9% (w/w) Vivapur ™ Alginate FD176 |
| | Whey Protein | 1.4% (w/w) Gluconolactone |
| | Isolate | 1.4% (w/w) Dicalcium phosphate |
| 41 | 83% (w/w) | 13.9% (w/w) Pectner APC210 |
| | Heat-Treated | 0.2% (w/w) Vivapur ™ Alginate FD176 |
| | Whey Protein | 1.4% (w/w) Gluconolactone |
| | Isolate | 1.5% (w/w) Dicalcium phosphate |
| 42 | 81.9% (w/w) | 13.7% (w/w) Pectner APC210 |
| | Heat-Treated | 1.5% (w/w) Vivapur ™ Alginate FD176 |
| | Whey Protein | 1.4% (w/w) Gluconolactone |
| | Isolate | 1.4% (w/w) Dicalcium phosphate |
| 43 | 80.7% (w/w) | 13.5% (w/w) Pectner APC210 |
| | Heat-Treated | 3% (w/w) Vivapur ™ Alginate FD176 |
| | Whey Protein | 1.4% (w/w) Gluconolactone |
| | Isolate | 1.4% (w/w) Dicalcium phosphate |
| 44 | 82.4 (w/w) | 9.9% (w/w) Pectner APC210 |
| | Heat-Treated | 0.7% (w/w) Vivapur ™ Alginate FD176 |
| | Whey Protein | 3.5% (w/w) Gluconolactone |
| | Isolate | 3.5% (w/w) Dicalcium phosphate |
| 45 | 82.4 (w/w) | 11.4% (w/w) Pectner APC210 |
| | Heat-Treated | 0.8% (w/w) Vivapur ™ Alginate FD176 |
| | Whey Protein | 2.7% (w/w) Gluconolactone |
| | Isolate | 2.7% (w/w) Dicalcium phosphate |
| 46 | 82.4 (w/w) | 12.8% (w/w) Pectner APC210 |
| | Heat-Treated | 0.9% (w/w) Vivapur ™ Alginate FD176 |
| | Whey Protein | 2% (w/w) Gluconolactone |
| | Isolate | 1.9% (w/w) Dicalcium phosphate |
| 47 | 82.4 (w/w) | 13.5% (w/w) Pectner APC210 |
| | Heat-Treated | 0.9% (w/w) Vivapur ™ Alginate FD176 |
| | Whey Protein | 1.6% (w/w) Gluconolactone |
| | Isolate | 1.6% (w/w) Dicalcium phosphate |
| 48 | 82.4 (w/w) | 14.3% (w/w) Pectner APC210 |
| | Heat-Treated | 1% (w/w) Vivapur ™ Alginate FD176 |
| | Whey Protein | 1.1% (w/w) Gluconolactone |
| | Isolate | 1.2% (w/w) Dicalcium phosphate |
| 49 | 82.4 (w/w) | 14.5% (w/w) Pectner APC210 |
| | Heat-Treated | 1% (w/w) Vivapur ™ Alginate FD176 |
| | Whey Protein | 1% (w/w) Gluconolactone |
| | Isolate | 1% (w/w) Dicalcium phosphate |
| 50 | 82.4% (w/w) | 13.8% (w/w) Pectner ™ APC169B |
| | Heat-treated | 0.9% (w/w) Vivapharm ™ sodium |
| | Whey Protein | alginate PH 176 |
| | Isolate | 1.45% (w/w) Gluconolactone |
| | | 1.45% (w/w) Calcium hydrogen |
| | | phosphate anhydrous |
| 51 | 82.4% (w/w) | 13.8% (w/w) Pectner ™ APC210 |
| | Heat-treated | 0.9% (w/w) Vivapharm ™ sodium |
| | Whey Protein | alginate PH 176 |
| | Isolate | 1.45% (w/w) Gluconolactone |
| | | 1.45% (w/w) Calcium hydrogen |
| | | phosphate anhydrous |
| 52 | 82% w/w | 13.7% (w/w) Pectner ™ APC210 pectin |
| | Heat-treated | 0.9% (w/w) Vivapharm ™ sodium |
| | Whey Protein | alginate PH 176 |
| | Isolate | 1.45% (w/w) Gluconolactone |
| | | 1.45% (w/w) Calcium hydrogen |
| | | phosphate anhydrous, 0.5% (w/v) |
| | | SIPERNAT 22S |
| 53 | 80% w/w | 13.4% (w/w) Kappa Carrageenan |
| | Heat-treated | 0.86% (w/w) Vivapur ™ Alginate FD176 |
| | Whey Protein | 2.9% (w/w) Chitosan |
| | Isolate (e.g., | 1.4% (w/w) Gluconolactone |
| | Glanbia ™ | 1.4% (w/w) Dicalcium Phosphate |
| | Provon 190) | |
| 54 | 80% w/w | 13.4% (w/w) Pectner ™ APC210 |
| | Heat-treated | 0.86% (w/w) Vivapur ™ Alginate FD176 |
| | Whey Protein | 2.9% (w/w) Chitosan |
| | Isolate (e.g., | 1.4% (w/w) Gluconolactone |
| | Glanbia ™ | 1.4% (w/w) Dicalcium Phosphate |
| | Provon 190) | |
| 55 | 80% w/w | 7.1% (w/w) Pectner ™ APC210 |
| | Heat-treated | 7.1% (w/w) Vivapharm ™ Sodium |
| | Whey Protein | alginate PH176 |
| | Isolate (e.g., | 2.9% (w/w) Chitosan |
| | Glanbia ™ | 1.4% (w/w) Gluconolactone |
| | Provon 190) | 1.4% (w/w) Dicalcium Phosphate |
| 56 | 80.2% (w/w) | 13.3% (w/w) Pectner ™ APC21 |
| | Heat-treated | 0, 1% (w/w) Vivapur ™ Alginate FD176 |
| | Whey Protein | 3.2% (w/w) Gluconolactone |
| | Isolate | 2.1% (w/w) Dicalcium phosphate |
| 57 | 78.3% (w/w) | 13.1% (w/w) Pectner ™ APC210 |
| | Heat-treated | 1% (w/w) Vivapur ™ Alginate FD176 |
| | Whey Protein | 3.1% (w/w) Gluconolactone |
| | Isolate | 2.1% (w/w) Dicalcium phosphate |
| | | 2.4% (w/w) Sipernat ™ 22S |
| 58 | 78.3% (w/w) | 13.1% (w/w) Pectner ™ APC210 |
| | Heat-treated | 1% (w/w) Vivapur ™ Alginate FD176 |
| | Whey Protein | 3.1% (w/w) Gluconolactone |
| | Isolate | 2.1% (w/w) Dicalcium phosphate |
| | | 1.2% (w/w) Sipernat ™ 22S |
| | | 1.2% (w/w) Aquafaba |
| 59 | 77% (w/w) | 12.8% (w/w) Pectner ™ APC210 |
| | Heat-treated | 1% (w/w) Vivapur ™ Alginate FD176 |
| | Whey Protein | 3.1% (w/w) Gluconolactone |
| | Isolate | 2% (w/w) Dicalcium phosphate |
| | | 1.2% (w/w) Chitosan |
| | | 1.2% (w/w) Sipernat ™ 22S |
| | | 1.2% (w/w) Aquafaba |
| 60 | 77% (w/w) | 12.8% (w/w) Pectner ™ APC210 |
| | Heat-treated | 1% (w/w) Vivapur ™ Alginate FD176 |
| | Whey Protein | 3.1% (w/w) Gluconolactone |
| | Isolate | 2% (w/w) Dicalcium phosphate |
| | | 1.2% (w/w) Chitosan |
| | | 1.2% (w/w) HiCap100 ™ |
| | | 1.2% (w/w) Aquafaba |
| 61 | 83.8% (w/w) | 4.0% (w/w) Ticaloid ™ 710H |
| | Heat-treated | 1% (w/w) Vivapur ™ Alginate FD176 |
| | Whey Protein | 3.4% (w/w) Gluconolactone |
| | Isolate | 2.2% (w/w) Dicalcium phosphate |
| | | 5.5% (w/w) Chitosan |
| 62 | 81.1% (w/w) | 3.9% (w/w) Ticaloid ™ 710H |
| | Heat-treated | 1% (w/w) Vivapur ™ Alginate FD176 |
| | Whey Protein | 3.3% (w/w) Gluconolactone |
| | Isolate | 2.2% (w/w) Dicalcium phosphate |
| | | 5.4% (w/w) Chitosan |
| | | 2.6% (w/w) SIPERNAT ™ 50S |
| 63 | 81.1% (w/w) | 3.9% (w/w) Ticaloid ™ 710H |
| | Heat-treated | 1% (w/w) Vivapur ™ Alginate FD176 |
| | Whey Protein | 3.3% (w/w) Gluconolactone |
| | Isolate | 2.2% (w/w) Dicalcium phosphate |
| | | 5.4% (w/w) Chitosan |
| | | 1.3% (w/w) SIPERNAT ™ 50S |
| | | 1.3% (w/w) Aquafaba |
| 64 | 77.6% (w/w) | 6.5% (w/w) Pectner ™ APC210 |
| | Heat-treated | 0.5% (w/w) Vivapur ™ Alginate FD176 |
| | Whey Protein | 6.2% (w/w) Gluconolactone |
| | Isolate, | 4.1% (w/w) Dicalcium phosphate |
| | | 2.7% (w/w) Chitosan |
| | | 1.2% (w/w) Sipernat ™ 50S |
| | | 1.2% (w/w) Aquafaba |
| 65 | 77.6% (w/w) | 6.5% (w/w) Pectner ™ APC210 |
| | Heat-treated | 0.5% (w/w) Vivapur ™ Alginate FD176 |
| | Whey Protein | 6.2% (w/w) Gluconolactone |
| | Isolate, | 4.1% (w/w) Dicalcium phosphate |
| | | 2.7% (w/w) Chitosan |
| | | 1.2% (w/w)) Globe DE42 ™ Corn Syrup |
| | | Solids |
| | | 1.2% (w/w) NZorbit-M 2144 |
| 66 | 76.4% (w/w) | 12.8% (w/w) Pectner ™ APC 210 |
| | Heat-treated | 0.9% (w/w) VIVAPUR ™ FD 176 |
| | Whey Protein | 2.0% (w/w) Gluconolactone |
| | Isolate (e.g., | 2.0% (w/w) Dicalcium phosphate |
| | Glanbia | 5.9% (w/w) Chitolytic chitosan |
| | Provon ™190) | |
| | 74.1% (w/w) | 12.4% (w/w) Pectner ™ APC 210 |
| | Heat-treated | 0.9% (w/w) VIVAPUR ™ FD 176 |
| | Whey Protein | 1.9% (w/w) Gluconolactone |
| | Isolate (e.g., | 2.0% (w/w) Dicalcium phosphate |
| | Glanbia | 5.7% (w/w) Chitolytic chitosan |
| | Provon ™ 190) | 3% (w/w) Sodium hexametaphosphate |

### F. Characterizing Formulations

In some embodiments, formulated preparations, formulated compositions described herein, one or more food components, one or more release modulators, and/or one or more excipients are subjected to one or more assessments, for example, to characterize one or more structural features and/or functional properties thereof (e.g., for quality control and/or after storage under particular conditions and for a particular period of time). In some embodiments, formulated preparations (e.g., batches) that do not meet designated criteria may be discarded or not further utilized.

### III. Methods of Producing Formulated Compositions

In some embodiments, the present disclosure provides a method of manufacturing (e.g., formulating) one or more formulated compositions (and/or formulated preparations) comprising one or more food components and one or more release modulators.

### 1. Size Reduction

In some embodiments, the present disclosure provides a method of reducing a size of a formulated composition (e.g., core, shell, matrix, solute, core-shell, particle) (and/or formulated preparation) comprising one or more food components and one or more release modulators, as described herein, to a size amenable to homogeneous formulation. In some embodiments, reducing the size of a formulated composition (e.g., core, shell, matrix, solute, core-shell, particle) (and/or formulated preparation) mitigates any negative sensory aspects (e.g., unpleasant texture, grit, taste, etc.).

In some embodiments, methods of producing a formulated composition (and/or formulated preparations) of the present disclosure includes one or more steps of size reduction of a formulated composition (and/or formulated preparation) comprising, but not limited to, planetary milling, ball milling, burr milling, roller milling, media milling, impact milling, jet milling, high-pressure homogenization, cryo-milling, hammer milling, conical milling, hand screening, granulation/extrusion, extrusion, spray drying, fluid bed agglomeration, spray congealing, high-shear granulation, tableting, pouring, roller compaction, crosslinking, prilling, spinning disc atomization, or a combination thereof.

In some embodiments of the present disclosure, formulated compositions (and/or formulated preparations) prepared using spray drying are characterized as drying of a liquid slurry using a spray drier apparatus. In some embodiments, liquid slurries used to prepare formulated compositions with spray drying are maintained at a temperature of at least about 15° C., at least about 17° C., at least about 18° C., at least about 20° C., at least about 25° C., at least about 30° C., at least about 35° C., at least about 40° C., at least about 45° C., at least about 50° C., at least about 55° C., at least about 60° C., or at least about 65° C. In some embodiments, liquid slurries used to prepare formulated compositions (and/or formulated preparations) with spray drying are agitated by techniques including, but not limited to, overhead stirring, high shear homogenization, high pressure homogenization, ultrasonic homogenization, recirculating flow, and/or magnetic stir bar.

In some embodiments of the present disclosure, formulated compositions (and/or formulated preparations) prepared using spray drying are characterized by an inlet temperature. In some embodiments, inlet temperatures used to prepare formulated compositions (and/or formulated preparations) are at least about 105° C., at least about 110° C., at least about 115° C., at least about 125° C., at least about 135° C., at least about 145° C., at least about 155° C., at least about 175° C., at least about 195° C., at least about 205° C., at least about 215° C., at least about 225° C., at least about 235° C., at least about 245° C. In some embodiments of the present disclosure, formulated compositions (and/or formulated preparations) prepared using spray drying are characterized by a product temperature of at least about 50° C., at least about 55° C., at least about 60° C., at least about 65° C., at least about 70° C., at least about 75° C., at least about 80° C., at least about 85° C., at least about 90° C., at least about 95° C., or at least about 100° C. In some embodiments of the present disclosure, formulated compositions (and/or formulated preparations) prepared using spray drying are characterized by an atomization pressure. In some embodiments, atomization pressures used to prepare formulated compositions (and/or formulated preparations) are at least about 0.75 bar, at least about 1 bar, at least about 1.25 bar, at least about 1.5 bar, at least about 1.75 bar, at least about 2 bar, at least about 2.25 bar, at least about 2.5 bar, at least about 2.75 bar, at least about 3 bar, at least about 3.25 bar, or at least about 3.5 bar.

In some embodiments, spray drying may be performed using, but not limited to, a Buchi B-290 Mini Spray Drier, a Niro Mobile Minor spray drier, a Freund Vector VSD-LAB Micro Spray Drier, or a combination thereof.

### 2. Mixing

In some embodiments, one or more food components, one or more core components, one or more shell components, one or more matrix components, one or more excipient components, one or more release modulators, one or more solute components, or a combination thereof, are mixed to form a homogeneous mixture. In some embodiments one or more food components, one or more core components, one or more shell components, one or more matrix components, one or more excipient components, one or more release modulators, one or more solute components, or a combination thereof are incubated in a solvent including, but not limited to, water, ethanol, acetone, acetic acid, hydrochloric acid, ammonia, triethyl citrate, triacetin, vegetable oil, seed oil, fish oil, or ketone esters, for a period of at least about 15 minutes, at least about 30 minutes, at least about 45 minutes, at least about 60 minutes, at least about 90 minutes, at least about 120 minutes, at least about 180 minutes, at least about 240 minutes, or at least about 360 minutes prior to homogeneous mixing. In some embodiments one or more food components, one or more core components, one or more shell components, one or more matrix components, one or more excipient components, one or more release modulators, one or more solute components, or a combination thereof are incubated in a solvent characterized by a pH of no more than about 7.0, no more than about 6.5, no more than about 6.0, no more than about 5.5, no more than about 5.0, no more than about 4.5, no more than about 4.0, no more than about 3.5, no more than about 3.0, or no more than about 2.5 prior to homogeneous mixing. In some embodiments one or more food components, one or more core components, one or more shell components, one or more matrix components, one or more excipient components, one or more release modulators, one or more solute components, or a combination thereof are brought to a temperature of at least about 20° C., at least about 25° C., at least about 30° C., at least about 35° C., at least about 40° C., at least about 45° C., at least about 50° C., at least about 55° C., at least about 60° C., at least about 65° C., or at least about 70° C. prior to homogeneous mixing. In some embodiments, one or more excipient components are added to one or more food components, one or more core components, one or more shell components, one or more matrix components, one or more excipient components, one or more release modulators, one or more solute components, or a combination thereof prior to homogeneous mixing. In some embodiments, one or more release modulators characterized as an acidifying agent is the terminal addition to a homogeneous mixing process. For example, in some embodiments, gluconolactone is the terminal addition to a homogeneous mixing process. In some embodiments, homogeneous mixing mitigates any negative sensory aspects (e.g., unpleasant texture, grit, taste, etc.).

In some embodiments, homogeneous mixtures of one or more food components, one or more core components, one or more shell components, one or more matrix components, one or more excipient components, one or more release modulators, one or more solute components, or a combination thereof, are filtered through a mesh of pore size of at least about 0.2 μm, at least about 0.4 μm, at least about 1 μm, at least about 10 μm, at least about 50 μm, at least about 100 μm, at least about 200 μm, at least about 400 μm, at least about 800 μm, at least about 1600 μm, or at least about 5000 μm.

In some embodiments, methods of producing a formulated composition (and/or formulated preparations) of the present disclosure includes one or more steps of mixing including, but not limited to, stir-bar mixing, overhead stirring, ultrasonic mixing, high-shear mixing, high-pressure mixing, turbulent mixing, or a combination thereof.

### 3. Preparing Matrix Preparations

In some embodiments, the present disclosure provides methods for preparing one or more matrix components and/or matrix preparations. In some embodiments, one or more matrix components and/or matrix preparations are characterized as solids, gels, particles, liquids, or a combination thereof. In some embodiments, one or more matrix components and/or matrix preparations are characterized as having partial, high, and/or complete solubility in water (e.g., hydrophilic). In some embodiments, one or more matrix components and/or matrix preparations are characterized as having no, low, and/or moderate solubility in water (e.g., hydrophobic). In some embodiments, one or more matrix components and/or matrix preparations are characterized as being amphiphilic. In some embodiments, methods of preparing one or more matrix components and/or matrix preparation(s) include, but are not limited to, steps of solidification, crystallization, self-assembly, gelation, and/or hydration.

In some embodiments, methods of preparing one or more matrix components and/or matrix preparations, comprise steps of heating (to about 20° C. to about 200° C.), mixing of a formulated composition, and subsequent cooling (to about −40° C. to about 20° C.). In some embodiments, methods of preparing one or more matrix components and/or matrix preparations, comprise the action of a trigger (e.g., exposure to water, pH, light, physical forces, chemical reaction, enzymatic reaction) as provided herein. In some embodiments, methods of preparing one or more matrix components and/or matrix preparations comprise incubation without agitation for a period of at least about 30 minutes, at least about 1 hour, at least about 2 hours, at least about 3 hours, at least about 4 hours, at least about 6 hours, at least about 8 hours, at least about 10 hours, at least about 12 hours, at least about 16 hours, at least about 20 hours, or at least about 24 hours at a temperature of about 2° C. to 40° C., about 4° C. to 40° C., about 8° C. to 40° C., about 10° C. to 40° C., about 13° C. to 40° C., about 16° C. to 40° C., about 19° C. to 40° C., about 23° C. to 40° C., about 26° C. to 40° C., about 29° C. to 40° C., about 34° C. to 40° C., about 38° C. to 40° C., about 2° C. to 30° C., about 4° C. to 30° C., about 8° C. to 30° C., about 10° C. to 30° C., about 13° C. to 30° C., about 16° C. to 30° C., about 19° C. to 30° C., about 23° C. to 30° C., about 26° C. to 30° C., about 29° C. to 30° C., about 2° C. to 20° C., about 4° C. to 20° C., about 8° C. to 20° C., about 10° C. to 20° C., about 13° C. to 20° C., about 16° C. to 20° C., or about 19° C. to 20° C.

### 4. Preparing Core-Shell Preparations

In some embodiments, the present disclosure provides methods of preparing one or more shell components and/or core-shell preparations. In some embodiments, one or more shell components and/or core-shell preparations are characterized as solids, gels, particles, liquids, or a combination thereof. In some embodiments, one or more shell components and/or core-shell preparations are characterized as having partial, high, and/or complete solubility in water (e.g., hydrophilic). In some embodiments, one or more shell components and/or core-shell preparations are characterized as having no, low, and/or moderate solubility in water (e.g., hydrophobic). In some embodiments, one or more shell components and/or core-shell preparations are characterized as being amphiphilic. In some embodiments, methods of preparing one or more shell components and/or core-shell preparation(s) comprise one or more coating and/or layering steps.

In some embodiments, methods of preparing a core-shell preparation includes a single coating and/or layering step. In some embodiments, methods of preparing a core-shell preparation includes a two or more (e.g., 1, 2, 3, 4, 5, 6, 7, 8, 9, 10) successive steps of coating and/or layering.

In some embodiments, methods of coating a core-shell preparation comprise, but are not limited to, steps of spray pan coating, fluidized bed coating, dip coating, roller coating, sputter coating, self-assembly, or a combination thereof.

In some embodiments, methods of layering a core-shell preparation comprise, but are not limited to, dip coating, roller coating, layered deposition, vapor deposition, physical arrangement, or a combination thereof.

In some embodiments, methods of preparing one or more shell components and/or core-shell preparations comprise homogeneous coating and/or layering of one or more core components, solute components, and/or matrix preparations with an aqueous solution. In some embodiments, methods of preparing one or more shell components and/or core-shell preparations comprise heterogeneous coating and/or layering of one or more core components, solute components, and/or matrix preparations with an aqueous solution. In some embodiments, methods of preparing one or more shell components and/or core-shell preparations comprise homogeneous coating and/or layering of one or more core components, solute components, and/or matrix preparations with an organic solution. In some embodiments, methods of preparing one or more shell components and/or core-shell preparations comprise heterogeneous coating and/or layering of one or more core components, solute components, and/or matrix preparations with an organic solution.

### 5. Drying

In some embodiments, methods for preparing a formulated composition of the present disclosure includes one or more drying steps. In some embodiments, one or more steps of drying a formulated composition comprise reducing moisture content of said formulation. In some embodiments, one or more steps of drying a formulated composition comprise reducing water activity.

In some embodiments, one or more steps of drying a formulated composition comprise the use of chemical drying agents, elevated temperature, vacuum, or a combination thereof. For example, in some embodiments, one or more steps of drying a formulated composition comprise the use of drierite, heating, vacuum, molecular sieves, sodium sulfate, magnesium sulfate, calcium carbonate, calcium chloride, or a combination thereof.

### 6. Quantifying Food Component Loading

In some embodiments, the present disclosure provides methods for quantifying an amount of one or more food components present in a formulated composition (e.g., loading). Without wishing to be bound by any particular theory, quantifying loading can be used to characterize the efficiency of a manufacturing process. In some embodiments, quantifying loading can be used to characterize the relative composition of one or more food components in a formulated composition. In some embodiments, quantifying loading can be used to characterize the nutritional content of a formulated composition.

In some embodiments, methods of quantifying an amount of one or more food components contained in (e.g., loaded in) and formulated composition comprise contacting a formulated composition to one or more dissolution solvents and/or triggers to effect complete release of one or more food components.

In some embodiments, quantification of one or more food components is achieved using nuclear magnetic resonance, mass spectrometry, liquid chromatography, intrinsic colorimetry, intrinsic fluorimetry, enzymatic colorimetry, enzymatic fluorimetry, enzymatic amperometry, antibody-mediated assays (e.g., ELISA, Western blot), or a combination thereof.

### 7. Exemplary Methods of Production

In some embodiments, the present disclosure provides a method of preparing a formulated composition (and/or formulated preparations), the method comprising: mixing one of more food components and one or more release modulators; adding gluconolactone to the food components/release modulators mixture; allowing gelation overnight; heat treating the gelated mixture at a temperature about 85° C. to about 100° C. for about 0.5 hours to about 5 hours; diluting the heat-treated mixture with HCl or acetic acid solution at pH 6.5 comprising chitosan at 2.5% (w/v); high-shear homogenization at 14,000 rpm; filtration through a 850 μm sieve; spray drying at an inlet temperature of 200° C. with outlet temperature of 85° C. and atomization pressure of 1.85 bar, or oven-drying/milling.

### NUMBERED EMBODIMENTS

Embodiment 1. An extended-release preparation comprising:
(i) a protein payload; and
(ii) a release modulator,
wherein the release modulator interacts with the protein payload to prevent access of water molecules to the protein payload in the preparation, and wherein the protein payload is released in a subject over an extended period in acidic and neutral environments following ingestion of the preparation by the subject as compared to ingestion of the protein payload alone.

Embodiment 2. An extended-release preparation wherein the protein payload is heat-treated.

Embodiment 3. An extended-release preparation wherein the protein payload is heat-treated for about 72° C. to about 112° C. for about 60 minutes to about 120 minutes.

Embodiment 4. An extended-release preparation wherein the protein payload is heat-treated for about 77° C. to about 107° C. for about 75 minutes to about 105 minutes.

Embodiment 5. An extended-release preparation wherein the protein payload is heat-treated for about 92° C. for about 90 minutes.

Embodiment 6. An extended-release preparation wherein the protein payload is heat-treated at a pH of about 4 to about 8.

Embodiment 7. An extended-release preparation wherein the protein payload is heat-treated at a pH of about 4.5 to about 7.5.

Embodiment 8. An extended-release preparation wherein the protein payload is heat-treated at a pH of about 5 to about 7.

Embodiment 9. An extended-release preparation wherein the protein payload is pea protein isolate, whey protein isolate, oat protein isolate, soy protein isolate, wheat protein isolate, egg protein isolate, casein, bovine serum albumin, ovalbumin, α-lactalbumin, β-lactoglobulin, collagen, glutanin, gliadin, kefirin, avenin, zein, silk, gelatin, hordein, sodium carboxymethylcellulose, or a combination thereof.

Embodiment 10. An extended-release preparation wherein the release modulator is Tween 85, Span 60, Gum Arabic, Triacetin, sucrose palmitate, Pluronic F68, glucose, or a combination thereof.

Embodiment 11. An extended-release preparation wherein the protein payload is about 60% w/w of the preparation and the release modulator is about 40% w/w of the preparation.

Embodiment 12. An extended-release preparation wherein the protein payload is about 65% w/w of the preparation and the release modulator is about 35% w/w of the preparation.

Embodiment 13. An extended-release preparation wherein the protein payload is about 70% w/w of the preparation and the release modulator is about 30% w/w of the preparation.

Embodiment 14. An extended-release preparation wherein the protein payload is about 75% w/w of the preparation and the release modulator is about 25% w/w of the preparation.

Embodiment 15. An extended-release preparation wherein the protein payload is about 80% w/w of the preparation and the release modulator is about 20% w/w of the preparation.

Embodiment 16. An extended-release preparation wherein the protein payload is about 85% w/w of the preparation and the release modulator is about 15% w/w of the preparation.

Embodiment 17. An extended-release preparation wherein the protein payload is about 90% w/w of the preparation and the release modulator is about 10% w/w of the preparation.

Embodiment 18. An extended-release preparation wherein the protein payload is about 92% w/w of the preparation and the release modulator is about 8% w/w of the preparation.

Embodiment 19. An extended-release preparation wherein the protein payload is about 94% w/w of the preparation and the release modulator is about 6% w/w of the preparation.

Embodiment 20. An extended-release preparation wherein the protein payload is about 96% w/w of the preparation and the release modulator is about 4% w/w of the preparation.

Embodiment 21. An extended-release preparation wherein the protein payload is about 98% w/w of the preparation and the release modulator is about 2% w/w of the preparation.

Embodiment 22. An extended-release preparation wherein the protein payload is about 99% w/w of the preparation and the release modulator is about 1% w/w of the preparation.

Embodiment 23. An extended-release preparation further comprising one or more coatings.

Embodiment 24. An extended-release preparation wherein the one or more coatings encapsulate the protein payload and release modulator.

Embodiment 25. An extended-release preparation wherein the one or more coatings comprise a hydrophobic coat.

Embodiment 26. An extended-release preparation wherein the hydrophobic coat comprises ethyl cellulose, a basic methacrylic copolymer, aqueous latex, or methylcellulose acetate succinate.

Embodiment 27. An extended-release preparation wherein the one or more coatings comprise a hydrophilic coat encapsulating the hydrophobic coat.

Embodiment 28. An extended-release preparation wherein the hydrophilic coat comprises hydroxypropyl methylcellulose or sodium alginate.

Embodiment 29. An extended-release preparation wherein 50% of the protein payload is released into simulated gastric fluid containing 60 units/ml of pepsin at 37° C. by at least about 50 minutes.

Embodiment 30. An extended-release preparation wherein 50% of the protein payload is released into simulated gastric fluid containing 60 units/ml of pepsin at 37° C. by at least about 100 minutes.

Embodiment 31. An extended-release preparation wherein 50% of the protein payload is released into simulated gastric fluid containing 60 units/ml of pepsin at 37° C. by at least about 150 minutes.

Embodiment 32. An extended-release preparation wherein 50% of the protein payload is released into simulated gastric fluid containing 60 units/ml of pepsin at 37° C. by at least about 250 minutes.

Embodiment 33. An extended-release preparation wherein 50% of the protein payload is released into simulated gastric fluid containing 60 units/ml of pepsin at 37° C. by at least about 500 minutes.

Embodiment 34. An extended-release preparation wherein 50% of the protein payload is released into simulated gastric fluid containing 60 units/ml of pepsin at 37° C. by at least about 1000 minutes.

Embodiment 35. A food or beverage product comprising an extended-release preparation.

Embodiment 36. A food or beverage product wherein the food or beverage product is a yogurt, carbonated beverage, sports drink, snack bar, or gummy.

Embodiment 35. A food or beverage product comprising an extended-release

Embodiment 37. A method of preparing an extended-release preparation comprising heat-treating a protein payload, adding a solubilized release modulator to the heat-treated protein payload, drying the heat-treated payload/release modulator combination overnight, milling.

Embodiment 38. A method of preparing an extended-release preparation further comprising a step of fluidized bed spray coating, after milling.

### EXAMPLES

The following examples are intended to illustrate but not limit the disclosed embodiments. The following examples are useful to confirm aspects of the disclosure described above and to exemplify certain embodiments of the disclosure.

These non-limiting examples demonstrate particular features and advantages of provided technologies—e.g., of ingested formulations described herein.

Among other things, provided formulated composition may be characterized by significant improvements, including, for example, (i) improved absorption and/or bioavailability of one or more food components, (ii) improved shelf-life and resistance to degradation at decreased temperatures (e.g., −80° C., −20° C., and/or 4° C.), elevated temperatures (e.g., 22° C., 25° C., 30° C., 35° C., and/or 40° C.), in food and/or food products, in beverages and/or beverage products, in supplements, in dry powders (e.g., protein powders), in the presence of high relative humidity (e.g., up to 100%) or moisture, or a combination thereof; (iii) prolonged residence time or transit time in the gastrointestinal tract or gastrointestinal tract compartments, (iv) controlled release, delayed release or extended-release over a duration of time of one or more food components in the gastrointestinal tract of a subject, (v) controlled spatial distribution of one or more food components in the gastrointestinal tract of a subject, (vi) controlled concentration of one or more food components in the gastrointestinal tract of a subject (e.g., in the stomach, in the intestines, at the epithelial surface, in the mucus, etc.); (vii) improved shelf-life in one or more food and/or beverage products (e.g., protein powders, protein bars, dry powders, milk powders, whey powders, yogurt, drinkable yogurt, water, sports drinks, etc.); (viii) improved compatibility with other components of nutraceutical products and/or compositions that include them (e.g., supplements, foods, drinks, or other edible materials), (ix) stability of formulated compositions, and/or one or more food components in an environment comprising heat, acid, protons, salt, light, water, oxidation, or a combination thereof; (x) improved resistance of one or more food components to losses during manufacturing processes such as pasteurization, shear mixing, elevated pressurized processes, elevated temperature processes, etc.; (xi) stability of one or more food components in, or as, a dry powder (e.g., protein powder) in an environment comprising heat, acid, protons, salt, light, water, moisture, humidity, oxidation, antimicrobial peptides, or a combination thereof; (xii) tunable properties including size, coating thickness, morphology, geometry, loading, dose, interactions with the surrounding environment, and release conditions, etc.; (xiii) improved anti-caking, anti-clumping, anti-agglomerating, and/or anti-aggregating functionality at elevated temperatures; (xiv) maintenance and preservation of composition morphology (e.g., particle geometry) when exposed to typically degrading conditions, such as: decreased temperatures (e.g., −80° C., −20° C., and/or 4° C.), elevated temperatures (e.g., 22° C., 25° C., 30° C., 35° C., and/or 40° C.), in foods and/or food products, in beverages and/or beverage products, in supplements, in dry powders (e.g., protein powder), in the presence of high relative humidity (e.g., up to 100%) or moisture, or a combination thereof; (xv) mitigation of changes to taste, texture, or scents upon addition, storage, or ingestion of the formulation (xiii) controlled satiety.

### Example 1: Exemplary Whey Protein Formulation Preparation

The present example describes preparation of an exemplary heat-treated whey protein formulation preparation of the present disclosure.

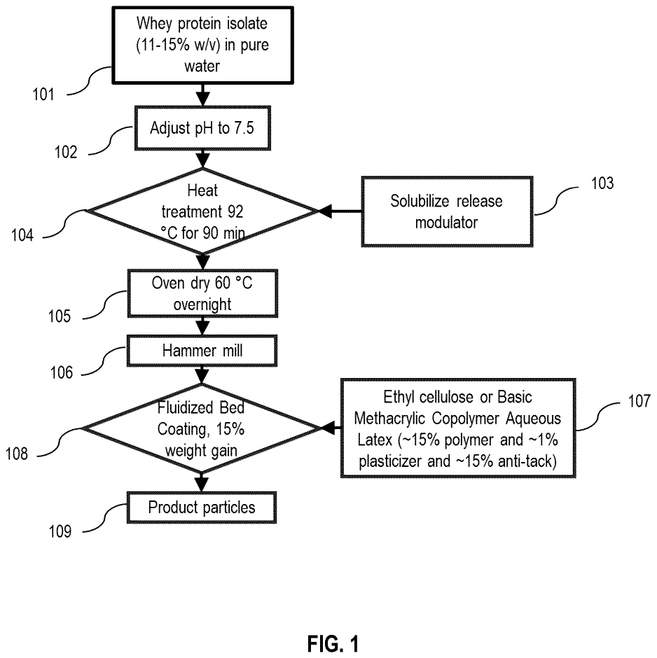

FIG. 1 shows a flow diagram of an exemplary production process for whey protein preparations. At step 101, whey protein isolate (11-15% w/v) was dissolved in pure water and the pH of the solution was adjusted to pH 7.5 at step 102. Optionally, at step 103, a soluble release modulator was added to the solution. At step 104, the solution underwent heat treatment at 92° C. for 90 min. At step 105, the solution was then oven-dried overnight at 60° C. to form a powder. After drying, the powder was hammer milled in step 106. The powder then underwent fluidized bed coating at step 107, optionally with a coating mixture 108, resulting in 15% weight gain for final microparticles 109. The coating mixture 108 comprised ethyl cellulose or basic methacrylic copolymer aqueous latex (about 15% polymer and about 1% plasticized and about 15% anti-tack).

### Example 2: Whey Protein Release

This example describes release properties of exemplary heat-treated whey protein formulation preparations.

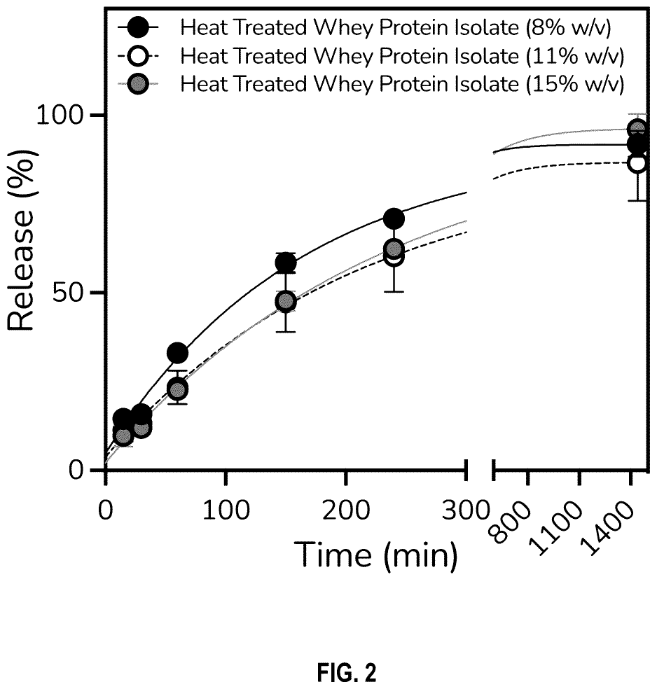

As shown in FIG. 2, heat-treated whey protein isolate formulation preparations comprising 11%, (w/v) and 15% (w/v) of whey protein exhibited extended-release (as assessed at multiple times over the full 1400 min. time course) of amino acids or peptides into simulated gastric fluid with 60 U/mL of pepsin as compared to heat-treated whey protein isolate formulations comprising 8% w/v of heat-treated whey protein. For example, after 150 min, the 8% (w/v) heat-treated whey protein isolate formulation released about 60% of total amino acids or peptides, whereas about 50% of total amino acids or peptides were released from 11% (w/v) and 15% (w/v) heat-treated whey protein isolate formulations. These data indicate that exemplary heat-treated whey protein formulation preparations of the present disclosure can extend amino acid or peptide release to achieve prolonged amino acid or peptide release following ingestion of the preparation by a subject.

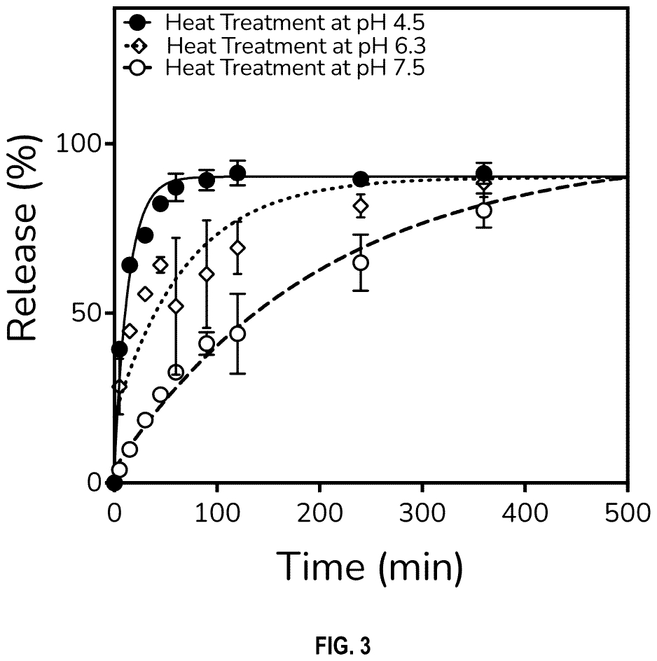

As shown in FIG. 3, an 11% (w/v) heat-treated whey protein isolate formulation at a pH of 7.5 exhibited extended (as assessed at multiple times over the full 500 min. time course) release of amino acids into simulated gastric fluid with 60 U/mL of pepsin as compared to 11% (w/v) heat-treated whey protein isolate formulations at a pH of 4.5 or 6.3. For example, after 240 min, 11% (w/v) heat-treated whey protein isolate formulation at pH 7.5 released about 65% of amino acids or peptides as compared to about 80% and 90% for 11% (w/v) heat-treated whey protein isolate formulation at pH 6.3 and pH 4.5, respectively. These data indicate that exemplary heat-treated whey protein isolate formulation preparations can extend the duration of amino acid or peptide release in a pH-dependent manner.

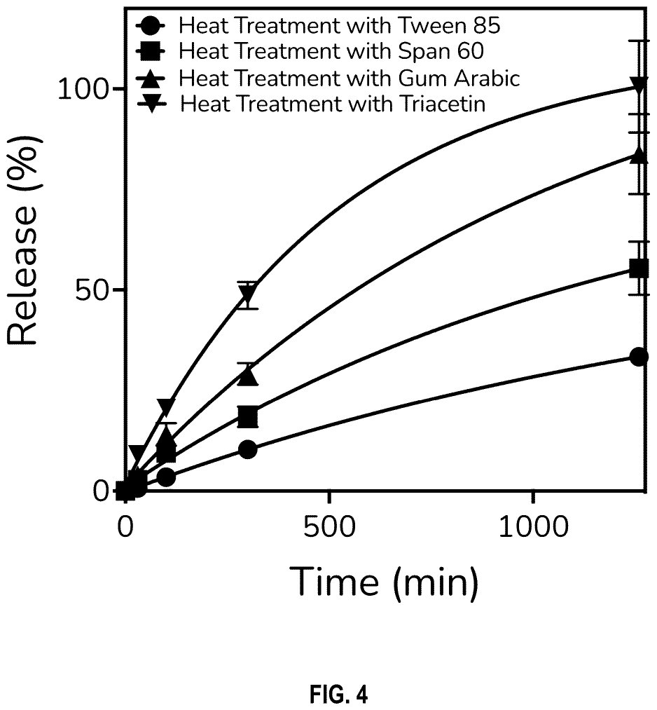

As shown in FIG. 4, exemplary heat-treated whey protein isolate formulation preparations comprising release modulators characterized as having greater water solubility (e.g., Triacetin, Gum Arabic) exhibited extended-release (as assessed at multiple times over the full 1260 min. time course) of amino acids or peptides into simulated gastric fluid with 60 U/mL of pepsin as compared to heat-treated whey protein isolate formulation preparations comprising release modulators characterized as having lower water solubility (e.g., Tween™ 85, Span™ 60). For example, after 1260 min, 11% (w/v) heat-treated whey protein isolate formulations at pH 7.5 including Triacetin or Gum Arabic released about 100% and 85% of total amino acids or peptides, respectively, as compared to 11% (w/v) heat-treated whey protein formulations at pH 7.5 including Tween™ 85 or Span™ 60, which released about 55% and 35% of total amino acids or peptides, respectively. These data indicate that the rate of release of amino acids or peptides from exemplary heat-treated whey protein isolate formulation preparations of the present disclosure can be tuned based on the release modulators included in the preparation.

### Example 3: Exemplary Formulated Compositions with Release Modulators

This example describes release properties of additional exemplary heat-treated whey protein isolate formulation preparations prepared with various release modulators.

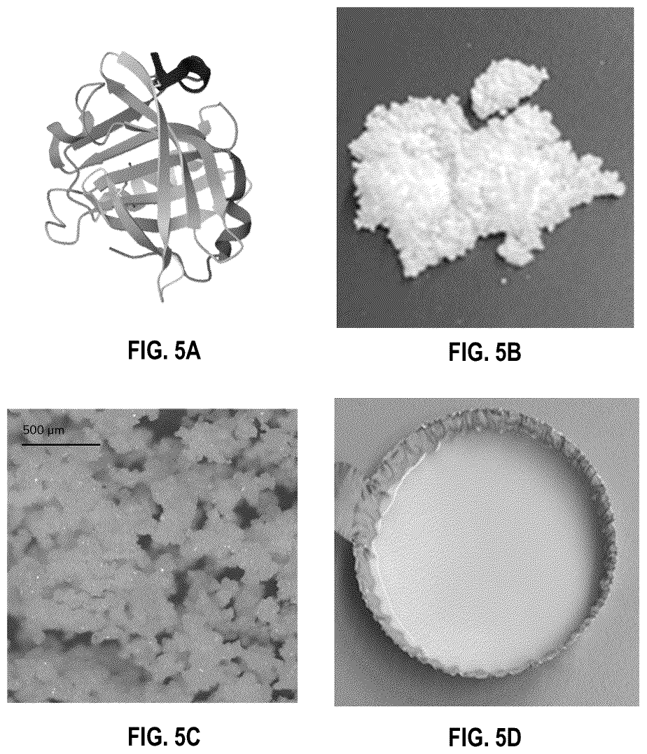

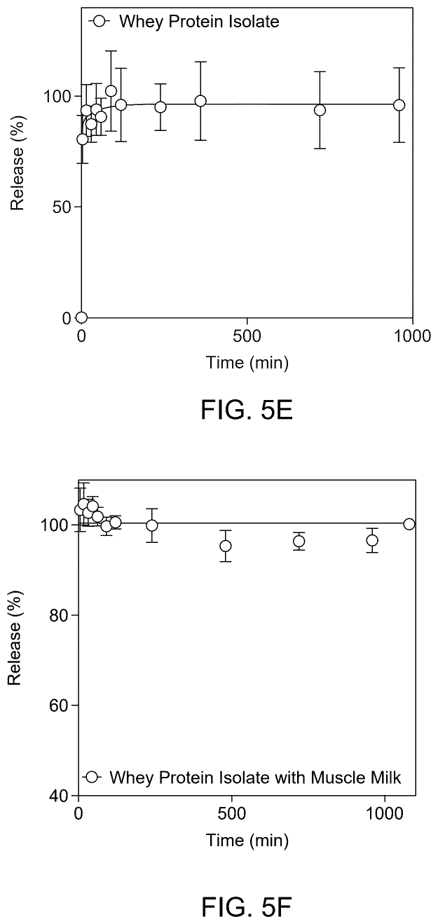

FIGS. 5A-5C depict whey protein isolate. FIG. 5A shows a ribbon diagram of whey protein structure. FIG. 5B and FIG. 5C show a macroscopic and microscopic image, respectively, of unformulated whey protein isolate powder. FIG. 5D is a macroscopic image showing the incorporation of unformulated whey protein isolate powder into a conventional milkshake (Muscle Milk® vanilla shake).

As shown in FIG. 5E, when placed in 500 mL of simulated gastric fluid with 60 U/mL pepsin at a pH of 1.2 and temperature of 37° C., unformulated whey protein isolate powder released 100% of total amino acids in less than an hour. Similarly, as shown in FIG. 5F, a mixture of unformulated whey protein isolate and a conventional milkshake (Muscle Milk® vanilla shake) in a 1:1 ratio released 100% of amino acids in less than an hour when placed in 500 mL of simulated gastric fluid with 60 U/mL pepsin at a pH of 1.2 and temperature of 37° C.

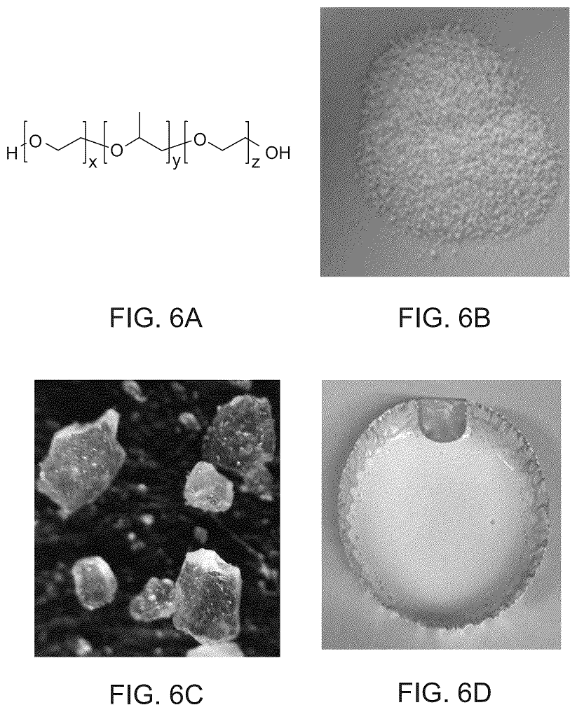

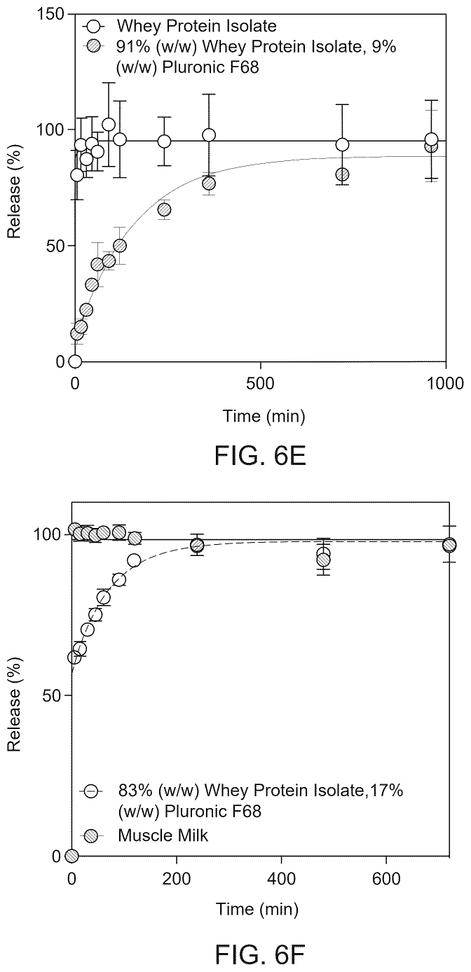

FIGS. 6A-6C depict Pluronic™ F68. FIG. 6A shows the chemical structure of Pluronic™ F68. FIG. 6B and FIG. 6C show a macroscopic and microscopic image, respectively, of Pluronic™ F68. FIG. 6D is a macroscopic image showing the incorporation of Pluronic™ F68 into a conventional milkshake (Muscle Milk® vanilla shake).

As shown in FIG. 6E, after 60 min in simulated gastric fluid with 60 U/mL of pepsin, an exemplary formulated preparation of the present disclosure comprising 91% (w/w) heat-treated whey protein isolate and 9% (w/w) Pluronic™ F68 released about 50% of total amino acids as compared to about 100% total amino acids released by unformulated whey protein isolate powder. As shown in FIG. 6F, after 60 min in simulated gastric fluid with 60 U/mL of pepsin, an exemplary formulated composition of the present disclosure comprising 83% (w/w) heat-treated whey protein isolate and 17% (w/w) Pluronic™ F68 mixed with a conventional milkshake (Muscle Milk® vanilla shake) at a 1:1 ratio released about 80% of total amino acids as compared to about 100% of amino acids released from a conventional milkshake (Muscle Milk® vanilla shake). These data indicate that exemplary heat-treated whey protein isolate formulation preparations of the present disclosure prepared with Pluronic F68 as a release modulator can extend the duration of amino acid release and that this extended-release is maintained when the preparations are incorporated into a food product (e.g., Muscle Milk®).

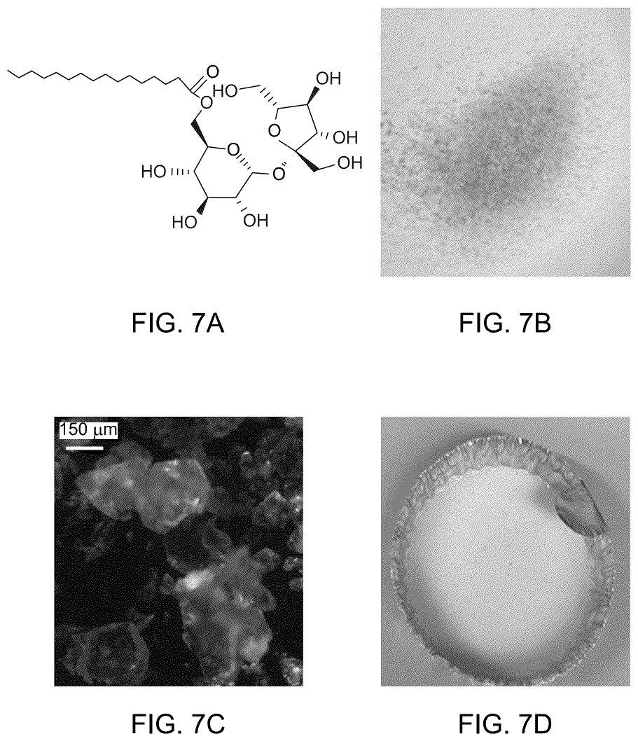

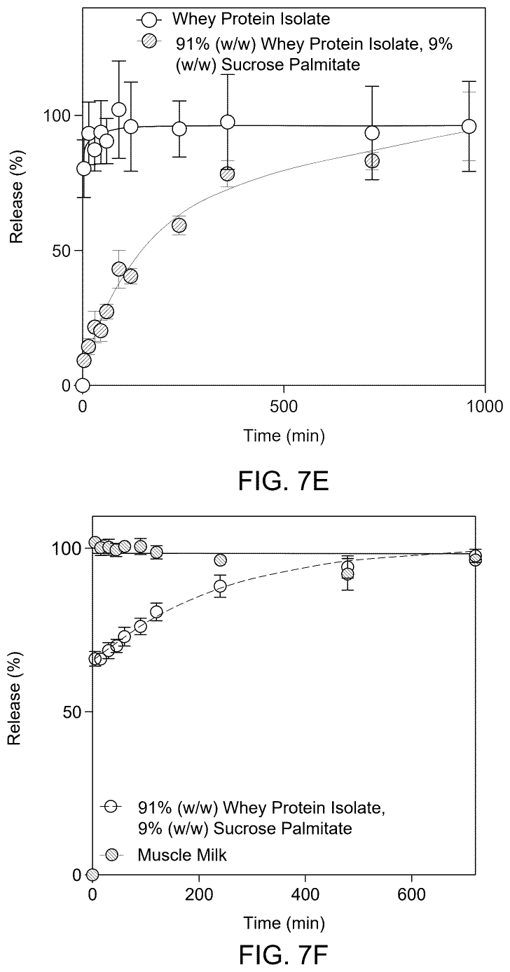

FIGS. 7A-7C depict sucrose palmitate. FIG. 7A shows the chemical structure of sucrose palmitate. FIG. 7B and FIG. 7C show a macroscopic and microscopic image, respectively, of sucrose palmitate. FIG. 7D is a macroscopic image showing the incorporation of sucrose palmitate into a conventional milkshake (Muscle Milk® vanilla shake).

As shown in FIG. 7E, after 60 min in simulated gastric fluid with 60 U/mL of pepsin, an exemplary formulated preparation of the present disclosure comprising 91% (w/w) heat-treated whey protein isolate and 9% (w/w) sucrose palmitate released about 25% of total amino acids as compared to about 100% total amino acids released by unformulated whey protein isolate powder. As shown in FIG. 7F, after 60 min in simulated gastric fluid with 60 U/mL of pepsin, an exemplary formulated preparation of the present disclosure comprising 91% (w/w) heat-treated whey protein isolate and 9% (w/w) sucrose palmitate mixed with a conventional milkshake (Muscle Milk® vanilla shake) at a 1:1 ratio released about 70% of total amino acids as compared to about 100% of amino acids released from a conventional milkshake (Muscle Milk® vanilla shake). These data indicate that exemplary heat-treated whey protein isolate formulations of the present disclosure prepared with sucrose palmitate as a release modulator can extend the duration of amino acid release and that this extended-release is maintained when the formulations is incorporated into a food product (e.g., Muscle Milk®).

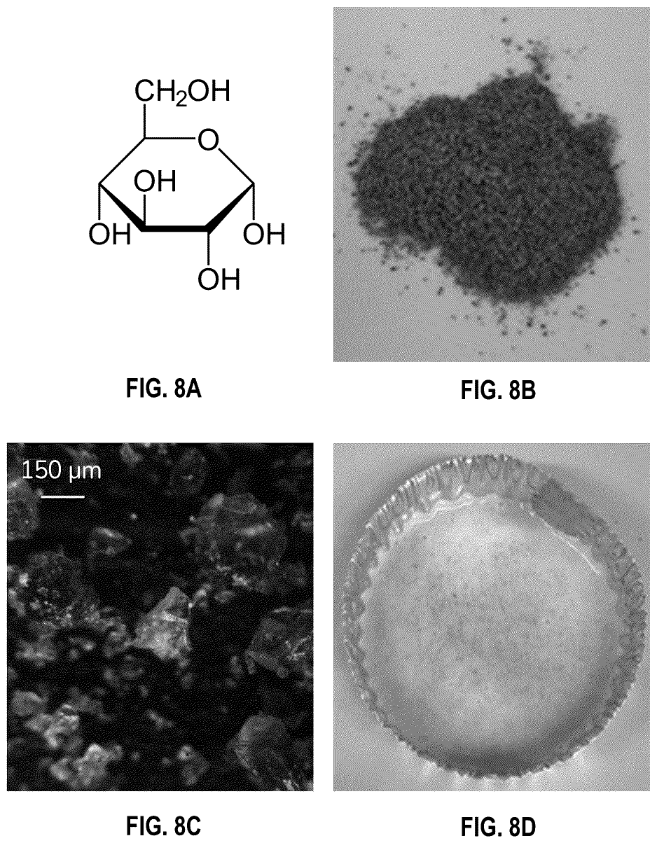

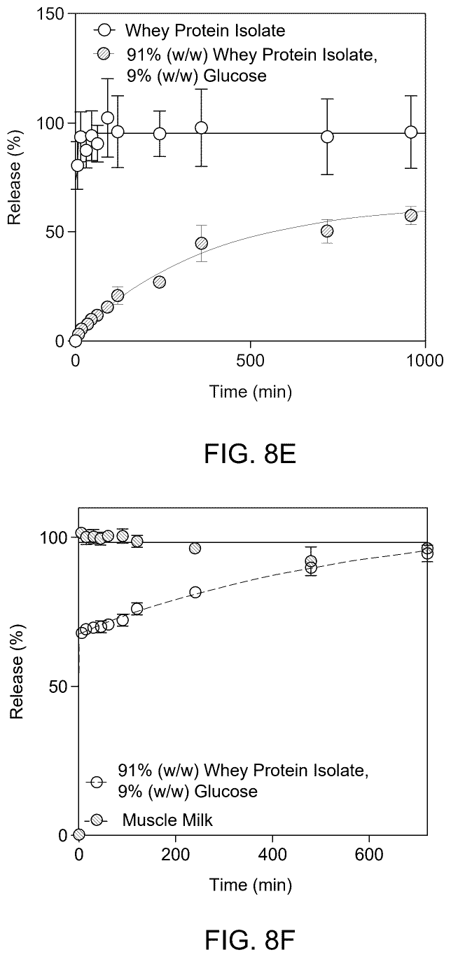

FIGS. 8A-8C depict glucose. FIG. 8A shows the chemical structure of sucrose palmitate. FIG. 8B and FIG. 8C show a macroscopic and microscopic image, respectively, of glucose. FIG. 8D is a macroscopic image showing the incorporation of sucrose palmitate into a conventional milkshake (Muscle Milk® vanilla shake).

As shown in FIG. 8E, after 120 min in simulated gastric fluid with 60 U/mL of pepsin, an exemplary formulated preparation of the present disclosure comprising 91% (w/w) heat-treated whey protein isolate and 9% (w/w) glucose released about 20% of total amino acids as compared to about 100% total amino acids released by unformulated whey protein isolate powder. As shown in FIG. 8F, after 60 min in simulated gastric fluid with 60 U/mL of pepsin, an exemplary formulated preparation of the present disclosure comprising 91% (w/w) heat-treated whey protein isolate and 9% (w/w) glucose mixed with a conventional milkshake (Muscle Milk® vanilla shake) at a 1:1 ratio released about 70% of total amino acids as compared to about 100% of amino acids released from a conventional milkshake (Muscle Milk® vanilla shake). These data indicate that exemplary heat-treated whey protein isolate preparations of the present disclosure prepared with glucose as a release modulator can extend the duration of amino acid release and that this extended-release is maintained when the preparation is incorporated into a food product (e.g., Muscle Milk®).

### Example 4: Exemplary Coated Formulated Preparations

This example describes the morphological and release properties of an exemplary coated heat-treated whey protein isolate preparation.

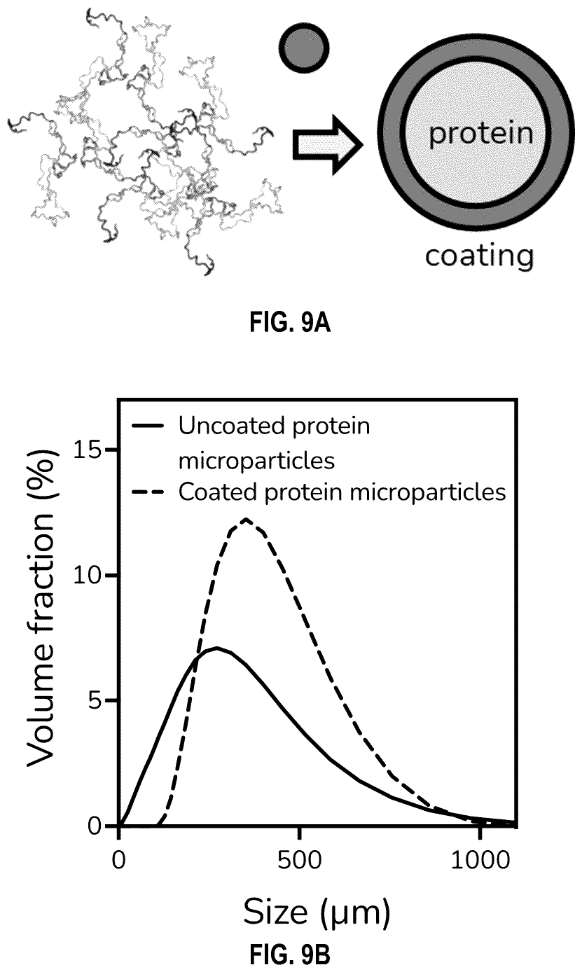

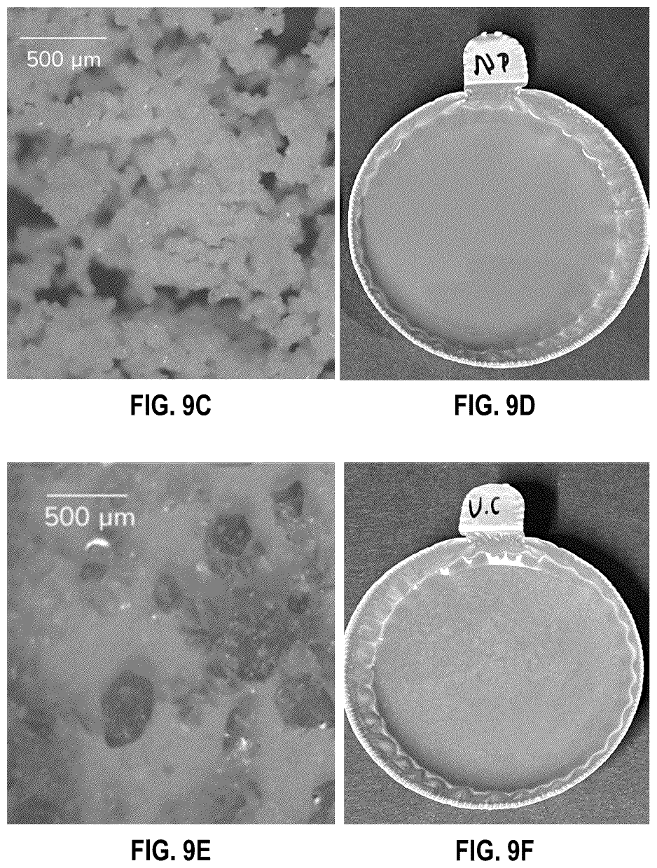

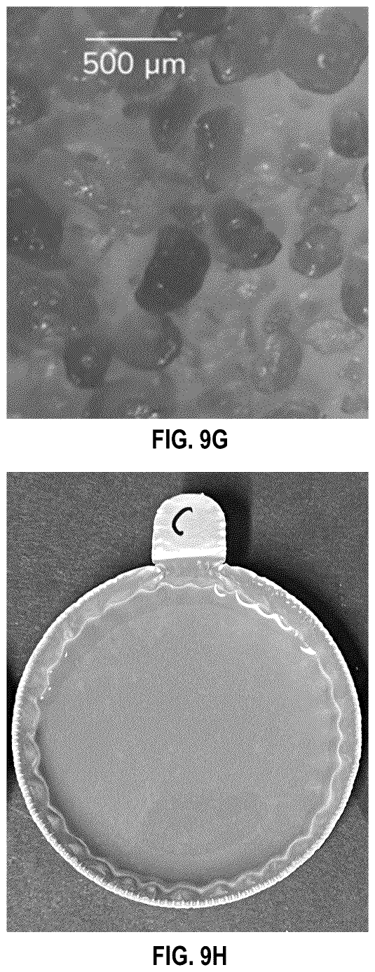

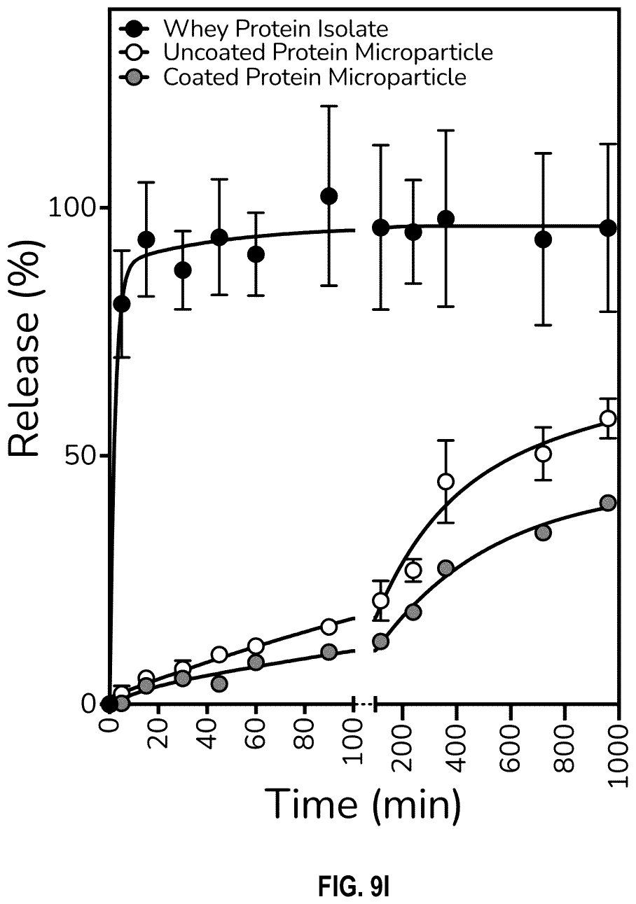

As shown in FIG. 9A, heat-treated whey protein isolate formulations can include one or more coatings to form a core-shell structure. Coated heat-treated whey protein isolate formulations had, on average, a larger particle size as compared to uncoated heat-treated whey protein isolate as shown in FIG. 9B. FIG. 9C shows a microscopic image of unformulated whey protein isolate powder. FIG. 9D shows a macroscopic image of unformulated whey protein isolate powder incorporated into a conventional milkshake (Muscle Milk® chocolate shake) at a 1:1 ratio. FIG. 9E shows a microscopic image of heat-treated whey protein isolate. FIG. 9F shows a macroscopic image of heat-treated whey protein isolate incorporated into a conventional milkshake (Muscle Milk® chocolate shake) at a 1:1 ratio. FIG. 9G shows a microscopic image of heat-treated whey protein isolate coated with 15% (w/w) ETHOCEL standard 300. FIG. 9H shows a macroscopic image of coated heat-treated whey protein isolate incorporated into a conventional milkshake (Muscle Milk® chocolate shake) at a 1:1 ratio.

As shown in FIG. 9I, a coated heat-treated whey protein isolate preparation mixed with a conventional milkshake (Muscle Milk® chocolate shake) at a 1:1 ratio released about 10% and 25% of total amino acids after 90 min, and 360 min. in simulated gastric fluid with 60 U/mL of pepsin, respectively. In comparison, an uncoated heat-treated whey protein isolate formulation mixed with a conventional milkshake (Muscle Milk® chocolate shake) at a 1:1 ratio released about 20% and 45% of total amino acids after 90 min, and 360 min. in simulated gastric fluid with 60 U/mL of pepsin, respectively. In comparison, about 20% and 45% of total amino acids were released at 90 min, and 360 min., respectively, from an uncoated heat-treated whey protein isolate formulation mixed with a conventional milkshake (Muscle Milk® chocolate shake), and 100% of total amino acids were released after 90 min. from unformulated whey protein isolate powder mixed with a conventional milkshake (Muscle Milk® chocolate shake). These data indicate that exemplary coated heat-treated whey protein isolate preparations of the present disclosure can extend the duration of amino acid release and that this extended-release is maintained when the preparations are incorporated into a food product (e.g., Muscle Milk®).

### Example 5: Assessing Nutritional Content of Exemplary Heat-Treated Whey Protein Isolate Formulation Preparations

The present example describes mass spectrometry studies of exemplary heat-treated whey protein isolate formulation preparations.

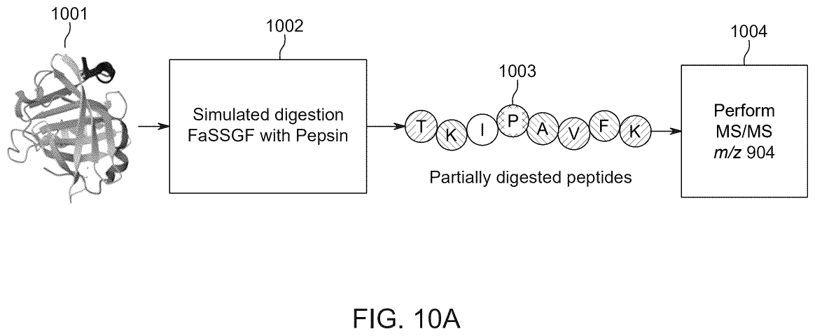

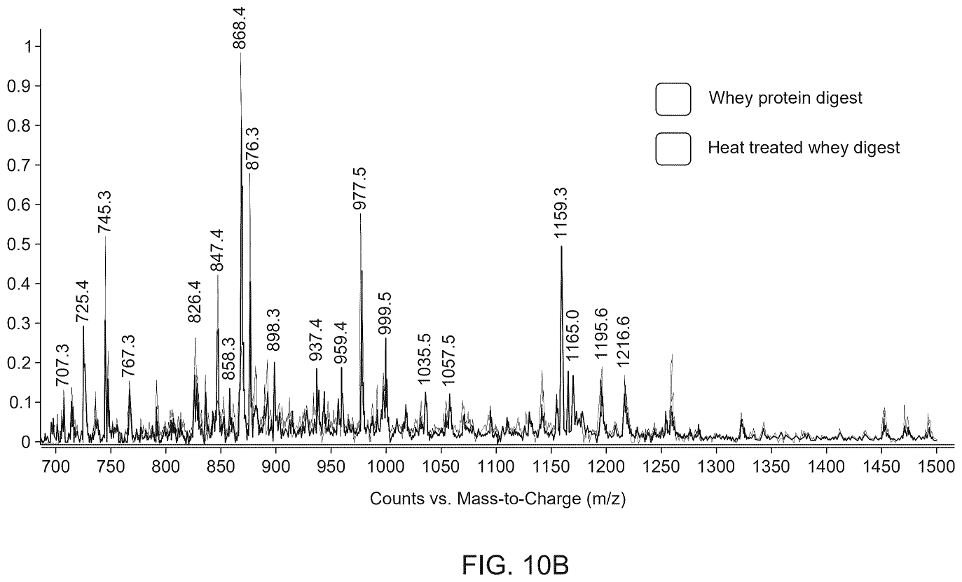

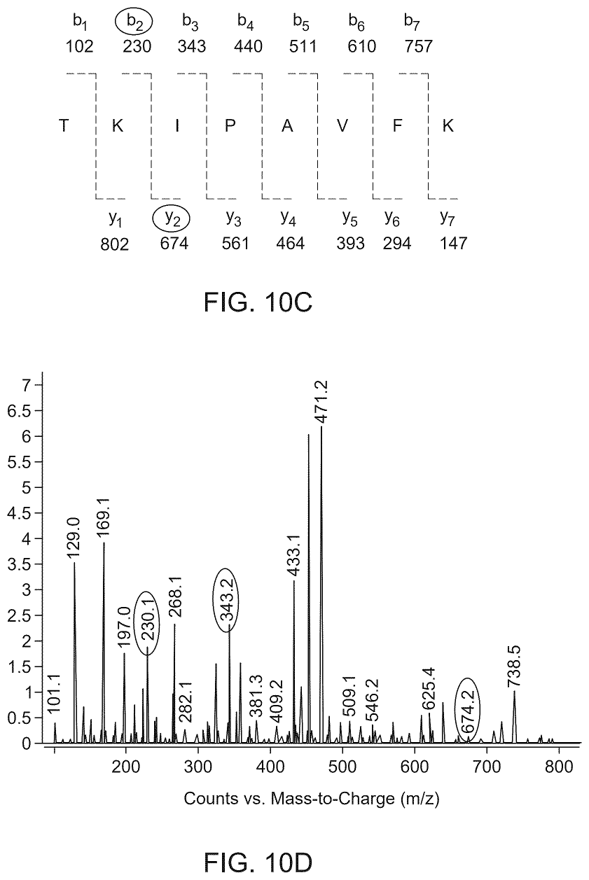

Mass spectrometry was performed on simulated gastric fluid digests of exemplary heat-treated whey protein isolate preparations as described herein to evaluate the protein, peptide, and/or amino acid (e.g., nutritional) content. FIG. 10A shows a flow diagram for assessing protein, peptide, and/or amino acid (e.g., nutritional) nutritional content. Sample proteins 1001 (heat-treated or untreated whey protein isolate) underwent simulated digestion at step 1002 in a fasted state simulated gastric fluid (FaSSGF) with 60 U/mL pepsin overnight, resulting in partially digested peptides 1003. At step 1004, the partially digested peptides were analyzed in a triple quadrupole tandem mass spectrometer (MS/MS or TQMS) selecting for fragmentation of m/z 904.

FIG. 10B shows overlayed mass-spectrometry plots for two samples: whey protein isolate digest and heat-treated whey protein isolate digest. The plots show that the two samples comprise the same peptide sequences (having identical mass) as well as having the same abundance of said peptides. Furthermore, as shown in FIG. 10C, whey protein isolate possesses characteristic peptides, such as lactoglobulin “TKIPAVFK” with b₂ and y₂ fragments. Without wishing to be bound by any particular theory, these and other characteristic peptides may be used to characterize the nutritional content of a formulated preparation of the present disclosure. To assess the presence of these characteristic fragments in heat-treated whey protein isolate digests, TQMS was used to sequence peptide fragments by selection and fragmentation. As shown in the mass-spectrometry plot of FIG. 10D, peaks corresponding to the characteristic b₂ and y₂ fragments were present in heat-treated whey protein isolate digests. These data indicate that nutritional content is preserved during the heat-treatment and/or formulation steps for preparing exemplary heat-treated whey protein isolate preparations of the present disclosure.

### Example 6: Exemplary Formulated Preparations with Release Modulators (Interpenetrating Polymer Networks)

This example describes release properties of additional exemplary heat-treated whey protein isolate preparations prepared with various release modulators (e.g., hydrocolloids, calcium-crosslinked polymers, amino acids, etc.).

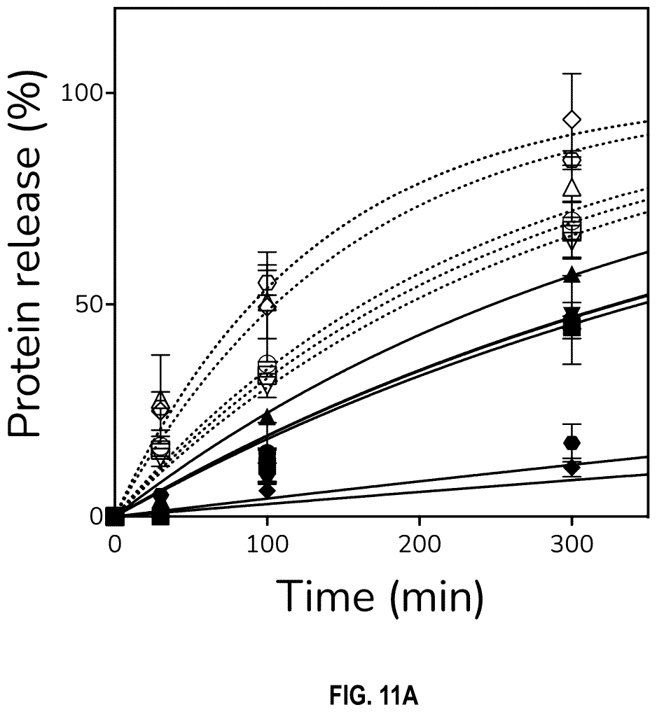

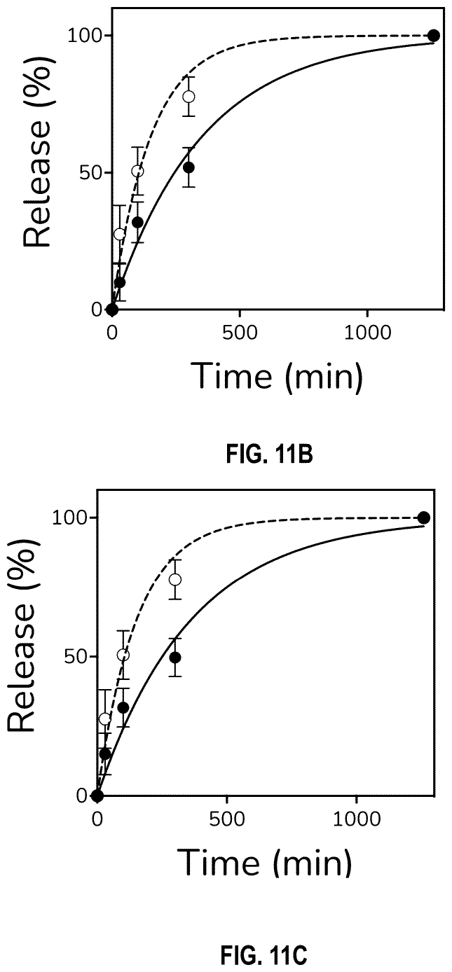

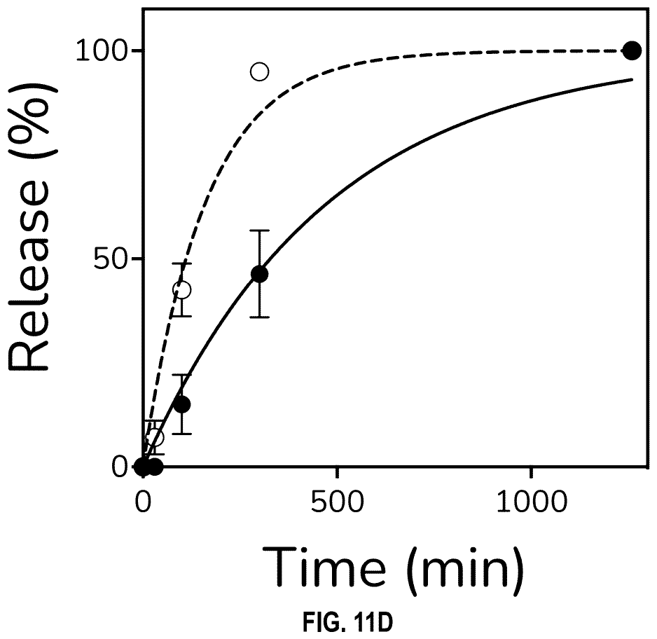

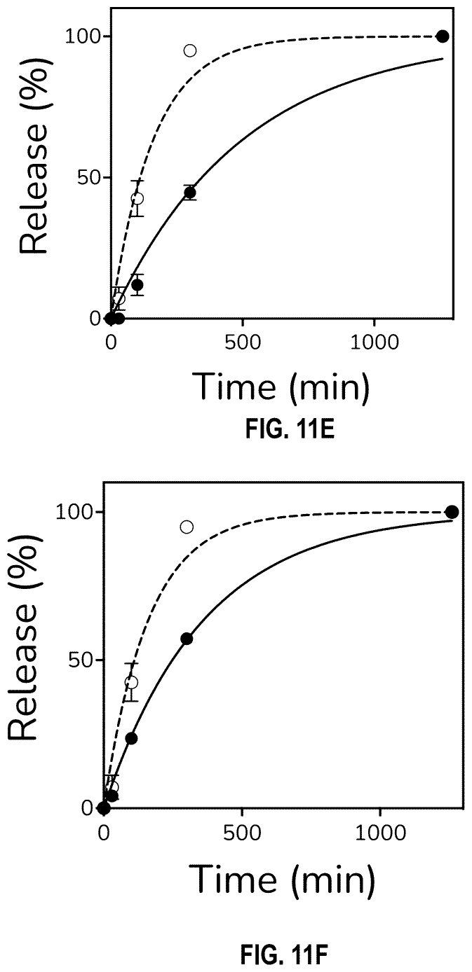

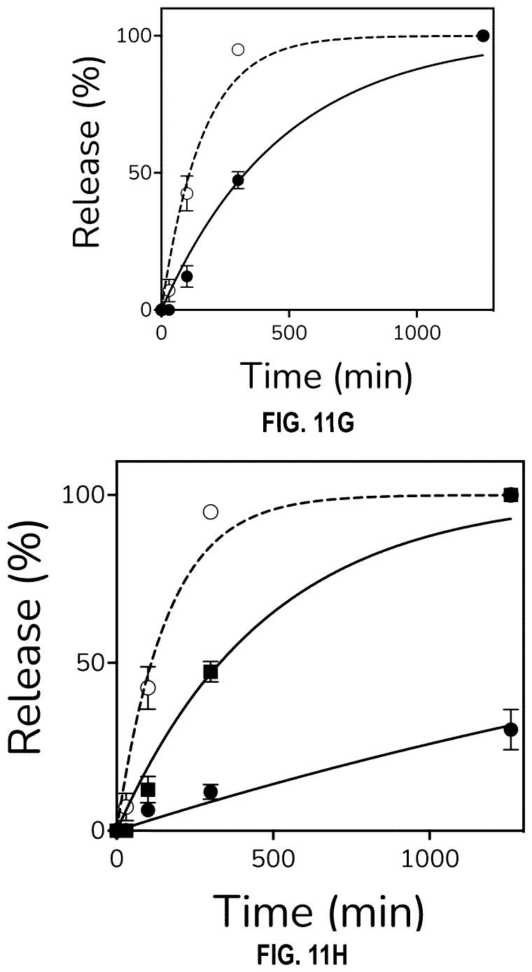

As shown in FIG. 11A, exemplary formulated preparations comprising one or more release modulators that form an interpenetrating polymer network (e.g. 76% (w/w) heat-treated whey protein, 15% (w/w) Vivapharm™ PHR5 (closed circles); 76% (w/w) heat-treated whey protein isolate, 6% (w/w) gluconolactone, 3% (w/w) calcium carbonate, 15% (w/w) Vivapharm™ PH155 (closed squares); 76% (w/w) heat-treated whey protein isolate, 6% (w/w) gluconolactone, 3% (w/w) calcium carbonate, 15% (w/w) Vivapharm™ PH176 (down-turned closed triangles); 76% (w/w) heat-treated whey protein isolate, 6% (w/w) gluconolactone, 3% (w/w) dicalcium phosphate, 15% (w/w) Vivapharm™ PH155 (closed diamonds); 76% (w/w) heat-treated whey protein isolate, 6% (w/w) gluconolactone, 3% (w/w) calcium carbonate, 15% (w/w) Pectner™ APC210 (up-turned closed triangles); 66% (w/w) heat-treated whey protein isolate, 5% (w/w) gluconolactone, 3% (w/w) calcium carbonate, 26% (w/w) Pectner™ APC220 (closed hexagons)) exhibited prolonged extended-release (as assessed at multiple times over the full 300 min. time course) of proteins/peptides into simulated gastric fluid with 60 U/mL of pepsin as compared to heat-treated whey protein isolate formulations comprising release modulators that do not form interpenetrating networks (e.g., 91% heat-treated whey protein isolate, 9% (w/v) Methocel™ 15LV (open circles); 91% heat-treated whey protein isolate, 9% (w/v) Methocel™ 4C (open squares); 91% heat-treated whey protein isolate, 9% (w/v) Methocel™ 15C (down-turned open triangles); 91% heat-treated whey protein isolate, 9% (w/v) Methocel™ 4M (open diamonds); 91% heat-treated whey protein isolate, 9% (w/v) glycine (up-turned open triangles); or 91% heat-treated whey protein isolate, 9% (w/v) lysine (open hexagons))

As shown in FIG. 11B, an exemplary formulated preparation comprising a release modulator that forms a hydrocolloid interpenetrating polymer network (e.g., 91% (w/w) heat-treated whey protein isolate, 9% (w/w) iota carrageenan (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a release modulator that does not form an interpenetrating network (e.g., 91% (w/w) heat-treated whey protein isolate, 9% (w/w) glycine (open circles)).

Similarly, as shown in FIG. 11C, an exemplary formulated preparation comprising a release modulator that forms a hydrocolloid interpenetrating polymer network (e.g., 91% (w/w) heat-treated whey protein isolate, 9% (w/w) lambda carrageenan (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a release modulator that does not form an interpenetrating network (e.g., 91% (w/w) heat-treated whey protein isolate, 9% (w/w) glycine (open circles)).

As shown in FIG. 11D, an exemplary formulated preparation comprising a release modulator that forms a calcium cross-linked interpenetrating polymer network gel (e.g., 76% (w/w) heat-treated whey protein isolate, 6% (w/w) gluconolactone, 3% (w/w) calcium carbonate, 15% (w/w) Vivapharm™ PH R5 (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a release modulator that does not form a interpenetrating polymer network gel in the presence of calcium (e.g., 76% (w/w) heat-treated whey protein isolate, 6% (w/w) gluconolactone, 3% (w/w) calcium carbonate, 15% (w/w) Pectner™ 169B (open circles)).

Similarly, as shown in FIG. 11E, an exemplary formulated preparation comprising a release modulator that forms a calcium cross-linked interpenetrating polymer network gel (e.g., 76% (w/w) heat-treated whey protein isolate, 6% (w/w) gluconolactone, 3% (w/w) calcium carbonate, 15% (w/w) Vivapharm™ PH 155 (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a release modulator that does not form an interpenetrating polymer network gel in the presence of calcium (e.g., 76% (w/w) heat-treated whey protein isolate, 6% (w/w) gluconolactone, 3% (w/w) calcium carbonate, 15% (w/w) Pectner™ 169B (open circles)).

As shown in FIG. 11F, an exemplary formulated preparation comprising a release modulator that forms a calcium cross-linked interpenetrating polymer network gel (e.g., 76% (w/w) heat-treated whey protein isolate, 6% (w/w) gluconolactone, 3% (w/w) calcium carbonate, 15% (w/w) Pectner™ APC210 (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a release modulator that does not form a interpenetrating polymer network gel in the presence of calcium (e.g., 76% (w/w) heat-treated whey protein isolate, 6% (w/w) gluconolactone, 3% (w/w) calcium carbonate, 15% (w/w) Pectner™ 169B (open circles)).

Similarly, as shown in FIG. 11G, an exemplary formulated preparation comprising a release modulator that forms a calcium cross-linked interpenetrating polymer network gel (e.g., 76% (w/w) heat-treated whey protein isolate, 6% (w/w) gluconolactone, 3% (w/w) calcium carbonate, 15% (w/w) Vivapharm™ PH176 (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a release modulator that does not form a interpenetrating polymer network gel in the presence of calcium (e.g., 76% (w/w) heat-treated whey protein isolate, 6% (w/w) gluconolactone, 3% (w/w) calcium carbonate, 15% (w/w) Pectner™ 169B (open circles)).

As shown in FIG. 11H, an exemplary formulated preparation comprising a release modulator that forms a calcium cross-linked interpenetrating polymer network gel (e.g., 76% (w/w) heat-treated whey protein isolate, 6% (w/w) gluconolactone, 3% (w/w) dicalcium phosphate, 15% (w/w) Vivapharm™ PH155 (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a release modulator that does not form a interpenetrating polymer network gel in the presence of calcium (e.g., 76% (w/w) heat-treated whey protein isolate, 6% (w/w) gluconolactone, 3% (w/w) calcium carbonate, 15% (w/w) Pectner™ 169B (open circles)).

These data further indicate that exemplary heat-treated whey protein isolate formulations of the present disclosure comprising interpenetrating polymer networks can extend the duration of amino acid release.

### Example 7: Exemplary Formulated Preparations with Release Modulators (Carbohydrates)

This example describes release properties of additional exemplary heat-treated whey protein isolate preparations prepared with various release modulators (e.g., carbohydrates).

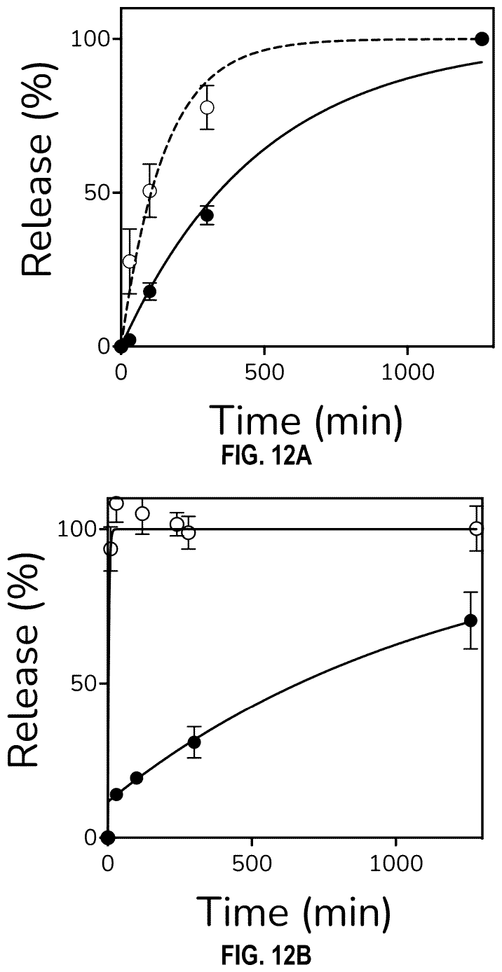

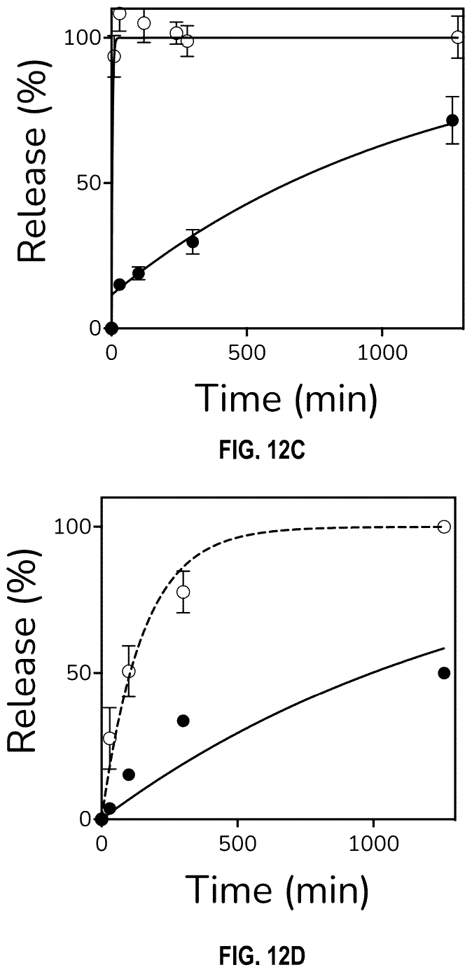

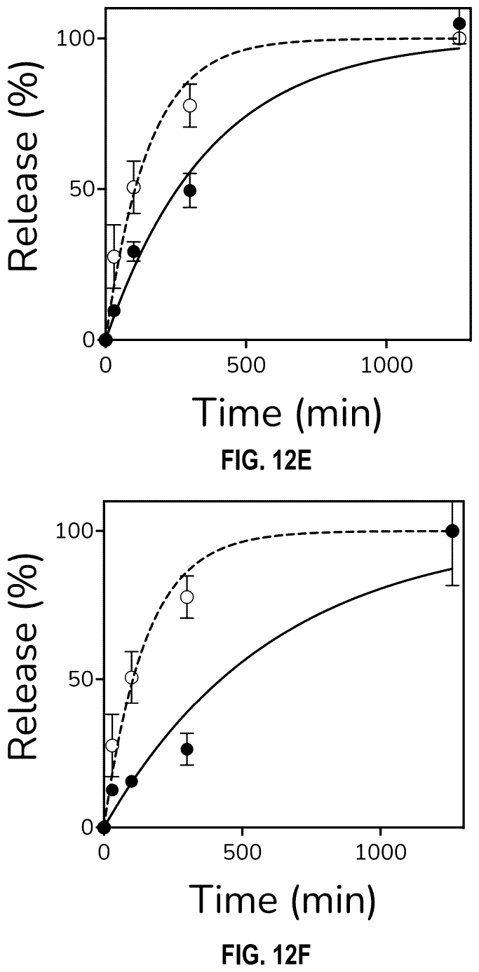

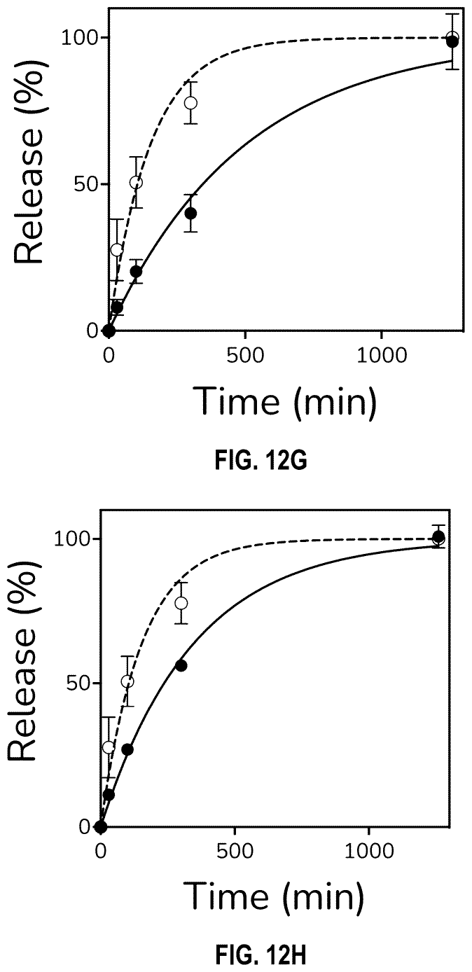

As shown in FIG. 12A, an exemplary formulated preparation comprising a carbohydrate release modulator (e.g., 91% (w/w) heat-treated whey protein isolate, 9% (w/w) hypromellose acetate succinate (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a non-carbohydrate release modulator (e.g., 91% (w/w) heat-treated whey protein isolate, 9% (w/w) glycine (open circles)).

As further shown in FIG. 12B, an exemplary formulated preparation comprising a carbohydrate release modulator (e.g., 71.4% (w/w) heat-treated whey protein isolate, 14.3% (w/w) maltodextrin DE16, 14.3% (w/w) functional rice starch (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a non-carbohydrate release modulator (e.g., 71% (w/w) heat-treated whey protein isolate, 27% (w/w) nutritional yeast, 2% (w/w) transglutaminase (open circles)).

Similarly, as shown in FIG. 12C, an exemplary formulated preparation comprising a carbohydrate release modulator (e.g., 71.4% (w/w) heat-treated whey protein isolate, 14.3% (w/w) maltodextrin DE16, 14.3% (w/w) corn starch (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a non-carbohydrate release modulator (e.g., 71% (w/w) heat-treated whey protein isolate, 27% (w/w) nutritional yeast, 2% (w/w) transglutaminase (open circles)).

Similarly, as shown in FIG. 12D, an exemplary formulated preparation comprising a carbohydrate release modulator (e.g., 91% (w/w) heat-treated whey protein isolate, 9% (w/w) ORAFTI Inulin GR (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a non-carbohydrate release modulator (e.g., 91% (w/w) heat-treated whey protein isolate, 9% (w/w) glycine (open circles)).

As shown in FIG. 12E, an exemplary formulated preparation comprising a carbohydrate release modulator (e.g., 91% (w/w) heat-treated whey protein isolate, 9% (w/w) Promoat™ (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a non-carbohydrate release modulator (e.g., 91% (w/w) heat-treated whey protein isolate, 9% (w/w) glycine (open circles))

As shown in FIG. 12F, an exemplary formulated preparation comprising a carbohydrate release modulator (e.g., 91% (w/w) heat-treated whey protein isolate, 9% (w/w) glucose (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a non-carbohydrate release modulator (e.g., 91% (w/w) heat-treated whey protein isolate, 9% (w/w) glycine (open circles)).

As shown in FIG. 12G, an exemplary formulated preparation comprising a carbohydrate release modulator (e.g., 91% (w/w) heat-treated whey protein isolate, 9% (w/w) functional rice starch (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a non-carbohydrate release modulator (e.g., 91% (w/w) heat-treated whey protein isolate, 9% (w/w) glycine (open circles)).

As further shown in FIG. 12H, an exemplary formulated preparation comprising a carbohydrate release modulator (e.g., 91% (w/w) heat-treated whey protein isolate, 9% (w/w) tapioca polydextrose (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a non-carbohydrate release modulator (e.g., 91% (w/w) heat-treated whey protein isolate, 9% (w/w) glycine (open circles)).

These data indicate that exemplary heat-treated whey protein isolate formulations comprising of the present disclosure comprising carbohydrate release modulators can extend the duration of amino acid release.

### Example 8: Additional Exemplary Formulated Preparations with Release Modulators (Calcium-Crosslinked Matrices)

This example describes release properties of additional exemplary heat-treated whey protein isolate formulations prepared with various release modulators (e.g., calcium-crosslinked matrices).

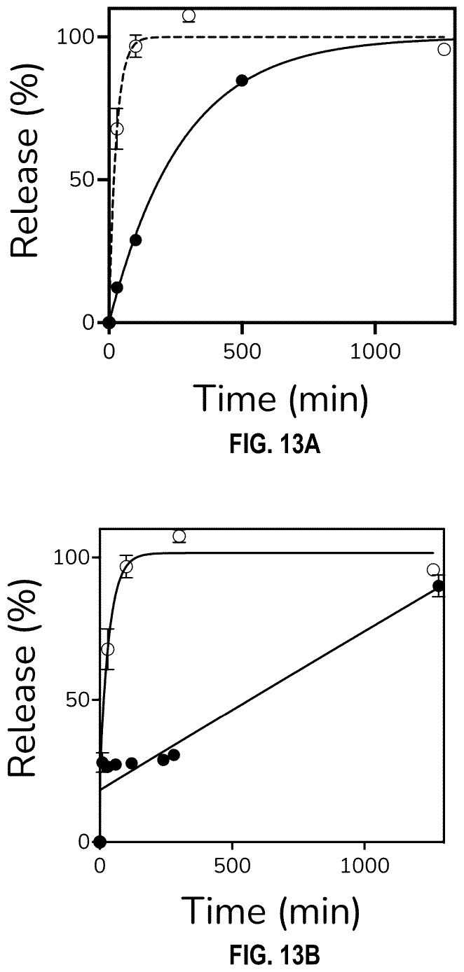

As shown in FIG. 13A, an exemplary formulated preparation comprising a release modulator that forms a calcium cross-linked interpenetrating polymer network gel (e.g., 82.6% (w/w) heat-treated whey protein isolate, 15.1% (w/w) Vivapur™ Alginate FD176, 1.5% (w/w) gluconolactone, 0.8% (w/w) dicalcium phosphate (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a release modulator that does not form a interpenetrating polymer network gel in the presence of calcium (e.g., 83% (w/w) heat-treated whey protein isolate, 8% (w/w) sodium silicate, 9% (w/w) calcium carbonate (open circles)).

Similarly, as shown in FIG. 13B, an exemplary formulated preparation comprising a release modulator that forms a calcium cross-linked interpenetrating polymer network gel (e.g., 80% (w/w) heat-treated whey protein isolate, 13.4% (w/w) Pectner™ APC210, 0.9% Vivapur™ FD176, 2.9% (w/w) Swanlac™ ASL, 1.4% (w/w) gluconolactone, 1.4% (w/w) dicalcium phosphate (closed circles)) exhibited prolonged extended-release (as assessed at multiple times over the full 1250 min. time course) of proteins/peptides into simulated gastric fluid with 60 U/mL of pepsin as compared to a heat-treated whey protein isolate formulation comprising a release modulator that does not form a interpenetrating polymer network gel in the presence of calcium (e.g., 83% (w/w) heat-treated whey protein isolate, 8% (w/w) sodium silicate, 9% (w/w) calcium carbonate (open circles)).

These data further indicate that exemplary heat-treated whey protein isolate formulations of the present disclosure comprising interpenetrating polymer networks can extend the duration of amino acid release.

### Example 9: Physical Characteristics of Exemplary Formulated Preparations

This example describes physical characteristics of additional exemplary heat-treated whey protein isolate formulation preparations comprising various release modulators.

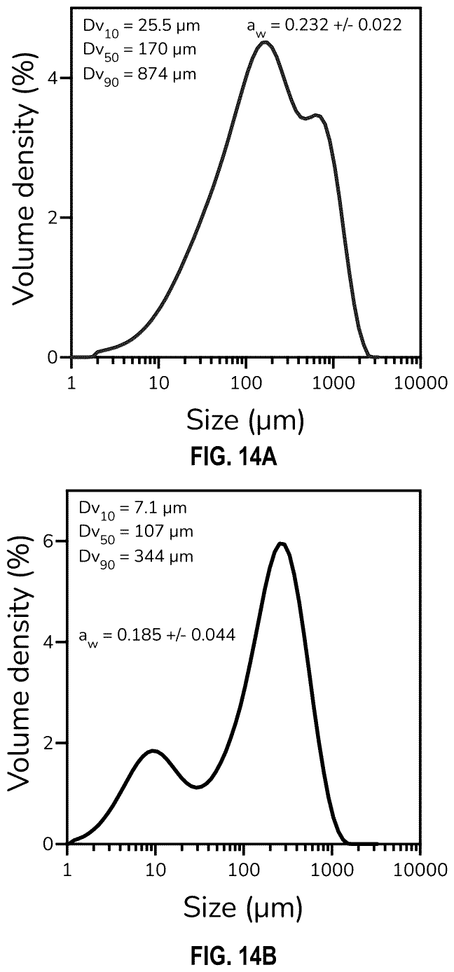

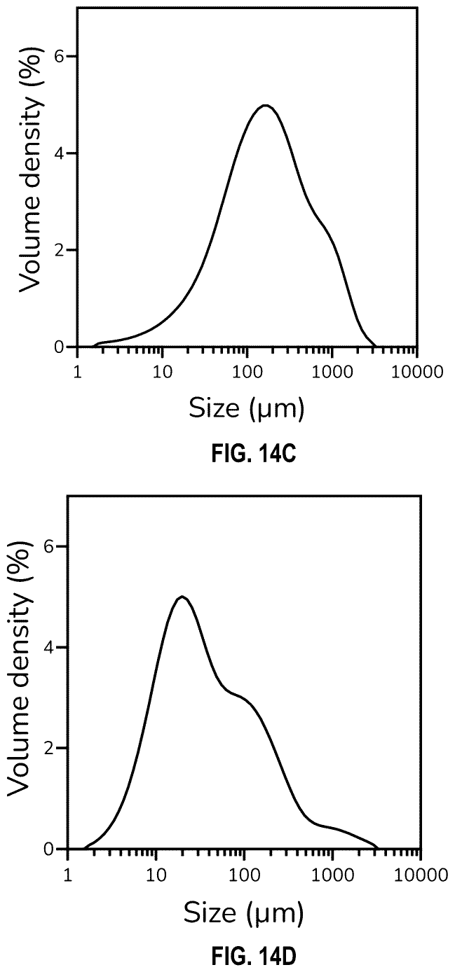

As shown in FIG. 14A, a preparation of an exemplary formulated preparation of the present disclosure (e.g., 81.2% (w/w) heat-treated whey protein isolate, 16.2% (w/w) rice starch, 1% (w/w) chitosan, 1% (w/w) tannic acid, 0.6% (w/w) laccase)) has Dv₁₀, Dv₅₀, and Dv₉₀ values of 25.5 μm, 170 μm, and 874 μm, respectively, and a water activity of 0.232±0.022.

As shown in FIG. 14B, a preparation of an exemplary formulated preparation of the present disclosure (e.g., 78% (w/w) heat-treated whey protein isolate, 15% (w/w) Vivapharm™ PH176, 5% (w/w) dicalcium phosphate, 2% (w/w) gluconolactone)) has Dv₁₀, Dv₅₀, and Dv₉₀ values of 7.1 μm, 107 μm, and 344 μm, respectively.

As shown in FIG. 14C, a preparation of an exemplary formulated preparation of the present disclosure (e.g., 71% (w/w) heat-treated whey protein isolate, 27% (w/w) nutritional yeast, 2% (w/w) transglutaminase)) has Dv₁₀, Dv₅₀, and Dv₉₀ values of 30.8 μm, 164 μm, and 850 μm, respectively.

As shown in FIG. 14D, a preparation of an exemplary formulated preparation of the present disclosure (e.g., 82.4% (w/w) heat-treated whey protein isolate, 13.8% (w/w) Pectner™ APC169B, 0.9% Vivapharm™ sodium alginate PH176, 1.45% (w/w) gluconolactone, 1.45% (w/w) calcium hydrogen phosphate anhydrous) has Dv₁₀, Dv₅₀, and Dv₉₀ values of 7.4 μm, 31.1 μm, and 233 μm, respectively.

### Example 10: Additional Exemplary Manufacturing of Exemplary Formulated Preparations

This example describes an additional manufacturing protocol for producing exemplary formulated preparations of the present disclosure.

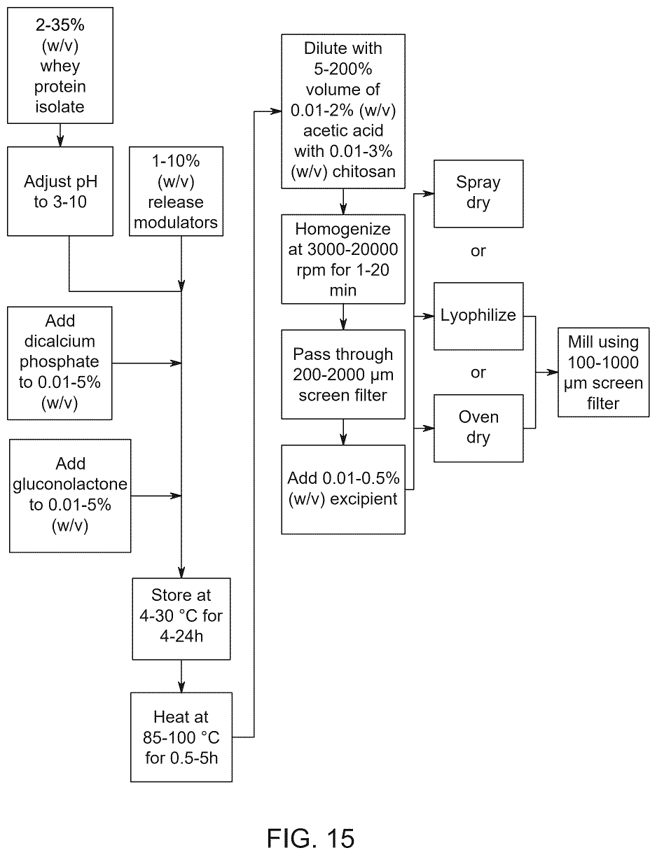

FIG. 15 shows a flow chart describing an exemplary manufacturing protocol for exemplary formulated preparations of the present disclosure. In brief, about 2% (w/v) to 35% (w/w) whey protein isolate was adjusted to pH 3-10 and mixed with about 1% (w/v) to 10% (w/v) of one or more release modulators. About 0.01% (w/v) to 5% (w/v) of dicalcium phosphate is added to the mixture, followed by addition of about 0.01% (w/v) to 5% (w/v) gluconolactone. The mixture was then stored at about 4° C. to 30° C. for about 4 hrs to 24 hrs. After storage, the mixture was heat-treated at about 85° C. to 100° C. for about 0.5 hrs to 5 hrs. After heat-treatment, samples were diluted with 5 to 200% volumes of 0.01-2% (w/v) acetic acid. Optionally, about 0.01%-3% (w/v) chitosan was added to the pH-adjusted solution. The samples were then homogenized for about 1 min. to 20 min. at about 3000 to 20,000 RPM. Samples were then passed through a 200 to 2000 μm screen filter. 0.01% (w/v) to 0.5% (w/v) excipient were added and samples were spray dried, lyophilized, or oven dried. Lyophilized and oven-dried samples were then milled using about a 100 to 1000 μm screen filter.

TABLE 2 and TABLE 3 describes yields from exemplary 10 min. spray dry processes of 9% (w/v) solids and 0.5% (w/v) of excipients (CRISP-FILM, SIPERNAT380, ULTRA-SPERSE M) to yield a 80% (w/w) heat-treated protein (Glanbia™ Provon 190), 13.4% (w/w) Pectner™ APC210, 0.86% (w/w) Vivapur™ alginate FD176, 2.9% (w/w) chitosan, 1.4% (w/w) gluconolactone, 1.4% (w/w) dicalcium phosphate formulation.

TABLE 2
Exemplary Spray Dry Yields

| | | CRISP-FILM | | SIPERNAT 380 | | ULTRA-SPERSE M | |
|---|---|---|---|---|---|---|---|
| | | Inlet Temperature (° C.) | | | | | |
| | | 110° C. | 135° C. | 110° C. | 135° C. | 110° C. | 135° C. |
| Pump | 10% | 353 mg | 486 mg | 479 mg | 475 mg | 205 mg | 904 mg |
| rate | 20% | 934 mg | 1323 mg | 1295 mg | 1940 mg | 640 mg | 850 mg |
| | 30% | 2145 mg | 1690 mg | 680 mg | 1580 mg | 721 mg | 1643 mg |

TABLE 3
Exemplary Spray Dry Yields (Normalized to Pump Rate)

| | | CRISP-FILM | | | SIPERNAT 380 | | | | ULTRA-SPERSE M | | | |
|---|---|---|---|---|---|---|---|---|---|---|---|---|
| | | Inlet Temperature (° C.) | | | | | | | | | | |
| | | 110° C. | 135° C. | | 110° C. | | 135° C. | | 110° C. | | 135° C. | |
| Pump | 10% | 353 mg | 486 | mg | 479 | mg | 475 | mg | 205 | mg | 904 | mg |
| rate | 20% | 467 mg | 661.5 | mg | 647.5 | mg | 970 | mg | 320 | mg | 425 | mg |
| | 30% | 715 mg | 563.3 | mg | 226.7 | mg | 526.7 | mg | 240.3 | mg | 547.7 | mg |

TABLE 4 describes yields and physical properties of preparations resulting from exemplary 10 min. spray dry processes of 9% (w/v) solids and 0.5% (w/v) excipients (Pea Starch, Aquafaba, Aquafaba and SIPERNAT 380, CRISP-FILM, SIPERNAT380, ULTRA-SPERSE M) to yield a 80% (w/w) heat-treated protein (Glanbia™ Provon 190), 13.4% (w/w) Pectner™ APC210, 0.86% (w/w) Vivapur™ alginate FD176, 2.9% (w/w) chitosan, 1.4% (w/w) gluconolactone, 1.4% (w/w) dicalcium phosphate formulation.

TABLE 4
Physical Properties of Exemplary Spray
Dry Preparations with Additives

| | | | | Aquafaba |
|---|---|---|---|---|
| | | | | and |
| | | | | SIPERNAT |
| | No Additives | Pea Starch | Aquafaba | 380 |
| Inlet | 135° C. | 135° C. | 135° C. | 135° C. |
| Temperature | | | | |
| (° C.) | | | | |
| Pump | Yield: | Yield: | Yield: | Yield: |
| Rate 15% | 996 mg | 1827 mg | 3524 mg | 3902 mg |
| | Bulk density: | Bulk density: | Bulk density: | Bulk density: |
| | 0.21 g/mL | 0.28 g/mL | 0.24 g/mL | 0.32 g/mL |
| | Tap density: | Tap density: | Tap density: | Tap density: |
| | 0.38 | 0.35 g/mL | 0.39 g/mL | 0.43 g/mL |
| | Hausner | Hausner | Hausner | Hausner |
| | Ratio: 1.8 | Ratio: 1.2 | Ratio: 1.6 | Ratio: 1.3 |
| | Not | Flowable | Not | Flowable |
| | Flowable | | Flowable | |

TABLE 5 and TABLE 6 describes yields from exemplary 5 min. spray dry processes of 9% (w/v) solids and 0.5% (w/v) of excipients (Aquafaba and SIPERNAT 380) to yield a 80% (w/w) heat-treated protein (Glanbia™ Provon 190), 13.4% (w/w) Pectner™ APC210, 0.86% (w/w) Vivapur™ alginate FD176, 2.9% (w/w) chitosan, 1.4% (w/w) gluconolactone, 1.4% (w/w) dicalcium phosphate formulation.

TABLE 5
Exemplary Spray Dry Yields

Aquafaba and Sipernat 380

| | | | | |
|---|---|---|---|---|
| | Inlet | 110° C. | 135° C. | 165° C. |
| | Tempe | | | |
| | rature | | | |
| | (° C.) | | | |
| Pump | 10% | 353 mg | 486 mg | 479 mg |
| rate | 20% | 934 mg | 1323 mg | 1295 mg |
| | 30% | 2145 mg | 1690 mg | 680 mg |

TABLE 6
Exemplary Spray Dry Yields (Normalized to Pump Rate)

Aquafaba and Sipernat 380

| | | | | |
|---|---|---|---|---|
| | Inlet | 110° C. | 135° C. | 165° C. |
| | Tempe | | | |
| | rature | | | |
| | (° C.) | | | |
| Pump | 10% | 108 mg | 450 mg | 277 mg |
| rate | 20% | 210.5 | 304 mg | 293.5 mg |
| | 30% | 154 mg | 194 mg | 182 mg |

### Example 11: Exemplary Incorporation into Food Products

This example describes the incorporation of exemplary formulated preparations of the present disclosure into food and/or beverage products (e.g., Crystal Lite™ powder, whey protein isolate powder).

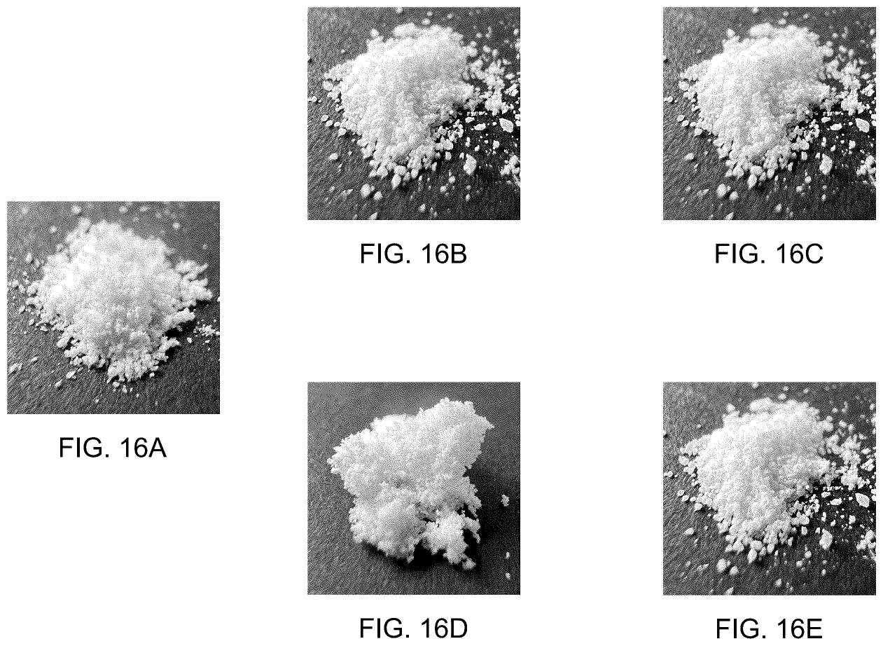

FIG. 16A, FIG. 16B, and FIG. 16D show a macroscopic image of an exemplary formulated preparation (82.6% (w/w) heat-treated protein isolate, 15.1% (w/w) Vivapur™ alginate FD176, 1.5% (w/w) gluconolactone, 0.8% (w/w) dicalcium phosphate), Crystal Lite™ powder, and commercial whey protein isolate respectively.

FIG. 16C and FIG. 16E are macroscopic images showing a 2:1 mixture of Crystal Lite™ powder and exemplary formulation, and a 1:1 mixture of commercial whey protein powder and exemplary formulation, respectively. Both mixtures could be processed to form particles less than 1 mm in diameter.

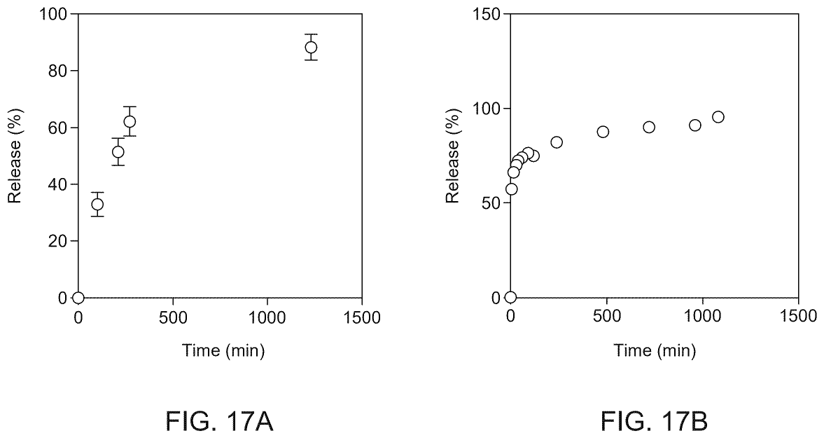

As shown in FIG. 17A, an exemplary formulated preparation comprising a release modulator that forms a calcium cross-linked interpenetrating polymer network gel (e.g., 82.6% (w/w) heat-treated whey protein isolate, 15.1% (w/w) Vivapur™ Alginate FD176, 1.5% (w/w) gluconolactone, 0.8% (w/w) dicalcium phosphate) incorporated with commercial whey protein powder at a 1:1 ratio, exhibited prolonged extended-release (as assessed at multiple times over the full 1250 min. time course)

Similarly, as shown in FIG. 17B, an exemplary formulated preparation comprising a release modulator that forms a calcium cross-linked interpenetrating polymer network gel (e.g., 82.6% (w/w) heat-treated whey protein isolate, 15.1% (w/w) Vivapur™ Alginate FD176, 1.5% (w/w) gluconolactone, 0.8% (w/w) dicalcium phosphate) incorporated with Crystal Lite™ powder at a 1:2 ratio (formulation: Crystal Lite™), exhibited prolonged extended-release (as assessed at multiple times over the full 1250 min. time course).

These data indicate that exemplary heat-treated whey protein isolate formulations of the present disclosure are amenable to incorporation into powder food and/or beverage products (e.g., protein powder) and maintain their extended duration of protein release even after processing steps to incorporate the formulated preparations into food and/or beverage products.

### Example 12: Exemplary Formulated Preparations after Storage

This example describes the release of one or more food components from exemplary formulated preparations of the present disclosure after storage under various conditions.

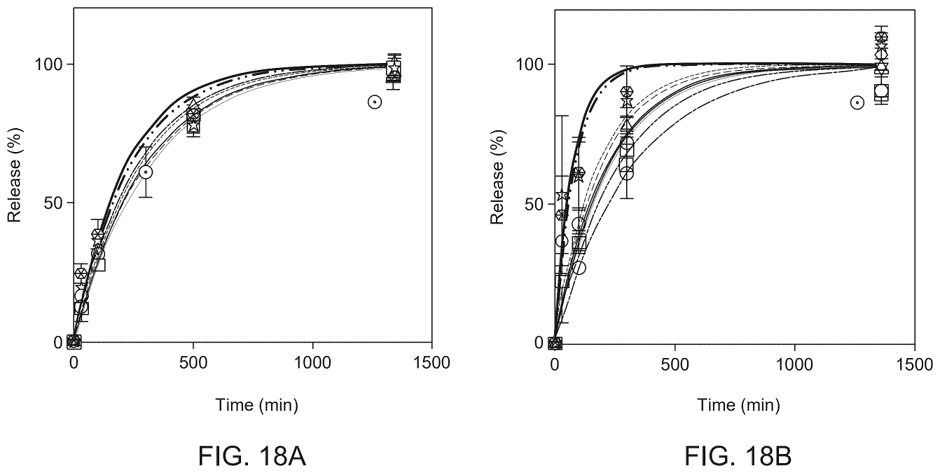

As shown in FIG. 18A, an exemplary formulated preparation comprising a release modulator that forms a calcium cross-linked interpenetrating polymer network gel (e.g., 82.6% (w/w) heat-treated whey protein isolate, 15.1% (w/w) Vivapur™ Alginate FD176, 1.5% (w/w) gluconolactone, 0.8% (w/w) dicalcium phosphate) exhibited prolonged extended-release (as assessed at multiple times over the full 1400 min. time course) after 14 days of storage under various conditions (e.g., temperature, vacuum, humidity, moisture, container materials).

As shown in FIG. 18B, after 28 days of storage under various conditions (e.g., temperature, vacuum, humidity, moisture, container materials), the extended duration of release of the exemplary formulated preparation (e.g., 82.6% (w/w) heat-treated whey protein isolate, 15.1% (w/w) Vivapur™ Alginate FD176, 1.5% (w/w) gluconolactone, 0.8% (w/w) dicalcium phosphate) was less stable at 80° C., 40% relative humidity, under nitrogen, air, or vacuum, ±desiccant, and stored in mylar bags or glass vials.

These data indicate that exemplary heat-treated whey protein isolate formulations of the present disclosure are capable of maintaining extended duration of amino acid release, even after storage under various conditions.

### Example 13: Additional Exemplary Formulated Preparations with Release Modulators (Calcium-Crosslinked Matrices)

This example describes release properties of additional exemplary heat-treated whey protein isolate preparations prepared with various loading of one or more food components (e.g., heat-treated whey protein) and various concentrations of release modulator components (e.g., pectin, alginate).

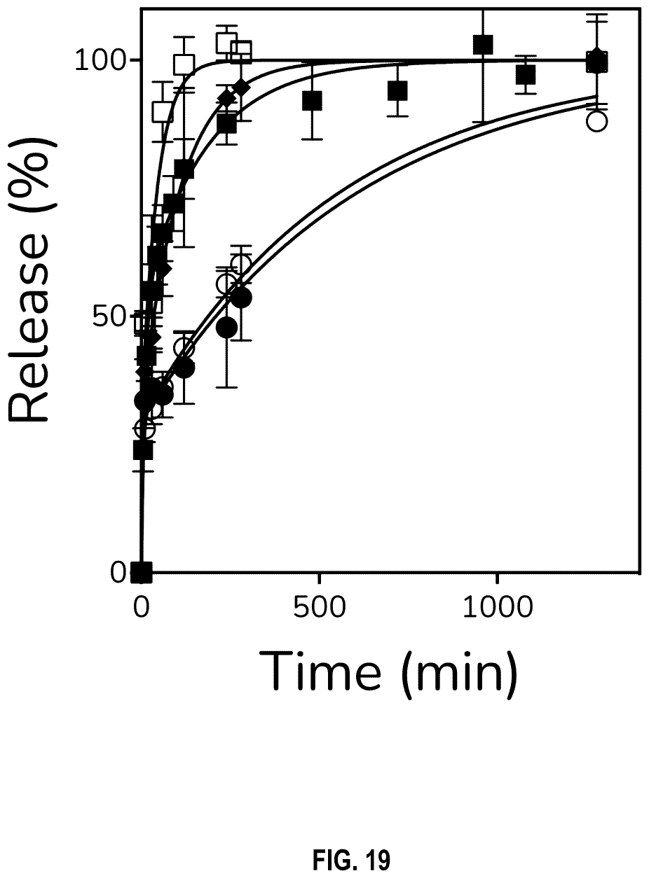

As shown in FIG. 19, exemplary formulated preparations comprising heat-treated whey protein below 80% (w/w) (e.g., 70% (w/w) heat-treated whey protein isolate, 23.5% (w/w) Pectner™ APC210, 1.6% (w/w) Vivapur™ FD176, 2.4% (w/w) gluconolactone, 2.4% (w/w) dicalcium phosphate; 75% (w/w) heat-treated whey protein isolate, 19.6% (w/w) Pectner™ APC210, 1.3% (w/w) Vivapur™ FD176, 2% (w/w) gluconolactone, 2% (w/w) dicalcium phosphate; 80% (w/w) heat-treated whey protein isolate, 15.7% (w/w) Pectner™ APC210, 1% (w/w) Vivapur™ FD176, 1.6% (w/w) gluconolactone, 1.6% (w/w) dicalcium phosphate) exhibited prolonged extended-release (as assessed at multiple times over the full 1250 min. time course) of proteins/peptides into simulated gastric fluid with 60 U/mL of pepsin as compared to exemplary formulated preparations comprising heat-treated whey protein above 80% (w/w) (e.g., 85% (w/w) heat-treated whey protein isolate, 11.7% (w/w) Pectner™ APC210, 0.8% (w/w) Vivapur™ FD176, 1.2% (w/w) gluconolactone, 1.2% (w/w) dicalcium phosphate; 90% (w/w) heat-treated whey protein isolate, 7.8% (w/w) Pectner™ APC210, 0.5% (w/w) Vivapur™ FD176, 0.8% (w/w) gluconolactone, 0.8% (w/w) dicalcium phosphate).

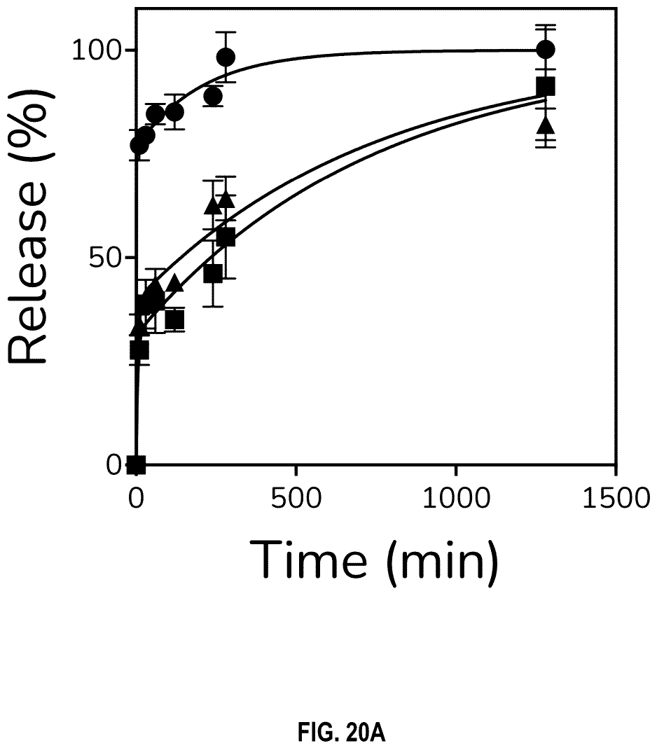

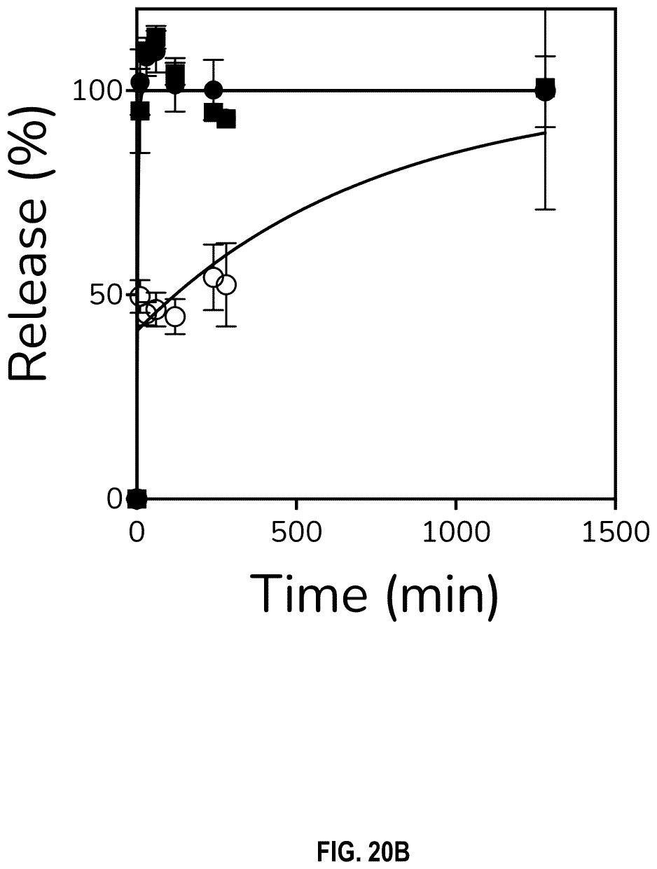

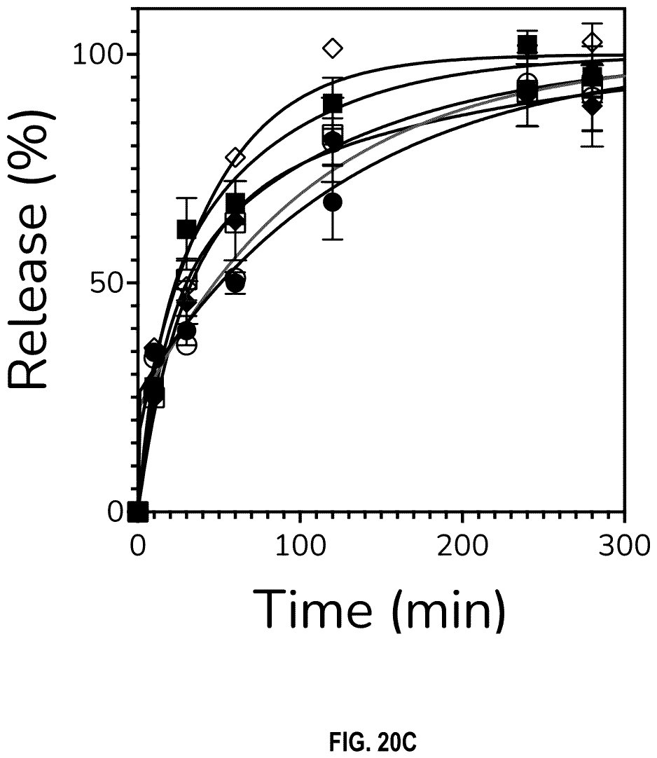

As shown in FIG. 20A, exemplary formulated preparations comprising pectin concentrations above 10% (w/w) (e.g., 86% (w/w) heat-treated whey protein isolate, 10% (w/w) Pectner™ APC210, 1% (w/w) Vivapur™ FD176, 1.5% (w/w) gluconolactone, 1.5% (w/w) dicalcium phosphate; 81.2% (w/w) heat-treated whey protein isolate, 15% (w/w) Pectner™ APC210, 0.9% (w/w) Vivapur™ FD176, 1.4% (w/w) gluconolactone, 1.4% (w/w) dicalcium phosphate) exhibited prolonged extended-release (as assessed at multiple times over the full 1250 min. time course) of proteins/peptides into simulated gastric fluid with 60 U/mL of pepsin as compared to exemplary formulated preparations comprising pectin concentrations below 10% (w/w) (e.g., 90.8% (w/w) heat-treated whey protein isolate, 5% (w/w) Pectner™ APC210, 1% (w/w) Vivapur™ FD176, 1.6% (w/w) gluconolactone, 1.6% (w/w) dicalcium phosphate).

As shown in FIG. 20B, it was observed that exemplary formulated preparations comprising hydrocolloids (e.g., mixtures of pectins, alginates and/or carrageenans) and gelators (e.g. mixtures of dicalcium phosphate and gluconolactone) having ratios below 7.5:1 (hydrocolloids: gelators) (e.g., 82.4% (w/w) heat-treated whey protein isolate, 9.9% (w/w) Pectner™ APC210, 0.7% (w/w) Vivapur™ FD176, 3.5% (w/w) gluconolactone, 3.5% (w/w) dicalcium phosphate; 82.4% (w/w) heat-treated whey protein isolate, 11.4% (w/w) Pectner™ APC210, 0.8% (w/w) Vivapur™ FD176, 2.7% (w/w) gluconolactone, 2.7% (w/w) dicalcium phosphate; 82.4% (w/w) heat-treated whey protein isolate, 12.8% (w/w) Pectner™ APC210, 0.9% (w/w) Vivapur™ FD176, 2% (w/w) gluconolactone, 1.9% (w/w) dicalcium phosphate; 82.4% (w/w) heat-treated whey protein isolate, 13.5% (w/w) Pectner™ APC210, 0.9% (w/w) Vivapur™ FD176, 1.6% (w/w) gluconolactone, 1.6% (w/w) dicalcium phosphate; 82.4% (w/w) heat-treated whey protein isolate, 14.3% (w/w) Pectner™ APC210, 1% (w/w) Vivapur™ FD176, 1.1% (w/w) gluconolactone, 1.2% (w/w) dicalcium phosphate) exhibited prolonged extended-release (as assessed at multiple times over the full 300 min. time course) of proteins/peptides into simulated gastric fluid with 60 U/mL of pepsin as compared to exemplary formulated preparations comprising hydrocolloid: gelator ratios above 7.5:1 (82.4% (w/w) heat-treated whey protein isolate, 14.5% (w/w) Pectner™ APC210, 1% (w/w) Vivapur™ FD176, 1% (w/w) gluconolactone, 1% (w/w) dicalcium phosphate).

As shown in FIG. 20C, it was observed that exemplary formulated preparations comprising hydrocolloids (e.g., mixtures of pectins and alginates) and gelators (e.g. mixtures of dicalcium phosphate and gluconolactone) (e.g., 82.4% (w/w) heat-treated whey protein isolate, 9.9% (w/w) Pectner™ APC210, 0.7% (w/w) Vivapur™ alginate FD176, 3.5% (w/w) gluconolactone, 3.5% (w/w) dicalcium phosphate; 82.4% (w/w) heat-treated whey protein isolate, 11.4% (w/w) Pectner™ APC210, 0.8% (w/w) Vivapur™ alginate FD176, 2.7% (w/w) gluconolactone, 2.7% (w/w) dicalcium phosphate; 82.4% (w/w) heat-treated whey protein isolate, 12.8% (w/w) Pectner™ APC210, 0.9% (w/w) Vivapur™ alginate FD176, 2% (w/w) gluconolactone, 1.9% (w/w) dicalcium phosphate); 82.4% (w/w) heat-treated whey protein isolate, 13.5% (w/w) Pectner™ APC210, 0.9% (w/w) Vivapur™ alginate FD176, 1.6% (w/w) gluconolactone, 1.6% (w/w) dicalcium phosphate); 82.4% (w/w) heat-treated whey protein isolate, 14.3% (w/w) Pectner™ APC210, 1% (w/w) Vivapur™ alginate FD176, 1.1% (w/w) gluconolactone, 1.2% (w/w) dicalcium phosphate) exhibited prolonged extended-release (as assessed at multiple times over the full 300 min. time course) of proteins/peptides into simulated gastric fluid with 60 U/mL of pepsin as compared to exemplary formulated preparations comprising higher alginate concentrations (e.g., 82.4% (w/w) heat-treated whey protein isolate, 14.5% (w/w) Pectner™ APC210, 1% (w/w) Vivapur™ alginate FD176, 1% (w/w) gluconolactone, 1% (w/w) dicalcium phosphate).

These data indicate that the amount of one or more food components (e.g., heat-treated whey protein isolate) loaded (on a dry weight basis) and or amount of one or more release modulators (e.g., pectin, alginate, carageenans, etc.) into exemplary formulated preparations of the present disclosure can affect the duration of extended-release of the formulated preparations.

### Example 14: Water Activities of Exemplary Formulated Preparations

This example describes the water activities of exemplary formulated preparations of the present disclosure.

TABLE 7 describes the water activities of various commercial food components, food products, beverage products, and exemplary formulated preparations of the present disclosure.

TABLE 7
Water Activities of Exemplary Formulated Preparations

| Aw | Stdev | Formulation |
|---|---|---|
| 0.111 | 0.009 | Glanbia Provon ™ 190 |
| 0.242 | 0.001 | Premier ™ Protein Vanilla |
| 0.251 | 0.001 | Optimum Nutrition Gold ™ Vanilla |
| 0.308 | 0.001 | Muscle Milk ™ Vanilla |
| 0.220 | 0.001 | Muscle Milk ™ Chocolate |
| 0.217 | 0.003 | C4 Vanilla |
| 0.091 | 0.002 | 100% (w/w) Heat-treated Whey Protein Isolate (Glanbia ™ |
| | | Provon 190) |
| 0.139 | 0.003 | 82.4% (w/w) Heat-treated Whey Protein Isolate, 13.8% |
| | | (w/w) Pectner ™ APC169B, 0.9% (w/w) Vivapharm ™ |
| | | sodium alginate PH 176, 1.45% (w/w) gluconolactone, |
| | | 1.45% (w/w) calcium hydrogen phosphate anhydrous |
| 0.074 | 0.001 | 82.4% (w/w) Heat-treated Whey Protein Isolate, 13.8% |
| | | (w/w) Pectner ™ APC210, 0.9% (w/w) Vivapharm ™ |
| | | sodium alginate PH 176, 1.45% (w/w) gluconolactone, |
| | | 1.45% (w/w) calcium hydrogen phosphate anhydrous |
| 0.065 | 0.001 | 82% w/w Heat-treated Whey Protein Isolate, 13.7% w/w |
| | | Pectner ™ APC210 pectin, 0.9% (w/w) Vivapharm ™ |
| | | sodium alginate PH 176, 1.45% (w/w) gluconolactone, |
| | | 1.45% (w/w) calcium hydrogen phosphate anhydrous, 0.5% |
| | | (w/v) SIPERNAT 22S |

Without wishing to be bound by any particular theory, it is observed that exemplary formulated preparations characterized as having water activities less than 0.2 are more stable during storage under various conditions.

### Example 15: Additional Exemplary Formulated Preparations and Incorporation into Food and/or Beverage Products

This example describes additional formulated preparations of the present disclosure and their incorporation into food and/or beverage products.

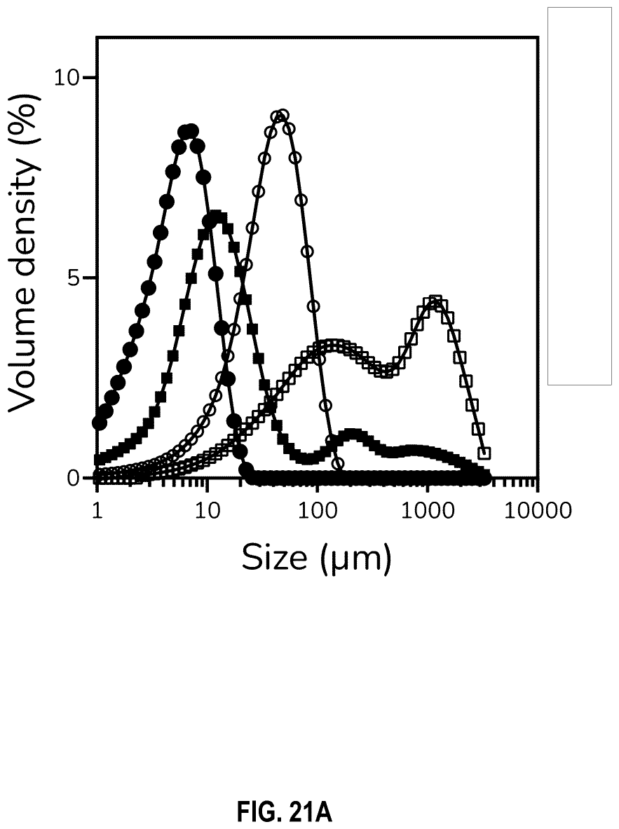

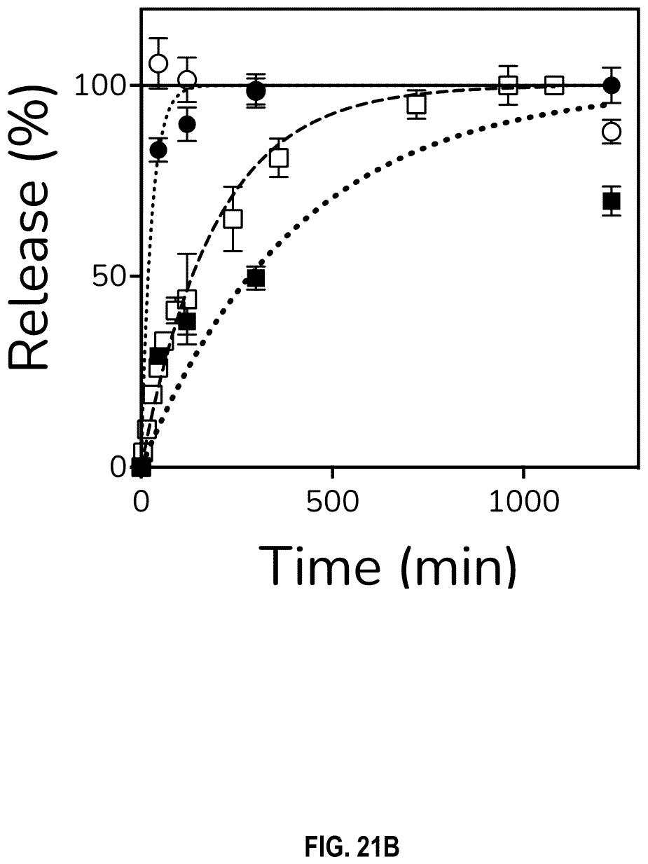

As shown in FIG. 21A, a preparation of an exemplary formulated preparation of the present disclosure (80% (w/w) heat-treated whey protein isolate, 13.4% (w/w) kappa carrageenan, 0.86 (w/w) Vivapur™ alginate FD176, 2.9% (w/w) chitosan, 1.4% (w/w) gluconolactone. 1.4% (w/w) dicalcium phosphate)) was characterized as having relatively uniform particle size (Dv₅₀<100 μm).

As further shown in FIG. 21B, a heat-treated whey protein isolate preparation having larger particle sizes (Dv₅₀>100 μm) was observed to have an extended-release of proteins/peptides into simulated gastric fluid with 60 U/mL of pepsin, where 100% protein/peptide release from the preparation occurred after 120 min. However, without wishing to be bound by any particular theory, preparations having larger (e.g., Dv₅₀>100 μm) are not amenable to incorporation into most food and/or beverage products (e.g., providing granularity and/or an unpleasant mouth feel). Preparations having smaller particles (e.g., Dv₅₀<100 μm), while being more amenable to incorporation into most food and/or beverage products, were not observed to have an extended-release of proteins/peptides into simulated gastric fluid with 60 U/mL of pepsin (e.g., 100% protein/peptide release from the preparation occurred before 120 min. In contrast, a preparation of the exemplary formulated preparation (80% (w/w) heat-treated whey protein isolate, 13.4% (w/w) kappa carrageenan, 0.86% (w/w) Vivapur™ alginate FD176, 2.9% (w/w) chitosan, 1.4% (w/w) gluconolactone. 1.4% (w/w) dicalcium phosphate) was observed to exhibit a prolonged extended-release (as assessed at multiple times over the full 1250 min. time course) of proteins/peptides into simulated gastric fluid with 60 U/mL of pepsin as well as a small particle size (e.g., Dv50<100 μm) that was amenable to homogeneous incorporation into various food and/or beverage products (e.g., Clif™ bars, Perfect™ bars, Red Bull™ beverage, Body Armor™ beverage, data not shown).

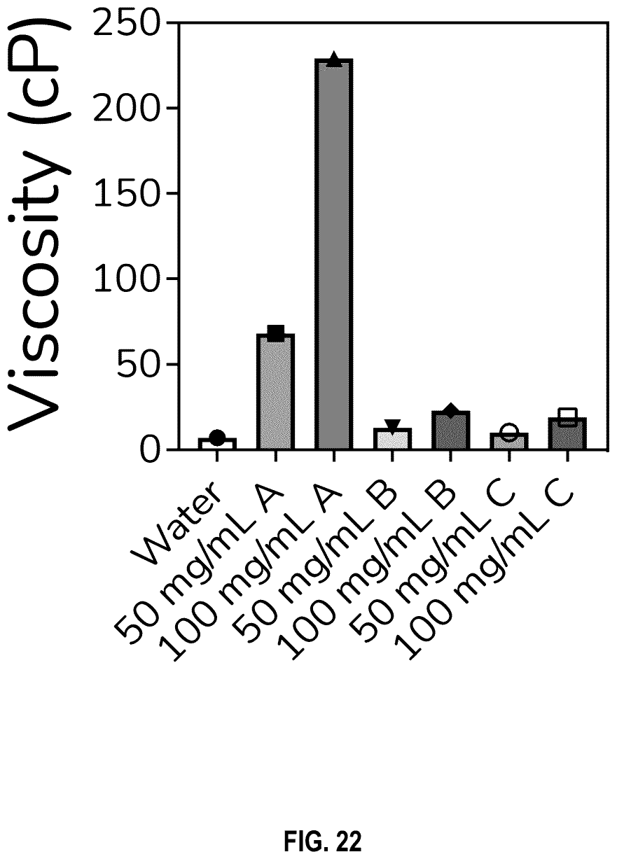

FIG. 22 shows viscosity measurements of 50 mg/ml and 100 mg/ml dilutions of commercial whey protein isolate powder (“C”; Premier whey protein powder, Vanilla), a preparation of an exemplary formulated preparation of the present disclosure without chitosan (“A”; 80% (w/w) heat-treated whey protein isolate (e.g., Glanbia™ Provon 190), 13.8% (w/w) Pectner™ APC169B, 0.9% (w/w) Vivapharm™ sodium alginate PH176, 1.4% (w/w) gluconolactone. 1.4% (w/w) dicalcium phosphate), and a preparation of an exemplary formulated preparation of the present disclosure with chitosan (“B”; 80% (w/w) heat-treated whey protein isolate (e.g., Glanbia™ Provon 190), 13.4% (w/w) Pectner™ APC210, 0.86% (w/w) Vivapur™ alginate FD176, 2.9% (w/w) chitosan, 1.4% (w/w) gluconolactone. 1.4% (w/w) dicalcium phosphate). Without wishing to be bound by any particular theory, while the presence of chitosan would be expected to increase the viscosity of a preparation, it is contemplated that a hydrocolloid comprising greater charge (e.g., Pectner™ APC210 as compared to Pectner™ APC169B) may be capable of interacting with chitosan to lower viscosity of a preparation.

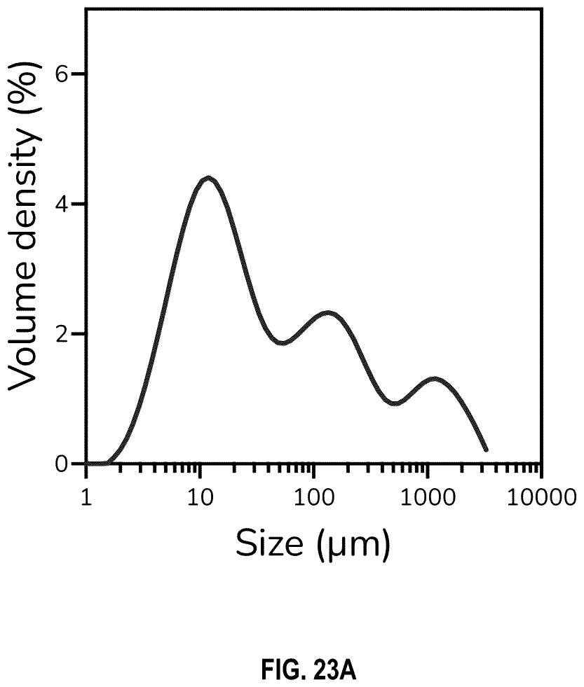

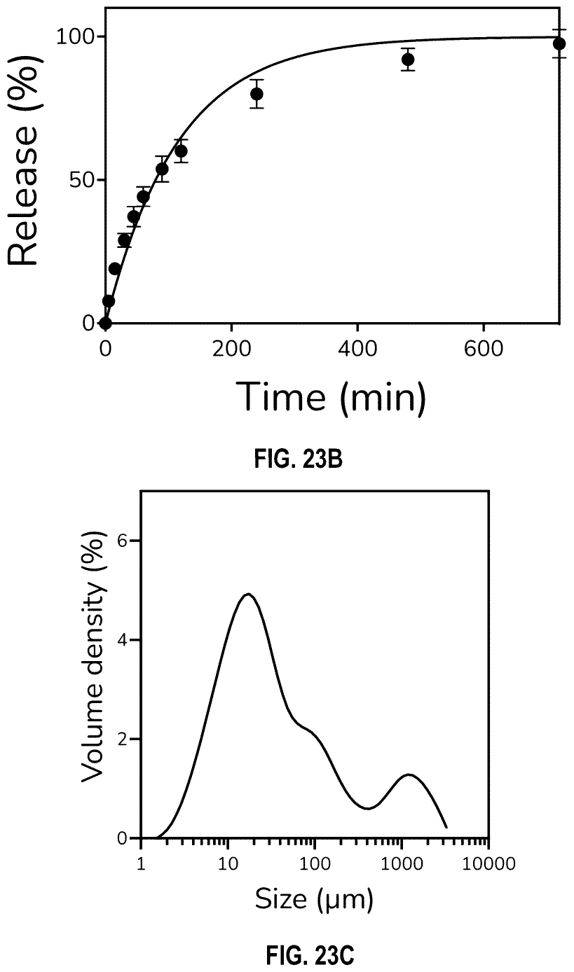

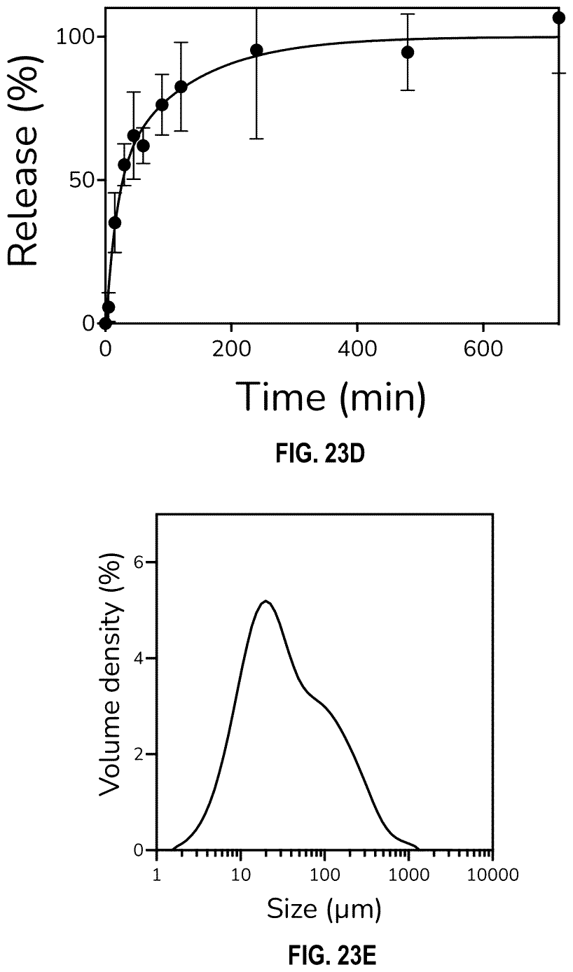

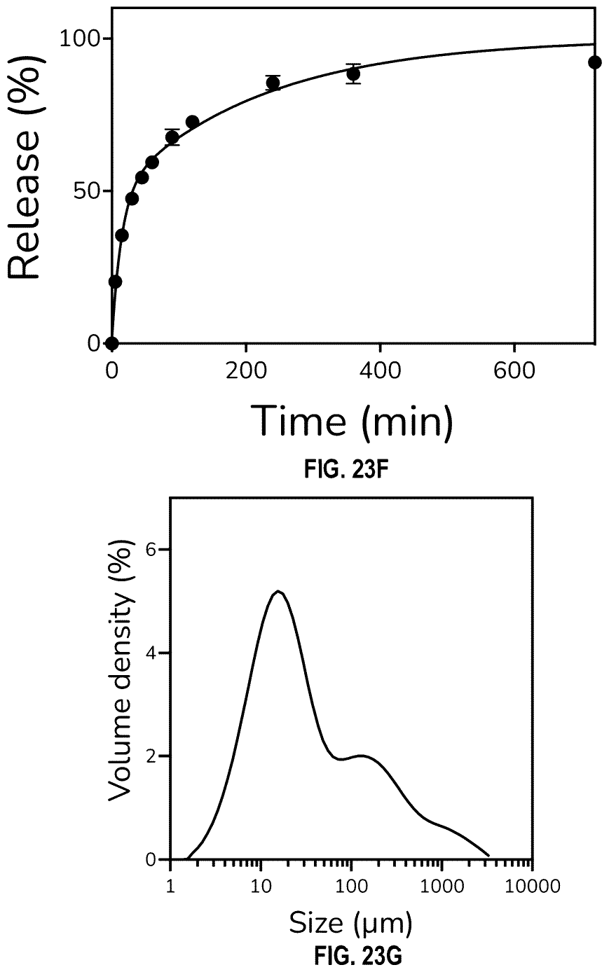

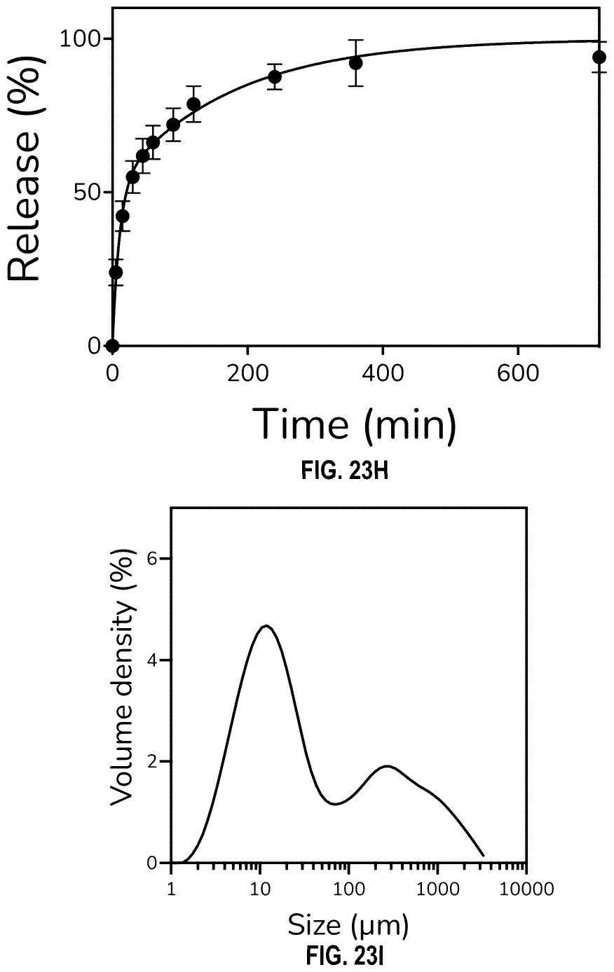

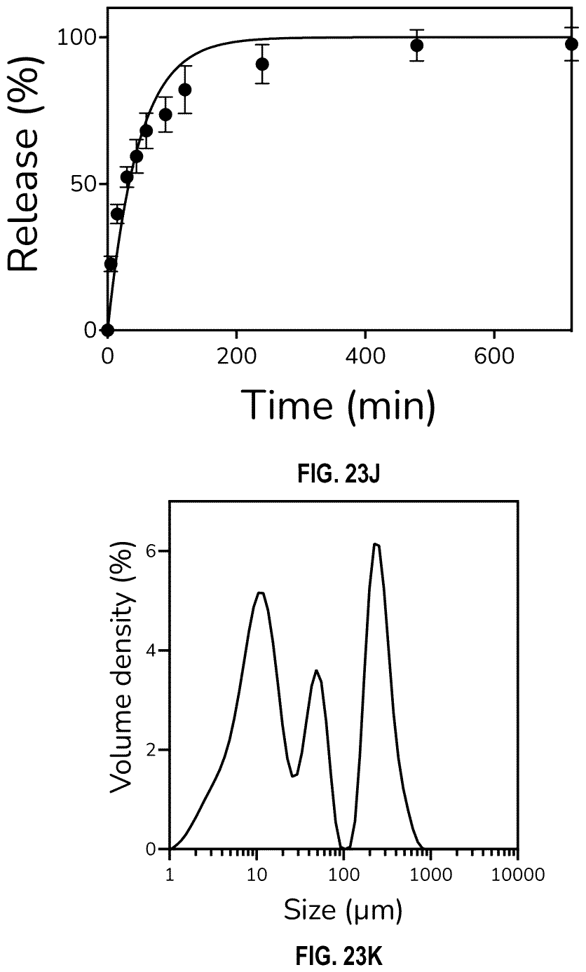

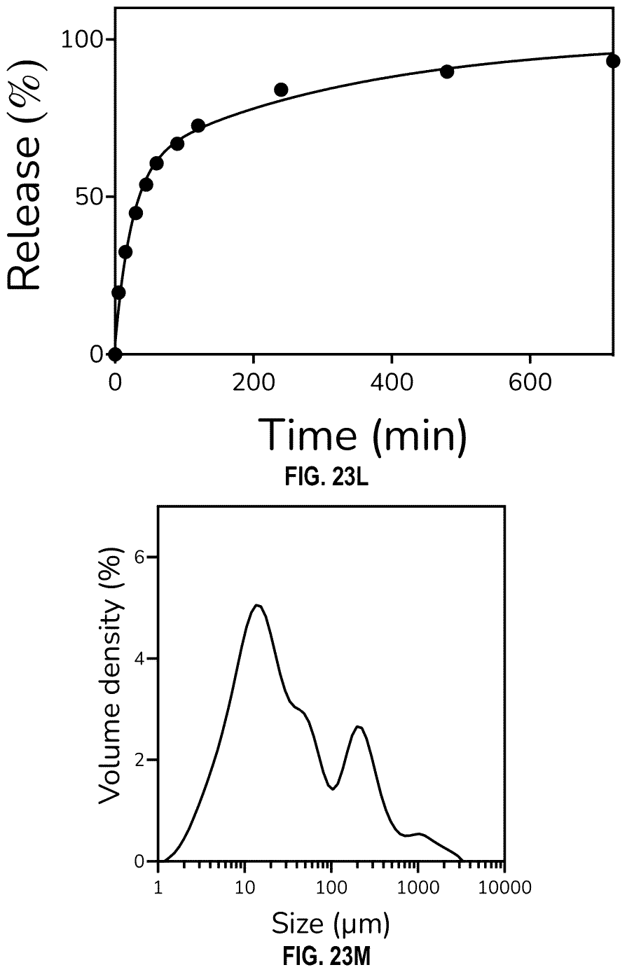

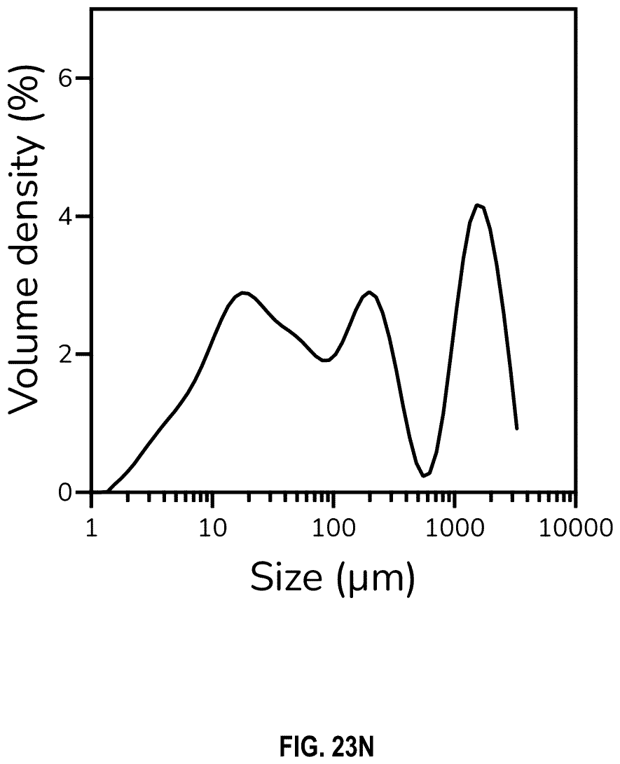

As shown in FIGS. 23B, 23D, 23F, 23H, 23J, and 23L, preparations of exemplary formulated preparations of the present disclosure produced using a method in accordance with FIG. 15, all exhibited extended-release (as assessed at multiple times over the full 700 min. time course) of proteins/peptides into simulated gastric fluid with 60 U/mL of pepsin, where 100% protein/peptide release from the preparations occurred after 120 min. Corresponding particle size distribution plots are shown in FIGS. 23A, 23C, 23E, 23G, 23I, 23K, 23M, and 23N.

TABLE 8 describes various physical properties of the exemplary formulated preparations according to FIGS. 23A to 23N, where said physical properties are conducive for incorporation into one or more food and/or beverage product.

TABLE 8
Physical Properties of Exemplary Formulated Preparations

| | Dv₁₀ | Dv₅₀ | Dv₉₀ | Water | Bulk | Tapped | Hausner |
|---|---|---|---|---|---|---|---|
| Formulation | (μm) | (μm) | (μm) | Activity | Density | Density | Ratio |
| 80.2% (w/w) Heat Treated | 5.0 | 25.4 | 725 | 0.074 ± | 0.417 ± | 0.717 ± | 1.718 |
| Whey Protein Isolate, 13.3% | | | | 0.001 | 0.017 | 0.051 | |
| (w/w) Pectner ™ APC210, | | | | | | | |
| 1% (w/w) Vivapur ™ | | | | | | | |
| Alginate FD176, 3.2% | | | | | | | |
| (w/w) Gluconolactone, 2.1% | | | | | | | |
| (w/w) Dicalcium phosphate | | | | | | | |
| 78.3% (w/w) Heat Treated | 5.6 | 14.6 | 705 | 0.065 ± | 0.423 ± | 0.667 ± | 1.578 |
| Whey Protein Isolate, 13.1% | | | | 0.001 | 0.005 | 0.000 | |
| (w/w) Pectner ™ APC210, | | | | | | | |
| 1% (w/w) Vivapur ™ | | | | | | | |
| Alginate FD176, 3.1% | | | | | | | |
| (w/w) Gluconolactone, 2.1% | | | | | | | |
| (w/w) Dicalcium phosphate, | | | | | | | |
| 2.4% (w/w) Sipernat ™ 22S | | | | | | | |
| 78.3% (w/w) Heat Treated | 7.3 | 29.5 | 193 | 0.096 ± | 0.463 ± | 0.641 ± | 1.383 |
| Whey Protein Isolate, 13.1% | | | | 0.001 | 0.033 | 0.045 | |
| (w/w) Pectner ™ APC210, | | | | | | | |
| 1% (w/w) Vivapur ™ | | | | | | | |
| Alginate FD176, 3.1% | | | | | | | |
| (w/w) Gluconolactone, 2.1% | | | | | | | |
| (w/w) Dicalcium phosphate, | | | | | | | |
| 1.2% (w/w) Sipernat ™ 22S, | | | | | | | |
| 1.2% (w/w) Aquafaba | | | | | | | |
| 77% (w/w) Heat Treated | 5.6 | 23.1 | 321 | 0.073 ± | 0.419 ± | 0.751 ± | 1.794 |
| Whey Protein Isolate, 12.8% | | | | 0.004 | 0.035 | 0.001 | |
| (w/w) Pectner ™ APC210, | | | | | | | |
| 1% (w/w) Vivapur ™ | | | | | | | |
| Alginate FD176, 3.1% | | | | | | | |
| (w/w) Gluconolactone, 2% | | | | | | | |
| (w/w) Dicalcium phosphate, | | | | | | | |
| 1.2% (w/w) Chitosan, 1.2% | | | | | | | |
| (w/w) Sipernat ™ 22S, 1.2% | | | | | | | |
| (w/w) Aquafaba | | | | | | | |
| 77% (w/w) Heat Treated | 4.4 | 20.0 | 639 | 0.064 ± | 0.342 ± | 0.527 ± | 1.544 |
| Whey Protein Isolate, 12.8% | | | | 0.001 | 0.018 | 0.028 | |
| (w/w) Pectner ™ APC210, | | | | | | | |
| 1% (w/w) Vivapur ™ | | | | | | | |
| Alginate FD176, 3.1% | | | | | | | |
| (w/w) Gluconolactone, 2% | | | | | | | |
| (w/w) Dicalcium phosphate, | | | | | | | |
| 1.2% (w/w) Chitosan, 1.2% | | | | | | | |
| (w/w) HiCap100 ™, 1.2% | | | | | | | |
| (w/w) Aquafaba | | | | | | | |
| 83.8% (w/w) Heat Treated | 5.3 | 29.8 | 302 | 0.111 ± | 0.283 ± | 0.566 ± | 2.001 |
| Whey Protein Isolate, 4.0% | | | | 0.002 | 0.005 | 0.019 | |
| (w/w) Ticaloid ™ 710H, 1% | | | | | | | |
| (w/w) Vivapur ™ Alginate | | | | | | | |
| FD176, 3.4% (w/w) | | | | | | | |
| Gluconolactone, 2.2% (w/w) | | | | | | | |
| Dicalcium phosphate, 5.5% | | | | | | | |
| (w/w) Chitosan | | | | | | | |
| 81.1% (w/w) Heat Treated | 5.6 | 23.7 | 283 | 0.133 ± | 0.247 ± | 0.519 ± | 2.101 |
| Whey Protein Isolate, 3.9% | | | | 0.003 | 0.018 | 0.032 | |
| (w/w) Ticaloid ™ 710H, 1% | | | | | | | |
| (w/w) Vivapur ™ Alginate | | | | | | | |
| FD176, 3.3% (w/w) | | | | | | | |
| Gluconolactone, 2.2% (w/w) | | | | | | | |
| Dicalcium phosphate, 5.4% | | | | | | | |
| (w/w) Chitosan, 2.6% (w/w) | | | | | | | |
| SIPERNAT ™ 50S | | | | | | | |
| 81.1% (w/w) Heat Treated | 8.4 | 110 | 1880 | 0.100 ± | 0.326 ± | 0.579 ± | 1.773 |
| Whey Protein Isolate, 3.9% | | | | 0.002 | 0.012 | 0.040 | |
| (w/w) Ticaloid ™ 710H, 1% | | | | | | | |
| (w/w) Vivapur ™ Alginate | | | | | | | |
| FD176, 3.3% (w/w) | | | | | | | |
| Gluconolactone, 2.2% (w/w) | | | | | | | |
| Dicalcium phosphate, 5.4% | | | | | | | |
| (w/w) Chitosan, 1.3% (w/w) | | | | | | | |
| SIPERNAT ™ 50S, 1.3% | | | | | | | |
| (w/w) Aquafaba | | | | | | | |
| 77.6% (w/w) Heat Treated | — | — | — | 0.217 ± | 0.322 ± | 0.555 ± | 1.721 |
| Whey Protein Isolate, 6.5% | | | | 0.003 | 0.010 | 0.000 | |
| (w/w) Pectner ™ APC210, | | | | | | | |
| 0.5% (w/w) Vivapur ™ | | | | | | | |
| Alginate FD176, 6.2% | | | | | | | |
| (w/w) Gluconolactone, 4.1% | | | | | | | |
| (w/w) Dicalcium phosphate, | | | | | | | |
| 2.7% (w/w) Chitosan, 1.2% | | | | | | | |
| (w/w) Sipernat ™ 22S, 1.2% | | | | | | | |
| (w/w) Aquafaba | | | | | | | |
| 77.6% (w/w) Heat Treated | — | — | — | 0.190 ± | 0.259 ± | 0.566 ± | 2.189 |
| Whey Protein Isolate, 6.5% | | | | 0.002 | 0.007 | 0.019 | |
| (w/w) Pectner ™ APC210, | | | | | | | |
| 0.5% (w/w) Vivapur ™ | | | | | | | |
| Alginate FD176, 6.2% | | | | | | | |
| (w/w) Gluconolactone, 4.1% | | | | | | | |
| (w/w) Dicalcium phosphate, | | | | | | | |
| 2.7% (w/w) Chitosan, 1.2% | | | | | | | |
| (w/w) Globe DE42 ™ Corn | | | | | | | |
| Syrup Solids, 1.2% (w/w) | | | | | | | |
| NZorbi ™ 2144 | | | | | | | |

### Example 16: Intestinal Epithelial Absorption

This example describes the absorption capacity of intestinal epithelial cells for amino acids released from an exemplary formulated preparation of the present disclosure.

To study the permeation capacity (%) of amino acids across a human intestinal epithelial cell layer, a Transwell® cell culture insert system was used. After 30 min, 120 min, and 240 min incubation following application of various samples to the apical side of the cell layer. Samples applied were pH-neutralized gastric fluid digests (500 mL gastric fluid with 60 U/mL pepsin, at pH 1.2, 37° C.) of untreated whey protein isolate (after 1080 min. incubation), heat-treated whey protein isolate (after 1080 min. incubation), or a formulated preparation including 80% (w/w) heat-treated whey protein isolate (Glanbia™ Provon 190), 13.4% (w/w) Pectner™ APC210, 0.86% (w/w) Vivapur™ alginate FD176, 2.9% (w/w) chitosan, 1.4% (w/w) gluconolactone, 1.4% (w/w) dicalcium phosphate (after incubation at various time points (e.g., between 5 min, and 1080 min).

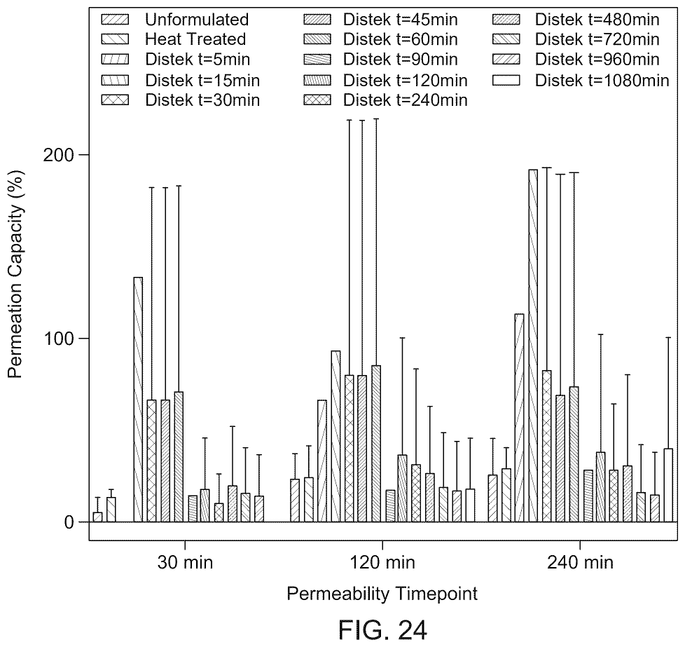

As shown in FIG. 24, only a small percentage of total amino acids released from unformulated whey protein isolate powder and heat-treated whey protein isolate were transported across the human intestinal epithelial layer. In contrast, a greater percentage of total amino acids released from the exemplary formulated preparation (80% (w/w) heat-treated whey protein isolate (Glanbia™ Provon 190), 13.4% (w/w) Pectner™ APC210, 0.86% (w/w) Vivapur™ alginate FD176, 2.9% (w/w) chitosan, 1.4% (w/w) gluconolactone, 1.4% (w/w) dicalcium phosphate) was transported across the human intestinal epithelial layer. Without wishing to be bound by any particular theory, these data suggest that permeability across the intestinal epithelial layer is a function of the amount of amino acids released from a formulation. It is contemplated that an extended duration of release at a lower concentration achieves greater amino acid absorption than a bolus of release at a higher concentration. Accordingly, exemplary formulations of the present disclosure characterized as having an extended duration of release of one of more food components (e.g., where 100% release occurs after 120 min.), may be capable of providing greater nutrition to a subject ingesting said formulation as compared to one or more unformulated food components and/or elicit a sensation of satisfaction and/or satiety.

### Example 17: Exemplary Formulated Preparations Comprising Excipient Components

This example describes the addition of excipient components to exemplary formulated preparations of the present disclosure. In some embodiments, one or more added excipient components improve the mixing of exemplary formulated preparations with foods and/or beverages. In some embodiments, one or more added excipient components improve the mouthfeel of exemplary formulated preparations upon consumption by a subject. In some embodiments, one or more excipient components impart a pleasing flavor when consumed by a subject.

An exemplary formulated preparation comprising 76.4% (w/w) heat treated Glanbia Provon™ 190 whey protein isolate, 12.8% (w/w) Pectner™ APC 210, 0.9% (w/w) VIVAPUR™ FD 176, 2.0% (w/w) gluconolactone, 2.0% (w/w) dicalcium phosphate, and 5.9% (w/w) Chitolytic chitosan was prepared by preparing an aqueous mixture of whey protein, pectin, alginate, and dicalcium phosphate, followed by the addition of dicalcium phosphate and incubation overnight at 4° C. The resulting gel was removed from 4° C. and heat treated by placing in a 95° C. water bath for 3 hours, followed by dilution in a 0.2% (w/v) acetic acid solution with 2.9% (w/v) chitosan and high shear homogenization and passed through a 850 μm sieve mesh. The slurry was spray dried at an inlet temperature of 195° C. and outlet temperature of 75° C. to yield the exemplary formulated preparation as a powder.

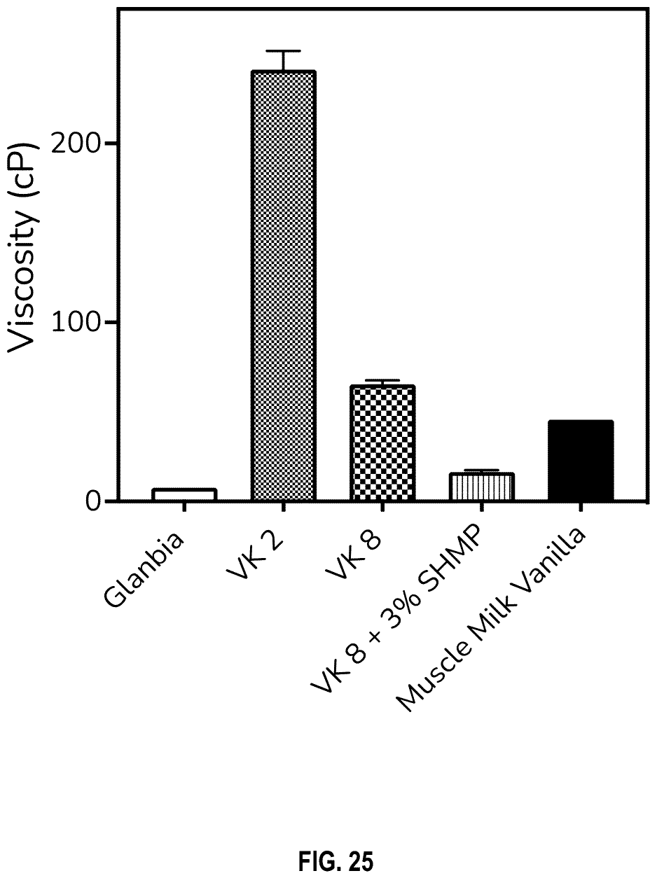

Without wishing to be bound by any particular theory, it is contemplated that the inclusion of 2.9% (w/v) chitosan improves the utility of formulated preparations by reducing the viscosity when mixed with foods and/or beverages. As shown in FIG. 25, the viscosity of exemplary formulated preparations without chitosan place in in water at 100 mg/mL (VK2; 78.3% (w/w) Heat Treated Whey Protein Isolate, 13.1% (w/w) Pectner™ APC210, 1% (w/w) Vivapur™ Alginate FD176, 3.1% (w/w) Gluconolactone, 2.1% (w/w) Dicalcium phosphate, 2.4% (w/w) Sipernat™ 22S) is about 30-fold greater than that of unformulated Glanbia whey protein solution of equal protein concentration. The viscosity of exemplary formulated preparations comprising chitosan placed in water at 100 mg/mL (VK8; 76.4% (w/w) heat treated Glanbia Provon™ 190 whey protein isolate, 12.8% (w/w) Pectner™ APC 210, 0.9% (w/w) VIVAPUR™ FD 176, 2.0% (w/w) gluconolactone, 2.0% (w/w) dicalcium phosphate, and 5.9% (w/w) Chitolytic chitosan) is about 4-fold lower than exemplary formulated preparations without chitosan, however, still 8-fold greater than that of unformulated protein. The addition of sodium hexametaphosphate to exemplary formulated preparations comprising chitosan (VK8+3% SHMP; 74.1% (w/w) heat treated Glanbia Provon™ 190 whey protein isolate, 12.4% (w/w) Pectner™ APC 210, 0.9% (w/w) VIVAPUR™ FD 176, 1.9% (w/w) gluconolactone, 2.0% (w/w) dicalcium phosphate, 5.7% (w/w) Chitolytic chitosan, and 3% (w/w) sodium hexametaphosphate mixed at 100 mg/mL in water yields a viscosity nearly 15-fold lower than that of exemplary formulated preparations without chitosan or sodium hexametaphosphate and only 2-fold greater than that of unformulated protein. Moreover, exemplary formulated preparations with chitosan and sodium hexametaphosphate exhibit a viscosity nearly 3-fold lower than that of a commercial Muscle Milk™ powder.

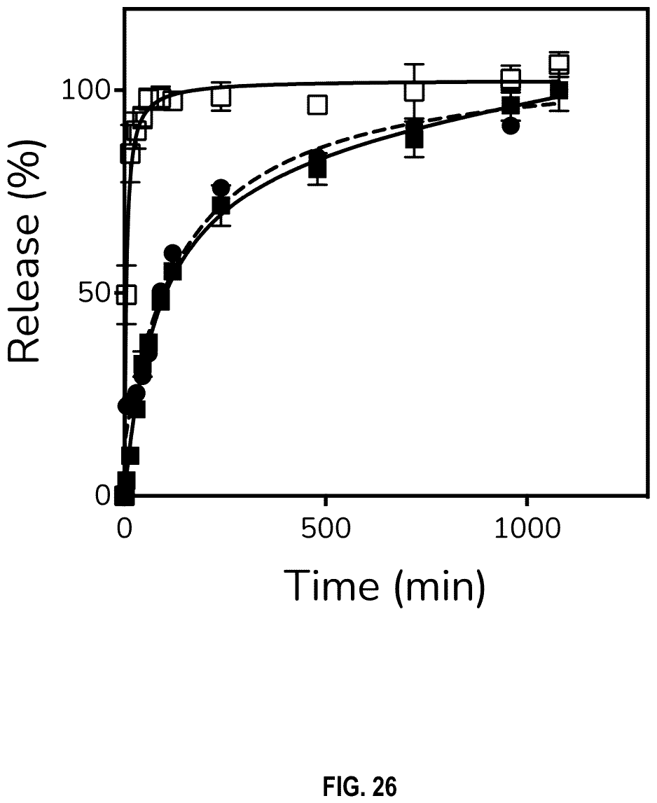

As shown in FIG. 26 the addition of 3% (w/w) sodium hexametaphosphate to exemplary formulated preparations (closed squares; 74.1% (w/w) heat treated Glanbia Provon™ 190 whey protein isolate, 12.4% (w/w) Pectner™ APC 210, 0.9% (w/w) VIVAPUR™ FD 176, 1.9% (w/w) gluconolactone, 2.0% (w/w) dicalcium phosphate, 5.7% (w/w) Chitolytic chitosan, and 3% (w/w) sodium hexametaphosphate) of the present disclosure minimally affects the rate of release of amino acids and peptide food components as compared to exemplary formulated preparations without sodium hexametaphosphate (closed circles; 76.4% (w/w) heat treated Glanbia Provon™ 190 whey protein isolate, 12.8% (w/w) Pectner™ APC 210, 0.9% (w/w) VIVAPUR™ FD 176, 2.0% (w/w) gluconolactone, 2.0% (w/w) dicalcium phosphate, and 5.9% (w/w) Chitolytic chitosan). Moreover, the addition of 3% (w/w) sodium hexametaphosphate to unformulated Glanbia Provon™ 190 whey protein isolate does not provide for extended duration of release of amino acids and peptide food component(s) (open squares).

These data suggest that exemplary formulated preparations comprising chitosan and/or sodium hexametaphosphate added as excipients reduce viscosity of preparations without having significant effects on the extended-release of one or more food products from formulated compositions therein.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1-82. (canceled)

83. A preparation of particles comprising:
60% (w/w) to 85% (w/w) heat-treated protein;
5% (w/w) to 20% (w/w) pectin or carrageenan;
0.5% (w/w) to 1.5% (w/w) alginate; and
0.5% (w/w) to 8% (w/w) gelator,
which particle preparation is an extended-release particle preparation in that, when the particles are incubated in a solution of simulated gastric fluid comprising 60 units/ml of pepsin at 37° C., the protein is released over a period of time that extends for at least 2 hours, and further wherein:
at least 5% of the protein is released over a duration of 2 hours; and
at least 50% of the protein is released over a duration of 6 hours.

84. The preparation of claim 83, wherein the heat-treated protein comprises heat-treated whey protein, heat-treated pea protein, heat-treated milk protein, heat-treated casein, heat-treated oat protein, heat treated gelatin, or heat-treated soy protein.

85. The preparation of claim 83, wherein the heat-treated protein comprises heat-treated whey protein.

86. The preparation of claim 83, wherein the gelator comprises gluconolactone, dicalcium phosphate, calcium carbonate, or a combination thereof.

87. The preparation of claim 83, wherein the preparation further comprises 0.01% (w/w) to 10% (w/w) chitosan.

88. The preparation of claim 83, wherein the preparation further comprises 0.1% (w/w) to 5% (w/w) of an anti-caking aid.

89. The preparation of claim 88, wherein the anti-caking aid comprises silica, bamboo fiber, carrot fiber, rice hull powder, or calcium carbonate.

90. The preparation of claim 83, wherein the preparation further comprises 1% (w/w) to 5% (w/w) of a sequestrant.

91. The preparation of claim 90, wherein the sequestrant comprises sodium hexametaphosphate, sodium tripolyphosphate, potassium hexametaphosphate, potassium tripolyphosphate, calcium hexametaphosphate, or calcium tripolyphosphate.

92. The preparation of claim 83, wherein the preparation further comprises 0.1% (w/w) to 5% (w/w) of a stabilizer.

93. The preparation of claim 92, wherein the stabilizer comprises xanthan gum, guar gum, or modified cellulose gum.

94. The preparation of claim 83, wherein the preparation further comprises one or more probiotics.

95. The preparation of claim 94, wherein the preparation comprises 1×10⁵ to 1×10⁹ CFUs of the one or more probiotics per gram of the preparation.

96. The preparation of claim 94, wherein the one or more probiotics comprise Lactobacillus.

97. The preparation of claim 83, wherein the preparation further comprises 1% (w/w) to 20% (w/w) of a dietary fiber.

98. The preparation of claim 97, wherein the dietary fiber comprises inulin, dextrin, whole grain fiber, sodium alginate, potassium alginate, calcium alginate, ammonium alginate, or propylene glycol alginate.

99. The preparation of claim 83, wherein the preparation is characterized as having a Dv50 less than 100 μm.

100. The preparation of claim 83, wherein the preparation is characterized as having a water activity of less than 0.2.

101-103. (canceled)

104. A food, beverage, or supplement product comprising a particle preparation that releases protein in simulated gastric fluid over a period of time that extends for at least two hours so that the particle preparation is an extended-release particle preparation, wherein particles of the preparation comprise:
60% (w/w) to 85% (w/w) heat-treated protein;
5% (w/w) to 20% (w/w) pectin or carrageenan;
0.5% (w/w) to 1.5% (w/w) alginate; and
0.5% (w/w) to 8% (w/w) gelator,
and further wherein the extended-release particle preparation is characterized in that, when the particles are incubated in a solution of simulated gastric fluid comprising 60 units/ml of pepsin at 37° C., the protein is released over a period of time that extends for at least 2 hours, and further wherein:
at least 5% of the protein is released over 2 hours; and
at least 50% of the protein is released over 6 hours.

105. The food, beverage, or supplement product of claim 104, wherein the heat-treated protein comprises heat-treated whey protein, heat-treated pea protein, heat-treated milk protein, heat-treated casein, heat-treated oat protein, or heat-treated soy protein.

106. The food, beverage, or supplement product of claim 104, wherein the heat-treated protein comprises heat-treated whey protein.

107. The food, beverage, or supplement product of claim 104, wherein the gelator comprises gluconolactone, dicalcium phosphate, calcium carbonate, or a combination thereof.

108. The food, beverage, or supplement product of claim 104, wherein the food, beverage, or supplement product is a protein powder, snack bar, carbonated beverage, salty snack, confectionary, sports drink, yogurt, or gummy.

109-111. (canceled)

112. The preparation of claim 83, wherein, when the particles are incubated in a solution of simulated gastric fluid comprising 60 units/ml of pepsin at 37° C., the protein is released over a period of time that extends for at least 6 hours.

113. The preparation of claim 112, wherein not more than 90% of the protein is released in 6 hours.

114. The preparation of claim 112, wherein not more than 80% of the protein is released in 6 hours.

115. The preparation of claim 112, wherein not more than 75% of the protein is released in 6 hours.

116. The preparation of claim 83, wherein, when the particles are incubated in a solution of simulated gastric fluid comprising 60 units/ml of pepsin at 37° C., the protein is released over a period of time that extends for at least 10 hours.

117. The preparation of claim 116, wherein not more than 95% of the protein is released in 10 hours.

118. The preparation of claim 116, wherein not more than 85% of the protein is released in 10 hours.

119. The preparation of claim 116, wherein not more than 75% of the protein is released in 10 hours.

120. The preparation of claim 83, wherein the protein release is assessed using a bicinchoninic acid (BCA) assay.

121. The food, beverage, or supplement product of claim 104, wherein, when the particles are incubated in a solution of simulated gastric fluid comprising 60 units/ml of pepsin at 37° C., the protein is released over a period of time that extends for at least 6 hours.

122. The food, beverage, or supplement product of claim 121, wherein not more than 90% of the protein is released in 6 hours.

123. The food, beverage, or supplement product of claim 121, wherein not more than 80% of the protein is released in 6 hours.

124. The food, beverage, or supplement product of claim 121, wherein not more than 75% of the protein is released in 6 hours.

125. The food, beverage, or supplement product of claim 104, wherein, when the particles are incubated in a solution of simulated gastric fluid comprising 60 units/ml of pepsin at 37° C., the protein is released over a period of time that extends for at least 10 hours.

126. The food, beverage, or supplement product of claim 125, wherein not more than 95% of the protein is released in 10 hours.

127. The food, beverage, or supplement product of claim 125, wherein not more than 85% of the protein is released in 10 hours.

128. The food, beverage, or supplement product of claim 125, wherein not more than 75% of the protein is released in 10 hours.

129. The food, beverage, or supplement product of claim 104, wherein the protein release is assessed using a bicinchoninic acid (BCA) assay.

130. The preparation of claim 83, wherein the preparation comprises 0.5% (w/w) to 5% (w/w) gelator.

131. The food, beverage, or supplement product of claim 104, wherein the particles comprise 0.5% (w/w) to 5% (w/w) gelator.
